SEQUENCE LISTING (130 sequences)

SEQ ID NO: 1 - protein, 33 aa, synthetic construct
  REGION 1..33: note = Description of Artificial Sequence: Synthetic polypeptide
SHVQRLVHRDSTISNDAFISVDDLEPSGPQDLE

SEQ ID NO: 2 - protein, 33 aa, synthetic construct
  REGION 1..33: note = Description of Artificial Sequence: Synthetic polypeptide
DHLQLKDILRKQDEYHMVHLLCASRSPPSSPKS

SEQ ID NO: 3 - protein, 33 aa, synthetic construct
  REGION 1..33: note = Description of Artificial Sequence: Synthetic polypeptide
WNTWLYAPGLPPVKPNYDATLTNACIALSQRWV

SEQ ID NO: 4 - protein, 33 aa, synthetic construct
  REGION 1..33: note = Description of Artificial Sequence: Synthetic polypeptide
AQRNSNPIIAELSQAMNSGTLLSKPSPPLPPKR

SEQ ID NO: 5 - protein, 33 aa, synthetic construct
  REGION 1..33: note = Description of Artificial Sequence: Synthetic polypeptide
GESKGPLDSSFSQYLGRSCLLDQREAKRKGEGL

SEQ ID NO: 6 - protein, 33 aa, synthetic construct
  REGION 1..33: note = Description of Artificial Sequence: Synthetic polypeptide
QGKYILKVCGCDEYFLEKFPLSQYKYIRSCIML

SEQ ID NO: 7 - protein, 33 aa, synthetic construct
  REGION 1..33: note = Description of Artificial Sequence: Synthetic polypeptide
KFISVLCTSLATMLHVELPHVNLLSKMDLIEHY

SEQ ID NO: 8 - protein, 33 aa, synthetic construct
  REGION 1..33: note = Description of Artificial Sequence: Synthetic polypeptide
LRPSTLSQEVYKTVSMRLNVVSPDLSGKGLVLP

SEQ ID NO: 9 - protein, 33 aa, synthetic construct
  REGION 1..33: note = Description of Artificial Sequence: Synthetic polypeptide
ACSSSYNSAVMESSSVNVSMVHSSSKENLCPKK

SEQ ID NO: 10 - protein, 33 aa, synthetic construct
  REGION 1..33: note = Description of Artificial Sequence: Synthetic polypeptide
REIAVEHNNLGKAVYSRMARICKNDMGGSQRVL

SEQ ID NO: 11 - protein, 33 aa, synthetic construct
  REGION 1..33: note = Description of Artificial Sequence: Synthetic polypeptide
PGLLSVDLFHVLVSAVLAFPSLYWDDTVDLQPS

SEQ ID NO: 12 - protein, 33 aa, synthetic construct
  REGION 1..33: note = Description of Artificial Sequence: Synthetic polypeptide
LCLMGHKPVLLRTQCRCHPAISAIANDLFYEGS

SEQ ID NO: 13 - protein, 33 aa, synthetic construct
  REGION 1..33: note = Description of Artificial Sequence: Synthetic polypeptide
TSAAETKPPLAPSSPPAPPGTMVAGSSLEGPAP

SEQ ID NO: 14 - protein, 33 aa, synthetic construct
  REGION 1..33: note = Description of Artificial Sequence: Synthetic polypeptide
RSLQELLARRTYYSGDPLFSVDILRNVTDTFKR

SEQ ID NO: 15 - protein, 33 aa, synthetic construct
  REGION 1..33: note = Description of Artificial Sequence: Synthetic polypeptide
GGHVCWMREGVELCPGNKYEMRRHGTTHSLVIH

SEQ ID NO: 16 - protein, 33 aa, synthetic construct
  REGION 1..33: note = Description of Artificial Sequence: Synthetic polypeptide
EPIEVNLFGLINVTPNMLPLVKKARGRVINVSS

SEQ ID NO: 17 - protein, 33 aa, synthetic construct
  REGION 1..33: note = Description of Artificial Sequence: Synthetic polypeptide
VDFNKICGQAYDSATNFRVKLNEVVAEFKKEEP

SEQ ID NO: 18 - protein, 33 aa, synthetic construct
  REGION 1..33: note = Description of Artificial Sequence: Synthetic polypeptide
NCNFDFPEETCGWVYDHAKLLRSTWISSANPND

SEQ ID NO: 19 - protein, 33 aa, synthetic construct
  REGION 1..33: note = Description of Artificial Sequence: Synthetic polypeptide
ISMGGHMASLAVCNWPKPMPLIPCLSWSTASGV

SEQ ID NO: 20 - protein, 33 aa, synthetic construct
  REGION 1..33: note = Description of Artificial Sequence: Synthetic polypeptide
GLSLNIKKYLLVSMPLWAKHMSDEQIQGFVENL

SEQ ID NO: 21 - protein, 33 aa, synthetic construct
  REGION 1..33: note = Description of Artificial Sequence: Synthetic polypeptide
ANAVHSMEKVMDIHSKYCRCLQRLSSTVGHSLI

SEQ ID NO: 22 - protein, 33 aa, synthetic construct
  REGION 1..33: note = Description of Artificial Sequence: Synthetic polypeptide
EVPKPLFPVAGVPMIQYHIEACAQVPGMQEILL

SEQ ID NO: 23 - protein, 33 aa, synthetic construct
  REGION 1..33: note = Description of Artificial Sequence: Synthetic polypeptide
SNEDREWHESFSLVLGPDDPVEAVLGDVTTATV

SEQ ID NO: 24 - protein, 33 aa, synthetic construct
  REGION 1..33: note = Description of Artificial Sequence: Synthetic polypeptide
MSVMVLRTQGPEAPFEDHRLILHTSSCDAERAR

SEQ ID NO: 25 - protein, 33 aa, synthetic construct
  REGION 1..33: note = Description of Artificial Sequence: Synthetic polypeptide
CPHMTEDNKDLIQGKDLLTACYDVDYEKNAKGS

SEQ ID NO: 26 - protein, 33 aa, synthetic construct
  REGION 1..33: note = Description of Artificial Sequence: Synthetic polypeptide
TRGLAYLHTELPQGDHYKPAISHRDLNSRNVLV

SEQ ID NO: 27 - protein, 33 aa, synthetic construct
  REGION 1..33: note = Description of Artificial Sequence: Synthetic polypeptide
IHREQNSLSLLEASEADGDAVNDKKRTPNEAPS

SEQ ID NO: 28 - protein, 33 aa, synthetic construct
  REGION 1..33: note = Description of Artificial Sequence: Synthetic polypeptide
EPKRDRRDPSTDKSGPDTFPVEVLERKPPEKTY

SEQ ID NO: 29 - protein, 33 aa, synthetic construct
  REGION 1..33: note = Description of Artificial Sequence: Synthetic polypeptide
SGHVGLRNLGNTLPQCFLNAMLQCLSSTRPLRD

SEQ ID NO: 30 - protein, 33 aa, synthetic construct
  REGION 1..33: note = Description of Artificial Sequence: Synthetic polypeptide
AEDTQENLHSGKSQSRSFPKVLADSDESDMEET

SEQ ID NO: 31 - protein, 33 aa, synthetic construct
  REGION 1..33: note = Description of Artificial Sequence: Synthetic polypeptide
ECKEAAVSHAGSMHRERRTFLRSALKELATVLS

SEQ ID NO: 32 - protein, 33 aa, synthetic construct
  REGION 1..33: note = Description of Artificial Sequence: Synthetic polypeptide
RMREEQLAREAEAWAEREAEARRREEQEAREKA

SEQ ID NO: 33 - protein, 33 aa, synthetic construct
  REGION 1..33: note = Description of Artificial Sequence: Synthetic polypeptide
LSQTPPPAPPPSAGSEPLARLPQKDSVGKHSGA

SEQ ID NO: 34 - protein, 33 aa, synthetic construct
  REGION 1..33: note = Description of Artificial Sequence: Synthetic polypeptide
NTQIQKLTGSPFRSREYVQTMESESEHSYPPPG

SEQ ID NO: 35 - protein, 33 aa, synthetic construct
  REGION 1..33: note = Description of Artificial Sequence: Synthetic polypeptide
TTTSRAICQLVKEYIGYRDGIWDVSVTRTQPIV

SEQ ID NO: 36 - protein, 33 aa, synthetic construct
  REGION 1..33: note = Description of Artificial Sequence: Synthetic polypeptide
NGYDTYVHDAYGLFQECRSRVAHWGWPLGPAPL

SEQ ID NO: 37 - protein, 33 aa, synthetic construct
  REGION 1..33: note = Description of Artificial Sequence: Synthetic polypeptide
IQGWLGTKVRTNLRREPFSLVPKNAKDGNSFQG

SEQ ID NO: 38 - protein, 33 aa, synthetic construct
  REGION 1..33: note = Description of Artificial Sequence: Synthetic polypeptide
GGKVWNEQEKNTYADKLFDCIEAYAPGFKRSVL

SEQ ID NO: 39 - protein, 33 aa, synthetic construct
  REGION 1..33: note = Description of Artificial Sequence: Synthetic polypeptide
WMKCVKGQPHDHKHLMPTQIIPGSVLTDLLDAM

SEQ ID NO: 40 - protein, 33 aa, synthetic construct
  REGION 1..33: note = Description of Artificial Sequence: Synthetic polypeptide
SILRNHRYRRDARTLEDKEEMWFNTDEDDMEDG

SEQ ID NO: 41 - protein, 7 aa, synthetic construct
  REGION 1..7: note = Description of Artificial Sequence: Synthetic peptide
RGRKRRS

SEQ ID NO: 42 - protein, 394 aa, synthetic construct
  REGION 1..394: note = Description of Artificial Sequence: Synthetic polypeptide
MSHVQRLVHRDSTISNDAFISVDDLEPSGPQDLERGRKRRSDHLQLKDILRKQDEYHMVHLLCASRSPPSSPKSRGRKRRSWNTWLYAPGLPPVKPNYDATLTNACIALSQRWVRGRKRRSACSSSYNSAVMESSSVNVSMVHSSSKENLCPKKRGRKRRSPGLLSVDLFHVLVSAVLAFPSLYWDDTVDLQPSRGRKRRSGGHVCWMREGVELCPGNKYEMRRHGTTHSLVIHRGRKRRSEPIEVNLFGLINVTPNMLPLVKKARGRVINVSSRGRKRRSVDFNKICGQAYDSATNFRVKLNEVVAEFKKEEPRGRKRRSNCNFDFPEETCGWVYDHAKLLRSTWISSANPNDRGRKRRSISMGGHMASLAVCNWPKPMPLIPCLSWSTASGV

SEQ ID NO: 43 - protein, 394 aa, synthetic construct
  REGION 1..394: note = Description of Artificial Sequence: Synthetic polypeptide
MDHLQLKDILRKQDEYHMVHLLCASRSPPSSPKSRGRKRRSWNTWLYAPGLPPVKPNYDATLTNACIALSQRWVRGRKRRSACSSSYNSAVMESSSVNVSMVHSSSKENLCPKKRGRKRRSPGLLSVDLFHVLVSAVLAFPSLYWDDTVDLQPSRGRKRRSGGHVCWMREGVELCPGNKYEMRRHGTTHSLVIHRGRKRRSEPIEVNLFGLINVTPNMLPLVKKARGRVINVSSRGRKRRSVDFNKICGQAYDSATNFRVKLNEVVAEFKKEEPRGRKRRSNCNFDFPEETCGWVYDHAKLLRSTWISSANPNDRGRKRRSISMGGHMASLAVCNWPKPMPLIPCLSWSTASGVRGRKRRSSHVQRLVHRDSTISNDAFISVDDLEPSGPQDLE

SEQ ID NO: 44 - protein, 794 aa, synthetic construct
  REGION 1..794: note = Description of Artificial Sequence: Synthetic polypeptide
MSHVQRLVHRDSTISNDAFISVDDLEPSGPQDLERGRKRRSDHLQLKDILRKQDEYHMVHLLCASRSPPSSPKSRGRKRRSWNTWLYAPGLPPVKPNYDATLTNACIALSQRWVRGRKRRSAQRNSNPIIAELSQAMNSGTLLSKPSPPLPPKRRGRKRRSGESKGPLDSSFSQYLGRSCLLDQREAKRKGEGLRGRKRRSQGKYILKVCGCDEYFLEKFPLSQYKYIRSCIMLRGRKRRSKFISVLCTSLATMLHVELPHVNLLSKMDLIEHYRGRKRRSLRPSTLSQEVYKTVSMRLNVVSPDLSGKGLVLPRGRKRRSACSSSYNSAVMESSSVNVSMVHSSSKENLCPKKRGRKRRSREIAVEHNNLGKAVYSRMARICKNDMGGSQRVLRGRKRRSPGLLSVDLFHVLVSAVLAFPSLYWDDTVDLQPSRGRKRRSLCLMGHKPVLLRTQCRCHPAISAIANDLFYEGSRGRKRRSTSAAETKPPLAPSSPPAPPGTMVAGSSLEGPAPRGRKRRSRSLQELLARRTYYSGDPLFSVDILRNVTDTFKRRGRKRRSGGHVCWMREGVELCPGNKYEMRRHGTTHSLVIHRGRKRRSEPIEVNLFGLINVTPNMLPLVKKARGRVINVSSRGRKRRSVDFNKICGQAYDSATNFRVKLNEVVAEFKKEEPRGRKRRSNCNFDFPEETCGWVYDHAKLLRSTWISSANPNDRGRKRRSISMGGHMASLAVCNWPKPMPLIPCLSWSTASGVRGRKRRSGLSLNIKKYLLVSMPLWAKHMSDEQIQGFVENL

SEQ ID NO: 45 - protein, 794 aa, synthetic construct
  REGION 1..794: note = Description of Artificial Sequence: Synthetic polypeptide
MDHLQLKDILRKQDEYHMVHLLCASRSPPSSPKSRGRKRRSWNTWLYAPGLPPVKPNYDATLTNACIALSQRWVRGRKRRSAQRNSNPIIAELSQAMNSGTLLSKPSPPLPPKRRGRKRRSGESKGPLDSSFSQYLGRSCLLDQREAKRKGEGLRGRKRRSQGKYILKVCGCDEYFLEKFPLSQYKYIRSCIMLRGRKRRSKFISVLCTSLATMLHVELPHVNLLSKMDLIEHYRGRKRRSLRPSTLSQEVYKTVSMRLNVVSPDLSGKGLVLPRGRKRRSACSSSYNSAVMESSSVNVSMVHSSSKENLCPKKRGRKRRSREIAVEHNNLGKAVYSRMARICKNDMGGSQRVLRGRKRRSSHVQRLVHRDSTISNDAFISVDDLEPSGPQDLERGRKRRSPGLLSVDLFHVLVSAVLAFPSLYWDDTVDLQPSRGRKRRSLCLMGHKPVLLRTQCRCHPAISAIANDLFYEGSRGRKRRSTSAAETKPPLAPSSPPAPPGTMVAGSSLEGPAPRGRKRRSRSLQELLARRTYYSGDPLFSVDILRNVTDTFKRRGRKRRSGGHVCWMREGVELCPGNKYEMRRHGTTHSLVIHRGRKRRSEPIEVNLFGLINVTPNMLPLVKKARGRVINVSSRGRKRRSVDFNKICGQAYDSATNFRVKLNEVVAEFKKEEPRGRKRRSNCNFDFPEETCGWVYDHAKLLRSTWISSANPNDRGRKRRSISMGGHMASLAVCNWPKPMPLIPCLSWSTASGVRGRKRRSGLSLNIKKYLLVSMPLWAKHMSDEQIQGFVENL

SEQ ID NO: 46 - protein, 794 aa, synthetic construct
  REGION 1..794: note = Description of Artificial Sequence: Synthetic polypeptide
MDHLQLKDILRKQDEYHMVHLLCASRSPPSSPKSRGRKRRSWNTWLYAPGLPPVKPNYDATLTNACIALSQRWVRGRKRRSAQRNSNPIIAELSQAMNSGTLLSKPSPPLPPKRRGRKRRSGESKGPLDSSFSQYLGRSCLLDQREAKRKGEGLRGRKRRSQGKYILKVCGCDEYFLEKFPLSQYKYIRSCIMLRGRKRRSKFISVLCTSLATMLHVELPHVNLLSKMDLIEHYRGRKRRSLRPSTLSQEVYKTVSMRLNVVSPDLSGKGLVLPRGRKRRSACSSSYNSAVMESSSVNVSMVHSSSKENLCPKKRGRKRRSREIAVEHNNLGKAVYSRMARICKNDMGGSQRVLRGRKRRSPGLLSVDLFHVLVSAVLAFPSLYWDDTVDLQPSRGRKRRSLCLMGHKPVLLRTQCRCHPAISAIANDLFYEGSRGRKRRSTSAAETKPPLAPSSPPAPPGTMVAGSSLEGPAPRGRKRRSRSLQELLARRTYYSGDPLFSVDILRNVTDTFKRRGRKRRSGGHVCWMREGVELCPGNKYEMRRHGTTHSLVIHRGRKRRSEPIEVNLFGLINVTPNMLPLVKKARGRVINVSSRGRKRRSVDFNKICGQAYDSATNFRVKLNEVVAEFKKEEPRGRKRRSNCNFDFPEETCGWVYDHAKLLRSTWISSANPNDRGRKRRSISMGGHMASLAVCNWPKPMPLIPCLSWSTASGVRGRKRRSGLSLNIKKYLLVSMPLWAKHMSDEQIQGFVENLRGRKRRSSHVQRLVHRDSTISNDAFISVDDLEPSGPQDLE

SEQ ID NO: 47 - protein, 1594 aa, synthetic construct
  REGION 1..1594: note = Description of Artificial Sequence: Synthetic polypeptide
MSHVQRLVHRDSTISNDAFISVDDLEPSGPQDLERGRKRRSANAVHSMEKVMDIHSKYCRCLQRLSSTVGHSLIRGRKRRSDHLQLKDILRKQDEYHMVHLLCASRSPPSSPKSRGRKRRSEVPKPLFPVAGVPMIQYHIEACAQVPGMQEILLRGRKRRSWNTWLYAPGLPPVKPNYDATLTNACIALSQRWVRGRKRRSSNEDREWHESFSLVLGPDDPVEAVLGDVTTATVRGRKRRSAQRNSNPIIAELSQAMNSGTLLSKPSPPLPPKRRGRKRRSMSVMVLRTQGPEAPFEDHRLILHTSSCDAERARRGRKRRSGESKGPLDSSFSQYLGRSCLLDQREAKRKGEGLRGRKRRSCPHMTEDNKDLIQGKDLLTACYDVDYEKNAKGSRGRKRRSQGKYILKVCGCDEYFLEKFPLSQYKYIRSCIMLRGRKRRSTRGLAYLHTELPQGDHYKPAISHRDLNSRNVLVRGRKRRSKFISVLCTSLATMLHVELPHVNLLSKMDLIEHYRGRKRRSIHREQNSLSLLEASEADGDAVNDKKRTPNEAPSRGRKRRSLRPSTLSQEVYKTVSMRLNVVSPDLSGKGLVLPRGRKRRSEPKRDRRDPSTDKSGPDTFPVEVLERKPPEKTYRGRKRRSACSSSYNSAVMESSSVNVSMVHSSSKENLCPKKRGRKRRSSGHVGLRNLGNTLPQCFLNAMLQCLSSTRPLRDRGRKRRSREIAVEHNNLGKAVYSRMARICKNDMGGSQRVLRGRKRRSAEDTQENLHSGKSQSRSFPKVLADSDESDMEETRGRKRRSPGLLSVDLFHVLVSAVLAFPSLYWDDTVDLQPSRGRKRRSECKEAAVSHAGSMHRERRTFLRSALKELATVLSRGRKRRSLCLMGHKPVLLRTQCRCHPAISAIANDLFYEGSRGRKRRSRMREEQLAREAEAWAEREAEARRREEQEAREKARGRKRRSTSAAETKPPLAPSSPPAPPGTMVAGSSLEGPAPRGRKRRSLSQTPPPAPPPSAGSEPLARLPQKDSVGKHSGARGRKRRSRSLQELLARRTYYSGDPLFSVDILRNVTDTFKRRGRKRRSNTQIQKLTGSPFRSREYVQTMESESEHSYPPPGRGRKRRSGGHVCWMREGVELCPGNKYEMRRHGTTHSLVIHRGRKRRSTTTSRAICQLVKEYIGYRDGIWDVSVTRTQPIVRGRKRRSEPIEVNLFGLINVTPNMLPLVKKARGRVINVSSRGRKRRSNGYDTYVHDAYGLFQECRSRVAHWGWPLGPAPLRGRKRRSVDFNKICGQAYDSATNFRVKLNEVVAEFKKEEPRGRKRRSIQGWLGTKVRTNLRREPFSLVPKNAKDGNSFQGRGRKRRSNCNFDFPEETCGWVYDHAKLLRSTWISSANPNDRGRKRRSGGKVWNEQEKNTYADKLFDCIEAYAPGFKRSVLRGRKRRSISMGGHMASLAVCNWPKPMPLIPCLSWSTASGVRGRKRRSWMKCVKGQPHDHKHLMPTQIIPGSVLTDLLDAMRGRKRRSGLSLNIKKYLLVSMPLWAKHMSDEQIQGFVENLRGRKRRSSILRNHRYRRDARTLEDKEEMWFNTDEDDMEDG

SEQ ID NO: 48 - protein, 1594 aa, synthetic construct
  REGION 1..1594: note = Description of Artificial Sequence: Synthetic polypeptide
MANAVHSMEKVMDIHSKYCRCLQRLSSTVGHSLIRGRKRRSDHLQLKDILRKQDEYHMVHLLCASRSPPSSPKSRGRKRRSEVPKPLFPVAGVPMIQYHIEACAQVPGMQEILLRGRKRRSWNTWLYAPGLPPVKPNYDATLTNACIALSQRWVRGRKRRSSNEDREWHESFSLVLGPDDPVEAVLGDVTTATVRGRKRRSAQRNSNPIIAELSQAMNSGTLLSKPSPPLPPKRRGRKRRSMSVMVLRTQGPEAPFEDHRLILHTSSCDAERARRGRKRRSGESKGPLDSSFSQYLGRSCLLDQREAKRKGEGLRGRKRRSCPHMTEDNKDLIQGKDLLTACYDVDYEKNAKGSRGRKRRSSHVQRLVHRDSTISNDAFISVDDLEPSGPQDLERGRKRRSQGKYILKVCGCDEYFLEKFPLSQYKYIRSCIMLRGRKRRSTRGLAYLHTELPQGDHYKPAISHRDLNSRNVLVRGRKRRSKFISVLCTSLATMLHVELPHVNLLSKMDLIEHYRGRKRRSIHREQNSLSLLEASEADGDAVNDKKRTPNEAPSRGRKRRSLRPSTLSQEVYKTVSMRLNVVSPDLSGKGLVLPRGRKRRSEPKRDRRDPSTDKSGPDTFPVEVLERKPPEKTYRGRKRRSACSSSYNSAVMESSSVNVSMVHSSSKENLCPKKRGRKRRSSGHVGLRNLGNTLPQCFLNAMLQCLSSTRPLRDRGRKRRSREIAVEHNNLGKAVYSRMARICKNDMGGSQRVLRGRKRRSAEDTQENLHSGKSQSRSFPKVLADSDESDMEETRGRKRRSPGLLSVDLFHVLVSAVLAFPSLYWDDTVDLQPSRGRKRRSECKEAAVSHAGSMHRERRTFLRSALKELATVLSRGRKRRSLCLMGHKPVLLRTQCRCHPAISAIANDLFYEGSRGRKRRSRMREEQLAREAEAWAEREAEARRREEQEAREKARGRKRRSTSAAETKPPLAPSSPPAPPGTMVAGSSLEGPAPRGRKRRSLSQTPPPAPPPSAGSEPLARLPQKDSVGKHSGARGRKRRSRSLQELLARRTYYSGDPLFSVDILRNVTDTFKRRGRKRRSNTQIQKLTGSPFRSREYVQTMESESEHSYPPPGRGRKRRSGGHVCWMREGVELCPGNKYEMRRHGTTHSLVIHRGRKRRSTTTSRAICQLVKEYIGYRDGIWDVSVTRTQPIVRGRKRRSEPIEVNLFGLINVTPNMLPLVKKARGRVINVSSRGRKRRSNGYDTYVHDAYGLFQECRSRVAHWGWPLGPAPLRGRKRRSVDFNKICGQAYDSATNFRVKLNEVVAEFKKEEPRGRKRRSIQGWLGTKVRTNLRREPFSLVPKNAKDGNSFQGRGRKRRSNCNFDFPEETCGWVYDHAKLLRSTWISSANPNDRGRKRRSGGKVWNEQEKNTYADKLFDCIEAYAPGFKRSVLRGRKRRSISMGGHMASLAVCNWPKPMPLIPCLSWSTASGVRGRKRRSWMKCVKGQPHDHKHLMPTQIIPGSVLTDLLDAMRGRKRRSGLSLNIKKYLLVSMPLWAKHMSDEQIQGFVENLRGRKRRSSILRNHRYRRDARTLEDKEEMWFNTDEDDMEDG

SEQ ID NO: 49 - protein, 1594 aa, synthetic construct
  REGION 1..1594: note = Description of Artificial Sequence: Synthetic polypeptide
MANAVHSMEKVMDIHSKYCRCLQRLSSTVGHSLIRGRKRRSDHLQLKDILRKQDEYHMVHLLCASRSPPSSPKSRGRKRRSEVPKPLFPVAGVPMIQYHIEACAQVPGMQEILLRGRKRRSWNTWLYAPGLPPVKPNYDATLTNACIALSQRWVRGRKRRSSNEDREWHESFSLVLGPDDPVEAVLGDVTTATVRGRKRRSAQRNSNPIIAELSQAMNSGTLLSKPSPPLPPKRRGRKRRSMSVMVLRTQGPEAPFEDHRLILHTSSCDAERARRGRKRRSGESKGPLDSSFSQYLGRSCLLDQREAKRKGEGLRGRKRRSCPHMTEDNKDLIQGKDLLTACYDVDYEKNAKGSRGRKRRSQGKYILKVCGCDEYFLEKFPLSQYKYIRSCIMLRGRKRRSTRGLAYLHTELPQGDHYKPAISHRDLNSRNVLVRGRKRRSKFISVLCTSLATMLHVELPHVNLLSKMDLIEHYRGRKRRSIHREQNSLSLLEASEADGDAVNDKKRTPNEAPSRGRKRRSLRPSTLSQEVYKTVSMRLNVVSPDLSGKGLVLPRGRKRRSEPKRDRRDPSTDKSGPDTFPVEVLERKPPEKTYRGRKRRSACSSSYNSAVMESSSVNVSMVHSSSKENLCPKKRGRKRRSSGHVGLRNLGNTLPQCFLNAMLQCLSSTRPLRDRGRKRRSREIAVEHNNLGKAVYSRMARICKNDMGGSQRVLRGRKRRSAEDTQENLHSGKSQSRSFPKVLADSDESDMEETRGRKRRSSHVQRLVHRDSTISNDAFISVDDLEPSGPQDLERGRKRRSPGLLSVDLFHVLVSAVLAFPSLYWDDTVDLQPSRGRKRRSECKEAAVSHAGSMHRERRTFLRSALKELATVLSRGRKRRSLCLMGHKPVLLRTQCRCHPAISAIANDLFYEGSRGRKRRSRMREEQLAREAEAWAEREAEARRREEQEAREKARGRKRRSTSAAETKPPLAPSSPPAPPGTMVAGSSLEGPAPRGRKRRSLSQTPPPAPPPSAGSEPLARLPQKDSVGKHSGARGRKRRSRSLQELLARRTYYSGDPLFSVDILRNVTDTFKRRGRKRRSNTQIQKLTGSPFRSREYVQTMESESEHSYPPPGRGRKRRSGGHVCWMREGVELCPGNKYEMRRHGTTHSLVIHRGRKRRSTTTSRAICQLVKEYIGYRDGIWDVSVTRTQPIVRGRKRRSEPIEVNLFGLINVTPNMLPLVKKARGRVINVSSRGRKRRSNGYDTYVHDAYGLFQECRSRVAHWGWPLGPAPLRGRKRRSVDFNKICGQAYDSATNFRVKLNEVVAEFKKEEPRGRKRRSIQGWLGTKVRTNLRREPFSLVPKNAKDGNSFQGRGRKRRSNCNFDFPEETCGWVYDHAKLLRSTWISSANPNDRGRKRRSGGKVWNEQEKNTYADKLFDCIEAYAPGFKRSVLRGRKRRSISMGGHMASLAVCNWPKPMPLIPCLSWSTASGVRGRKRRSWMKCVKGQPHDHKHLMPTQIIPGSVLTDLLDAMRGRKRRSGLSLNIKKYLLVSMPLWAKHMSDEQIQGFVENLRGRKRRSSILRNHRYRRDARTLEDKEEMWFNTDEDDMEDG

SEQ ID NO: 50 - protein, 1594 aa, synthetic construct
  REGION 1..1594: note = Description of Artificial Sequence: Synthetic polypeptide
MANAVHSMEKVMDIHSKYCRCLQRLSSTVGHSLIRGRKRRSDHLQLKDILRKQDEYHMVHLLCASRSPPSSPKSRGRKRRSEVPKPLFPVAGVPMIQYHIEACAQVPGMQEILLRGRKRRSWNTWLYAPGLPPVKPNYDATLTNACIALSQRWVRGRKRRSSNEDREWHESFSLVLGPDDPVEAVLGDVTTATVRGRKRRSAQRNSNPIIAELSQAMNSGTLLSKPSPPLPPKRRGRKRRSMSVMVLRTQGPEAPFEDHRLILHTSSCDAERARRGRKRRSGESKGPLDSSFSQYLGRSCLLDQREAKRKGEGLRGRKRRSCPHMTEDNKDLIQGKDLLTACYDVDYEKNAKGSRGRKRRSQGKYILKVCGCDEYFLEKFPLSQYKYIRSCIMLRGRKRRSTRGLAYLHTELPQGDHYKPAISHRDLNSRNVLVRGRKRRSKFISVLCTSLATMLHVELPHVNLLSKMDLIEHYRGRKRRSIHREQNSLSLLEASEADGDAVNDKKRTPNEAPSRGRKRRSLRPSTLSQEVYKTVSMRLNVVSPDLSGKGLVLPRGRKRRSEPKRDRRDPSTDKSGPDTFPVEVLERKPPEKTYRGRKRRSACSSSYNSAVMESSSVNVSMVHSSSKENLCPKKRGRKRRSSGHVGLRNLGNTLPQCFLNAMLQCLSSTRPLRDRGRKRRSREIAVEHNNLGKAVYSRMARICKNDMGGSQRVLRGRKRRSAEDTQENLHSGKSQSRSFPKVLADSDESDMEETRGRKRRSPGLLSVDLFHVLVSAVLAFPSLYWDDTVDLQPSRGRKRRSECKEAAVSHAGSMHRERRTFLRSALKELATVLSRGRKRRSLCLMGHKPVLLRTQCRCHPAISAIANDLFYEGSRGRKRRSRMREEQLAREAEAWAEREAEARRREEQEAREKARGRKRRSTSAAETKPPLAPSSPPAPPGTMVAGSSLEGPAPRGRKRRSLSQTPPPAPPPSAGSEPLARLPQKDSVGKHSGARGRKRRSRSLQELLARRTYYSGDPLFSVDILRNVTDTFKRRGRKRRSNTQIQKLTGSPFRSREYVQTMESESEHSYPPPGRGRKRRSGGHVCWMREGVELCPGNKYEMRRHGTTHSLVIHRGRKRRSTTTSRAICQLVKEYIGYRDGIWDVSVTRTQPIVRGRKRRSSHVQRLVHRDSTISNDAFISVDDLEPSGPQDLERGRKRRSEPIEVNLFGLINVTPNMLPLVKKARGRVINVSSRGRKRRSNGYDTYVHDAYGLFQECRSRVAHWGWPLGPAPLRGRKRRSVDFNKICGQAYDSATNFRVKLNEVVAEFKKEEPRGRKRRSIQGWLGTKVRTNLRREPFSLVPKNAKDGNSFQGRGRKRRSNCNFDFPEETCGWVYDHAKLLRSTWISSANPNDRGRKRRSGGKVWNEQEKNTYADKLFDCIEAYAPGFKRSVLRGRKRRSISMGGHMASLAVCNWPKPMPLIPCLSWSTASGVRGRKRRSWMKCVKGQPHDHKHLMPTQIIPGSVLTDLLDAMRGRKRRSGLSLNIKKYLLVSMPLWAKHMSDEQIQGFVENLRGRKRRSSILRNHRYRRDARTLEDKEEMWFNTDEDDMEDG

SEQ ID NO: 51 - protein, 1594 aa, synthetic construct
  REGION 1..1594: note = Description of Artificial Sequence: Synthetic polypeptide
MANAVHSMEKVMDIHSKYCRCLQRLSSTVGHSLIRGRKRRSDHLQLKDILRKQDEYHMVHLLCASRSPPSSPKSRGRKRRSEVPKPLFPVAGVPMIQYHIEACAQVPGMQEILLRGRKRRSWNTWLYAPGLPPVKPNYDATLTNACIALSQRWVRGRKRRSSNEDREWHESFSLVLGPDDPVEAVLGDVTTATVRGRKRRSAQRNSNPIIAELSQAMNSGTLLSKPSPPLPPKRRGRKRRSMSVMVLRTQGPEAPFEDHRLILHTSSCDAERARRGRKRRSGESKGPLDSSFSQYLGRSCLLDQREAKRKGEGLRGRKRRSCPHMTEDNKDLIQGKDLLTACYDVDYEKNAKGSRGRKRRSQGKYILKVCGCDEYFLEKFPLSQYKYIRSCIMLRGRKRRSTRGLAYLHTELPQGDHYKPAISHRDLNSRNVLVRGRKRRSKFISVLCTSLATMLHVELPHVNLLSKMDLIEHYRGRKRRSIHREQNSLSLLEASEADGDAVNDKKRTPNEAPSRGRKRRSLRPSTLSQEVYKTVSMRLNVVSPDLSGKGLVLPRGRKRRSEPKRDRRDPSTDKSGPDTFPVEVLERKPPEKTYRGRKRRSACSSSYNSAVMESSSVNVSMVHSSSKENLCPKKRGRKRRSSGHVGLRNLGNTLPQCFLNAMLQCLSSTRPLRDRGRKRRSREIAVEHNNLGKAVYSRMARICKNDMGGSQRVLRGRKRRSAEDTQENLHSGKSQSRSFPKVLADSDESDMEETRGRKRRSPGLLSVDLFHVLVSAVLAFPSLYWDDTVDLQPSRGRKRRSECKEAAVSHAGSMHRERRTFLRSALKELATVLSRGRKRRSLCLMGHKPVLLRTQCRCHPAISAIANDLFYEGSRGRKRRSRMREEQLAREAEAWAEREAEARRREEQEAREKARGRKRRSTSAAETKPPLAPSSPPAPPGTMVAGSSLEGPAPRGRKRRSLSQTPPPAPPPSAGSEPLARLPQKDSVGKHSGARGRKRRSRSLQELLARRTYYSGDPLFSVDILRNVTDTFKRRGRKRRSNTQIQKLTGSPFRSREYVQTMESESEHSYPPPGRGRKRRSGGHVCWMREGVELCPGNKYEMRRHGTTHSLVIHRGRKRRSTTTSRAICQLVKEYIGYRDGIWDVSVTRTQPIVRGRKRRSEPIEVNLFGLINVTPNMLPLVKKARGRVINVSSRGRKRRSNGYDTYVHDAYGLFQECRSRVAHWGWPLGPAPLRGRKRRSVDFNKICGQAYDSATNFRVKLNEVVAEFKKEEPRGRKRRSIQGWLGTKVRTNLRREPFSLVPKNAKDGNSFQGRGRKRRSNCNFDFPEETCGWVYDHAKLLRSTWISSANPNDRGRKRRSGGKVWNEQEKNTYADKLFDCIEAYAPGFKRSVLRGRKRRSISMGGHMASLAVCNWPKPMPLIPCLSWSTASGVRGRKRRSWMKCVKGQPHDHKHLMPTQIIPGSVLTDLLDAMRGRKRRSGLSLNIKKYLLVSMPLWAKHMSDEQIQGFVENLRGRKRRSSILRNHRYRRDARTLEDKEEMWFNTDEDDMEDGRGRKRRSSHVQRLVHRDSTISNDAFISVDDLEPSGPQDLE

SEQ ID NO: 52 - protein, 194 aa, synthetic construct
  REGION 1..194: note = Description of Artificial Sequence: Synthetic polypeptide
MSHVQRLVHRDSTISNDAFISVDDLEPSGPQDLERGRKRRSDHLQLKDILRKQDEYHMVHLLCASRSPPSSPKSRGRKRRSWNTWLYAPGLPPVKPNYDATLTNACIALSQRWVRGRKRRSAQRNSNPIIAELSQAMNSGTLLSKPSPPLPPKRRGRKRRSGESKGPLDSSFSQYLGRSCLLDQREAKRKGEGL

SEQ ID NO: 53 - protein, 994 aa, synthetic construct
  REGION 1..994: note = Description of Artificial Sequence: Synthetic polypeptide
MSHVQRLVHRDSTISNDAFISVDDLEPSGPQDLERGRKRRSDHLQLKDILRKQDEYHMVHLLCASRSPPSSPKSRGRKRRSWNTWLYAPGLPPVKPNYDATLTNACIALSQRWVRGRKRRSAQRNSNPIIAELSQAMNSGTLLSKPSPPLPPKRRGRKRRSGESKGPLDSSFSQYLGRSCLLDQREAKRKGEGLRGRKRRSSHVQRLVHRDSTISNDAFISVDDLEPSGPQDLERGRKRRSDHLQLKDILRKQDEYHMVHLLCASRSPPSSPKSRGRKRRSWNTWLYAPGLPPVKPNYDATLTNACIALSQRWVRGRKRRSAQRNSNPIIAELSQAMNSGTLLSKPSPPLPPKRRGRKRRSGESKGPLDSSFSQYLGRSCLLDQREAKRKGEGLRGRKRRSSHVQRLVHRDSTISNDAFISVDDLEPSGPQDLERGRKRRSDHLQLKDILRKQDEYHMVHLLCASRSPPSSPKSRGRKRRSWNTWLYAPGLPPVKPNYDATLTNACIALSQRWVRGRKRRSAQRNSNPIIAELSQAMNSGTLLSKPSPPLPPKRRGRKRRSGESKGPLDSSFSQYLGRSCLLDQREAKRKGEGLRGRKRRSSHVQRLVHRDSTISNDAFISVDDLEPSGPQDLERGRKRRSDHLQLKDILRKQDEYHMVHLLCASRSPPSSPKSRGRKRRSWNTWLYAPGLPPVKPNYDATLTNACIALSQRWVRGRKRRSAQRNSNPIIAELSQAMNSGTLLSKPSPPLPPKRRGRKRRSGESKGPLDSSFSQYLGRSCLLDQREAKRKGEGLRGRKRRSSHVQRLVHRDSTISNDAFISVDDLEPSGPQDLERGRKRRSDHLQLKDILRKQDEYHMVHLLCASRSPPSSPKSRGRKRRSWNTWLYAPGLPPVKPNYDATLTNACIALSQRWVRGRKRRSAQRNSNPIIAELSQAMNSGTLLSKPSPPLPPKRRGRKRRSGESKGPLDSSFSQYLGRSCLLDQREAKRKGEGL

SEQ ID NO: 54 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 55 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 56 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 57 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 58 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 59 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 60 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 61 - protein, 141 aa, unidentified
  REGION 1..141: note = Description of Unknown: Survivin sequence
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKEFEETAEKVRRAIEQLAAMD

SEQ ID NO: 62 - protein, 574 aa, unidentified
  REGION 1..574: note = Description of Unknown: MAGE sequence
MATSQADIETDPGISEPDGATAQTSADGSQAQNLESRTIIRGKRTRKINNLNVEENSSGDQRRAPLAAGTWRSAPVPVTTQNPPGAPPNVLWQTPLAWQNPSGWQNQTARQTPPARQSPPARQTPPAWQNPVAWQNPVIWPNPVIWQNPVIWPNPIVWPGPVVWPNPLAWQNPPGWQTPPGWQTPPGWQGPPDWQGPPDWPLPPDWPLPPDWPLPTDWPLPPDWIPADWPIPPDWQNLRPSPNLRPSPNSRASQNPGAAQPRDVALLQERANKLVKYLMLKDYTKVPIKRSEMLRDIIREYTDVYPEIIERACFVLEKKFGIQLKEIDKEEHLYILISTPESLAGILGTTKDTPKLGLLLVILGVIFMNGNRASEAVLWEALRKMGLRPGVRHPLLGDLRKLLTYEFVKQKYLDYRRVPNSNPPEYEFLWGLRSYHETSKMKVLRFIAEVQKRDPRDWTAQFMEAADEALDALDAAAAEAEARAEARTRMGIGDEAVSGPWSWDDIEFELLTWDEEGDFGDPWSRIPFTFWARYHQNARSRFPQTFAGPIIGPGGTASANFAANFGAIGFFWVE

SEQ ID NO: 63 - protein, 189 aa, unidentified
  REGION 1..189: note = Description of Unknown: A10 sequence
MTDKTEKVAVDPETVFKRPRECDSPSYQKRQRMALLARKQGAGDSLIAGSAMSKEKKLMTGHAIPPSQLDSQIDDFTGFSKDGMMQKPGSNAPVGGNVTSNFSGDDLECRGIASSPKSQQEINADIKCQVVKEIRCLGRKYEKIFEMLEGVQGPTAVRKRFFESIIKEAARCMRRDFVKHLKKKLKRMI

SEQ ID NO: 64 - protein, 661 aa, unidentified
  REGION 1..661: note = Description of Unknown: gp100 sequence
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 65 - protein, 943 aa, unidentified
  REGION 1..943: note = Description of Unknown: EGFRvIII sequence
MRPSGTAGAALLALLAALCPASRALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 66 - protein, 417 aa, unidentified
  REGION 1..417: note = Description of Unknown: Calreticulin sequence
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 67 - protein, 502 aa, unidentified
  REGION 1..502: note = Description of Unknown: WT1 sequence
MDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 68 - DNA, 2998 bp, synthetic construct
  misc_feature 1..2998: note = Description of Artificial Sequence: Synthetic polynucleotide
  source 1..2998: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 69 - protein, 16 aa, synthetic construct
  REGION 1..16: note = Description of Artificial Sequence: Synthetic peptide
MDWTWILFLVAAATRV

SEQ ID NO: 70 - protein, 27 aa, synthetic construct
  REGION 1..27: note = Description of Artificial Sequence: Synthetic peptide
REGVELCPGNKYEMRRHGTTHSLVIHD

SEQ ID NO: 71 - protein, 15 aa, synthetic construct
  REGION 1..15: note = Description of Artificial Sequence: Synthetic peptide
REGVELCPGNKYEMR

SEQ ID NO: 72 - protein, 15 aa, synthetic construct
  REGION 1..15: note = Description of Artificial Sequence: Synthetic peptide
ELCPGNKYEMRRHGT

SEQ ID NO: 73 - protein, 15 aa, synthetic construct
  REGION 1..15: note = Description of Artificial Sequence: Synthetic peptide
GNKYEMRRHGTTHSL

SEQ ID NO: 74 - protein, 15 aa, synthetic construct
  REGION 1..15: note = Description of Artificial Sequence: Synthetic peptide
EMRRHGTTHSLVIHD

SEQ ID NO: 75 - protein, 11 aa, synthetic construct
  REGION 1..11: note = Description of Artificial Sequence: Synthetic peptide
HGTTHSLVIHD

SEQ ID NO: 76 - protein, 27 aa, synthetic construct
  REGION 1..27: note = Description of Artificial Sequence: Synthetic peptide
PSKPSFQEFVDWENVSPELNSTDQPFL

SEQ ID NO: 77 - protein, 15 aa, synthetic construct
  REGION 1..15: note = Description of Artificial Sequence: Synthetic peptide
PSKPSFQEFVDWENV

SEQ ID NO: 78 - protein, 15 aa, synthetic construct
  REGION 1..15: note = Description of Artificial Sequence: Synthetic peptide
SFQEFVDWENVSPEL

SEQ ID NO: 79 - protein, 15 aa, synthetic construct
  REGION 1..15: note = Description of Artificial Sequence: Synthetic peptide
FVDWENVSPELNSTD

SEQ ID NO: 80 - protein, 15 aa, synthetic construct
  REGION 1..15: note = Description of Artificial Sequence: Synthetic peptide
ENVSPELNSTDQPFL

SEQ ID NO: 81 - protein, 11 aa, synthetic construct
  REGION 1..11: note = Description of Artificial Sequence: Synthetic peptide
PELNSTDQPFL

SEQ ID NO: 82 - protein, 27 aa, synthetic construct
  REGION 1..27: note = Description of Artificial Sequence: Synthetic peptide
DSGSPFPAAVILRDALHMARGLKYLHQ

SEQ ID NO: 83 - protein, 15 aa, synthetic construct
  REGION 1..15: note = Description of Artificial Sequence: Synthetic peptide
DSGSPFPAAVILRDA

SEQ ID NO: 84 - protein, 15 aa, synthetic construct
  REGION 1..15: note = Description of Artificial Sequence: Synthetic peptide
PFPAAVILRDALHMA

SEQ ID NO: 85 - protein, 15 aa, synthetic construct
  REGION 1..15: note = Description of Artificial Sequence: Synthetic peptide
AVILRDALHMARGLK

SEQ ID NO: 86 - protein, 15 aa, synthetic construct
  REGION 1..15: note = Description of Artificial Sequence: Synthetic peptide
RDALHMARGLKYLHQ

SEQ ID NO: 87 - protein, 11 aa, synthetic construct
  REGION 1..11: note = Description of Artificial Sequence: Synthetic peptide
HMARGLKYLHQ

SEQ ID NO: 88 - protein, 27 aa, synthetic construct
  REGION 1..27: note = Description of Artificial Sequence: Synthetic peptide
QAIVRGCSMPGPWRSGRLLVSRRWSVE

SEQ ID NO: 89 - protein, 15 aa, synthetic construct
  REGION 1..15: note = Description of Artificial Sequence: Synthetic peptide
QAIVRGCSMPGPWRS

SEQ ID NO: 90 - protein, 15 aa, synthetic construct
  REGION 1..15: note = Description of Artificial Sequence: Synthetic peptide
RGCSMPGPWRSGRLL

SEQ ID NO: 91 - protein, 15 aa, synthetic construct
  REGION 1..15: note = Description of Artificial Sequence: Synthetic peptide
MPGPWRSGRLLVSRR

SEQ ID NO: 92 - protein, 15 aa, synthetic construct
  REGION 1..15: note = Description of Artificial Sequence: Synthetic peptide
WRSGRLLVSRRWSVE

SEQ ID NO: 93 - protein, 11 aa, synthetic construct
  REGION 1..11: note = Description of Artificial Sequence: Synthetic peptide
RLLVSRRWSVE

SEQ ID NO: 94 - protein, 27 aa, synthetic construct
  REGION 1..27: note = Description of Artificial Sequence: Synthetic peptide
PLLPFYPPDEALEIGLELNSSALPPTE

SEQ ID NO: 95 - protein, 15 aa, synthetic construct
  REGION 1..15: note = Description of Artificial Sequence: Synthetic peptide
PLLPFYPPDEALEIG

SEQ ID NO: 96 - protein, 15 aa, synthetic construct
  REGION 1..15: note = Description of Artificial Sequence: Synthetic peptide
FYPPDEALEIGLELN

SEQ ID NO: 97 - protein, 15 aa, synthetic construct
  REGION 1..15: note = Description of Artificial Sequence: Synthetic peptide
DEALEIGLELNSSAL

SEQ ID NO: 98 - protein, 15 aa, synthetic construct
  REGION 1..15: note = Description of Artificial Sequence: Synthetic peptide
EIGLELNSSALPPTE

SEQ ID NO: 99 - protein, 11 aa, synthetic construct
  REGION 1..11: note = Description of Artificial Sequence: Synthetic peptide
ELNSSALPPTE

SEQ ID NO: 100 - protein, 27 aa, synthetic construct
  REGION 1..27: note = Description of Artificial Sequence: Synthetic peptide
VILPQAPSGPSYATYLQPAQAQMLTPP

SEQ ID NO: 101 - protein, 15 aa, synthetic construct
  REGION 1..15: note = Description of Artificial Sequence: Synthetic peptide
VILPQAPSGPSYATY

SEQ ID NO: 102 - protein, 15 aa, synthetic construct
  REGION 1..15: note = Description of Artificial Sequence: Synthetic peptide
QAPSGPSYATYLQPA

SEQ ID NO: 103 - protein, 15 aa, synthetic construct
  REGION 1..15: note = Description of Artificial Sequence: Synthetic peptide
GPSYATYLQPAQAQM

SEQ ID NO: 104 - protein, 15 aa, synthetic construct
  REGION 1..15: note = Description of Artificial Sequence: Synthetic peptide
ATYLQPAQAQMLTPP

SEQ ID NO: 105 - protein, 11 aa, synthetic construct
  REGION 1..11: note = Description of Artificial Sequence: Synthetic peptide
QPAQAQMLTPP

SEQ ID NO: 106 - DNA, 66 bp, synthetic construct
  misc_feature 1..66: note = Description of Artificial Sequence: Synthetic oligonucleotide
  source 1..66: mol_type = other DNA
  CDS 1..66
ggaagcggagctactaacttcagcctgctgaagcaggctggagacgtggaggagaaccctggacct

SEQ ID NO: 107 - protein, 22 aa, synthetic construct
  REGION 1..22: note = Description of Artificial Sequence: Synthetic peptide
GSGATNFSLLKQAGDVEENPGP

SEQ ID NO: 108 - DNA, 63 bp, synthetic construct
  misc_feature 1..63: note = Description of Artificial Sequence: Synthetic oligonucleotide
  source 1..63: mol_type = other DNA
  CDS 1..63
ggaagcggagagggcagaggaagtctgctaacatgcggtgacgtcgaggagaatcctggacct

SEQ ID NO: 109 - protein, 21 aa, synthetic construct
  REGION 1..21: note = Description of Artificial Sequence: Synthetic peptide
GSGEGRGSLLTCGDVEENPGP

SEQ ID NO: 110 - DNA, 69 bp, synthetic construct
  misc_feature 1..69: note = Description of Artificial Sequence: Synthetic oligonucleotide
  source 1..69: mol_type = other DNA
  CDS 1..69
ggaagcggacagtgtactaattatgctctcttgaaattggctggagatgttgagagcaaccctggacct

SEQ ID NO: 111 - protein, 23 aa, synthetic construct
  REGION 1..23: note = Description of Artificial Sequence: Synthetic peptide
GSGQCTNYALLKLAGDVESNPGP

SEQ ID NO: 112 - DNA, 75 bp, synthetic construct
  misc_feature 1..75: note = Description of Artificial Sequence: Synthetic oligonucleotide
  source 1..75: mol_type = other DNA
  CDS 1..75
ggaagcggagtgaaacagactttgaattttgaccttctcaagttggcgggagacgtggagtccaaccctggacct

SEQ ID NO: 113 - protein, 25 aa, synthetic construct
  REGION 1..25: note = Description of Artificial Sequence: Synthetic peptide
GSGVKQTLNFDLLKLAGDVESNPGP

SEQ ID NO: 114 - protein, 6 aa, synthetic construct
  REGION 1..6: note = Description of Artificial Sequence: Synthetic 6xHis tag
HHHHHH

SEQ ID NO: 115 - DNA, 1446 bp, synthetic construct
  misc_feature 1..1446: note = Plasmid 1
  source 1..1446: mol_type = other DNA
ggatccgccaccatgcacctcggcaacttcgcttcctcactgtctgggggtcagatgtatggtcctggttctccactcgggggagcacctctcggcggcgcccctaccgccgctaggggcagaaagaggagatctgctcagagaaacagcaacccaatcatcgccgagctgtcccaggctatgaactctggcacactgctgtccaagccctctccccctctgccacccaagaggaggggaaggaagaggagaagcggagagtccaagggacctctggacagctccttcagccagtacctgggccggtcctgcctgctggatcagagagaggccaagcggaagggagagggactgaggggaaggaagcggcgcagcctggccagggacgagttcaacctgcagaagatgatggtcatggtgaccgcttccggcaagctgtttggaatcgagtctagctccggcacaatcctgtggaggggaagaaagaggagaagcgagacaacagtgaagtacctggacggcgagaagctgagcgtgctgtgccacgacaactacctgacccaggattccgaggagatggtgtgcaaggatggaagaggcaggaagcggcgctctgacagcttccagatcatcctggattctctgctgctgtttggccagtgctctagcacacctctgagcgaggccttccacccaaccgtgacaggcgagtctaggggacgcaaaaggagaagcgagtgtaaggaggccgccgtgagccacgctggatctatgcacagggagaggcgcacctttctgaggtccgccctgaaggagctggctacagtgctgtcccggggccgcaagaggagatctcagggaaagtacatcctgaaggtgtgcggctgtgacgagtacttcctggagaagtttccactgagccagtacaagtacatcagatcctgcatcatgctgaggggaagaaagcggcgcagcaagtttatcagcgtgctgtgcacctccctggccacaatgctgcacgtggagctgccccacgtgaacctgctgtctaagatggacctgatcgagcactatagaggtagaaagaggagatctgccgaggatacccaggagaacctgcacagcggaaagtcccagtctagaagcttccccaaggtgctggctgactctgatgagagcgatatggaggagacaaggggcagaaagcggcgctcctctggacacgtgggactgaggaacctgggaaacaccctgccacagtgctttctgaacgctatgctgcagtgtctgagctccacaagacccctgcgggaccgcggaaggaagaggagatccgagccaaagcgggaccggcgcgatccttctaccgataagagcggtcctgataccttccccgtggaagtgctggagagaaagccccctgaaaaaacatactgataactcgag

SEQ ID NO: 116 - protein, 474 aa, synthetic construct
  REGION 1..474: note = peptide encoded plasmid1
MHLGNFASSLSGGQMYGPGSPLGGAPLGGAPTAARGRKRRSAQRNSNPIIAELSQAMNSGTLLSKPSPPLPPKRRGRKRRSGESKGPLDSSFSQYLGRSCLLDQREAKRKGEGLRGRKRRSLARDEFNLQKMMVMVTASGKLFGIESSSGTILWRGRKRRSETTVKYLDGEKLSVLCHDNYLTQDSEEMVCKDGRGRKRRSDSFQIILDSLLLFGQCSSTPLSEAFHPTVTGESRGRKRRSECKEAAVSHAGSMHRERRTFLRSALKELATVLSRGRKRRSQGKYILKVCGCDEYFLEKFPLSQYKYIRSCIMLRGRKRRSKFISVLCTSLATMLHVELPHVNLLSKMDLIEHYRGRKRRSAEDTQENLHSGKSQSRSFPKVLADSDESDMEETRGRKRRSSGHVGLRNLGNTLPQCFLNAMLQCLSSTRPLRDRGRKRRSEPKRDRRDPSTDKSGPDTFPVEVLERKPPEKTY

SEQ ID NO: 117 - DNA, 1446 bp, synthetic construct
  misc_feature 1..1446: note = plasmid 2
  source 1..1446: mol_type = other DNA
ggatccgccaccatgaactcaggggtcgataacgccattacaaaaagcgagatgctggtcgatcagtatttcccactcactcaggaagagctggagatggaggccaagaaggtgcggggaaggaagaggagaagcctgcggccttccaccctgtctcaggaggtgtacaagacagtgtccatgcgcctgaacgtggtgtctccagacctgagcggcaagggactggtgctgcccaggggcagaaagcggcgctccttcctgcagctgaagctggtggtgaagaagggaaagcagaccgagaacctgtacatcgagctgacactgccacagttctactcctttctgcacgagatgcggggccgcaagaggagatctctgacccacccttacggcaagggatgggacctgatgcagaacatcatggacgatgtgccaatctacatgtacagcgtgtgcaacgtggtgagcggcgataggggaagaaagcggcgctccgcctgtagctcctcttacaactctgctgtgatggagagctcctctgtgaacgtgtccatggtgcacagctcctctaaggagaacctgtgccccaagaagcggggtagaaagaggagatctcagcacgtgatccacaagtacttcctgcctagggcctacgctgtgctgctgccactgcctgctggcctgctgctgctgctgtttgtgggcaagagccccaggggaaggaaacggcgctcccagcacggcatccagctgctgaacggacacgctcctgctgctggaccaaacctgagcggactgccacaggctaacaggcaccacggcctgctgggcggaaggggaaggaagaggaggagcagggacccctacgctctggacctgatcgataagctgctggtgccagaccccgctcagaggatcgatagcgacgatgctctgaaccacgatttcttttggagaggtagaaagcggcgctctcgcgagatcgctgtggagcacaacaacctgggaaaggccgtgtacagcaggatggctagaatctgtaagaacgacatgggcggatctcagagagtgctgaggggacgcaagaggagaagcaacacccagatccagaagctgacaggcagccccttcaggagcagggagtacgtgcagaccatggagagcgagtccgagcactcttaccctcctcccggaaggggaaggaagcggcgctctctgagccagacaccccctccagcccctcctcccagcgccggctccgagccactggctaggctgcctcagaaggatagcgtgggaaagcactccggcgctaggggaagaaagaggagaagccggatgagggaggagcagctggccagagaggctgaggcttgggcagaacgggaggcagaggcaagacgcagggaggaacaggaagcaagggaaaaagcatgataactcgag

SEQ ID NO: 118 - protein, 474 aa, synthetic construct
  REGION 1..474: note = peptide from plasmid 2
MNSGVDNAITKSEMLVDQYFPLTQEELEMEAKKVRGRKRRSLRPSTLSQEVYKTVSMRLNVVSPDLSGKGLVLPRGRKRRSFLQLKLVVKKGKQTENLYIELTLPQFYSFLHEMRGRKRRSLTHPYGKGWDLMQNIMDDVPIYMYSVCNVVSGDRGRKRRSACSSSYNSAVMESSSVNVSMVHSSSKENLCPKKRGRKRRSQHVIHKYFLPRAYAVLLPLPAGLLLLLFVGKSPRGRKRRSQHGIQLLNGHAPAAGPNLSGLPQANRHHGLLGGRGRKRRSRDPYALDLIDKLLVPDPAQRIDSDDALNHDFFWRGRKRRSREIAVEHNNLGKAVYSRMARICKNDMGGSQRVLRGRKRRSNTQIQKLTGSPFRSREYVQTMESESEHSYPPPGRGRKRRSLSQTPPPAPPPSAGSEPLARLPQKDSVGKHSGARGRKRRSRMREEQLAREAEAWAEREAEARRREEQEAREKA

SEQ ID NO: 119 - DNA, 1446 bp, synthetic construct
  misc_feature 1..1446: note = plasmid 3
  source 1..1446: mol_type = other DNA
ggatccgccaccatggagggggagtcaaccaatacatcaaaagtcgctaaaaagtcatcagtccactacagcctgctcgccgaacacgaagaaacccagtccctgggcaccaagcggggacgcaagaggagatccacaggctctatgaacgtgatccacatgctggagtctttcacctttaggaacaacgtgtgcatggccttcgagctgctgtctatcgacctgtacgagctgaggggcagaaagcggcgcagcaactgcaacttcgactttcccgaggagacatgtggatgggtgtacgatcacgccaagctgctgaggtctacatggatcagctccgctaaccctaacgacaggggaaggaagaggagaagcctgccagccagactggagatccagggaaccgatcctatgtccgctgtgacactgtctctgctggaccctgagacacaggaggaccccagcagctggacaaggggcaggaaacggcgcagcggactgtccctgaacatcaagaagtacctgctggtgtctatgcctctgtgggctaagcacatgagcgatgagcagatccagggctttgtggagaacctgagaggccgcaagaggagaagcatctccatgggcggacacatggctagcctggccgtgtgcaactggcccaagcctatgccactgatcccatgtctgtcttggagcaccgcttccggcgtgaggggaagaaagcggcgctctgagcccatcgaggtgaacctgttcggcctgatcaacgtgacacccaacatgctgcctctggtgaagaaggctcggggccgcgtgatcaacgtgtcctctaggggaagaaagaggagatccgtggactttaacaagatctgtggacaggcctacgattctgctacaaacttccgggtgaagctgaacgaggtggtggccgagtttaagaaggaggagccacggggcaggaagcggcgcagcgccgaggtgctgatcgacgctctggtggatggacaggtggtggccctgctggtgcagaacctggagcggctggacgagagcgtgcgcgaggaggctgataggggcagaaagaggagatccgacgaggaggagtctgtggatctgagcatccccggagcctgctacctgagactgctgaccatcacagctccatccgtgctgcccgctctggaggagttccggggacgcaagcggcgcagcaagctgctggtgtactccgctcacgacaccacactggtggccctgcagatggctctgaacctgtacaacggcaagcaggccccttacgctagctgtcacagaggcaggaaaaggagatccggcgaggacgagggagataagccagaggccaagcaggtggtgctgattgggtcatcctacctctgccagttttgtccttctaaattctccacctacttttgataactcgag

SEQ ID NO: 120 - protein, 474 aa, synthetic construct
  REGION 1..474: note = peptide from plasmid 3
MEGESTNTSKVAKKSSVHYSLLAEHEETQSLGTKRGRKRRSTGSMNVIHMLESFTFRNNVCMAFELLSIDLYELRGRKRRSNCNFDFPEETCGWVYDHAKLLRSTWISSANPNDRGRKRRSLPARLEIQGTDPMSAVTLSLLDPETQEDPSSWTRGRKRRSGLSLNIKKYLLVSMPLWAKHMSDEQIQGFVENLRGRKRRSISMGGHMASLAVCNWPKPMPLIPCLSWSTASGVRGRKRRSEPIEVNLFGLINVTPNMLPLVKKARGRVINVSSRGRKRRSVDFNKICGQAYDSATNFRVKLNEVVAEFKKEEPRGRKRRSAEVLIDALVDGQVVALLVQNLERLDESVREEADRGRKRRSDEEESVDLSIPGACYLRLLTITAPSVLPALEEFRGRKRRSKLLVYSAHDTTLVALQMALNLYNGKQAPYASCHRGRKRRSGEDEGDKPEAKQVVLIGSSYLCQFCPSKFSTYF

SEQ ID NO: 121 - DNA, 1446 bp, synthetic construct
  misc_feature 1..1446: note = plasmid 4
  source 1..1446: mol_type = other DNA
ggatccgccaccatgctcctcgctcgcactcaccctggttgtggtaatatcgtcacttttgctcatttcctctttatcgcagtcgaaggtttcctgttcgaggctaacctgggaagaggcaggaagcgcagatcttcccacgtgcagagactggtgcacagggacagcacaatctccaacgacgcctttatctccgtggacgatctggagccatccggaccacaggatctggagcggggaaggaagaggagatccgactacatcgctctgaacgaggatctgaagacctggacagctgccgctatggctgccctgatcacaaagcacaagtgggagcaggccggcgaggctgagaggggaagaaagcggaggtccggcggaaaggtgtggaacgagcaggagaagaacacctacgccgacaagctgttcgattgtatcgaggcttacgctcctggattcaagagaagcgtgctgagaggccggaagcgcagatccaagctggtgttcgtgagcacctctggcggatctctgtggaacctgaaggccatccactccatgtgcaggatcgagcaggagcaggagggaagcgtgcggagaggaaggaagcgcaggtccccaaagatgtgctctcagcctgctgtgagccagctgcagtctaacatcagatcccagtgcatctcccacaccgccctggtgctgcagggcagcctgacaagaggaaggaagagacggtccatccagggatggctgggcaccaaggtgaggacaaacctgcggagagagccattctctctggtgcctaagaacgctaaggacggaaactccttccagggacgcggaaggaagagacgctctaacaccatgacaaccaaggagtgcctgcacaggttttgtgccgactgtatcatcaccggcctgaggagcggaaacaaggagtgccctacctgcagaaagaggggcagaaagcggagaagcaacggatacgatacatacgtgcacgatgcctacggactgttccaggagtgcaggtctagggtggcccactggggctggcctctgggacccgccccactgagaggaagaaagaggaggtccgaccacctgcagctgaaggacatcctgagaaagcaggacgagtaccacatggtgcacctgctgtgcgcctctagatcccctcccagctcccccaagtccaggggacggaagaggcggagcgccccagagctgacattcgactttaccatcgagaccgtgctgtgcctgcaggacagcgtgctggccttctggagccacggaatgcagggacggtccctgagaggacgcaagaggagaagcacaacaaccagcagggccatctgccagctggtgaaggagtacattggatacagggatggcatttgggatgtcagcgtcacacggactcagcctattgtctgataactcgag

SEQ ID NO: 122 - protein, 474 aa, synthetic construct
  REGION 1..474: note = peptide from plasmid 4
MLLARTHPGCGNIVTFAHFLFIAVEGFLFEANLGRGRKRRSSHVQRLVHRDSTISNDAFISVDDLEPSGPQDLERGRKRRSDYIALNEDLKTWTAAAMAALITKHKWEQAGEAERGRKRRSGGKVWNEQEKNTYADKLFDCIEAYAPGFKRSVLRGRKRRSKLVFVSTSGGSLWNLKAIHSMCRIEQEQEGSVRRGRKRRSPKMCSQPAVSQLQSNIRSQCISHTALVLQGSLTRGRKRRSIQGWLGTKVRTNLRREPFSLVPKNAKDGNSFQGRGRKRRSNTMTTKECLHRFCADCIITGLRSGNKECPTCRKRGRKRRSNGYDTYVHDAYGLFQECRSRVAHWGWPLGPAPLRGRKRRSDHLQLKDILRKQDEYHMVHLLCASRSPPSSPKSRGRKRRSAPELTFDFTIETVLCLQDSVLAFWSHGMQGRSLRGRKRRSTTTSRAICQLVKEYIGYRDGIWDVSVTRTQPIV

SEQ ID NO: 123 - DNA, 1446 bp, synthetic construct
  misc_feature 1..1446: note = plasmid 5 insert
  source 1..1446: mol_type = other DNA
ggatccgccaccatggggaacaatatcactgtgaaaaccgagagcaccgtcaaaaccaatgtctttagctgtaatctgggggagaaattcgatgaaaccaccgccgacggcaggagaggaagaaagcggcgctccgtggagcacaagaagcggccaaaggtggagctgatgcccggcatgaggagactgcagtctctgctggataccagatgcgtgtacatcctggactgttggaggggaaggaagcggcgcagcacacagggatgcaagctggtggataccaacaagacacgcaccacatgcgcctgtaggcacctgacaaacttcgccatcctgatggctcaccgcgagatcaggggcagaaagaggagatctcagagcaagaagaggctgcacatcgaggattactccgtgtctcagcctaccctggaccaggtgttcgtgaactttgctaaggatcagagcgacgatgacagaggcaggaagcggcgctcctggaacacatggctgtacgcccctggactgccccctgtgaagccaaactacgacgctaccctgacaaacgcctgcatcgctctgtcccagagatgggtgaggggtaggaagaggagatccctgggatcttacaccgaggataacgcttctcaggctcgggtggacggacacagatgtatcgtgagggccctgaaggaccccaacgctttcctgtttgacagaggtaggaagcggcgcagccagagggacaacctgacacagttctttccccagaactacttccctctgctgggacaggagctggtgcaggccctgaaggctgtggtgaactttctgcagagaggtagaaagaggagaagcaccgcttccggaacacaggacccccactccgctgagacactgggagtgtgccgcagcccaccccctccatgttcccctatcctggaggcttctgatgacagagggaggaagcggcgctctggacaggtcatccaggctcagggaagagctagcccaaccatggccacactgcctgctccaatggcctccgctccccctctgtctgagctgtacccaaacaggggaagaaagaggagaagcgactccaagcggcgcaaggagcagttccccgtgctgtctcacctgctgagcatcgcccagctgccacccaacaaggatgctacctacctgtctcggagccgcggcaggaagaggagaagctccatcctgcgcaaccacaggtacaggagggatgctaggaccctggaggacaaggaggagatgtggtttaacacagatgaggatgacatggaggacggcagaggacggaagaggagaagctggatgaagtgcgtgaagggacagcctcacgaccacaagcacctcatgccaactcagattattcccggctccgtgctcacagacctcctggatgctatgtgataactcgag

SEQ ID NO: 124 - protein, 474 aa, synthetic construct
  REGION 1..474: note = peptide from insert 5
MGNNITVKTESTVKTNVFSCNLGEKFDETTADGRRGRKRRSVEHKKRPKVELMPGMRRLQSLLDTRCVYILDCWRGRKRRSTQGCKLVDTNKTRTTCACRHLTNFAILMAHREIRGRKRRSQSKKRLHIEDYSVSQPTLDQVFVNFAKDQSDDDRGRKRRSWNTWLYAPGLPPVKPNYDATLTNACIALSQRWVRGRKRRSLGSYTEDNASQARVDGHRCIVRALKDPNAFLFDRGRKRRSQRDNLTQFFPQNYFPLLGQELVQALKAVVNFLQRGRKRRSTASGTQDPHSAETLGVCRSPPPPCSPILEASDDRGRKRRSGQVIQAQGRASPTMATLPAPMASAPPLSELYPNRGRKRRSDSKRRKEQFPVLSHLLSIAQLPPNKDATYLSRSRGRKRRSSILRNHRYRRDARTLEDKEEMWFNTDEDDMEDGRGRKRRSWMKCVKGQPHDHKHLMPTQIIPGSVLTDLLDAM

SEQ ID NO: 125 - DNA, 1443 bp, synthetic construct
  misc_feature 1..1443: note = plasmid insert 6
  source 1..1443: mol_type = other DNA
ggatccgccaccatggcaactgatatgtcccagggagaactgattcatcctaaggcttttccactcattgtcggggcacagctcattcacgcagataagctgggcgagaagaggggaagaaagaggagaagcacatgctacctggacgctggcctggctaggaccacaaccggaaacaagttctttggcgccctgaagggagctgtggatggcggactgtccatcccacacaggggaaggaagcggcgcagctggctgctgtccagacctgaggccccagaggacttccagacctttcacgctagcctgcagctgaggaagcagtccctggctcgcaagcacatctacctgaggggcagaaagaggagatccgccaacgctgtgcactctatggagaaagtgatggatatccactccaagtactgcaggtgtctgcagagactgagctccacagtgggccactctctgattagaggaaggaagcggcgctctcccggcctgctgagcgtggacctgttccacgtgctggtgtctgccgtgctggcttttccaagcctgtactgggacgataccgtggatctgcagccctccaggggcaggaaaaggagatctgaggtgcccaagcctctgttccctgtggccggagtgccaatgatccagtaccacatcgaggcctgcgctcaggtgcctggcatgcaggagatcctgctgcgcggcaggaagcggcgctctagcaacgaggacagggagtggcacgagtccttttctctggtgctgggaccagacgatcctgtggaggccgtgctgggcgatgtgacaaccgctacagtgaggggtaggaagaggaggagcctgtgcctgatgggacacaagcccgtgctgctgaggacccagtgcaggtgtcaccctgccatctctgccatcgctaacgacctgttctacgagggcagccggggaagaaaacggcgcagcacatccgccgctgagacaaagccccctctggctccttcctctccacccgctcctccaggcacaatggtggctggaagctccctggagggaccagctcccaggggaaggaaaaggagatctatgagcgtgatggtgctgagaacacagggaccagaggctccctttgaggaccaccggctgatcctgcacacctctagctgcgatgctgagagggctagaagaggaagaaagaggagatccctgtctggaaaggacgagcaggagaacagcaacctggctacccctgagtaccaggtgtccgaggctgctagagagctgggcctggctacagctgaggagcgcggtaggaagcggcgctcctgtccacacatgaccgaggacaacaaggatctgatccagggtaaagacctcctgactgcttgctatgatgtggactacgaaaagaatgccaaaggctcttgataactcgag

SEQ ID NO: 126 - protein, 473 aa, synthetic construct
  REGION 1..473: note = peptide from insert 6
MATDMSQGELIHPKAFPLIVGAQLIHADKLGEKRGRKRRSTCYLDAGLARTTTGNKFFGALKGAVDGGLSIPHRGRKRRSWLLSRPEAPEDFQTFHASLQLRKQSLARKHIYLRGRKRRSANAVHSMEKVMDIHSKYCRCLQRLSSTVGHSLIRGRKRRSPGLLSVDLFHVLVSAVLAFPSLYWDDTVDLQPSRGRKRRSEVPKPLFPVAGVPMIQYHIEACAQVPGMQEILLRGRKRRSSNEDREWHESFSLVLGPDDPVEAVLGDVTTATVRGRKRRSLCLMGHKPVLLRTQCRCHPAISAIANDLFYEGSRGRKRRSTSAAETKPPLAPSSPPAPPGTMVAGSSLEGPAPRGRKRRSMSVMVLRTQGPEAPFEDHRLILHTSSCDAERARRGRKRRSLSGKDEQENSNLATPEYQVSEAARELGLATAEERGRKRRSCPHMTEDNKDLIQGKDLLTACYDVDYEKNAKGS

SEQ ID NO: 127 - DNA, 1443 bp, synthetic construct
  misc_feature 1..1443: note = plasmid insert 7
  source 1..1443: mol_type = other DNA
ggatccgccaccatggctgctacatcagaagggctcctgttcgccgcatcaatcgagaaaacttggggttcagtcgtcaggtcaccagagggtactccacagaaggtgaggagaggaaggaagcggcgctccacaagaggcctggcctacctgcacaccgagctgccacagggcgaccactacaagcccgctatctctcacagagatctgaacagccggaacgtgctggtgagaggccggaagaggagatccatccaccgggagcagaactccctgtctctgctggaggcctctgaggctgacggcgatgccgtgaacgacaagaagaggacacccaacgaggctccttctcgcggaaggaagcggcgcagctaccaggtgaagggcaagaagaacatcagagccacagagcgggctctgagctgggattccttcaacaccggcgactgcctgatcctggatctgggccagagaggacggaagaggagaagcaggtccctgcaggagctgctggctaggaggacctactactccggcgaccccctgttctctgtggatatcctgcggaacgtgacagacacctttaagagaaggggcagaaagcggcgctccggcggacacgtgtgctggatgagagagggcgtggagctgtgcccaggaaacaagtacgagatgaggagacacggaaccacacacagcctggtcatccacaggggccgcaagcggcgcagctccatggctctgtccctgcgcctggccctggctaggtctagcatcgagagatcctctacagctagcgacccccagggagatctgctgtttctgctggataggggtcggaagaggagatctctgagcaagcacaagcagatccctggactgatccagaacatgaccgtgaggtgctgtgagctgccctaccctgagcaggcttgctgtggagaggaggagaggggcaggaaacggcgcagctccaacagcggaccaagaccctccaacaccacaacctctagcaactcccagtctaacagcctgcctcgcccatctaacaggggcagcatctctaactacaggggacggaagaggagatctgctggcagcccaggaatgcagcggcgcaggagacgggtgctgaacacaagcgtggcctacgtgagaggcgaggagaacctggctggatggcggcccaggaggggacgcaagagacggtccatcaagtcttgccctgagtgtcggatcaccagcaacttcgtgatcccatccgagtactgggtggaggagaaggaggagaagcagaagctgatccagaagaggggaaggaagcgcaggagcgccgaggacgagatcgaggatatccagcaggagatcacagctctcagccagtgtgatagcccttacattaccagatacttcggttcctacctcaaatcctgataactcgag

SEQ ID NO: 128 - protein, 473 aa, synthetic construct
  REGION 1..473: note = peptide from insert 7
MAATSEGLLFAASIEKTWGSVVRSPEGTPQKVRRGRKRRSTRGLAYLHTELPQGDHYKPAISHRDLNSRNVLVRGRKRRSIHREQNSLSLLEASEADGDAVNDKKRTPNEAPSRGRKRRSYQVKGKKNIRATERALSWDSFNTGDCLILDLGQRGRKRRSRSLQELLARRTYYSGDPLFSVDILRNVTDTFKRRGRKRRSGGHVCWMREGVELCPGNKYEMRRHGTTHSLVIHRGRKRRSSMALSLRLALARSSIERSSTASDPQGDLLFLLDRGRKRRSLSKHKQIPGLIQNMTVRCCELPYPEQACCGEEERGRKRRSSNSGPRPSNTTTSSNSQSNSLPRPSNRGSISNYRGRKRRSAGSPGMQRRRRRVLNTSVAYVRGEENLAGWRPRRGRKRRSIKSCPECRITSNFVIPSEYWVEEKEEKQKLIQKRGRKRRSAEDEIEDIQQEITALSQCDSPYITRYFGSYLKS

SEQ ID NO: 129 - DNA, 3257 bp, synthetic construct
  misc_feature 1..3257: note = plasmid sequence 11
  source 1..3257: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacctggattctgttcctggtggccgctgccacacgggtgcacagccgcgagggagtggagctgtgcccaggaaacaagtacgagatgaggagacacggcaccacacactccctggtcatccacgacccatctaagcccagcttccaggagtttgtggattgggagaacgtgtcccctgagctgaactctaccgaccagccattcctggatagcggctccccctttcctgctgccgtgatcctgagggatgctctgcacatggcccggggactgaagtacctgcaccagtgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 130 - DNA, 3257 bp, synthetic construct
  misc_feature 1..3257: note = plasmid sequence 12
  source 1..3257: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacctggattctgttcctggtggccgctgccacaagggtgcactctcaggctatcgtgcggggatgcagcatgccaggaccttggcggtccggccgcctgctggtgtccaggagatggtctgtggagccactgctgcccttctaccctcccgatgaggctctggagatcggactggagctgaacagctccgccctgccacccaccgaagtgatcctgcctcaggctccatctggccccagctacgccacctacctgcagcctgctcaggcccagatgctgacacctccatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

(19) United States
(12) Patent Application Publication
(10) Pub. No.: US 2026/0285928 A1
(43) Pub. Date: Sep. 24, 2026

(54) Tumor-Specific Neoantigens and Methods of Using the Same

(71) Applicants: THE WISTAR INSTITUTE OF ANATOMY AND BIOLOGY, Philadelphia, PA (US); INOVIO PHARMACEUTICALS, INC., Plymouth Meeting, PA (US); GENEOS THERAPEUTICS, INC., Philadelphia, PA (US)

(72) Inventors: Elizabeth Kennedy Duperret, Philadelphia, PA (US); Alfredo Perales Puchalt, Philadelphia, PA (US); David B. Weiner, Merion Station, PA (US); Niranjan Y. Sardesai, Blue Bell, PA (US)

(73) Assignees: THE WISTAR INSTITUTE OF ANATOMY AND BIOLOGY, Philadelphia, PA (US); INOVIO PHARMACEUTICALS, INC., Plymouth Meeting, PA (US); GENEOS THERAPEUTICS, INC., Philadelphia, PA (US)

(21) Appl. No.: 19/698,768

(22) Filed: Jun. 4, 2026

Related U.S. Application Data
(63) Continuation of application No. 19/208,273, filed on May 14, 2025.
Application No. 19/208,273 is a continuation of application No. 17/058,999, filed on Nov. 25, 2020, now Pat. No. 12,528,848.

(51) Int. Cl. C07K 14/47; A61K 39/00; A61K 40/11; A61K 40/42; A61K 45/06; A61P 35/00; C12Q 1/6886

(52) U.S. Cl. CPC C07K 14/4748; A61K 39/00; A61K 39/0011; A61K 39/00119; A61K 40/11; A61K 40/4201; A61K 40/4271; A61K 45/06; A61P 35/00; C12Q 1/6886; A61K 2039/53; C12Q 2600/156

(57) ABSTRACT

The disclosure relates to methods of manufacturing individualized vaccines that comprise nucleic acid molecules that encode one or more neoantigens specific for antigens that are expressed by a tumor in a subject. Compositions comprising coding regions encoding neoantigens organized in a pattern of nucleic acid sequences are also disclosed as well as methods of immunizing a subject using the same.

### CROSS REFERENCE TO RELATED APPLICATIONS

This is a continuation of U.S. application Ser. No. 19/208,273, entitled “Methods of Antigen Production, filed May 14, 2025, which is a continuation of U.S. application Ser. No. 17/058,999, entitled “Tumor-Specific Neoantigens and Methods of Using the Same,” filed Nov. 25, 2020, and issued Jan. 20, 2026, as U.S. Pat. No. 12,528,848, which is a U.S. national stage application filed under 35 U.S.C. 371 based on International Patent Application No. PCT/US2019/034229, entitled “Tumor-Specific Neoantigens and Methods of Using the Same,” filed May 28, 2019, which claims the benefit of U.S. Provisional Application No. 62/676,563, titled “Tumor-Specific Neoantigens and Methods of Using the Same,” filed May 25, 2018, the disclosures of all of which are incorporated herein by reference in their entireties.

### SEQUENCE LISTING

The electronic sequence listing filed on Jun. 4, 2026, titled “WIST-022-US-Sequence Listing.xml,” created on Jun. 4, 2026, and having a file size of 205,525 bytes is incorporated herein by reference in its entirety.

### FIELD

The disclosure relates to vaccines, nucleic acid sequence that are components of vaccines and methods of manufacturing and using the same for inducing antigen-specific immune responses in a subject.

### BACKGROUND

The immune system can be classified into two functional subsystems: the innate and the acquired immune system. The innate immune system is the first line of defense against infections, and most potential pathogens are rapidly neutralized by this system before they can cause, for example, a noticeable infection. The acquired immune system reacts to molecular structures, referred to as antigens, of the intruding organism. There are two types of acquired immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T-cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells, and are then presented to naive or cytotoxic T-lymphocytes (CTLs). MHC class II proteins are present on dendritic cells, B-lymphocytes, macrophages and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself, and do not normally stimulate an immune reaction. Accordingly, cytotoxic T-lymphocytes (CTLs) that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and cluster of differentiation (CD) molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes.

The peptide antigens attach themselves to the molecules of MHC class I by competitive affinity binding within the endoplasmic reticulum, before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines.

One of the critical barriers to developing curative and tumor-specific immunotherapy is the identification and selection of highly specific and restricted tumor antigens to avoid autoimmunity. Cancer neoantigens, epitopes derived from tumor-specific somatic mutations that are presented on MHCs, are emerging as promising targets for personalized immunotherapy. These epitopes are thought to be more robust immunotherapy targets compared to shared, overexpressed tumor-associated self-antigens due to i) their high frequency in human cancers (ranging from approximately 33-163 expressed, non-synonymous mutations for common solid tumors in adults) (Vogelstein et al. Science (80-). 2013; 339:1546-58), ii) their lack of expression in normal somatic tissues, and iii) their high potential for immunogenicity due to lack of central and peripheral tolerance. Indeed, effective immune checkpoint blockade therapy has been associated with specific targeting of tumor neoantigens (Gubin et al. Nature. 2014; 515:577-81; McGranahan et al. Science. 2016; 351:1463-9). However, the same immunogenic neoantigens are rarely shared across multiple patients (Rech et al., Cancer Immunol Res. 2018); therefore, this type of therapy is highly personalized and requires rapid, efficient and affordable sequencing and manufacturing processes. Furthermore, the vast majority of these mutations are passenger mutations, and not drivers of the malignancy; thus, there is a high likelihood of tumor escape.

In 2017, there were an estimated 1,688,780 new cancer cases diagnosed, and 600,920 cancer deaths in the US. Over the past few decades there been significant improvements in the detection, diagnosis, and treatment of cancer, which have significantly increased the survival rate for many types of cancer. The number of cancer deaths (cancer mortality) is 171.2 per 100,000 men and women per year (based on 2008-2012 deaths), which makes cancer among the leading causes of death in the United States.

Existing cancer therapies include ablation techniques (e.g., surgical procedures, cryogenic/heat treatment, ultrasound, radiofrequency, and radiation) and chemical techniques (e.g., pharmaceutical agents, cytotoxic/chemotherapeutic agents, monoclonal antibodies, and various combinations thereof). Unfortunately, such therapies are frequently associated with serious risk, toxic side effects, and extremely high costs, as well as uncertain efficacy.

Cancer vaccines are typically composed of tumor antigens and immunostimulatory molecules (e.g., cytokines or TLR ligands) that work together to induce antigen-specific cytotoxic T cells that target and destroy tumor cells. Current cancer vaccines typically contain shared tumor antigens, which are native proteins (i.e.—proteins encoded by the DNA of ail the normal cells in the individual) that are selectively expressed or over-expressed in tumors found in many individuals. While such shared tumor antigens are useful in identifying particular types of tumors, they are not ideal as immunogens for targeting a T-cell response to a particular tumor type because they are subject to the immune dampening effects of self-tolerance.

Early clinical trials using synthetic long peptides (15-30mer) delivered with poly(I: C), dendritic cells loaded with short HLA class I restricted peptides, or RNA vaccines encoding long (27mer) neo-epitope peptides have shown immune responses directed against a significant fraction of mutated epitopes delivered (Ott et al. Nature. Nature Publishing Group; 2017; 547:217-21; Sahin et al. Nature. Nature Publishing Group; 2017; 547:222-6; Carreno et al. Science. 2015; 348:803-8). The vast majority of these responses driven by RNA or synthetic long peptides have been MHC class II restricted, both in these early clinical studies as well as in pre-clinical mouse studies (70-95% in mice and 72.5-79% in humans) (Ott et al. 2017; Sahin et al. 2017; Kretier et al. Nature; 2015; 520:692-6; Martin et al. PLOS One. 2016; 11). This strong induction of CD4⁺ T cell responses occurs despite the fact that the epitopes were selected in silico for high MHCI binding affinity (Ott et al. 2017; Sahin et al. 2017; Kreiter et al. 2015).

### SUMMARY OF THE DISCLOSURE

The disclosure describes the development of DNA vaccines encoding neoantigens that are capable of generating robust MHC class I-restricted immune responses against neoantigens in a greater proportion than those immune responses generated by RNA and peptide vaccine platforms. Neoantigen targeted immunotherapies are based on the specific activation of certain well-defined tumor antigens that have been mapped to the patient's specific tumor. These are antigens that have been shown to be expressed in the tumor and presented to the immune system of that patient, and thus are specifically targeted to the patient's tumor without the risk of non-specific/bystander targeting from a broad activation of innate immunity.

The DNA vaccines described in the present disclosure surprisingly generate a much larger proportion of CD8⁺ T cell responses for the immunogenic epitopes. The present disclosure describes for the first time that inclusion of only high affinity MHC class I epitopes selected for a larger proportion of immunogenic epitopes, and selected for 100% CD8⁺ or CD8⁺/CD4⁺ T cell epitopes. Moreover, the present disclosure described for the first time that DNA vaccines encoding neo-antigens were able to control tumor growth in vivo in a therapeutic setting, and T cells expanded from immunized mice were able to kill tumor cells ex vivo. Thus, the DNA vaccines targeting neoantigens described in the present disclosure can overcome many of the limitations of other vaccine platforms, and may be able to work synergistically with other platforms for effective immunotherapy approaches.

In some embodiments, a nucleic acid molecule comprising a nucleic acid sequence comprising Formula I: [(AEDⁿ)-(linker)]ₙ-[AEDⁿ⁺¹], wherein the AED is an antigen expression domain comprising an expressible nucleic acid sequence. Each linker is independently selectable from about 0 to about 125 natural or non-natural nucleic acids in length. The antigen expression domain 1 is independently selectable from about 24 to about 250 nucleotides in length and encodes an epitope. The antigen expression domain 2 is independently selectable from about 24 to about 250 nucleotides in length and encodes an epitope, and n is any positive integer from about 1 to about 500.

In some embodiments, the antigen expression domain 1 and the antigen expression domain 2 are independently selectable from about 20 to about 2,000 nucleotides in length.

In some embodiments, the antigen expression domain 1 or antigen expression domain 2 is independently selectable from about 50 to about 10,000 nucleotides in length and n is any positive integer from about 6 to about 26.

In some embodiments, the antigen expression domain 1 and/or the antigen expression domain 2 are independently selectable from about 15 to about 150, about 15 to about 100, or about 15 to about 50 nucleotides in length.

In some embodiments, n is a positive integer from about 5 to about 30, about 2 to about 100, about 2 to about 58, or about 2 to about 29.

In some embodiments, at least one linker comprises from about 15 to about 300 nucleotides and encodes a cleavage site. Optionally, the at least one linker comprises a furin protease cleavage site or a porcine teschovirus-1 2A (P2A) cleavage site.

In some embodiments, at least one linker comprises from about 15 to about 300 nucleotides, encodes a cleavage site, and Formula I comprises at least a first linker and a second linker, wherein the first and second linker comprise a furin protease cleavage site.

In some embodiments, at least one linker comprises from about 15 to about 300 nucleotides, encodes a cleavage site, n is a positive integer from about 5 to about 50, and each linker comprises a furin protease cleavage site. The nucleic acid molecule of this embodiment may further comprise at least one regulatory sequence, and wherein at least one nucleic acid sequence of Formula I is operably linked to the regulatory sequence.

In some embodiments, the antigen expression domain 1 is independently selectable from about 12 to about 15,000 nucleotides in length and encodes an epitope from one or a plurality of cancer cells from a subject. The antigen expression domain 2 is independently selectable from about 12 to about 15,000 nucleotides in length and encodes an epitope from one or a plurality of cancer cells from the subject.

In some embodiments, the nucleic acid molecule is in an amount sufficient to elicit a CD8+ T cell response and/or a CD4+ T cell response against any one or plurality of amino acid sequences encoded by one or plurality of antigen expression domains.

In some embodiments, the nucleic acid molecule further comprises a leader sequence, such as an IgE leader sequence.

In some embodiments, the nucleic acid molecule is a plasmid. In some embodiments, the nucleic acid sequence of Formula I is positioned with the multiple cloning site of a plasmid is (i) selected from the group consisting of: LLC, TC1, ID8, pGX4501, pGX4503, pGX4504, pGX4505, and pGX4506, or (ii) a plasmid having at least 70% homology with a plasmid selected from the group consisting of: LLC, TC1, ID8, pGX4501, pGX4503, pGX4504, pGX4505, and pGX4506. It should be appreciated that host cells can be transformed with such plasmids.

In some embodiments, a composition comprises one or a plurality of nucleic acid molecules as described herein.

In some embodiments, a pharmaceutical composition comprises (i) one or a plurality of any of the nucleic acid molecules described herein, or a pharmaceutically acceptable salt thereof and (ii) a pharmaceutically acceptable carrier. Optionally, the pharmaceutical composition may further comprise one or more therapeutic agents, such as a biologic therapeutic or a small molecule. In some embodiment, one of the therapeutic agents is (i) a checkpoint inhibitor or functional fragment thereof, or (ii) a nucleic acid sequence that encodes a checkpoint inhibitor or functional fragment thereof. In some embodiments, the checkpoint inhibitor or functional fragment thereof associates or inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof. In some embodiments, the checkpoint inhibitor is an inhibitor of the programmed death-1 (PD-1) pathway. In some embodiments, the checkpoint inhibitor is an anti-cytotoxic T-lymphocyte-associated antigen 4 (CTLA4) antibody. In some embodiments, a pharmaceutical composition comprises a pharmaceutically effective amount of: (i) one or a plurality of any of the nucleic acid molecules described herein or nucleic acid sequences that are about 70%, 75%, 80%, 85%, 90%, 91%, 92%, 93%, 94%, 95%, 96%, 97%, 98%, or 99% homologous to any nuclecui acid sequence listed herein, or a pharmaceutically acceptable salt thereof; and (ii) a pharmaceutically acceptable carrier.

In some embodiments, the therapeutic agent is an adjuvant or functional fragment thereof. In some embodiments, the adjuvant or functional fragment thereof is (i) selected from the group consisting of: poly-ICLC, 1018 ISS, aluminum salts, Amplivax. AS15, BCG, CP-870,893, CpG7909, CyaA, GM-CSF, IC30, IC31, Imiquimod, ImuFact 1MP321, IS Patch, ISS, ISCOMATRIX, JuvImmune, Lipo Vac, monophosphoryf lipid A, Montanide IMS 1312, Montanide ISA 206, Montanide ISA 50V, Montanide ISA-51, OK-432, OM-174, OM-197-MP-EC, ONTAK, PEPTEL, vector system, PLGA micropartieles, resiquimod, S L172, Virosomes and other Virus-like particles, YF-17D, VEGF trap, R848, beta-gluean, Pam3Cys, acrylic or methacrylic polymers, copolymers of maleic anhydride and Aquila's QS21 stimulon, and a functional fragment of any thereof; or (ii) a nucleic acid molecule encoding an adjuvant selected from the group consisting of: poly-ICLC, 1018 ISS, Amplivax AS15, BCG, CP-870,893, CpG7909, CyaA, GM-CSF, IC30, IC31, Imiquimod, ImuFact 1MP321, IS Patch, ISS, ISCOMATRIX, JuvImmune, Lipo Vac, monophosphoryf lipid A, Montanide IMS 1312, Montanide ISA 206, Montanide ISA 50V, Montanide ISA-51, OK-432, OM-174, OM-197-MP-EC, ONTAK, PEPTEL, vector system, resiquimod, S L172, Virosomes and other Virus-like particles, YF-17D, VEGF trap, R848, beta-gluean, Pam3Cys, and Aquila's QS21 stimulon, or functional fragment thereof. IL-12, IL-15 (protein or plasmid or NA)

In some embodiments, the therapeutic agent is: (i) an immunostimulatory agent or functional fragment thereof; or (ii) a nucleic acid sequence encoding an immunostimulatory agent or a functional fragment thereof. In some embodiments, the immunostimulatory agent is an interleukin or a functional fragment thereof. In some embodiments, the therapeutic agent is: (i) an chemotherapeutic agent or functional fragment thereof; or (ii) a nucleic acid sequence encoding an chemotherapeutic agent or a functional fragment thereof.

In some embodiments, a method of treating and/or preventing cancer in a subject comprises administering to the subject in need thereof a pharmaceutically effective amount of any of the nucleic acid molecules, or any of the pharmaceutical compositions described in this disclosure. In some embodiments, treatment is determined by a clinical outcome, an increase, enhancement or prolongation of anti-tumor activity by T cells, an increase in the number of anti-tumor T cells or activated T cells as compared with the number prior to treatment, or a combination thereof. In some embodiments, the clinical outcome is selected from the group consisting of tumor regression, tumor shrinkage, tumor necrosis, anti-tumor response by the immune system, tumor expansion, recurrence or spread, or a combination thereof. In some embodiments, the cancer has a high mutational load.

In some embodiments, the cancer is selected from the group consisting of: non-small cell lung cancer, melanoma, ovarian cancer, cervical cancer, glioblastoma, urogenital cancer, gynecological cancer, lung cancer, gastrointestinal cancer, head and neck cancer, non-metastatic or metastatic breast cancer, malignant melanoma, Merkel Cell Carcinoma or bone and soft tissue sarcomas, haematologic neoplasias, multiple myeloma, acute myelogenous leukemia, chronic myelogenous leukemia, myelodysplastic syndrome and acute lymphoblastic leukemia, breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer.

In some embodiments, a method of enhancing an immune response against a plurality of heterogeneous hyperproliferative cells in a subject comprises administering to the subject in need thereof a pharmaceutically effective amount of any of the nucleic acid molecules or any of the pharmaceutical composition of as described in this disclosure. In some embodiments, the immune response is of a sufficient magnitude or efficacy to inhibit or retard tumor growth, induce tumor cell death, induce tumor regression, prevent or delay tumor recurrence, prevent tumor growth, prevent tumor spread and/or induce tumor elimination.

In some embodiments, of enhancing an immune response against a plurality of heterogeneous hyperproliferative cells in a subject further comprises administration of one or more therapeutic agents, as disclosed herein. In some embodiments, the additional therapeutic agent is a biologic therapeutic or a small molecule. In some embodiments, the therapeutic agent is: (i) a checkpoint inhibitor or functional fragment thereof; or (ii) a nucleic acid molecule encoding a checkpoint inhibitor or a functional fragment thereof. In some embodiments, the checkpoint inhibitor associates with or inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands, IDO inhibitors, or a combination thereof. In some embodiments, the checkpoint inhibitor is an inhibitor of the programmed death-1 (PD-1) pathway. In some embodiments, the checkpoint inhibitor is an anti-cytotoxic T-lymphocyte-associated antigen 4 (CTLA4) antibody or functional fragment thereof. In some embodiments, the therapeutic agent is selected from the adjuvants as disclosed herein, or an immunostimulatory agent or functional fragment thereof. In some embodiments, the immunostimulatory agent is an interleukin or functional fragment thereof. In some embodiments, the therapeutic agent is a chemotherapeutic agent.

In some embodiments, the subject has cancer. In some embodiments, the subject has not responded to checkpoint inhibitor therapy.

In some embodiments, the nucleic acid molecule is administered to the subject by electroporation.

In some embodiments, a method of inducing an immune response in a subject comprises administering to the subject in need thereof a pharmaceutically effective amount of any of the nucleic acid molecules or any of the pharmaceutical composition of the present disclosure. In some embodiments, the immune response is a CD8+ T cell immune response. In some embodiments, inducing the CD8+ T cell immune response comprises activating 0.01% to about 50% CD8+ T cells. In some embodiments, inducing the CD8+ T cell immune response comprises expanding CD8+ T cells.

In some embodiments, a method of enhancing an immune response in a subject comprises administering to the subject in need thereof a pharmaceutically effective amount of any of the nucleic acid molecules or any of the pharmaceutical compositions disclosed herein.

In some embodiments, the immune response is a CD8+ T cell immune response. In some embodiments, enhancing the CD8+ T cell immune response comprises activating 0.01% to about 50% CD8+ T cells. In some embodiments, enhancing the CD8+ T cell immune response comprises expanding CD8+ T cells.

In some embodiments, a method of identifying one or more subject-specific DNA neoantigen mutations in a subject, wherein the subject has a cancer characterized by the presence or quantity of a plurality of neoantigen mutations comprises:
sequencing a nucleic acid sample from a tumor of the subject and of a non-tumor sample of the subject;
analyzing the sequence to determine coding and non-coding regions;
identifying sequences comprising tumor-specific non-synonymous or non-silent mutations not present in the non-tumor sample;
identifying single nucleotide variations and single nucleotide insertions and deletions;
producing subject-specific peptides encoded by the sequences comprising tumor-specific non-synonymous or non-silent mutations not present in the non-tumor sample; and
measuring the binding characteristics of the of the subject-specific peptides,
wherein each subject-specific peptide is an expression product of subject-specific DNA neoantigen not present in the non-tumor sample,
thereby identifying one or more subject-specific DNA neoantigens in a subject.

In some embodiments, the step of measuring the binding characteristics of the of the subject-specific peptides is carried out by one or more of:
measuring the binding of the subject-specific peptides to T-cell receptor;
measuring the binding of the subject-specific peptides to a HLA protein of the subject; or
measuring the binding of the subject-specific peptides to transporter associated with antigen processing (TAP).

In some embodiments, the subject-specific peptides bind to HLA proteins of the subject with an IC50 of less than about 500 nM. In some embodiments, the step of ranking the subject-specific peptides based on the binding characteristics. In some embodiments, the method further comprises the step of measuring the CD8+ T cell immune response generated by the subject-specific peptides. In some embodiments, the method further comprises formulating the subject-specific DNA neoantigens into a immunogenic composition for administration to the subject. In some embodiments, about 200 ranked neoantigen mutations are included in the immunogenic composition.

In some embodiments, the method further comprises steps of providing a culture comprising dendritic cells obtained from the subject, and contacting the dendritic cells with the immunogenic composition. In some embodiments, the method further comprises steps of: administering to the subject the dendritic cells, obtaining a population of CD8+ T cells from a peripheral blood sample from the subject, wherein the CD8+ cells recognize the at least one neoantigen, and expanding the population of CD8+ T cells that recognizes the neoantigen. In some embodiments, the method further comprises administering to the subject the expanded population of CD8+ T cells.

In some embodiments, a method of making an individualized cancer vaccine for a subject suspected of having or diagnosed with a cancer comprises:
identifying a plurality of mutations in a sample from the subject;
analyzing the plurality of mutations to identify one or more neoantigen mutations; and
producing, based on the identified subset, a personalized cancer vaccine.

In some embodiments, the step of identifying comprises sequencing the cancer. In some embodiments, the step of analyzing further comprises determining one or more binding characteristics associated with the neoantigen mutation, the binding characteristics selected from the group consisting of binding of the subject-specific peptides to T-cell receptor, binding of the subject-specific peptides to a HLA protein of the subject and binding of the subject-specific peptides to transporter associated with antigen processing (TAP), and ranking, based on the determined characteristics, each of the neo-antigenic mutations.

In some embodiments, the method comprises cloning nucleic acid sequences encoding the one or plurality of neoantigen mutations into a nucleic acid molecule, such as a plasmid. In some embodiments, the nucleic acid molecule comprises a nucleic acid sequence of Formula I that is positioned within the multiple cloning site of a plasmid selected from the group consisting of: pGX4501, pGX4503, pGX4504, pGX4505, and pGX4506.

### BRIEF DESCRIPTION OF THE DRAWINGS

FIG. 1A-FIG. 1E shows DNA vaccine neo-epitope dodecamers induce frequent immune responses. FIG. 1A. Experimental setup. C57Bl/6 mice were implanted with LLC, TC1 or ID8 tumor cells subcutaneously (LLC and TC1) or in the peritoneum (ID8). Tumors and normal tail tissue were harvested 3 weeks after implantation for RNA and DNA isolation and sequencing. Mutations were identified through comparison with normal tissue. FIG. 1B. Number of mutations identified for each tumor type with the indicated MHC class I affinity (NetMHCons v1.1). Mutations were included if they were expressed with an Alt allele depth in RNA-seq≥1, a proteasomal cleavage score≥10 and a TAP processing score≤0.5. FIG. 1C. Plasmid DNA design. The predicted 9mer epitopes were flanked by 12 amino acids on each side (SEQ ID NO:4). Each epitope was separated by a furin cleavage site (SEQ ID NO:41). FIG. 1D. Mice were immunized with each plasmid (n=5 mice per group) with 25 μg of DNA followed by electroporation (EP) three times at two week intervals, and were sacrificed for IFNγ ELISpot and flow cytometry analysis one week after final immunization. FIG. 1E. Percentage of epitopes that generated immune responses (>50 SFU/million splenocytes) for each tumor type.

FIG. 2A-FIG. 2F. FIG. 2A shows the nucleotide sequence (SEQ ID NO:115) of LLC Plasmid #1 and FIG. 2B shows the amino acid sequence (SEQ ID NO:116) of LLC Plasmid #1. FIG. 2C shows the nucleotide sequence (SEQ ID NO: 117) of LLC Plasmid #2 and FIG. 2D shows the amino acid sequence (SEQ ID NO: 118) of LLC Plasmid #2. FIG. 2E shows the nucleotide sequence (SEQ ID NO: 119) of LLC Plasmid #1 and FIG. 2F shows the amino acid sequence (SEQ ID NO: 120) of LLC Plasmid #3.

FIG. 3A-FIG. 3D. FIG. 3A shows the nucleotide sequence (SEQ ID NO: 121) of TC1 Plasmid #1 and FIG. 3B shows the amino acid sequence (SEQ ID NO: 122) of TC1 Plasmid #1. FIG. 3C shows the nucleotide sequence of (SEQ ID NO: 123) TC1 Plasmid #2 and FIG. 3D shows the amino acid sequence (SEQ ID NO: 124) of TC1 Plasmid #2.

FIG. 4A-FIG. 4D. FIG. 4A shows the nucleotide sequence (SEQ ID NO: 125) of ID8 Plasmid #1 and FIG. 4B shows the amino acid sequence (SEQ ID NO: 126) of ID8 Plasmid #1. FIG. 4C shows the nucleotide sequence (SEQ ID NO: 127) of ID8 Plasmid #2 and FIG. 4D shows the amino acid sequence (SEQ ID NO: 128) of ID8 Plasmid #2.

FIG. 5 shows allele frequency of neoantigen gene expression in tumor cell lines in vivo vs in vitro. Allele frequency of mutated genes in TC1, ID8 and LLC tumor cell lines growing in vivo (forming tumors in C57Bl/6) or in vitro (growing in 2D in cell culture flasks).

FIG. 6A-F shows CD4 and CD8 T cell responses to neoantigens. FIG. 6A-F shows intracellular cytokine staining of splenocytes isolated from control (pVax) or neoantigen immunized mice, stimulated with the corresponding neoantigen-specific peptides for 5 hours. Shown are the percentage of CD8+ T cells that are IFNγ+ (FIG. 6A), TNFα+ (FIG. 6B) and IL-2+ (FIG. 6C), and CD4+ T cells that are IFNγ+ (FIG. 6D), TNFα+ (FIG. 6E) and IL-2+ (FIG. 6F). N=5 mice per group. Significance was calculated using a two-tailed student's t-test. Error bars indicate ±SEM. *p<0.05, **p<0.01, ***p<0.001, ****p<0.0001.

FIG. 7A-FIG. 7E shows DNA vaccines generate predominantly CD8 T cell responses to neoantigens. FIG. 7A is a graph of IFNγ ELISpot responses for each epitope that generated a significant immune response (>50 SFU/million splenocytes, statistically significantly greater response compared to pVax immunized mice). Red bars indicate responses that are exclusively CD8⁺ T cells, blue bars indicate responses that are exclusively CD4+ T cells, and purple bars indicate responses that are both CD8⁺ and CD4⁺ T cells. FIG. 7B and FIG. 7C show example flow cytometry data from mice immunized with a TC1 plasmid containing Sgsm2 (FIG. 7B) or Lta4h (FIG. 7C). Shown are IFNγ and TNFα responses for CD4 and CD8 T cells. FIG. 7D shows IFNγ ELISpot responses from (FIG. 7A), displayed according to MHC class I affinity (NetMHCCons v1.1). Red bars indicate high affinity (<500 nM), orange bars indicate medium affinity (500 nM-2000 nM), and yellow bars indicate low affinity (>2000 nM). FIG. 7E shows percentage of epitopes that generate CD4 versus CD8 T cell responses, organized according to MHC class I affinity. All data shown is ±SEM. N=5 mice per group.

FIG. 8A-FIG. 8F shows polyfunctional T cell responses to neoantigens. FIG. 8A-FIG. 8F show polyfunctional cytokine analysis of data shown in FIG. 6. Shown are the percentage of CD8+ T cells that co-express IFNγ, T-bet and CD107a (FIG. 8A), CD8+ T cells that co-express IFNγ and TNFα (FIG. 8B), and CD8+ T cells that co-express IFNγ, TNFα and IL-2 (FIG. 5C). Also shown are the percentage of CD4+ T cells that co-express IFNγ, T-bet and CD107a (FIG. 8D), CD8+ T cells that co-express IFNγ and TNFα (FIG. 8E), and CD8+ T cells that co-express IFNγ, TNFα and IL-2 (FIG. 8F). N=5 mice per group. Significance was calculated using a two-tailed student's t-test. Error bars indicate ±SEM. *p<0.05, **p<0.01, ***p<0.001, ****p<0.0001.

FIG. 9A and FIG. 9B shows MHCII prediction of neoantigen responses. Percentage of epitopes that generate CD4 versus CD8 T cell responses, organized according to MHC class II affinity, predicted using the SMM align method (netMHCII-1.1, FIG. 9A) or the NN align method (netMHCII-2.2, FIG. 9B).

FIG. 10A and FIG. 10B shows most neoantigen responses are specific to the mutant peptide rather than the wild-type peptide. FIG. 10A shows a comparison of IFNγ ELISpot responses against the mutated neoantigen epitope (MUT) to responses against the corresponding wild-type, non-mutated epitope (WT). FIG. 10B. The data from A, expressed in terms of fold change between responses to the MUT and WT epitopes (MUT/WT). Red bars indicate epitopes with >2 fold increase in response, orange bars indicate epitopes with >1.5 fold increase in response, blue bars indicate epitopes with <1.5 fold change in response, and green bars indicate epitopes with >1.5 fold decrease in response. N=5 mice per group. Error bars indicate ±SEM.

FIG. 11A-FIG. 11F shows DNA vaccine primed T cells selectively kill mutated cells. FIG. 11A shows flow cytometry plots showing IFNg expressing CD4 and CD8 T cells that resulted from the expansion of Herpud2 and Lta4 h reactive T cells. Cytotoxicity of T-cells derived from TC1 plasmids. FIG. 11B Sgmsm2 FIG. 11C Herpud2 and FIG. 11C Lta4 h expanded with their corresponding overlapping peptides (5 μg/ml) four weeks co-cultured for 24 hours with either TC1 or ID8 tumor cells measured by luciferase expression after co-culture of 24 hours (triplicates, similar results with 5-hour incubation). FIG. 11D shows RNA expression levels of Sgmsm2, Herpud2 and Lta4 h in TC1 tumors grown in vivo compared to TC1 cells grown in vitro. FIG. 11E shows flow cytometry histograms showing surface expression of MHC class II on B16 melanoma, TC1, ID8 and LLC tumor cells in normal conditions or after being exposed to 50 ng/ml of interferon gamma. FIG. 11F Histograms showing basal and IFNγ-induced expression of MHC class II in the B16 melanoma, TC1, LLC and ID8 measured by flow cytometry. Two-way ANOVA. Error bars indicate ±SEM. ***p<0.001.

FIG. 12. Surface expression of MHC class II on tumor cells. Flow cytometric quantification of MHC class II surface expression in B16 melanoma, TC1, ID8 and LLC tumor cell lines upon exposure to different concentrations of interferon gamma (measured as mean fluorescent intensity, triplicates). Error bars indicate ±SEM.

FIG. 13A and FIG. 13B shows deletion of immunodominant epitopes in TC1 plasmids does not result in improved immunogenicity of sub-dominant epitopes. IFNγ ELISpot responses from mice immunized with a control plasmid (pVax), compared to mice immunized with the TC1 plasmid #1 in which Sgsm2 and Herpud2 immunodominant epitopes were deleted (FIG. 13A), or with the TC1 plasmid #2 in which the Lta4 h immunodominant epitope was deleted (FIG. 13B). N=5 mice per group. Error bars indicate ±SEM.

FIG. 14A-FIG. 14C shows DNA neoantigen vaccines delay tumor progression. FIG. 14A Schematic of tumor challenge experiments: we implanted 100,000 TC1 tumor cells subcutaneously and 7 days later started weekly immunizations with TC1 plasmid 1, 2, both or pVax empty vector. FIG. 14B Tumor volume of mice bearing TC1 treated TC1 plasmid 1, 2, both or pVax. FIG. 14C Survival curve of mice bearing TC1 treated TC1 plasmid 1, 2, both or p Vax (n=10 mice per group). Log rank and two-way ANOVA. Error bars indicate ±SEM. *p<0.05, ** p<0.01, ****p<0.0001.

FIG. 15 shows a schematic of the design of poly-neoepitope DNA vaccine against B16 model. pGX4501 and pGX4503 plasmids were used. F indicates a furin cleavage site, encoded by a linker region. P2A indicates a porcine teschovirus-1 cleavage site, encoded by the linker.

FIG. 16 shows the strategy for assessment of immune responses induced by a DNA vaccine against B16. Female C57/B6 mice were immunized with neoantigen constructs (n=8/group) or empty vector control (n=4). Immunization was carried out at weeks 0, 2, and 4. Splenic lymphocytes were stimulated for 18 hours with peptides for the full neoepitope sequence (MHC II) (from left to right, SEQ ID NOS: 70, 76, and 82) or 15-mer peptides overlapping by 11 amino acids (MHC I) (left from top to bottom, SEQ ID NOS: 71, 72, 73, 74, and 75; center from top to bottom, SEQ ID NOS: 77, 78, 79, 80, and 81; right from top to bottom, SEQ ID NOS: 83, 84, 85, 86, and 87). Peptides were design to not include cleavage sites. IFNγ ELISpot assay was carried out at week 5.

FIG. 17 shows a panel of graphs that show the results from the ELISpot assay, measuring the amount of IFNγ SFU/10e6 splenocytes, corresponding to T cell activation, for the tested full length peptide, pooled 15-mer peptides and individual neoepitopes. The B16 fusion neoantigen construct (top) and the B16 furin/P2A neoantigen construct (bottom) were tested.

FIG. 18 shows proof of concept neoantigen studies: B16 neoantigens. In the absence of an adjuvant, pGX4501 can induce an immune response in mice, particularly in the M33 region, which is a CD8 epitope.

FIG. 19 shows a schematic of the design of poly-neoepitope DNA vaccine against B16 model. pGX4504, pGX5405 and pGX4506 plasmids were used. F indicates a furin cleavage site, encoded by the linker. P2A indicates a porcine teschovirus-1 cleavage site, encoded by the linker.

FIG. 20 shows the strategy for assessment of immune responses induced by a DNA vaccine against B16. Female C57/B6 mice were immunized with neoantigen constructs (n=8/group) or empty vector control (n=4). Immunization was carried out at weeks 0, 2, and 4. Splenic lymphocytes were stimulated for 18 hours with peptides for the full neoepitope sequence (MHC II) (from left to right, SEQ ID NOS: 88, 94, and 100 or 15-mer peptides overlapping by 11 amino acids (MHC I) (left from top to bottom, SEQ ID NOS: 89, 90, 91, 92, and 93; center from top to bottom, SEQ ID NOS: 95, 96, 97, 98, and 99); right from top to bottom, SEQ ID NOS: 101, 102, 103, 104, and 105). Peptides were design to not include cleavage sites. IFNγ ELISpot assay was carried out at week 5.

FIG. 21 shows a panel of results from the ELISpot assay, measuring the amount of IFNγ SFU/10e6 splenocytes, corresponding to T cell activation, for the tested full length peptide, pooled 15-mer peptides and individual neoepitopes. The CT26 fusion neoantigen construct (top), the CT26 furin neoantigen (middle) and the CT26 furin/P2A neoantigen construct (bottom) were tested.

FIG. 22 shows proof of concept neoantigen studies: CT26 neoantigens.

FIG. 23A and FIG. 23B. FIG. 23A shows the pGX4501 Full Length DNA Sequence. (SEQ ID NO: 129) FIG. 23B shows the pGX4501 plasmid map.

FIG. 24A and FIG. 24B. FIG. 24A shows the pGX4503 Full Length DNA Sequence (SEQ ID NO: 131). FIG. 24B shows the pGX4503 plasmid map.

FIG. 25A and FIG. 25B. FIG. 25A shows the pGX4504 Full Length DNA Sequence (SEQ ID NO: 130). FIG. 25B shows the pGX4504 plasmid map.

FIG. 26A and FIG. 26B. FIG. 26A shows the pGX4505 Full Length DNA Sequence (SEQ ID NO: 132). FIG. 26B shows the pGX4505 plasmid map.

FIG. 27A and FIG. 27B. FIG. 27A shows the pGX4506 Full Length DNA Sequence (SEQ ID NO: 133). FIG. 27B shows the pGX4506 plasmid map.

FIG. 28 is a map of the 2999 basepair backbone vector plasmid pVAX1 (Invitrogen, Carlsbad Calif.). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993.

FIG. 29 depicts how the DNA neoantigen vaccine affects tumor growth. C57Bl/6 Mice were immunized three time at 3 week intervals with about 25 micrograms of ID8 plasmids 1 and 2 or pVAX without an insert and challenged with 2×106 ID8 tumor cells injected intraperitoneally 1 week following the final immunization. FIG. 29 depicts survival analysis for mice bearing ID8 tumors treated with ID8 plasmid cocktail (25 micrograms of ID8 plasmid 1 and 25 micrograms id ID8 plasmid 2 formulated together) or 50 microrams of p Vax control plasmid. For this experiment, mice were euthanized upon development of ascites. For all studies, N=10 mice/group. Two-way ANOVA. Gehan-Brelow-Wilcoxon test. *, P<0.01; ****, P<0.0001

FIG. 30 is a restriction map of the pVAX plasmid.

FIG. 31 depicts a both a vaccination protocol of an experiment performed with plasmids and the structure of plasmids in graphic form.

FIG. 32 depicts results of the experiment of Example 11 using the plasmids identified in FIG. 31.

### DETAILED DESCRIPTION

The present disclosure relates to personalized strategies for the treatment of cancer, by administering a therapeutically effective amount of a pharmaceutical composition (e.g., a cancer vaccine) comprising a plurality of tumor specific neo-antigens to a subject (e.g., a mammal such as a human).

### Definitions

The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, In some embodiments, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

As used herein, the terms “activating CD8+ T cells” or “CD8+ T cell activation” refer to a process (e.g., a signaling event) causing or resulting in one or more cellular responses of a CD8+ T cell (CTL), selected from: proliferation, differentiation, cytokine secretion, cytotoxic effector molecule release, cytotoxic activity, and expression of activation markers. As used herein, an “activated CD8+ T cell” refers to a CD8+ T cell that has received an activating signal, and thus demonstrates one or more cellular responses, selected from proliferation, differentiation, cytokine secretion, cytotoxic effector molecule release, cytotoxic activity, and expression of activation markers. Suitable assays to measure CD8+ T cell activation are known in the art and are described herein.

As used herein, the term “adjuvant” is meant to refer to any molecule added to the DNA plasmid vaccines described herein to enhance the immunogenicity of the antigens encoded by the DNA plasmids and the encoding nucleic acid sequences described hereinafter.

As used herein an “antigen” is meant to refer to any substance that will elicit an immune response.

As used herein, the term “anti-tumor response” refers to an immune system response including but not limited to activating T-cells to attack an antigen or an antigen presenting cell.

The term “cancer” as used herein is meant to refer to any disease that is caused by, or results in, inappropriately high levels of cell division, inappropriately low levels of apoptosis, or both. Specific examples of cancer include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood’, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In certain embodiments, the cancer is selected from the group consisting of non small cell lung cancer, melanoma, ovarian cancer, cervical cancer, glioblastoma, urogenital cancer, gynecological cancer, lung cancer, gastrointestinal cancer, head and neck cancer, non-metastatic or metastatic breast cancer, malignant melanoma, Merkel Cell Carcinoma or bone and soft tissue sarcomas, haematologic neoplasias, multiple myeloma, acute myelogenous leukemia, chronic myelogenous leukemia, myelodysplastic syndrome and acute lymphoblastic leukemia, breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer.

The term “checkpoint inhibitor” as used herein is meant to refer to any small molecule chemical compound, antibody, nucleic acid molecule, or polypeptide, or fragments thereof, that inhibits the inhibitory pathways, allowing more extensive immune activity. In certain embodiments, the checkpoint inhibitor is an inhibitor of the programmed death-1 (PD-1) pathway, for example an anti-PD1 antibody, such as, but not limited to Nivoiumab. In other embodiments, the checkpoint inhibitor is an anti-cytotoxic T-lymphocyte-associated antigen (CTLA-4) antibody. In further additional embodiments, the checkpoint inhibitor is targeted at a member of the TNF superfamily such as CD40, OX40, CD 137, GITR, CD27 or TIM-3. In some cases targeting a checkpoint inhibitor is accomplished with an inhibitory antibody or similar molecule. In other cases, it is accomplished with an agonist for the target; examples of this class include the stimulatory targets OX40 and GITR.

The term “combination therapy” as used herein is meant to refer to administration of one or more therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention may comprise a pooled sample of tumor specific neoantigens and a checkpoint inhibitor administered at the same or different times, or the)′ can be formulated as a single, co-formulated pharmaceutical composition comprising the two compounds. As another example, a combination of the present invention (e.g., DNA neoantigen vaccines and a checkpoint inhibitor) may be formulated as separate pharmaceutical compositions that can be administered at the same or different time. As used herein, the term “simultaneously” is meant to refer to administration of one or more agents at the same time. For example, in certain embodiments, a cancer vaccine or immunogenic composition and a checkpoint inhibitor are administered simultaneously). Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered orally. The components may be administered in any therapeutically effective sequence. A “combination” embraces groups of compounds or non-drug therapies useful as part of a combination therapy.

As used herein, the term “electroporation,” “electro-permeabilization,” or “electro-kinetic enhancement” (“EP”), are used interchangeably and are meant to refer to the use of a transmembrane electric field pulse to induce microscopic pathways (pores) in a bio-membrane; their presence allows biomolecules such as plasmids, oligonucleotides, siRNA, drugs, ions, and/or water to pass from one side of the cellular membrane to the other.

By “fragment” is meant a portion of a polypeptide or nucleic acid molecule. This portion contains, preferably, at least 5%, 10%, 20%, 30%, 40%, 50%, 60%, 70%, 80%, or 90% of the entire length of the reference nucleic acid molecule or polypeptide. A fragment may contain 5, 10, 20, 30, 40, 50, 60, 70, 80, 90, or 100, 200, 300, 400, 500, 600, 700, 800, 900, 1000 or more nucleotides or amino acids.

As used herein, the term “genetic construct” is meant to refer to the DNA or RNA molecules that comprise a nucleotide sequence which encodes protein. The coding sequence includes initiation and termination signals operably linked to regulatory elements including a promoter and polyadenylation signal capable of directing expression in the cells of the individual to whom the nucleic acid molecule is administered.

The term “host cell” as used herein is meant to refer to a cell that can be used to express a nucleic acid, e.g., a nucleic acid of the disclosure. can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “hybridize” as used herein is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The term “immune checkpoint” as used herein is meant to refer to inhibitory pathways that slow down or stop immune reactions and prevent excessive tissue damage from uncontrolled activity of immune cells.

The term “immune response” is used herein is meant to refer to the activation of a host's immune system, e.g., that of a mammal, in response to the introduction of nucleic acid molecules comprising a nucleotide sequence encoding neoantigens a described herein.

The term “isolated” as used herein means that the polynucleotide or polypeptide or fragment, variant, or derivative thereof has been essentially removed from other biological materials with which it is naturally associated, or essentially free from other biological materials derived, e.g., from a recombinant host cell that has been genetically engineered to express the polypeptide of the invention.

The term “ligand” as used herein is meant to refer to a molecule which has a structure complementary to that of a receptor and is capable of forming a complex with this receptor. According to embodiments of the invention, a ligand is to be understood as meaning in particular a peptide or peptide fragment which has a suitable length and suitable binding motives in its amino acid sequence, so that the peptide or peptide fragment is capable of forming a complex with proteins of MHC class I or MHC class II.

The terms “MHC molecules”, “MHC proteins” or “HLA proteins” as used herein are meant to refer to proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins and tumor antigens. The molecules of MHC class II present protein antigens originating from exogenous sources, for example bacterial products. The cellular biology and the expression patterns of the two MHC classes are adapted to these different roles.

MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin.

MHC molecules of class II consist of an α-chain and a β-chain and are capable of binding a peptide of about 15 to 24 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

The term “neoantigen” as used herein is meant to refer to a class of tumor antigens which arises from tumor-specific mutations in expressed protein of a subject. In some embodiments, the neoantigen is derived directly from a tumor of a subject. This is as opposed to a known tumor associated antigen which may be a consensus sequence known to elicit an immune response against a cell expressing the tumor antigen but not necessarily expressed by a tumor derived the subject.

The term “neoantigen mutation” as used herein refers to a mutation that is predicted to encode a neoantigenic peptide.

The term “pharmaceutically acceptable” as used herein refers to approved or approvable by a regulatory agency of the Federal or a state government or listed in the U.S. Pharmacopeia or other generally recognized pharmacopeia for use in animals, including humans.

The term “pharmaceutically acceptable excipient, carrier or diluent” as used herein is meant to refer to an excipient, carrier or diluent that can be administered to a subject, together with an agent, and which does not destroy the pharmacological activity thereof and is nontoxic when administered in doses sufficient to deliver a therapeutic amount of the agent.

The term “pharmaceutically acceptable salt” of tumor specific neoantigens as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonie, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenyiacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide of the invention. That is, e.g., a purified polypeptide of the present invention is a polypeptide that is at least from about 70 to about 100% pure, i.e., the polypeptide is present in a composition wherein the polypeptide constitutes from about 70 to about 100% by weight of the total composition. In some embodiments, the purified polypeptide of the present invention is from about 75% to about 99% by weight pure, from about 80% to about 99% by weight pure, from about 90 to about 99% by weight pure, or from about 95% to about 99% by weight pure.

The term “receptor” as used herein, is meant to refer to a biological molecule or a molecule grouping capable of binding a ligand. A receptor may serve, to transmit information in a cell, a cell formation or an organism. The receptor comprises at least one receptor unit and preferably two receptor units, where each receptor unit may consist of a protein molecule, in particular a glycoprotein molecule. The receptor has a structure which complements that of a ligand and may complex the ligand as a binding partner. The information is transmitted in particular by conformational changes of the receptor following complexation of the ligand on the surface of a cell. According to embodiments of the invention, a receptor is to be understood as meaning in particular proteins of MHC classes I and II capable of forming a receptor/ligand complex with a ligand, in particular a peptide or peptide fragment of suitable length.

As used herein, the term “small molecule” refers to a low molecular weight (<900 daltons) organic compound that may help regulate a biological process, with a size on the order of 10 9 m. Most drugs are small molecules.

As used herein, the terms “subject,” “individual,” “host,” and “patient,” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in other embodiments the subject is a human.

As used herein, “patient in need thereof” or “subject in need thereof” refers to a living organism suffering from or prone to a disease or condition that can be treated by administration of a vaccine (or pharmaceutical composition comprising a neoantigen DNA vaccine) according to the described invention. A “patient in need thereof” or “subject in need” may also refer to a living organism that is receiving a neoantigen DNA vaccine (or pharmaceutical composition comprising a neoantigen DNA vaccine) according to the described invention, or has received a neoantigen DNA vaccine (or pharmaceutical composition comprising a neoantigen DNA vaccine) according to the described invention; or has a tumor or Non-limiting examples include humans, other mammals, bovines, rats, mice, dogs, monkeys, goat, sheep, cows, deer, and other non-mammalian animals. In embodiments, a patient or subject is human.

The term “T-cell epitope” as used herein is meant to refer to a peptide sequence which can be bound by the MHC molecules of class I or II in the form of a peptide-presenting MHC molecule or MHC complex and then, in this form, be recognized and bound by cytotoxic T-lymphocytes or T-helper cells, respectively.

The term “therapeutic effect” as used herein is meant to refer to some extent of relief of one or more of the symptoms of a disorder (e.g., a neoplasia or tumor) or its associated pathology. A “therapeutically effective amount” as used herein is meant to refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved. In some embodiments, the therapeutically effective amount is an amount effective to shrink a solid tumor by about 2% in total mass as compared to its mass or estimated mass before treatment, by about 4% in total mass, by about 6% in total mass, by about 8% in total mass, by about 10% in total mass, by about 15% in total mass, by about 20% in total mass, by about 25% in total mass, by about 30% in total mass, by about 35% in total mass, by about 40% in total mass, by about 45% in total mass, or by about 50% in total mass as compared to the total mass of the solid tumor before the treatment.

The terms “treat,” “treated,” “treating,” “treatment,” and the like as used herein are meant to refer to reducing or ameliorating a disorder and/or symptoms associated therewith (e.g., a cancer or tumor). “Treating” may refer to administration of the neoantigen vaccines described herein to a subject after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

As used herein, the term “treating cancer” is not intended to be an absolute term. In some aspects, the compositions and methods of the invention seek to reduce the size of a tumor or number of cancer cells, cause a cancer to go into remission, or prevent growth in size or cell number of cancer cells. In some circumstances, treatment with the leads to an improved prognosis.

The term “therapeutic effect” as used herein is meant to refer to some extent of relief of one or more of the symptoms of a disorder (e.g., a neoplasia or tumor) or its associated pathology. A “therapeutically effective amount” as used herein is meant to refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered agent Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH.sub.2, NHR, N.sub.2 or CN, wherein R is C.sub.1-C.sub.6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in US20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

As used herein, the term “nucleic acid molecule” comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also includes a plasmid containing one or more nucleotide sequences that encode one or a plurality of neoantigens. In some embodiments, the disclosure relates to a pharmaceutical composition comprising a first, second, third or more nucleic acid molecule, each of which encoding one or a plurality of neoantigens and at least one of each plasmid comprising one or more of the Formulae disclosed herein.

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The phrase “stringent hybridization conditions” or “stringent conditions” as used herein is meant to refer to conditions under which a nucleic acid molecule will hybridize another nucleic acid molecule, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present in excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g. 10 to 50 nucleotides) and at least about 600 C for longer probes, primers or oligonucleotides. Stringent conditions may also be achieved with the addition of destabilizing agents, such as formamide.

By “substantially identical” is meant nucleic acid molecule (or polypeptide) exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif, and Baron et al., 1995, Nucleic Acids Res. 23:3605-06.

As used herein, the term “sample” refers generally to a limited quantity of something which is intended to be similar to and represent a larger amount of that something. In the present disclosure, a sample is a collection, swab, brushing, scraping, biopsy, removed tissue, or surgical resection that is to be testing for the absence, presence or grading of a hyperproliferative tissue, which, in some cases is cancerous tissue or one or a plurality of cells. In some embodiments, samples are taken from a patient or subject that is believed to have a cancer, hyperplasia, pre-cancerous or comprise one or more tumor cells. In some embodiments, a sample believed to contain one or more hyperproliferative cells is compared to a “control sample” that is known not to contain one or more hyperproliferative cells. This disclosure contemplates using any one or a plurality of disclosed samples herein to identify, detect, sequence and/or quantify the amount of neoantigens (highly or minimally immunogenic) within a particular sample. In some embodiments, the methods relate to the step of exposing a swab, brushing or other sample from an environment to a set of reagents sufficient to isolate and/or sequence the DNA and RNA of one or a plurality of cells in the sample.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. The disclosure relates to any one or plurality of vectors that comprise nucleic acid sequences encoding any one or plurality of amino acid sequence disclosed herein.

The term “vaccine” as used herein is meant to refer to a composition for generating immunity for the prophylaxis and/or treatment of diseases (e.g., cancer). Accordingly, vaccines are medicaments which comprise antigens and are intended to be used in humans or animals for generating specific defense and protective substance by vaccination. A “vaccine composition” or a “neoantigen vaccine composition” can include a pharmaceutically acceptable excipient, earner or diluent.

### Compositions

The present disclosure is based, at least in part, on the ability to identify all, or substantially all, of the mutations within a cancer/tumor (e.g., translocations, inversions, large and small deletions and insertions, missense mutations, splice site mutations, etc.). In particular, these mutations are present in the genome of cancer/tumor cells of a subject, but not in normal tissue from the subject. The disclosure relates to the innovative discovery that administering pharmaceutical compositions comprising the nucleic acid sequences that encode from about 1 to about 100 different amino acid sequences that represent a milleu of mutations in several different cancer cells Such mutations are of particular interest if they lead to changes that result in a protein with an altered amino acid sequence that is unique to the patient's cancer/tumor (e.g., a neo-antigen).

In one aspect, the present disclosure features a nucleic acid molecule comprising a nucleic acid sequence comprising Formula I:

[(AEDⁿ)-(linker)]ₙ-[AEDⁿ⁺¹],

wherein the AED is an independently selectable antigen expression domain comprising an expressible nucleic acid sequence, wherein AEDⁿ is referred to as antigen expression domain and wherein AEDⁿ⁺¹ is referred to as antigen expression domain 2; wherein the each linker is independently selectable from about 0 to about 300 natural or non-natural nucleic acids in length, wherein the antigen expression domain 1 is independently selectable from about 12 to about 15,000 nucleotides in length and encodes an epitope; wherein the antigen expression domain 2 is independently selectable from about 12 to about 15,000 nucleotides in length and encodes an epitope; and wherein n is any positive integer from about 1 to about 500.

In some embodiments, each linker is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, each linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, each linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, each linker is about 21 natural or non-natural nucleic acids in length.

In some embodiments, the length of each linker according to Formula I is different. For example, in some embodiments, the length of a first linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length, and the length of a second linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length, where the length of the first linker is different from the length of the second linker. Various configurations can be envisioned by the present disclosure, where Formula I comprises 1, 2, 3, 4, 5, 6, 7, 8, 9, 10 or more linkers wherein the linkers are of similar or different lengths.

In certain embodiments, two linkers can be used together, in a nucleotide sequence that encodes a fusion peptide. Accordingly, in some embodiments, the first linker is independently selectable from about 0 to about 25 natural or non-natural nucleic acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the second linker is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the first linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, the second linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length.

In certain embodiments, antigen expression domain 1 and antigen expression domain 2 comprise a nucleic acid sequence that encodes a particular tumor neoantigen. In some embodiments, antigen expression domain 1 encodes a CD4 neoepitope. In some embodiments, antigen expression domain 1 encodes a CD8 neoepitope. In some embodiments, antigen expression domain 2 encodes a CD4 neoepitope. In some embodiments, antigen expression domain 2 encodes a CD8 neoepitope. In some embodiments, antigen domain 1 encodes a CD8 neoepitope and antigen expression domain 2 encodes a CD8 neoepitope. A CD4 neoepitope is an epitope that is recognized by CD4+ T cells. A CD8 neoepitope is an epitope that is recognized by CD8+ T cells.

The disclosures relates to a nucleic acid sequence comprising a plurality of antigen expression domains encoding at least two neoantigens separated by one or a plurality of linkers. In some embodiments, the antigen expression domain encodes an amino acid sequence from about 3 to about 100 amino acids in length. In some embodiments, there is at least one linker encoding a linker from about 3 to about 25 amino acids in length. In some embodiment, the linker sequence separate each antigen expression domain. In some embodiments, the nucleic acid sequence comprises 1, 2, 3, 4, 5, 6, 7, 8, 9, or 10 or more linkers. In some embodiments, the nucleic acid sequence comprises 1, 2, 3, 4, 5, 6, 7, 8, 9, or 10 or more linkers, at least one or more are comprise furin linkers. In some embodiments, the nucleic acid sequence comprises at least 2, 3, 4, 5, 6, 7, 8, 9, or 10 or more linker domains and the nucleic acid sequence comprises Formula I(a):

(AED¹)-(linker)-(AED²)-(linker)]ₙ

wherein each AED is independently selectable from any one or plurality of tumor associated antigens from a subject and wherein nis any positive integer from about 1 to about 50 and wherein each “linker” is a nucleic acid sequence encoding one or a plurality of amino acid cleavage sites. Each linker may be the same or independently selectable to comprise one or a plurality of the linkers disclosed herein. In some embodiments, the linker is a furin cleavage site from about 9 to about 105 nucleotides in length and encodes an amino acid sequence that is an amino acid cleavage site. In some embodiments, the nucleic acid sequence is a component of a nucleic acid molecule. In some compositions contemplated herein, the composition comprises 1, 2, 3, 4, 5, or more nucleic acid molecules each of which expressing any of the patterns or formulae of AEDs disclosed herein.

The disclosures also relates to a nucleic acid sequence comprising a coding region and a non-coding region, the coding region consisting of the Formula I(b):

[(AED¹)-(linker)-(AED²)-(linker)]_{n.}-(AED³)]ₙ₊₁,

wherein n is a positive integer from about 1 to about 20, wherein each “linker” encode one or a plurality of amino acid cleavages sequences, and wherein the non-coding region comprises at least one regulatory sequence operably linked to one or more AEDs; and wherein, in the 5′ ot 3′ orientation, AED³ is the terminal antigen expression domain in a sequence of AEDs. In some embodiments, the regulatory is any of the regulatory sequences depicted in the Figures or a functional fragment that comprises at least 70%, 75%, 80%, 85%, 90%, 91%, 92%, 93%, 94%, 95%, 96% 97%, 98% or 99% homologous to the regulatory sequence depicted in the Figures.

In some embodiments, the nucleic acid molecule or sequence of the disclosure comprises a plurality of antigen expression domains encoding at least two neoantigens separated by one or a plurality of linkers. In some embodiments, the antigen expression domain encodes an amino acid sequence from about 3 to about 100 amino acids in length. In some embodiments, there is at least one linker encoding a linker from about 3 to about 25 amino acids in length. In some embodiment, the linker sequence separate each antigen expression domain. In some embodiments, the nucleic acid sequence comprises 1, 2, 3, 4, 5, 6, 7, 8, 9, or 10 or more linkers. In some embodiments, the nucleic acid sequence comprises 1, 2, 3, 4, 5, 6, 7, 8, 9, or 10 or more linkers, at least one or more are comprise furin linkers. In some embodiments, the nucleic acid sequence comprises at least 2, 3, 4, 5, 6, 7, 8, 9, or 10 or more linker domains and the nucleic acid sequence comprises Formula I(a):

(AED¹)-(linker)-(AED²)-(linker)]ₙ

wherein each AED is independently selectable from any one or plurality of tumor associated antigens from a subject and wherein n is any positive integer from about 1 to about 50 and wherein each “linker” is a nucleic acid sequence encoding one or a plurality of amino acid cleavage sites. Each linker may be the same or independently selectable to comprise one or a plurality of the linkers disclosed herein.

In some embodiments, the antigen expression domain 1 and/or 2 is independently selectable from about 12 to about 15,000 nucleotides in length, about 50 to about 15,000 nucleotides in length, about 100 to about 15,000 nucleotides in length, about 500 to about 15,000 nucleotides in length, about 1,000 to about 15,000 nucleotides in length, about 5,000 to about 15,000 nucleotides in length, about 10,000 to about 15,000 nucleotides in length. In other embodiments, the antigen expression domain 1 is about 12, about 25, about 50, about 75, about 100, about 200, about 300, about 400, about 500, about 600, about 700, about 800, about 900, about 1,000, about 2,000, about 3,000, about 4,000, about 5,000, about 6,000, about 7,000, about 8,000, about 9,000, about 10,000, about 11,000, about 12,000, about 13,000, about 14,000, about 15,000 nucleotides in length. In some embodiments, the antigen expression domain 2 is independently selectable from about 12 to about 15,000 nucleotides in length, about 50 to about 15,000 nucleotides in length, about 100 to about 15,000 nucleotides in length, about 500 to about 15,000 nucleotides in length, about 1,000 to about 15,000 nucleotides in length, about 5,000 to about 15,000 nucleotides in length, about 10,000 to about 15,000 nucleotides in length. In another embodiment, the antigen expression domain 2 is about 12, about 25, about 50, about 75, about 100, about 200, about 300, about 400, about 500, about 600, about 700, about 800, about 900, about 1,000, about 2,000, about 3,000, about 4,000, about 5,000, about 6,000, about 7,000, about 8,000, about 9,000, about 10,000, about 11,000, about 12,000, about 13,000, about 14,000 about 15,000 nucleotides in length.

In other embodiments, the antigen expression domain 1 or the antigen expression domain 2 are independently selectable from about 20 to about 2,000 nucleotides in length. In some embodiments, the antigen expression domain 1 is about 20 to about 2,000 nucleotides in length, about 50 to about 2,000 nucleotides in length, about 100 to about 2,000 nucleotides in length, about 500 to about 2,000 nucleotides in length, about 1500 to about 2,000 nucleotides in length. In other embodiments, the antigen expression domain 1 is about 20, about 30, about 40, about 50, about 60, about 70, about 80, about 90, about 100, about 200, about 300, about 400, about 500, about 600, about 700, about 800, about 900, about 1000, about 1,100, about 1,200, about 1,300, about 1,400, about 1,500, about 1,600, about 1,700, about 1,800, about 1900, about 2000 nucleotides in length. In some embodiments, the antigen expression domain 2 is about 20 to about 2,000 nucleotides in length, about 50 to about 2,000 nucleotides in length, about 100 to about 2,000 nucleotides in length, about 500 to about 2,000 nucleotides in length, about 1500 to about 2,000 nucleotides in length. In other embodiments, the antigen expression domain 2 is about 20, about 30, about 40, about 50, about 60, about 70, about 80, about 90, about 100, about 200, about 300, about 400, about 500, about 600, about 700, about 800, about 900, about 1000, about 1,100, about 1,200, about 1,300, about 1,400, about 1,500, about 1,600, about 1,700, about 1,800, about 1900, about 2000 nucleotides in length.

In some embodiments, the antigen expression domain 1 and/or the antigen expression domain 2 are independently selectable from about 15 to about 150 nucleotides in length, for example about 15 to about 150 nucleotides in length, about 15 to about 125 nucleotides in length, about 15 to about 100, about 15 to about 90 nucleotides in length, about 15 to about 90 nucleotides in length, about 15 to about 80 nucleotides in length, about 15 to about 70 nucleotides in length, about 15 to about 60 nucleotides in length, about 15 to about 50 nucleotides in length, about 15 to about 40 nucleotides in length, about 15 to about 30 nucleotides in length, about 15 to about 20 nucleotides in length.

In some embodiments, the antigen expression domain 1 and/or antigen expression domain 2 is independently selectable from about 15 to about 100 nucleotides in length, for example about 3 to about 120 nucleotides in length, from about 15 to about 100, from about 15 to about 90 nucleotides in length, about 15 to about 90 nucleotides in length, about 15 to about 80 nucleotides in length, about 15 to about 70 nucleotides in length, about 15 to about 60 nucleotides in length, about 15 to about 50 nucleotides in length, about 15 to about 40 nucleotides in length, about 15 to about 30 nucleotides in length, about 15 to about 20 nucleotides in length.

In some embodiments, the antigen expression domain 1 and/or antigen expression domain 2 is independently selectable from about 15 to about 50 nucleotides in length, for example about 15 to about 50 nucleotides in length, about 15 to about 40 nucleotides in length, about 15 to about 30 nucleotides in length, about 15 to about 20 nucleotides in length.

In some embodiments, n is any positive integer from about 1 to about 500. In some embodiments, n is any positive integer from about 1 to about 500, from about 10 to about 500, from about 50 to about 500, from about 100 to about 500, from about 200 to about 500, from about 300 to about 500, from about 400 to about 500. In other embodiments, n is any positive integer of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 120, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500.

In some embodiments, n is a positive integer from about 5 to about 30, from about 5 to about 25, from about 5 to about 20, from about 5 to about 15, from about 5 to about 10.

In some embodiments, n is a positive integer from about 2 to about 100, from about 2 to about 90, from about 2 to about 80, from about 2 to about 70, from about 2 to about 60, from about 2 to about 50, from about 2 to about 40, from about 2 to about 30, from about 2 to about 20, from about 2 to about 10.

In some embodiments, n is a positive integer from about 2 to about 58, from about 3 to about 58, from about 4 to about 58, from about 5 to about 58, from about 6 to about 58, from about 7 to about 58, from about 8 to about 58, from about 9 to about 58, from about 10 to about 58, from about 11 to about 58, from about 12 to about 58, from about 13 to about 58, from about 14 to about 58, from about 15 to about 58, from about 16 to about 58, from about 17 to about 58, from about 18 to about 58, from about 19 to about 58, from about 20 to about 58, from about 21 to about 58, from about 22 to about 58, from about 23 to about 58, from about 24 to about 58, from about 25 to about 58, from about 26 to about 58, from about 27 to about 58, from about 28 to about 58, from about 29 to about 58, from about 30 to about 58, from about 31 to about 58, from about 32 to about 58, from about 33 to about 58, from about 34 to about 58, from about 35 to about 58, from about 36 to about 58, from about 37 to about 58, from about 38 to about 58, from about 39 to about 58, from about 40 to about 58, from about 41 to about 58, from about 42 to about 58, from about 43 to about 58, from about 44 to about 58, from about 45 to about 58, from about 46 to about 58, from about 47 to about 58, from about 48 to about 58, from about 49 to about 58, from about 50 to about 58, from about 51 to about 58, from about 52 to about 58, from about 53 to about 58, from about 54 to about 58, from about 55 to about 58, from about 56 to about 58, from about 57 to about 58.

In one In some embodiments, n is a positive integer from about 2 to about 29, from about 3 to about 29, from about 4 to about 29, from about 5 to about 29, from about 6 to about 58, from about 7 to about 29, from about 8 to about 29, from about 9 to about 29, from about 10 to about 29, from about 11 to about 29, from about 12 to about 29, from about 13 to about 29, from about 14 to about 29, from about 15 to about 29, from about 16 to about 29, from about 17 to about 29, from about 18 to about 29, from about 19 to about 29, from about 20 to about 29, from about 21 to about 29, from about 22 to about 29, from about 23 to about 29, from about 24 to about 29, from about 25 to about 29, from about 26 to about 29, from about 27 to about 29, from about 28 to about 29.

In some embodiments, the antigen expression domain 1 or antigen expression domain 2 is independently selectable from about 50 to about 10,000 nucleotides in length, for example about 50 to about 15,000 nucleotides in length, about 100 to about 15,000 nucleotides in length, about 500 to about 15,000 nucleotides in length, about 1,000 to about 15,000 nucleotides in length, about 5,000 to about 15,000 nucleotides in length, about 10,000 to about 15,000 nucleotides in length, and n is any positive integer from about 6 to about 26, for example about 6, about 7, about 8, about 9, about 10, about 11, about 12, about 13, about 14, about 15, about 16, about 17, about 18, about 19, about 20, about 21, about 22, about 23, about 24, about 25, or about 26.

In some embodiments, a nucleic acid molecule comprises a nucleic acid sequence comprising Formula I ([(AEDⁿ)-(linker)]ₙ-[AEDⁿ⁺¹]), wherein the each linker is independently selectable from about 0 to about 25 natural or non-natural nucleic acids in length. In some embodiments, a nucleic acid molecule comprises a nucleic acid sequence comprising Formula I ([(AEDⁿ) (linker)]ₙ-[AEDⁿ⁺¹]), wherein the each linker is independently selectable from about 0 to about 25 natural or non-natural nucleic acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25. In some embodiments, each linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, each linker is about 21 natural or non-natural nucleic acids in length. In certain embodiments, two linkers can be used together, in a fusion. Accordingly, in some embodiments, the first linker is independently selectable from about 0 to about 25 natural or non-natural nucleic acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the second linker is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the first linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, the second linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length.

In some embodiments, the at least one linker comprises from about 15 to about 300 nucleotides and encodes a n amino acid cleavage site. In some embodiments, each linker positioned between each AED is the same nucleotide sequence comprising from about 15 to about 120 nucleotides and encodes an amino acid cleavage site

In some embodiments, the formula (e.g. [(AEDⁿ)-(linker)]ₙ-[AEDⁿ⁺¹]) comprises at least 1, 2, 3, 4, 5, 6, 7, 8, 9, 10 or more linkers.

In some embodiments, the formula comprises at least a first linker and a second linker.

In some embodiments, the formula comprises at least a first linker, a second linker, and a third linker.

In some embodiments, the formula comprises at least a first linker, a second linker, a third linker, and a fourth linker.

In some embodiments, the formula comprises at least a first linker, a second linker, a third linker, a fourth linker, and a fifth linker.

In a further embodiment, the at least one linker comprises a furin protease cleavage site.

Furin is a protease which resides in the trans-Golgi network of eukaryotic cells. Its function is to cleave proteins at a step just prior to their delivery to their final cellular destination. Furin recognizes a consensus amino acid sequence, RXRR, RXRK or KXKR (where X is any amino acid, Moehring et al., 1993, incorporated by reference in its entirety herein) and cuts proteins which contain these sequences when they reach the trans-Golgi network. Furin is a Ca2+-dependent serine endoprotease that cleaves protein precursors with a high specificity after the multiple basic motifs shown in Table 1 below.

TABLE 1

| | |
|---|---|
| Canonic | -RP⁴-XP³-(K/R)P²-RP¹↓XP¹′-XP²′-XP³′-XP⁴′ |
| Alternative | -RP⁶-XP⁵-XP⁴-XP³-(K/R)P²-RP¹↓XP¹′-XP²′-XP³′-XP⁴′ |
| Minimal | -RP⁴-XP³-XP²-XP³-RP¹↓XP¹′-XP²′-XP³′-XP⁴′ |

In certain embodiments, the one or plurality of nucleic acid molecules encode a furin-sensitive cleavage site selected from the sequence R-X-[R/K]-R, where R denotes arginine, X is any amino acid, and K is lysine. The “R/K” indicates that this amino acid may be either arginine or lysine.

In certain embodiments, a furin cleavage site is introduced after the antigen domain 1 and/or the antigen domain 2 (e.g. [(AEDⁿ)-(linker)]ₙ-[AEDⁿ⁺¹]).

In some embodiments, the at least one linker comprises from about 15 to about 300 nucleotides and encodes a cleavage site, wherein the at least one linker comprises a 2A cleavage site. In some embodiments, the at least one linker comprises from about 15 to about 300 nucleotides and encodes a cleavage site, wherein the at least one linker comprises a porcine teschovirus-1 2A (P2A) cleavage site.

A 2A peptide is a “self-cleaving” small peptide. The average length of 2A peptides is 18-22 amino acids. The designation “2A” refers to a specific region of picornavirus polyproteins. Of the 2A peptides identified to date, four are widely used in research: FMDV 2A (abbreviated herein as F2A); equine rhinitis A virus (ERAV) 2A (E2A); porcine teschovirus-1 2A (P2A) and Thoseaasigna virus 2A (T2A). The former three viruses belong to picornaviruses and the latter is an insect virus. DNA and corresponding amino acid sequences of various 2A peptides are shown below in Table 2. Underlined sequences encode amino acids GSG, which were added to improve cleavage efficiency. P2A indicates porcine teschovirus-1 2A; T2A, Thoseaasigna virus 2A; E2A, equine rhinitis A virus (ERAV) 2A; F2A, FMDV 2A.

TABLE 2

| | |
|---|---|
| P2A | GGA AGC GGA GCT ACT AAC TTC AGC CTG CTG AAG CAG GCT GGA |
| | G S G A T N F S L L K Q A G |
| | GAC GTG GAG GAG AAC CCT GGA CC SEQ ID NO: 106 |
| | D V E E N P G P SEQ ID NO: 107 |
| T2A | GGA AGC GGA GAG GGC AGA GGA AGT CTG CTA ACA TGC GGT GAC |
| | G A G E G R G A L L T C G D |
| | GTC GAG GAG AAT CCT GGA CC SEQ ID NO: 108 |
| | V E E N P G F SEQ ID NO: 109 |
| E2A | GGA AGC GGA CAG TGT ACT AAT TAT GCT CTC TTG AAA TTG GCT |
| | G S G Q C T N Y A L L K L A |
| | GGA GAT GTT GAG AGC AAC CCT GGA CC SEQ ID NO: 110 |
| | G D V E S N P G P SEQ ID NO: 111 |
| F2A | GGA AGC GGA GTG AAA CAG ACT TTG AAT TTT GAC CTT CTC AAG |
| | G S G V K Q T L N F D L L K |
| | ITG GCG GGA GAC GTG GAG TCC AAC CCT GGA C SEQ ID NO: 112 |
| | L A G D V E S N P G SEQ ID NO: 113 |

In some embodiments, the formula comprises at least a first linker and a second linker, wherein the first and second linker comprise a furin protease cleavage site.

In some embodiments, the formula comprises at least a first linker, a second linker, and a third linker, wherein the first, second and third linker comprise a furin protease cleavage site.

In some embodiments, the formula comprises at least a first linker, a second linker, a third linker, and a fourth linker, wherein the first, second, third and fourth linker comprise a furin protease cleavage site.

In some embodiments, the formula comprises at least a first linker, a second linker, a third linker, a fourth linker, and a fifth linker, wherein the first, second, third, fourth and fifth linker comprise a furin protease cleavage site.

In some embodiments, the formula comprises at least a first linker, a second linker, a third linker, a fourth linker, and a fifth linker, wherein the first, second, third, fourth and fifth linker comprise a furin protease cleavage site.

In another embodiment, the formula comprises at least a first linker and a second linker, wherein the first and second linker comprise a P2A protease cleavage site.

In some embodiments, the formula comprises at least a first linker, a second linker, and a third linker, wherein the first, second and third linker comprise a P2A cleavage site.

In some embodiments, the formula comprises at least a first linker, a second linker, a third linker, and a fourth linker, wherein the first, second, third and fourth linker comprise a P2A cleavage site.

In some embodiments, the formula comprises at least a first linker, a second linker, a third linker, a fourth linker, and a fifth linker, wherein the first, second, third, fourth and fifth linker comprise a P2A cleavage site.

In some embodiments, the formula comprises at least a first linker, a second linker, a third linker, a fourth linker, a fifth linker, or more wherein the first, second, third, fourth, fifth linker, or more linkers comprise a P2A protease cleavage site.

In some embodiments, the formula comprises at least a first linker and a second linker, wherein at least one of the first or second linkers comprise a furin protease cleavage site.

In some embodiments, the formula comprises at least a first linker, a second linker, and a third linker, wherein at least one of the first, second or third linkers comprise a furin protease cleavage site.

In some embodiments, the formula comprises at least a first linker, a second linker, a third linker, and a fourth linker, at least one of the first, second, third or fourth linkers comprise a furin protease cleavage site.

In some embodiments, the formula comprises at least a first linker, a second linker, a third linker, a fourth linker, and a fifth linker, at least one of the first, second, third, fourth or fifth linkers comprise a furin protease cleavage site.

In some embodiments, the formula comprises at least a first linker and a second linker, wherein at least one of the first or second linkers comprise a P2A protease cleavage site.

In some embodiments, the formula comprises at least a first linker, a second linker, and a third linker, wherein at least one of the first, second or third linkers comprise a P2A protease cleavage site.

In some embodiments, the formula comprises at least a first linker, a second linker, a third linker, and a fourth linker, at least one of the first, second, third or fourth linkers comprise a P2A protease cleavage site.

In some embodiments, the formula comprises at least a first linker, a second linker, a third linker, a fourth linker, and a fifth linker, at least one of the first, second, third, fourth or fifth linkers comprise a P2A protease cleavage site.

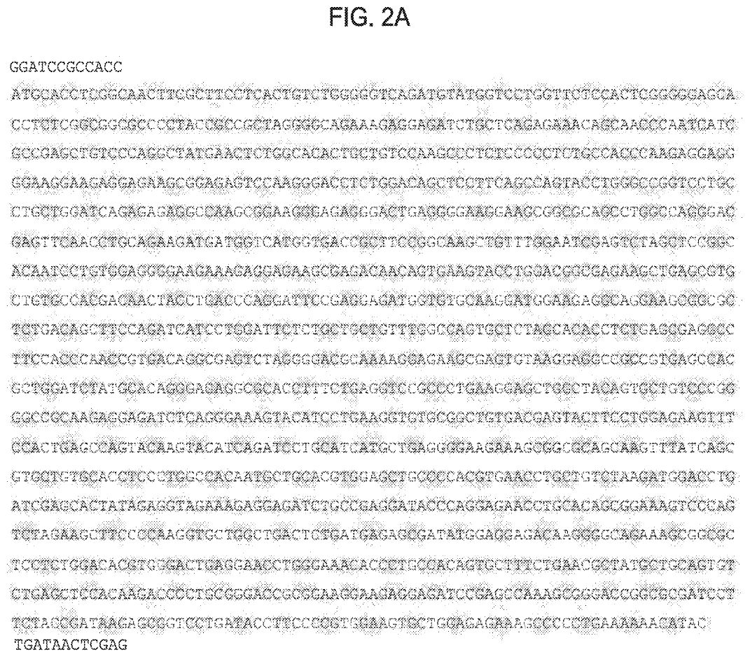

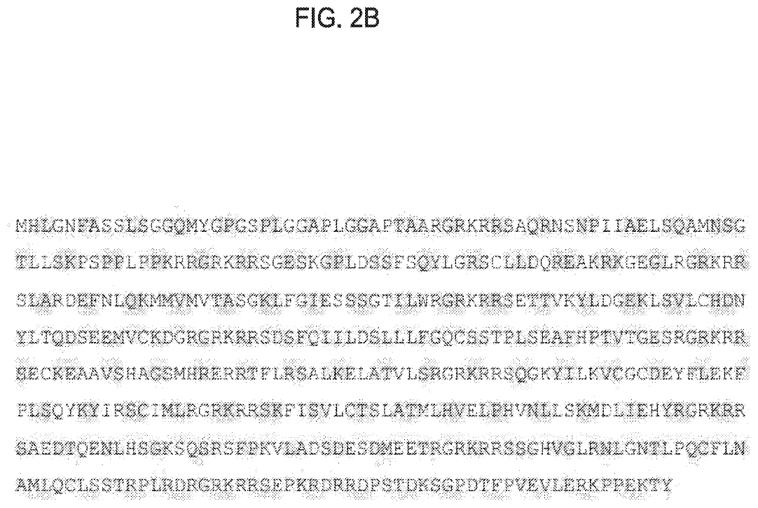

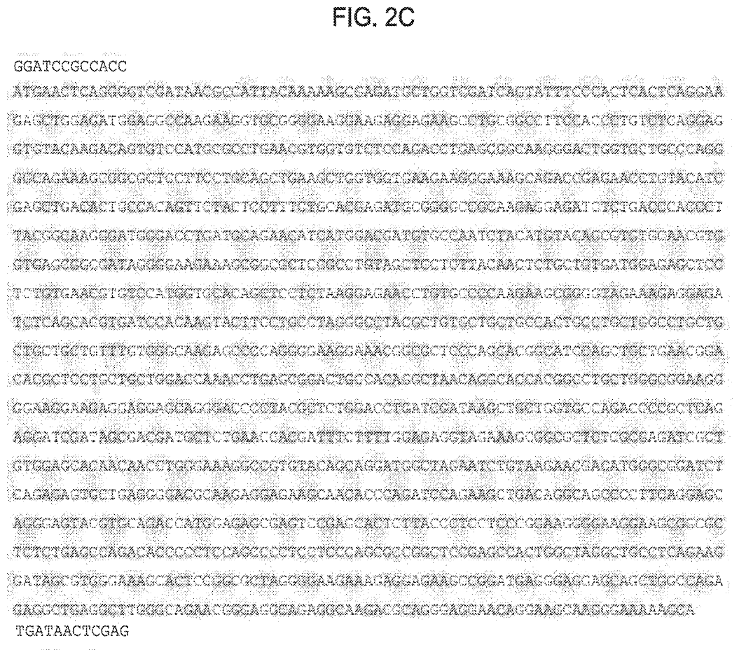

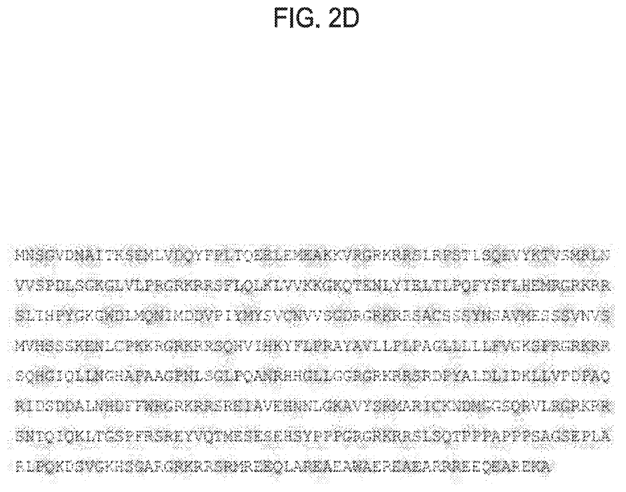

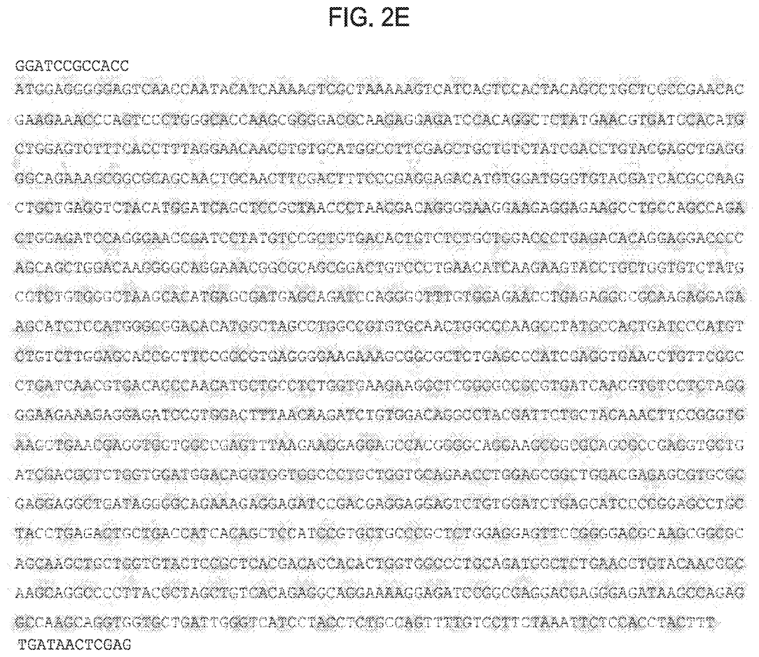

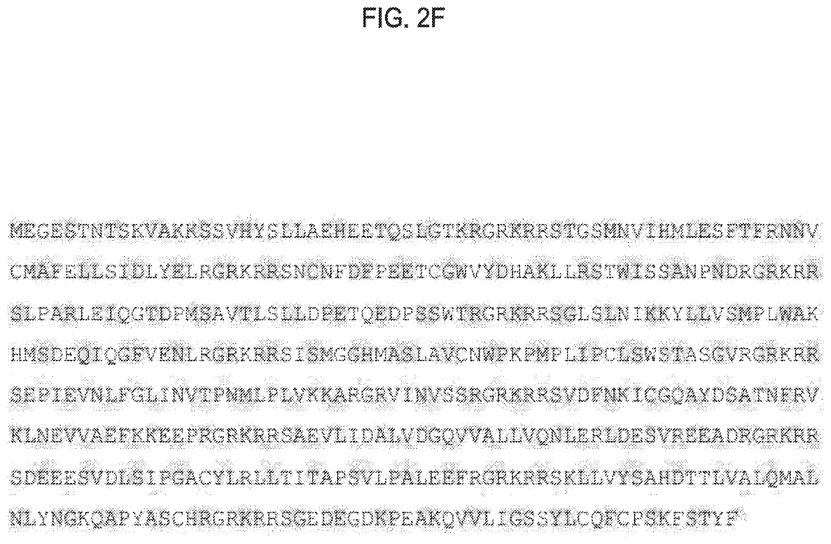

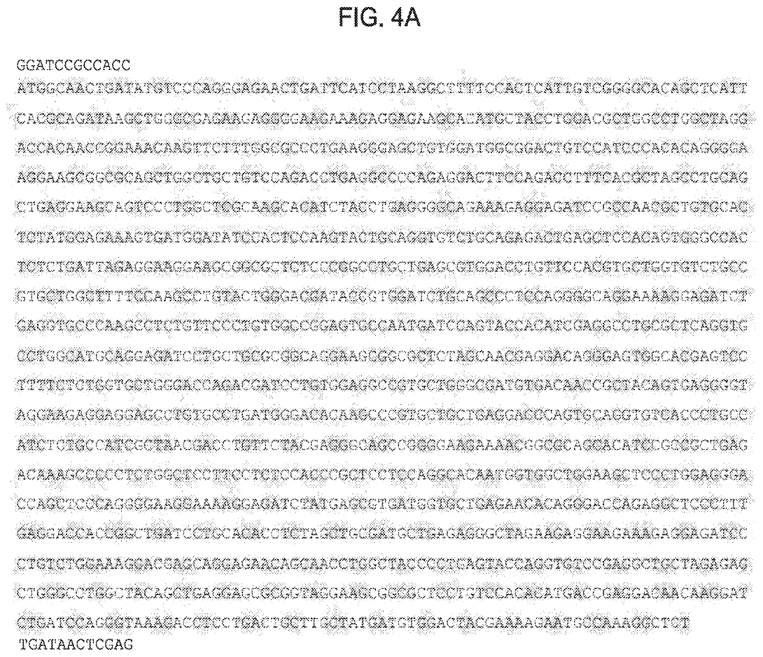

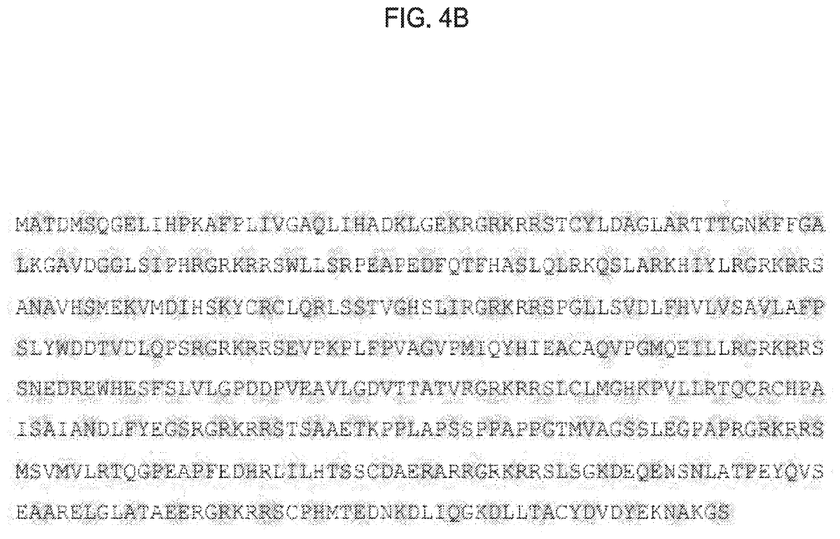

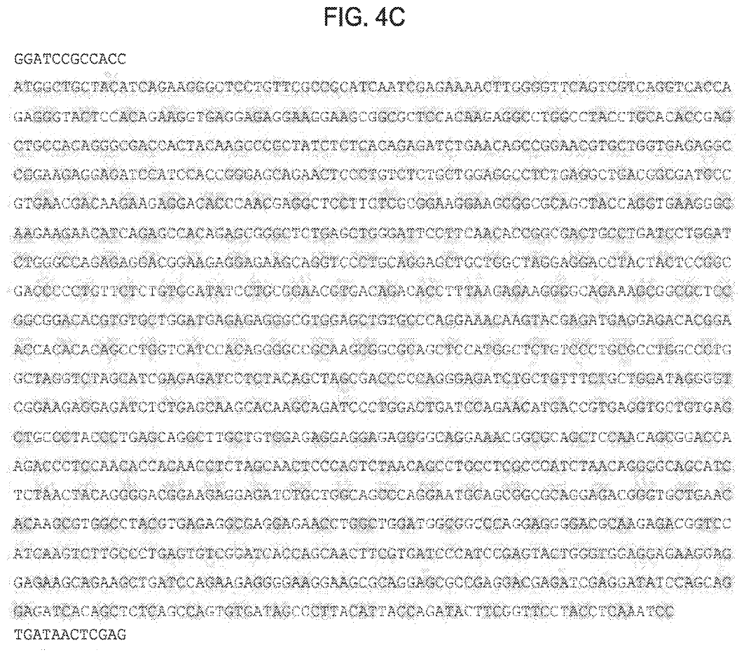

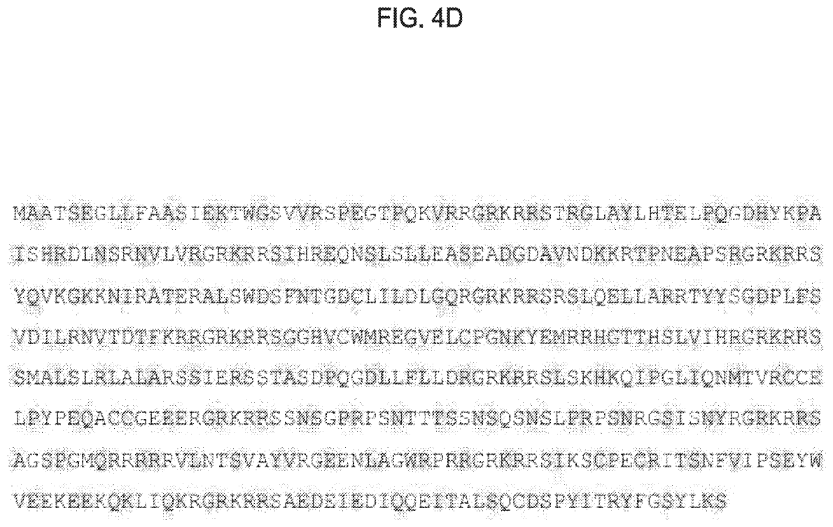

Nucleic acid molecules useful in the methods of the invention include any nucleic acid molecule that encodes a neoantigen, or a fragment thereof; any nucleic acid that encodes a linker, any nucleic acid that encodes a regulatory sequence, any nucleic acid that encodes a leader sequence. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. In some embodiments, the some embodiments, the nucleic acid sequence or molecules of the disclosure relate to nucleic acid sequences comprising a nucleic acid sequence at least about 70%, 80%, 85%, 90%, 95%, 96%, 97%, 98% 99% or 100% sequence identity to SEQ ID NO:68, the sequence of FIG. 2A, 2C, 2E, 3A, 3C, 4A or FIG. 4C. In some embodiments, the nucleic acid sequence or molecules of the disclosure relate to nucleic acid sequences comprising a nucleic acid sequence with at least about 70%, 80%, 85%, 90%, 95%, 96%, 97%, 98% 99% or 100% sequence identity to SEQ ID NO:68, the sequence of FIG. 2A, 2C, 2E, 3A, 3C, 4A or FIG. 4C, and comprise Formula I, I(a), or II(a) within their multiple cloning site. In some embodiments, the some embodiments, the nucleic acid sequence or molecules of the disclosure relate to nucleic acid sequences comprising a nucleic acid sequence encoding an amino acid sequence encoded by a nucleic acid sequence with at least about 70%, 80%, 85%, 90%, 95%, 96%, 97%, 98% 99% or 100% sequence identity to SEQ ID NO:68, the sequence of FIG. 2A, 2C, 2E, 3A, 3C, 4A or FIG. 4C.

In some embodiments, the disclosure relates to a nucleic acid molecule that is p Vax or with at least about 70%, 80%, 85%, 90%, 95%, 96%, 97%, 98% 99% or 100% sequence identity to SEQ ID NO:68. In some embodiments, the disclosure relates to a nucleic acid molecule that is p Vax or with at least about 70%, 80%, 85%, 90%, 95%, 96%, 97%, 98% 99% or 100% sequence identity to SEQ ID NO:68 comprising a coding sequence comprising any one or plurality of nucleic acid sequences with at least about 70%, 80%, 85%, 90%, 95%, 96%, 97%, 98% 99% or 100% sequence identity to SEQ ID Nos: 1-40. In some embodiments, the disclosure relates to a nucleic acid molecule that is p Vax or with at least about 70%, 80%, 85%, 90%, 95%, 96%, 97%, 98% 99% or 100% sequence identity to SEQ ID NO:68 comprising a coding sequence comprising any one or plurality of nucleic acid sequences with at least about 70%, 80%, 85%, 90%, 95%, 96%, 97%, 98% 99% or 100% sequence identity to SEQ ID Nos: 1-40, and, optionally, one or plurality of nucleic acid sequences encoding one or a plurality of amino acid sequences with at least about 70%, 80%, 85%, 90%, 95%, 96%, 97%, 98% 99% or 100% sequence identity to SEQ ID NO:61-66.

In some embodiments, an exemplary leader sequence is an IgE leader amino acid sequence as set forth in the sequence below and described in US20160175427, incorporated by reference in its entirety herein.

In some embodiments, the nucleic acid comprises a coding region consisting of any of Formulae I, I(a) and/or I(b) and one or a plurality of leader sequences. In some embodiments, the leader sequence is an IgE leader sequence: Met Asp Trp Thr Trp Ile Leu Phe Leu Val Ala Ala Ala Thr Arg Val (SEQ ID NO:69) or a leader sequence that is a functional fragment thereof comprising at least about 70%, 80%, 85%, 90%, 95%, 96%, 97%, 98% or 99% homologous to the IgE leader sequence identified in the aforementioned sentence. In some embodiments, the nucleic acid sequence or molecules of the disclosure relate to nucleic acid sequences comprising a leader with at least about 70%, 80%, 85%, 90%, 95%, 96%, 97%, 98% 99% or 100% sequence identity to SEQ ID NO:69.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The nucleic acid sequences may be used in association with other polynucleotide sequences coding for regulatory proteins that control the expression of the neo antigen sequence. For example, the nucleic acid molecule according to the invention may additionally contain recognition, regulatory, leader and promoter sequences.

In some embodiments, the nucleic acid molecule further comprises at least one regulatory sequence, wherein at least one nucleic acid sequence of Formula I is operably linked to the regulatory sequence.

In another embodiment, the nucleic acid molecule further comprises a leader sequence.

In some embodiments, an exemplary leader sequence is an IgE leader amino acid sequence as described in US20160175427, incorporated by reference in its entirety herein.

In some embodiments, the nucleic acid molecule comprises a nucleic acid sequence comprising Formula I ([(AEDⁿ)-(linker)]ₙ-[AEDⁿ⁺¹]), wherein the antigen expression domain 1 is independently selectable from about 12 to about 15,000 nucleotides in length and encodes an epitope from one or a plurality of cancer cells from a subject; and the antigen expression domain 2 is independently selectable from about 12 to about 15,000 nucleotides in length and encodes an epitope from one or a plurality of cancer cells from the subject.

In some embodiments, the nucleic acid molecule comprising a nucleic acid sequence comprising Formula I ([(AEDⁿ)-(linker)]ₙ-[AEDⁿ⁺¹]) is in an amount sufficient to elicit a cellular immune response. A “cellular immune response” is meant to include a cellular response directed to cells characterized by presentation of an antigen with class I or class II MHC. The cellular response relates to cells called T cells or T-lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill diseased cells such as cancer cells, preventing the production of more diseased cells. In preferred embodiments, the present invention involves the stimulation of an anti-tumor CTL response against tumor cells expressing one or more tumor expressed antigens and preferably presenting such tumor expressed antigens with class I MHC.

In some embodiments, the nucleic acid molecule comprising a nucleic acid sequence comprising Formula I ([(AEDⁿ)-(linker)]ₙ-[AEDⁿ⁺¹]) is in an amount sufficient to elicit a CD8+ T cell response against any one or plurality of amino acid sequences encoded by one or plurality of antigen expression domains. In some embodiments, the nucleic acid molecule comprising a nucleic acid sequence comprising Formula I ([(AEDⁿ)-[AEDⁿ⁺¹]) is in an amount sufficient to elicit a CD8+ T and/or CD4+ T cell response against any one or plurality of amino acid sequences encoded by one or plurality of antigen expression domains.

In some embodiments, the nucleic acid molecule comprising a nucleic acid sequence comprising Formula I ([(AEDⁿ)-(linker)]ₙ-[AEDⁿ⁺¹]) is in an amount sufficient to elicit a CD4+ T cell response against any one or plurality of amino acid sequences encoded by one or plurality of antigen expression domains. In some embodiments, the nucleic acid molecule comprising a nucleic acid sequence comprising Formula I ([(AEDⁿ)-(linker)]ₙ-[AEDⁿ⁺¹]) is in an amount sufficient to elicit a subpopulation of T cells that are greater than at least about 40% CD4⁺ T cells in response against any one or plurality of amino acid sequences encoded by one or plurality of antigen expression domains as compared to the response generated without the nucleic acid sequences disclosed herein. In some embodiments, the nucleic acid molecule comprising a nucleic acid sequence comprising Formula I ([(AEDⁿ)-(linker)]ₙ-[AEDⁿ⁺¹]) is in an amount sufficient to elicit a subpopulation of T cells that are greater than at least about 40% CD8+ T cells in response against any one or plurality of amino acid sequences encoded by one or plurality of antigen expression domains as compared to the response generated without the nucleic acid sequences disclosed herein. In some embodiments, the nucleic acid molecule comprising a nucleic acid sequence comprising Formula I ([(AEDⁿ)-(linker)]ₙ-[AEDⁿ⁺¹]) is in an amount sufficient to elicit a subpopulation of T cells that comprise greater than at least about 40% CD4+ T cells and that comprise greater than 40% CD8+ T cells in response against any one or plurality of amino acid sequences encoded by one or plurality of antigen expression domains as compared to the response generated without the nucleic acid sequences disclosed herein.

In a still further aspect, the nucleic acid molecule described in any of the aspects and embodiments herein is a plasmid. In certain embodiments, an expression vector comprises the nucleic acid molecule described in any of the aspects and embodiments. In certain embodiments, the nucleic acid expression vector is a plasmid. The vector can be capable of expressing one or a plurality of consensus neoantigen sequences in the cell of a mammal in a quantity effective to elicit an immune response in the mammal. The vector can be recombinant. The vector can comprise heterologous nucleic acid encoding the neoantigen. The vector can be a plasmid. The vector can be useful for transfecting cells with nucleic acid encoding a neoantigen, which the transformed host cell is cultured and maintained under conditions wherein expression of the neoantigen takes place. In some embodiments, the vector is capable of expressing one or a plurality of neoantigen sequences in the cell of a mammal in a quantity effective to elicit an immune response in the mammal. In some embodiments, a cell comprising the nucleic acid molecule is capable of expressing one or a plurality of consensus neoantigen sequences in the cell of a mammal in a quantity effective to elicit an immune response in the mammal that shrinks a tumor by more than about 5, 10, 15, 20, 30, 40, 50, 60, 70 or more percent. In some embodiments, a cell comprising the nucleic acid molecule is capable of expressing one or a plurality of neoantigen amino acid sequences in the cell of a mammal in a quantity effective to elicit an clonal expansion of CD8+ T cells from about 0.1 to about 50% of the total T cell stimulation against the one or plurality of neoantigens.

The vector can comprise heterologous nucleic acid encoding a neoantigen and can further comprise an initiation codon, which can be upstream of the neoantigen coding sequence, and a stop codon, which can be downstream of the neoantigen coding sequence. The initiation and termination codon can be in frame with the neoantigen coding sequence. The vector can also comprise a promoter that is operably linked to the neoantigen coding sequence. The promoter operably linked to the neoantigen coding sequence can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene such as human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the contents of which are incorporated herein in its entirety.

The vector can also comprise a polyadenylation signal, which can be downstream of the HA coding sequence. The polyadenylation signal can be a SV40 polyadenylation signal, LTR polyadenylation signal, bovine growth hormone (bGH) polyadenylation signal, human growth hormone (hGH) polyadenylation signal, or human .beta.-globin polyadenylation signal. The SV40 polyadenylation signal can be a polyadenylation signal from a pCEP4 vector (Invitrogen, San Diego, Calif.).

The vector can also comprise an enhancer upstream of the neoantigen coding. The enhancer can be necessary for DNA expression. The enhancer can be human actin, human myosin, human hemoglobin, human muscle creatine or a viral enhancer such as one from CMV, HA, RSV or EBV. Polynucleotide function enhances are described in U.S. Pat. Nos. 5,593,972, 5,962,428, and WO94/016737, the contents of each are fully incorporated by reference.

The vector can also comprise a mammalian origin of replication in order to maintain the vector extrachromosomally and produce multiple copies of the vector in a cell. In some embodiments, the vector can be LLC, TC1, ID8, pGX4501, pGX4503, pGX4504, pGX4505, and/or pGX4506 or any one or more regulatory or non-coding sequences of LLC, TC1, ID8, pGX4501, pGX4503, pGX4504, pGX4505, and/or pGX4506. In some embodiments, the vector comprises the sequence that is pVAX1. The backbone of the vector can be pAV0242. The vector can be a replication defective adenovirus type 5 (Ad5) vector.

The vector can also comprise a regulatory sequence, which can be well suited for gene expression in a mammalian or human cell into which the vector is administered. The neoantigen coding sequence can comprise a codon, which can allow more efficient transcription of the coding sequence in the host cell.

The vector can be pSE420 (Invitrogen, San Diego, Calif.), which can be used for protein production in Escherichia coli (E. coli). The vector can also be pYES2 (Invitrogen, San Diego, Calif.), which can be used for protein production in Saccharomyces cerevisiae strains of yeast. The vector can also be of the MAXBAC™ complete baculovirus expression system (Invitrogen, San Diego, Calif.), which can be used for protein production in insect cells. The vector can also be pcDNA I or pcDNA3 (Invitrogen, San Diego, Calif.), which can be used for protein production in mammalian cells such as Chinese hamster ovary (CHO) cells. The vector can be expression vectors or systems to produce protein by routine techniques and readily available starting materials including Sambrook et al., Molecular Cloning: A Laboratory Manual, Second Ed., Cold Spring Harbor (1989), which is incorporated fully by reference.

Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

In some embodiments, the nucleic acid sequence of Formula I is positioned with the multiple cloning site of a plasmid selected from the group consisting of LLC, TC1, ID8, pGX4501, pGX4503, pGX4504, pGX4505, and pGX4506. In some embodiments, the nucleic acid sequence of Formula I is positioned with the multiple cloning site of LLC. In some embodiments, the nucleic acid sequence of Formula I is positioned with the multiple cloning site of TC1. In some embodiments, the nucleic acid sequence of Formula I is positioned with the multiple cloning site of ID8. In some embodiments, the nucleic acid sequence of Formula I is positioned with the multiple cloning site of pGX4501. In some embodiments, the nucleic acid sequence of Formula I is positioned with the multiple cloning site of pGX4503. In some embodiments, the nucleic acid sequence of Formula I is positioned with the multiple cloning site of pGX4504. In some embodiments, the nucleic acid sequence of Formula I is positioned within the multiple cloning site of pGX4505. In some embodiments, the nucleic acid sequence of Formula I is positioned with the multiple cloning site of pGX4506. In preferred embodiments, the plasmid is pGX4505 or a sequence that is 70%, 80%, 90%, 91%, 92%, 93%, 94%, 95%, 96%, 97%, 98%, 99% homolgous to each of the above-identified nucleotide sequences.

In another embodiment, a host cell is transformed with the plasmids described herein.

The invention also provides that the one or more neo-antigenic peptides of the invention may be encoded by a single expression vector. The invention also provides that the one or more neo-antigenic peptides of the invention may be encoded and expressed in vivo using a viral based system (e.g., an adenovirus system).

The term “polynucleotide encoding a polypeptide” encompasses a polynucleotide which includes only coding sequences for the polypeptide as well as a polynucleotide which includes additional coding and/or non-coding sequences. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In some embodiments, the polynucleotides may comprise the coding sequence for the tumor specific neo-antigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In some embodiments, the polynucleotides can comprise the coding sequence for the tumor specific neo-antigenic peptide fused in the same reading frame to a marker sequence that allows, for example, for purification of the encoded polypeptide, which may then be incorporated into the personalized neoplasia vaccine. For example, the marker sequence can be a hexa-histidine (SEQ ID NO: 14) tag supplied by a pQE-9 vector to provide for purification of the mature polypeptide fused to the marker in the case of a bacterial host, or the marker sequence can be a hemagglutinin (HA) tag derived from the 115 influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In embodiments, the polynucleotides may comprise the coding sequence for one or more of the tumor specific neo-antigenic peptides fused in the same reading frame to create a single concatamerized neo-antigenic peptide construct capable of producing multiple neo-antigenic peptides.

In embodiments, the present invention provides isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide encoding a tumor specific neo-antigenic peptide of the present invention.

By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

The present disclosure also includes a composition comprising one or a plurality of nucleic acid molecules described herein.

The present disclosure also contemplates the use of nucleic acid molecules as vehicles for delivering neo-antigens to the subject in vivo in the form of, e.g., DNA/RNA vaccines (see, e.g., WO2012/159643, and WO2012/159754, hereby incorporated by reference in their entirety).

In some embodiments, the personalized neoplasia vaccine may include separate DNA plasmids encoding, for example, one or more neo-antigenic peptides/polypeptides as identified in according to the invention. As discussed above, the exact choice of expression vectors will depend upon the peptide/polypeptides to be expressed, and is well within the skill of the ordinary artisan. The expected persistence of the DNA constructs (e.g., in an episomal, non-replicating, non-integrated form in the muscle cells) is expected to provide an increased duration of protection. In some embodiments, the composition comprises the a first, second or third nucleic acid molecule, wherein at least the first nucleic acid molecule encodes one or more neoantigens. In some embodiments, the second nucleic acid molecule comprises one or more neoantigens. In some embodiments, the second nucleic acid molecule comprising a nucleic acid sequence that encodes one or more adjuvants. In other embodiments, the personalized neoplasia vaccine may include separate RNA or cDNA molecules encoding neo-antigenic peptides/polypeptides of the invention.

In another embodiment the personalized neoplasia vaccine may include a viral based vector for use in a human patient such as, for example, and adenovirus system (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207 (2): 240-7, hereby incorporated by reference in its entirety).

### Methods of Identifying Neoantigens

As described in more detail herein, a population of neoplasia/tumor specific neoantigens may be identified by sequencing the neoplasia/tumor and normal DNA of each patient to identify tumor-specific mutations, and determining the patient's HLA allotype. The population of neoplasia/tumor specific neo-antigens and their cognate native antigens may be subject to bioinformatic analysis using validated algorithms to predict which tumor-specific mutations create epitopes that could bind to the patient's HLA allotype, and in particular which tumor-specific mutations create epitopes that could bind to the patient's HLA allotype more effectively than the cognate native antigen. Based on this analysis, identified nucleotide sequences corresponding to these mutations may be designed for each patient, and used together for use as a cancer vaccine in immunizing the subject.

In one aspect, the disclosure features a method of identifying one or more subject-specific neoantigen mutations in a subject, wherein the subject has been diagnosed with, suspected of having or comprises one or more hyperproliferative cells (e.g. such as a tumor). In some embodiments, the disclosure features a method of identifying one or more subject-specific neoantigen mutations in a subject, wherein the subject has been diagnosed with, suspected of having or comprises one or more hyperproliferative cells (e.g. such as a tumor) characterized by the presence or quantity of a plurality of neoantigen mutations, the method comprising sequencing a nucleic acid sample from a tumor of the subject and of a non-tumor sample of the subject; analyzing the sequence to determine coding and non-coding regions; identifying sequences comprising tumor-specific non-synonymous or non-silent mutations not present in the non-tumor sample; identifying single nucleotide variations and single nucleotide insertions and deletions; producing subject-specific peptides encoded by the sequences comprising tumor-specific non-synonymous or non-silent mutations not present in the non-tumor sample; and measuring the binding characteristics of the of the subject-specific peptides, wherein each subject-specific peptide is an expression product of subject-specific DNA neoantigen not present in the non-tumor sample, thereby identifying one or more subject-specific DNA neoantigens in a subject.

measuring the binding characteristics of the of the subject-specific peptides is carried out by one or more of measuring the binding of the subject-specific peptides to T-cell receptor; measuring the binding of the subject-specific peptides to a HLA protein of the subject; or measuring the binding of the subject-specific peptides to transporter associated with antigen processing (TAP).

Efficiently choosing which particular mutations to utilize as immunogen requires identification of the patient HLA type and the ability to predict which mutated peptides would efficiently bind to the patient's HLA alleles. Therefore, In some embodiments, measuring the binding of the subject-specific peptides to T-cell receptor comprises measuring the binding of the subject-specific peptides to a HLA protein of the subject or sample.

In some embodiments, the subject-specific peptides bind to HLA proteins of the subject with an IC50 of less than about 550 nM. In some embodiments, the subject-specific peptides bind to HLA proteins of the subject with an IC50 of less than about 500 nM. In some embodiments, the subject-specific peptides bind to HLA proteins of the subject with an IC50 of less than about 450 nM. In some embodiments, the subject-specific peptides bind to HLA proteins of the subject with an IC50 of less than about 400 nM. In some embodiments, the subject-specific peptides bind to HLA proteins of the subject with an IC50 of less than about 350 nM. In some embodiments, the subject-specific peptides bind to HLA proteins of the subject with an IC50 of less than about 300 nM.

In another embodiment, the method of identifying one or more subject-specific DNA neoantigen mutations in a subject further comprises the step of ranking the subject-specific peptides based on the binding characteristics.

In another embodiment, the method of identifying one or more subject-specific DNA neoantigen mutations in a subject, further comprises the step of measuring the CD8+ T cell immune response generated by the subject-specific peptides. Methods of measuring the CD8+ T cell response are known in the art and described herein.

In a further embodiment, the method of identifying one or more subject-specific DNA neoantigen mutations in a subject further comprises formulating the subject-specific DNA neoantigens into a immunogenic composition for administration to the subject. In some embodiments, the top 200 ranked neo-antigen mutations are included or subcloned into the immunogenic composition, which in some embodiments, is one or a plurality of plasmids. In another embodiment, the top 150 ranked neo-antigen mutations are included in the immunogenic composition. In another embodiment, the top 100 ranked neo-antigen mutations are included in the immunogenic composition. In another embodiment, the top 50 ranked neo-antigen mutations are included in the immunogenic composition. In another embodiment, the top 25 ranked neo-antigen mutations are included in the immunogenic composition. In another embodiment, the top 10 ranked neo-antigen mutations are included in the immunogenic composition. In another embodiment, the top 5 ranked neo-antigen mutations are included in the immunogenic composition. In another embodiment, the top 5-20 ranked neo-antigen mutations are included in the immunogenic composition. In another embodiment, the top 10-50 ranked neo-antigen mutations are included in the immunogenic composition. In another embodiment, the top 25-100 ranked neo-antigen mutations are included in the immunogenic composition. In another embodiment, the top 50-100 ranked neo-antigen mutations are included in the immunogenic composition. In another embodiment, the top 100-200 ranked neo-antigen mutations are included in the immunogenic composition.

In another embodiment, the method of identifying one or more subject-specific DNA neoantigen mutations in a subject further comprises providing a culture comprising dendritic cells (DCs) obtained from the subject; and contacting the dendritic cells with the immunogenic composition. DCs are potent antigen-presenting cells that initiate T cell immunity and can be used as cancer vaccines when loaded with one or more neoantigens of interest. In a further embodiment, the method further comprises administering to the subject the dendritic cells; obtaining a population of CD8+ T cells from a peripheral blood sample from the subject, wherein the CD8+ cells recognize the at least one neoantigen; and expanding the population of CD8+ T cells that recognizes the neoantigen.

In some embodiments, the expanded population of CD8+ T cells is administered to the subject.

Preferably, any suitable sequencing-by-synthesis platform can be used to identify mutations. Four major sequencing-by-synthesis platforms are currently available: the Genome Sequencers from Roche/454 Life Sciences, the HiSeq Analyzer from Illumina/Solexa, the SOLID system from Applied BioSystems, and the Heliscope system from Helicos Biosciences. Sequencing-by-synthesis platforms have also been described by Pacific Biosciences and VisiGen Biotechnologies. Each of these platforms can be used in the methods of the invention. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids may be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., U.S. Patent Application No. 2006/0252077) may be linked to each fragment to be captured on a surface coated with a respective second member of that coupling pair. Subsequent to the capture, the sequence may be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or in a step-and-repeat mode. For real-time analysis, different optical labels to each nucleotide may be incorporated and multiple lasers may be utilized for stimulation of incorporated nucleotides.

Any cell type or tissue may be utilized to obtain nucleic acid samples for use in the sequencing methods described herein. In a preferred embodiment, the DNA or RNA sample is obtained from a neoplasia, a tumor or a bodily fluid, e.g., blood, obtained by known techniques (e.g. venipuncture) or saliva. Alternatively, nucleic acid tests can be performed on dry samples (e.g. hair or skin).

A variety of methods are available for detecting the presence of a particular mutation or allele in an individual's DNA or RNA. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

PCR based detection means may include multiplex amplification of a plurality of markers simultaneously. For example, it is well known in the art to select PCR primers to generate PCR products that do not overlap in size and can be analyzed simultaneously.

Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. In some embodiments, the single base polymorphism can be detected by using a specialized exonuclease-resistant nucleotide, as disclosed, e.g., U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen et al. (French Patent No. 2,650,840; PCT Application No. WO1991/02087). As in the method of U.S. Pat. No. 4,656,127, a primer may be employed that is complementary to allelic sequences immediately 3′ to a polymorphic site. The method determines the identity of the nucleotide of that site using labeled dideoxynucleotide derivatives, which, if complementary to the nucleotide of the polymorphic site, will become incorporated onto the terminus of the primer.

An alternative method, known as Genetic Bit Analysis or GBA is described in PCT Application No. WO1992/15712). GBA uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. WO1991/02087) the GBA method is preferably a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C, et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C, et al., Amer. J. Hum. Genet. 52:46-59 (1993)).

The disclosure generally relates to a method of identifying or selecting one or a plurality of neoantigens from a sample, the method comprising (a) sequencing the DNA/RNA from a sample, and (b) measuring the binding of the subject-specific peptides to T-cell receptor comprises measuring the binding of the subject-specific peptides to a HLA protein of the subject or sample, and (c) selecting or a plurality of neoantigens from a sample if the HLA protein from the subject binds to HLA proteins of the subject with an IC50 of less than about 500 nM, 400 nM, 300 nM, 200 nM, or 100 nM; and, optionally (d) ordering the neoantigens in order of lowest IC50 value to highest IC50 value.

In some embodiments, the disclosure relates to generating a vaccine or manufacturing a pharmaceutical composition comprising performing any one or more of the aforementioned steps and further comprising subcloning a nucleic acid sequence encoding the one or plurality of neoantigens into one or more nucleic acid molecules; and, optionally, suspending the nucleic acid molecules in one or more pharmaceutically acceptable carriers.

In some embodiments, the nucleic acid sequence encoding the neoantigens also comprises a linker. In some embodiments, the nucleic acid molecule is free of a nucleic acid sequence that encodes a P2A linker. In some embodiments, the nucleic acid molecule is free of a nucleic acid sequence that encodes two different linkers. In some embodiments, the nucleic acid molecule is free of a nucleic acid sequence that encodes a linker, such that at least two or a plurality of AED sequences, from the 5′ to 3′ orientation is encoded as a separate polypeptide or as a large contiguous fusion protein. In another embodiment, the method of identifying one or more subject-specific DNA neoantigen mutations in a subject further comprises providing a culture comprising dendritic cells (DCs) obtained from the subject; and contacting the dendritic cells with the immunogenic composition. DCs are potent antigen-presenting cells that initiate T cell immunity and can be used as cancer vaccines when loaded with one or more neoantigens of interest. In a further embodiment, the method further comprises administering to the subject the dendritic cells; obtaining a population of CD8+ T cells from a peripheral blood sample from the subject, wherein the CD8+ cells recognize the at least one neoantigen; and expanding the population of CD8+ T cells that recognizes the neoantigen.

In some embodiments, the expanded population of CD8+ T cells is administered to the subject.

Preferably, any suitable sequencing-by-synthesis platform can be used to identify mutations. Four major sequencing-by-synthesis platforms are currently available: the Genome Sequencers from Roche/454 Life Sciences, the HiSeq Analyzer from Illumina/Solexa, the SOLID system from Applied BioSystems, and the Heliscope system from Helicos Biosciences. Sequencing-by-synthesis platforms have also been described by Pacific Biosciences and VisiGen Biotechnologies. Each of these platforms can be used in the methods of the invention. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids may be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., U.S. Patent Application No. 2006/0252077) may be linked to each fragment to be captured on a surface coated with a respective second member of that coupling pair. Subsequent to the capture, the sequence may be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or in a step-and-repeat mode. For real-time analysis, different optical labels to each nucleotide may be incorporated and multiple lasers may be utilized for stimulation of incorporated nucleotides.

Any cell type or tissue may be utilized to obtain nucleic acid samples for use in the sequencing methods described herein. In a preferred embodiment, the DNA or RNA sample is obtained from a neoplasia/tumor or a bodily fluid, e.g., blood, obtained by known techniques (e.g. venipuncture) or saliva. Alternatively, nucleic acid tests can be performed on dry samples (e.g. hair or skin).

A variety of methods are available for detecting the presence of a particular mutation or allele in an individual's DNA or RNA. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

PCR based detection means may include multiplex amplification of a plurality of markers simultaneously. For example, it is well known in the art to select PCR primers to generate PCR products that do not overlap in size and can be analyzed simultaneously.

Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. In some embodiments, the single base polymorphism can be detected by using a specialized exonuclease-resistant nucleotide, as disclosed, e.g., U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen et al. (French Patent No. 2,650,840; PCT Application No. WO1991/02087). As in the method of U.S. Pat. No. 4,656,127, a primer may be employed that is complementary to allelic sequences immediately 3′ to a polymorphic site. The method determines the identity of the nucleotide of that site using labeled dideoxynucleotide derivatives, which, if complementary to the nucleotide of the polymorphic site, will become incorporated onto the terminus of the primer.

An alternative method, known as Genetic Bit Analysis or GBA is described in PCT Application No. WO1992/15712). GBA uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. WO1991/02087) the GBA method is preferably a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C, et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C, et al., Amer. J. Hum. Genet. 52:46-59 (1993)).

### Methods of Treating Cancer

The disclosure further provides a method of inducing a neoplasia/tumor-specific immune response in a subject, vaccinating against a neoplasia/tumor, treating and/or alleviating a symptom of cancer in a subject by administering to the subject the nucleic acid sequences as described herein.

In one aspect, the disclosure provides a method of treating and/or preventing cancer in a subject, the method comprising administering to the subject in need thereof a pharmaceutically effective amount of any of the nucleic acid molecules as described herein (e.g. a nucleic acid molecule comprising a nucleic acid sequence comprising Formula I: [(AEDⁿ)-(linker)]ₙ-[AEDⁿ⁺¹]) or any of the pharmaceutical compositions described herein.

In some embodiments, the nucleic acid molecule is administered to the subject by electroporation.

In some embodiments, treatment is determined by a clinical outcome, an increase, enhancement or prolongation of anti-tumor activity by T cells, an increase in the number of anti-tumor T cells or activated T cells as compared with the number prior to treatment, or a combination thereof. In a further embodiment, clinical outcome is selected from the group consisting of tumor regression, tumor shrinkage, tumor necrosis, anti-tumor response by the immune system, tumor expansion, recurrence or spread, or a combination thereof.

Examples of cancers and cancer conditions that can be treated with the combination therapy of this document include, but are not limited to a patient in need thereof that has been diagnosed as having cancer, or at risk of developing cancer.

In some embodiments, the subject has previously been treated, and not responded to checkpoint inhibitor therapy.

The therapy described herein is also applicable where the subject has no detectable neoplasia but is at high risk for disease recurrence.

According to the disclosure, the nucleic acid molecules described herein may be used for a patient that has been diagnosed as having cancer, or at risk of developing cancer.

In certain embodiments, the cancer is a solid tumor.

In some embodiments, the cancer has a high mutational load.

In another embodiment, the cancer has a moderate mutational load.

In other embodiments, the cancer has been shown to have a poor or low response to checkpoint inhibitor therapy.

In certain embodiments, the cancer is selected from, but not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood’, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland'Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In further embodiments, the cancer is selected from the group consisting of non small cell lung cancer, melanoma, ovarian cancer, cervical cancer, glioblastoma, urogenital cancer, gynecological cancer, lung cancer, gastrointestinal cancer, head and neck cancer, non-metastatic or metastatic breast cancer, malignant melanoma, Merkel Cell Carcinoma or bone and soft tissue sarcomas, haematologic neoplasias, multiple myeloma, acute myelogenous leukemia, chronic myelogenous leukemia, myelodysplastic syndrome and acute lymphoblastic leukemia, breast cancer, metastatic colorectal cancers, hormone sensitive or hormone refractory prostate cancer, colorectal cancer, ovarian cancer, hepatocellular cancer, renal cell cancer, pancreatic cancer, gastric cancer, oesophageal cancers, hepatocellular cancers, cholangiocellular cancers, head and neck squamous cell cancer soft tissue sarcoma, and small cell lung cancer.

In certain embodiments, the cancer is non-small cell lung carcinoma or melanoma, both of which have been shown to have a high mutational load.

In other embodiments, the cancer is ovarian cancer or glioblastoma multiforme, both of which show a moderate mutational load and have been shown to have a poor or low response to checkpoint inhibitor therapy.

### Methods of Inducing/Enhancing Immune Response

In one aspect, the present disclosure features a method of inducing an immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein. In some embodiments, the method comprises the steps of taking a sample from a subject, identifying one or more neoantigens expressed by hyperoliferative cells in a the sample, synthesizing one or more cDNA libraries based upon expression of neoantigens in the sample, cloning the one or more nucleic acid sequences that encode one or more epitopes of the neoantigens, into a nucleic acid molecule that comprises one or more components disclosed herein, and administering the nucleic acid molecule to the subject.

In one aspect, the present disclosure features a method of inducing a CD8+ T cell immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In one aspect, the present disclosure features a method of enhancing an immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In some aspects, the present disclosure features a method of enhancing a CD8+ T cell immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In some embodiments, the subject has cancer. In another embodiment, the subject has previously been treated, and not responded to checkpoint inhibitor therapy.

In some embodiments, the nucleic acid molecule is administered to the subject by electroporation.

In some embodiments, enhancing the CD8+ T cell immune response comprises activating from about 0.01% to about 50% CD8+ T cells. In some embodiments, enhancing the CD8⁺ T cell immune response comprises activating from about 0.01% to about 50% that are IFN-γ positive. In some embodiments, the activation of T cells is accomplished after no more than 1, 2, 3, 4, 5, 6, 7 8, 9, 10 or more hours of contact with antigen presenting cells expressing or plasmids comprising the nucleotic acid sequences disclosed herein or expressed by a hyperproliferative cell in a subject. CD8+ T cells. In some embodiments, enhancing the CD8+ T cell immune response comprises expanding CD8+ T cells. In some embodiments, enhancing the CD8+ T cell immune response comprises activating from about 0.05% to about 50% CD8+ T cells In some embodiments, enhancing the CD8+ T cell immune response comprises activating from about 0.10% to about 50% CD8+ T cells In some embodiments, enhancing the CD8+ T cell immune response comprises activating from about 0.2% to about 50% CD8+ T cells In some embodiments, enhancing the CD8+ T cell immune response comprises activating from about 0.3% to about 50% CD8+ T cells In some embodiments, enhancing the CD8+ T cell immune response comprises activating from about 0.4% to about 50% CD8+ T cells In some embodiments, enhancing the CD8+ T cell immune response comprises activating from about 0.5% to about 50% CD8+ T cells In some embodiments, enhancing the CD8+ T cell immune response comprises activating from about 0.6% to about 50% CD8+ T cells In some embodiments, enhancing the CD8+ T cell immune response comprises activating from about 0.7% to about 50% CD8+ T cells In some embodiments, enhancing the CD8+ T cell immune response comprises activating from about 0.8% to about 50% CD8+ T cells In some embodiments, enhancing the CD8+ T cell immune response comprises activating from about 0.9% to about 50% CD8+ T cells In some embodiments, enhancing the CD8+ T cell immune response comprises activating from about 1.00% to about 50% CD8+ T cells. In some embodiments, enhancing the CD8+ T cell immune response comprises activating from about 2.0% to about 50% CD8+ T cells In some embodiments, enhancing the CD8+ T cell immune response comprises activating from about 3.0% to about 50% CD8+ T cells. In some embodiments, enhancing the CD8+ T cell immune response comprises activating from about 5.0% to about 50% CD8+ T cells. In some embodiments, enhancing the CD8+ T cell immune response comprises activating from about 6.0% to about 50% CD8+ T cells. In some embodiments, enhancing the CD8+ T cell immune response comprises activating from about 7.0% to about 50% CD8+ T cells. In some embodiments, enhancing the CD8+ T cell immune response comprises activating from about 8.0% to about 50% CD8+ T cells. In some embodiments, enhancing the CD8+ T cell immune response comprises activating from about 9.0% to about 50% CD8+ T cells. In some embodiments, enhancing the CD8+ T cell immune response comprises activating from about 10% to about 50% CD8+ T cells. In some embodiments, enhancing the CD8+ T cell immune response comprises activating from about 15% to about 50% CD8+ T cells. In some embodiments, enhancing the CD8+ T cell immune response comprises activating from about 20% to about 50% CD8+ T cells.

T cell activation can be measured by various assays as described herein. For example, T cell activities that may be measured include the induction of proliferation of T cells, the induction of signaling in T cells, the induction of expression of activation markers in T cells, such as interferon-gamma (IFN-s), the induction of cytokine secretion by T cells, and the cytotoxic activity of T cells. For example, in certain embodiments, CD8+ T cell activation is measured by a proliferation assay. In some embodiments, the activation may be measured after stimulation of cells or cell sample by the encoded nucleic acid sequences.

### Cytokine Secretion

The activation of CD8+ T-cells may be assessed or measured by determining secretion of cytokines, such as gamma interferon (IFN-γ), tumor necrosis factor alpha (TNFa), interleukin-12 (IL-12) or interleukin 2 (IL-2). In some embodiments, ELISA is used to determine cytokine secretion, for example secretion of gamma interferon (IFN-γ), tumor necrosis factor alpha (TNFα), interleukin-12 (IL-12) or interleukin 2 (IL-2). The ELISPOT (enzyme-linked immunospot) technique may be used to detect T cells that secrete a given cytokine (e.g., gamma interferon (IFN-γ)) in response to stimulation with any of the nucleic acid molecules of any one of the aspects or embodiments herein, or any one of the pharmaceutical compositions of any one of the aspects and embodiments herein. T cells are cultured with, e.g. any of the nucleic acid molecules of any one of the aspects or embodiments herein wells which have been coated with anti-IFN-γ antibodies. The secreted IFN-γ is captured by the coated antibody and then revealed with a second antibody coupled to a chromogenic substrate. Thus, locally secreted cytokine molecules form spots, with each spot corresponding to one IFN-γ-secreting cell. The number of spots allows one to determine the frequency of IFN-γ-secreting cells in the analyzed sample. The ELISPOT assay has also been described for the detection of tumor necrosis factor alpha, interleukin-4 (IL-4), IL-5, IL-6, IL-10, IL-12, granulocyte-macrophage colony-stimulating factor, and granzyme B-secreting lymphocytes (Klinman D, Nutman T. Current protocols in immunology. New York, N.Y: John Wiley & Sons, Inc.; 1994. pp. 6.19.1-6.19.8, incorporated by reference in its entirety herein).

Flow cytometric analyses of intracellular cytokines may be used to measure the cytokine content in culture supernatants, but provides no information on the number of T cells that actually secrete the cytokine. When T cells are treated with inhibitors of secretion such as monensin or brefeldin A, they accumulate cytokines within their cytoplasm upon activation (e.g. with the nucleic acid molecules of the present invention). After fixation and permeabilization of the lymphocytes, intracellular cytokines can be quantified by cytometry. This technique allows the determination of the cytokines produced, the type of cells that produce these cytokines, and the quantity of cytokine produced per cell.

### Cytotoxicity

The activation of CD8+ T-cells by any of the nucleic acid molecules of any one of the aspects or embodiments herein, or any one of the pharmaceutical compositions of any one of the aspects and embodiments herein may be assessed by assaying the cytotoxic activity of the CD8+ T-cells.

The cytotoxic activity of T cells may be assessed by any suitable technique known to those of skill in the art. For example, a sample comprising T cells that have been exposed to the nucleic acid molecules according to the invention can be assayed for cytotoxic activity after an appropriate period of time, in a standard cytotoxic assay. Such assays may include, but are not limited to, the chromium release CTL assay and the Alamar Blue™ fluorescence assay known in the art.

### Proliferation/Expansion

The ability of the any of the nucleic acid molecules of any one of the aspects or embodiments herein, or any one of the pharmaceutical compositions of any one of the aspects and embodiments herein to expand T cells can be evaluated by using CFSE staining. To compare the initial rate of cell expansion, the cells are subject to CFSE staining to determine how well any of the nucleic acid molecules of any one of the aspects or embodiments herein, or any one of the pharmaceutical compositions of any one of the aspects and embodiments herein induced the proliferation of T cells. CFSE staining provides a much more quantitative endpoint and allows simultaneous phenotyping of the expanded cells. Every day after stimulation, an aliquot of cells is removed from each culture and analyzed by flow cytometry. CFSE staining makes cells highly fluorescent. Upon cell division, the fluorescence is halved and thus the more times a cell divides the less fluorescent it becomes. The ability of any of the nucleic acid molecules of any one of the aspects or embodiments herein, or any one of the pharmaceutical compositions of any one of the aspects and embodiments herein to induce T cell proliferation is quantitated by measuring the number of cells that divided once, twice, three times and so on. The nucleic acid molecules that induce the greatest number of cell divisions at a particular time point is deemed as the most potent expander.

To determine how well any of the nucleic acid molecules of any one of the aspects or embodiments herein, or any one of the pharmaceutical compositions of any one of the aspects and embodiments herein promote long-term growth of T cells, cell growth curves can be generated. These experiments are set up as the foregoing CFSE experiments, but no CFSE is used. Every 2-3 days of culture, T cells are removed from the respective cultures and counted using a Coulter counter which measures how many cells are present and the mean volume of the cells. The mean cell volume is the best predicator of when to restimulate the cells. In general, when T cells are properly stimulated they triple their cell volume. When this volume is reduced to more than about half of the initial blast, it may be necessary to restimulate the T cells to maintain a log linear expansion (Levine et al., 1996, Science 272:1939-1943; Levine et al., 1997, J. Immunol. 159:5921-5930). The time it takes the nucleic acid molecules of any one of the aspects or embodiments herein, or any one of the pharmaceutical compositions of any one of the aspects and embodiments herein to induce 20 population doublings is calculated. The relative differences of each nucleic acid molecule to induce this level of T cell expansion is an important criteria on which the nucleic acid molecules of any one of the aspects or embodiments herein, or any one of the pharmaceutical compositions of any one of the aspects and embodiments herein are assessed.

### Apoptosis Markers

In certain embodiments of the present invention, stimulation, activation, and expansion of T cells using the nucleic acid molecules of any one of the aspects or embodiments herein, or any one of the pharmaceutical compositions of any one of the aspects and embodiments herein enhances expression of certain key molecules in T cells that protect again apoptosis or otherwise prolong survival in vivo or in vitro. Apoptosis usually results from induction of a specific signal in the T cell. Thus, the nucleic acid molecules of any one of the aspects or embodiments herein, or any one of the pharmaceutical compositions of any one of the aspects and embodiments herein may provide for protecting a T cell from cell death resulting from stimulation of the T cell. Therefore, also included in the present invention is the enhanced T cell growth by protection from premature death or from absence or depletion of recognized T cell growth markers, such as Bcl-xL, growth factors, cytokines, or lymphokines normally necessary for T cell survival, as well as from Fas or Tumor Necrosis Factor Receptor (TNFR) cross-linking or by exposure to certain hormones or stress.

In another aspect, the disclosure features a method of enhancing an immune response against a plurality of heterogeneous hyperproliferative cells in a subject, the method comprising administering to the subject in need thereof a pharmaceutically effective amount of any of the nucleic acid molecules described herein (e.g. a nucleic acid molecule comprising a nucleic acid sequence comprising Formula I: [(AEDⁿ)-(linker)]ₙ-[AEDⁿ⁺¹]), or any of the pharmaceutical compositions described herein.

In some embodiments, the subject has cancer. In another embodiment, the subject has previously been treated, and not responded to checkpoint inhibitor therapy.

In some embodiments, the nucleic acid molecule is administered to the subject by electroporation.

In some embodiments, the immune response is of a sufficient magnitude or efficacy to inhibit or retard tumor growth, induce tumor cell death, induce tumor regression, prevent or delay tumor recurrence, prevent tumor growth, prevent tumor spread and/or induce tumor elimination.

In some embodiments, the method of enhancing an immune response against a plurality of heterogeneous hyperproliferative cells in a subject further comprises administration of one or more therapeutic agents.

In some embodiments, the additional therapeutic agent is a biologic therapeutic or a small molecule.

In another embodiment, the therapeutic agent is (i) a checkpoint inhibitor or functional fragment thereof; or (ii) a nucleic acid molecule encoding a checkpoint inhibitor or a functional fragment thereof. In a further embodiment, the checkpoint inhibitor associates with or inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof. In an exemplary embodiment, the checkpoint inhibitor is an inhibitor of the programmed death-1 (PD-1) pathway. In another exemplary embodiment, the checkpoint inhibitor is an anti-cytotoxic T-lymphocyte-associated antigen 4 (CTLA4) antibody or functional fragment thereof.

In another embodiment, the therapeutic agent is an adjuvant. The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response. In some embodiments, the adjuvant can be other genes that are expressed in alternative plasmid or are delivered as proteins in combination with the plasmid above in the vaccine.

In some embodiments, the adjuvant can be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNF.alpha., TNF.beta., GM-CSF, epidermal growth factor (EGF), IL-1, IL-2, IL-4, IL-5, IL-6, IL-10, IL-12, IL-18, or a combination thereof.

Other genes which can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

Human IL-12 alpha subunit is set forth in GenBank® Accession Nos. NP_000873.2, NM_000882.3, incorporated by reference in their entireties herein. An exemplary human IL-12 alpha subunit amino acid sequence is shown below:

(SEQ ID NO: 54)
MCPARSLLLV ATLVLLDHLS LARNLPVATP DPGMFPCLHH
SQNLLRAVSN MLQKARQTLE FYPCTSEEID HEDITKDKTS
TVEACLPLEL TKNESCLNSR ETSFITNGSC LASRKTSFMM
ALCLSSIYED LKMYQVEFKT MNAKLLMDPK RQIFLDQNML
AVIDELMQAL NFNSETVPQK SSLEEPDFYK TKIKLCILLH
AFRIRAVTID RVMSYLNAS

Human IL-12 beta subunit is set forth in GenBank® Accession No. NP_002178.2, incorporated by reference in its entirety herein. An exemplary human IL-12 beta subunit amino acid sequence is shown below:

(SEQ ID NO: 55)
MCHQQLVISW FSLVFLASPL VAIWELKKDV YVVELDWYPD
APGEMVVLTC DTPEEDGITW TLDQSSEVLG SGKTLTIQVK
EFGDAGQYTC HKGGEVLSHS LLLLHKKEDG IWSTDILKDQ
KEPKNKTFLR CEAKNYSGRF TCWWLTTIST DLTFSVKSSR
GSSDPQGVTC GAATLSAERV RGDNKEYEYS VECQEDSACP
AAEESLPIEV MVDAVHKLKY ENYTSSFFIR DIIKPDPPKN
LQLKPLKNSR QVEVSWEYPD TWSTPHSYFS LTFCVQVQGK
SKREKKDRVF TDKTSATVIC RKNASISVRA QDRYYSSSWS
EWASVPCS

Human IL-15 is set forth in GenBank® Accession Nos. NP_000576.1, NP_751915.1, AAI00962.1 incorporated by reference in their entireties herein. An exemplary human IL-15 amino acid sequence is shown below:

(SEQ ID NO: 56)
MRISKPHLRS ISIQCYLCLL LNSHFLTEAG IHVFILGCFS
AGLPKTEANW VNVISDLKKI EDLIQSMHID ATLYTESDVH
PSCKVTAMKC FLLELQVISL ESGDASIHDT VENLIILANN
SLSSNGNVTE SGCKECEELE EKNIKEFLQS FVHIVQMFIN TS

Human IL-17 is set forth in GenBank® Accession Nos. NP_002181.1, NM_002190.2, incorporated by reference in their entireties herein. An exemplary human IL-17 amino acid sequence is shown below:

(SEQ ID NO: 57)
MTPGKTSLVS LLLLLSLEAI VKAGITIPRN PGCPNSEDKN
FPRTVMVNLN IHNRNTNTNP KRSSDYYNRS TSPWNLHRNE
DPERYPSVIW EAKCRHLGCI NADGNVDYHM NSVPIQQEIL
VLRREPPHCP NSFRLEKILV SVGCTCVTPI VHHVA

Human IL-8 is set forth in GenBank® Accession Nos. NP_000575.1, NM_000584.3, incorporated by reference in their entireties herein. An exemplary human IL-8 amino acid sequence is shown below:

(SEQ ID NO: 58)
MTSKLAVALL AAFLISAALC EGAVLPRSAK ELRCQCIKTY
SKPFHPKFIK ELRVIESGPH CANTEIIVKL SDGRELCLDP
KENWVQRVVE KFLKRAENS

Human C—C motif chemokine 5 (processed form RANTES (3-68)) is set forth in GenBank® Accession Nos. NP_002976.2, NM_002985.2, incorporated by reference in their entireties herein. An exemplary human C—C motif chemokine 5 amino acid sequence is shown below:

(SEQ ID NO: 59)
MKVSAAALAV ILIATALCAP ASASPYSSDT TPCCFAYIAR
PLPRAHIKEY FYTSGKCSNP AVVFVTRKNR QVCANPEKKW
VREYINSLEM S

Human Macrophage inflammatory protein 1-alpha (MIP-1a) is set forth in GenBank® Accession Nos. NP_002974.1, NM_002983.2, incorporated by reference in their entireties herein. An exemplary human C—C motif chemokine 5 amino acid sequence is shown below:

(SEQ ID NO: 60)
MQVSTAALAV LLCTMALCNQ FSASLAADTP TACCFSYTSR
QIPQNFIADY FETSSQCSKP GVIFLTKRSR QVCADPSEEW
VQKYVSDLEL SA

Other exemplary adjuvants include, but are not limited to, poly-ICLC (see Pharmacol Ther. 2015 February; 146:120-31, incorporated by reference in its entirety herein), 1018 ISS (see Vaccine. 2003 Jun. 2; 21 (19-20): 2461-7, incorporated by reference in its entirety herein), aluminum salts, Amplivax AS15, Bacillus Colmette-Guérin (BCG) (see Clin Immunol. 2000 January; 94 (1): 64-72, incorporated by reference in its entirety herein), CP-870,893, CpG7909 (GenBank® Accession No. CS576603.1), CyaA (GenBank® Accession No. KP670536.1), GM-CSF (GenBank® Accession No. M11220.1), IC30 (see Expert Rev Vaccines. 2007 October; 6 (5): 741-6, incorporated by reference in its entirety herein), IC31 (see Expert Rev Vaccines. 2007 October; 6 (5): 741-6, incorporated by reference in its entirety herein), Imiquimod (see Vaccine. 2006 Mar. 10; 24 (11): 1958-6, incorporated by reference in its entirety herein), ImuFact 1MP321, IS Patch, ISS, ISCOMATRIX, JuvImmune, LipoVac, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA micropartieles, resiquimod, S L172, Virosomes and other Virus-like particles, YF-17D, VEGF trap, R848, beta-glucan, Pam3Cys, acrylic or methacrylic polymers, copolymers of maleic anhydride and Aquila's QS21 stimulon, and a functional fragment of any thereof; or (ii) a nucleic acid molecule encoding an adjuvant selected from the group consisting of: (i) poly-ICLC, 1018 ISS, aluminum salts, Amplivax AS15, BCG, CP-870,893, CpG7909, CyaA, GM-CSF, IC30, IC31, Imiquimod, ImuFact 1MP321, IS Patch, ISS, ISCOMATRIX, JuvImmune, LipoVac, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, S L172, Virosomes and other Virus-like particles, YF-17D, VEGF trap, R848, beta-glucan, Pam3Cys, acrylic or methacrylic polymers, copolymers of maleic anhydride and Aquila's QS21 stimulon, or functional fragment thereof.

In another embodiment, the therapeutic agent is an immunostimulatory agent or functional fragment thereof. For example, in some embodiments, the imunostimulatory agent is an interleukin or functional fragment thereof.

In another embodiment, the therapeutic agent is a chemotherapeutic agent. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. For prostate cancer treatment, a preferred chemotherapeutic agent with which anti-CTLA-4 can be combined is paclitaxel (Taxol®).

In some embodiments, the adjuvant can include a nucleic acid plasmid that encodes any cytokine or functional fragment thereof that is administered sequentially with a pharmaceutical composition comprising a plasmid encoding a plurality of neoantigens, optionally with one or a plurality of tumor associated antigens not derived from a subject. In some embodiments, the cytokine is IL-12 or a subunit of IL-12. In some embodiments, adjuvant is a nucleic acid sequence that encodes an amino acid sequence that comprises at least about 70%, 75%, 80%, 85%, 90%, 95%, 96%, 97%, 98%, 99% or 100% sequence identity to SEQ NO: 54 or a functional fragment thereof. In some embodiments, adjuvant is a nucleic acid sequence that encodes an amino acid sequence that comprises at least about 70%, 75%, 80%, 85%, 90%, 95%, 96%, 97%, 98%, 99% or 100% sequence identity to SEQ NO: 55 or a functional fragment thereof. In some embodiments, adjuvant is a first nucleic acid sequence that encodes an amino acid sequence that comprises at least about 70%, 75%, 80%, 85%, 90%, 95%, 96%, 97%, 98%, 99% or 100% sequence identity to SEQ NO: 54 and a second amino acid sequence that comprises at least about 70%, 75%, 80%, 85%, 90%, 95%, 96%, 97%, 98%, 99% or 100% sequence identity to SEQ NO: 55 or a functional fragment thereof. In some embodiments, if the nucleic acid sequence encoding a cytokine or functional fragment thereof comprise two subunits, the disclosure relates to nucleic acid molecule comprises a first nucleic acid sequence encoding the first subunit and a second nucleic acid encoding the second subunit, each of the first or second nucleic acid sequences operably linked to at least a first promoter, such as a CMV promoter. In some embodiments, if the nucleic acid sequence encoding a cytokine or functional fragment thereof comprise two subunits, the disclosure relates to nucleic acid molecule comprises a first nucleic acid sequence encoding the first subunit and a second nucleic acid encoding the second subunit, the first nucleic acid sequence is operably linked to at least a first promoter and the second nucleic acid sequence is operably linked to at least a second promoter.

In some embodiments, the IL-12 sequences and nucleic acids sequences encoding the same can be found in U.S. Pat. Nos. 9,981,036 and 9,272,024, each of which is incorporated by reference in its entirety.

### Therapeutic Compositions and Administration

The present disclosure is also directed to pharmaceutical compositions comprising an effective amount of one or more nucleic acid molecules according to the present invention (including a pharmaceutically acceptable salt, thereof), optionally in combination with a pharmaceutically acceptable carrier, excipient or additive.

In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for treating and/or preventing viral infection and/or autoimmune disease.

A thorough discussion of pharmaceutically acceptable carriers, diluents, and other excipients is presented in Remington's Pharmaceutical Sciences (17th ed., Mack Publishing Company) and Remington: The Science and Practice of Pharmacy (21 st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic.

In one aspect, the disclosure provides a pharmaceutical composition comprising (i) one or a plurality of nucleic acid molecules as described herein (e.g. a nucleic acid molecule comprising a nucleic acid sequence comprising Formula I: [([(AEDⁿ)-(linker)]ₙ-[AEDⁿ⁺¹]); and (ii) a pharmaceutically acceptable carrier. In some embodiments, the nucleic acid molecule or nucleic acid sequence is free of a linker segment and the resulting plasmid comprises one or more successive nucleic acid sequences that encodes neoantigens amino acid sequences or epitopes that are from about 3 to about 30 amino acids in length. In some embodiments, the pharmaceutical composition comprises a pharmaceutically effective amount of: (i) one or a plurality of any of the nucleic acid molecules described herein comprising one or a combination of any component of a plasmid disclosed herein or nucleic acid sequences that are about 70%, 75%, 80%, 85%, 90%, 91%, 92%, 93%, 94%, 95%, 96%, 97%, 98%, or 99% homologous to any nucleic acid sequence that is a component of the plasmid listed herein. In some embodiments, the nucleic acid molecule comprises a nucleic acid sequence that encodes one or more nucleic acids that encode neoantigens and one or more linkers. In some embodiments, the nucleic acid molecule encodes one or a plurality of furin cleavage sequences separating one or more of the AEDs. In some embodiments, the disclosure relates to a pharmaceutical composition comprising a nucleic acid molecule that is pGX4505 or a nucleic acid sequence that is at least 70% homologous to the sequence of pGX4505, wherein its multiple cloning site is replaced by any of the Formulae disclosed herein.

In some embodiments, the pharmaceutical composition further comprises one or more therapeutic agents.

In some embodiments, the additional therapeutic agent is a biologic therapeutic or a small molecule.

In another embodiment, the therapeutic agent is (i) a checkpoint inhibitor or functional fragment thereof; or (ii) a nucleic acid molecule encoding a checkpoint inhibitor or a functional fragment thereof. In a further embodiment, the checkpoint inhibitor associates with or inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof. In an exemplary embodiment, the checkpoint inhibitor is an inhibitor of the programmed death-1 (PD-1) pathway. In another exemplary embodiment, the checkpoint inhibitor is an anti-cytotoxic T-lymphocyte-associated antigen 4 (CTLA4) antibody or functional fragment thereof.

In another embodiment, the therapeutic agent is an adjuvant. The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response. Exemplary adjuvants include, but are not limited to, poly-ICLC, 1018 ISS, aluminum salts, Amplivax AS15, BCG, CP-870,893, CpG7909, CyaA, GM-CSF, IC30, IC31, Imiquimod, ImuFact 1MP321, IS Patch, ISS, ISCOMATRIX, JuvImmune, Lipo Vac, monophosphoryf lipid A, Montanide IMS 1312, Montanide ISA 206, Montanide ISA 50V, Montanide ISA-51, OK-432, OM-174, OM-197-MP-EC, ONTAK, PEPTEL, vector system, PLGA micropartieles, resiquimod, S L172, Virosomes and other Virus-like particles, YF-17D, VEGF trap, R848, beta-gluean, Pam3Cys, acrylic or methacrylic polymers, copolymers of maleic anhydride and Aquila's QS21 stimulon, and a functional fragment of any thereof; or (ii) a nucleic acid molecule encoding an adjuvant selected from the group consisting of: (i) poly-ICLC, 1018 ISS, aluminum salts, Amplivax AS15, BCG, CP-870,893, CpG7909, CyaA, GM-CSF, IC30, IC31, Imiquimod, ImuFact 1MP321, IS Patch, ISS, ISCOMATRIX, JuvImmune, Lipo Vac, monophosphoryf lipid A, Montanide IMS 1312, Montanide ISA 206, Montanide ISA 50V, Montanide ISA-51, OK-432, OM-174, OM-197-MP-EC, ONTAK, PEPTEL, vector system, PLGA micropartieles, resiquimod, S L172, Virosomes and other Virus-like particles, YF-17D, VEGF trap, R848, beta-gluean, Pam3Cys, acrylic or methacrylic polymers, copolymers of maleic anhydride and Aquila's QS21 stimulon, or functional fragment thereof.

In another embodiment, the therapeutic agent is an immunostimulatory agent or functional fragment thereof. For example, in some embodiments, the imunostimulatory agent is an interleukin or functional fragment thereof.

In another embodiment, the therapeutic agent is a chemotherapeutic agent. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. For prostate cancer treatment, a preferred chemotherapeutic agent with which anti-CTLA-4 can be combined is paclitaxel (Taxol®).

One of skill in the art can determine which therapeutic regimen is appropriate on a subject by subject basis, depending, for example, on their cancer and their immune status (e.g., T-cell, B cell or NK cell activity and/or numbers).

According to the present disclosure, a host cell can be transfected in vivo (i.e., in an animal) or ex vivo (i.e., outside of an animal). Transfection of a nucleic acid molecule into a host cell can be accomplished by any method by which a nucleic acid molecule can be inserted into the cell. Transfection techniques include, but are not limited to, transfection, electroporation, microinjection, lipofection, adsorption, and protoplast fusion.

In some embodiments, the disclosure relates to a composition comprising one, two, three or more nucleic acid molecules, each nucleic acid molecule comprising at least one coding sequence comprising Formula I. In some embodiments, the first second and/or third nucleic acid molecule comprises at least one AED that is a neoantigen and at least AED that is a tumor associated antigen that is not derived from a subject. In some embodiments, the first second and/or third nucleic acid molecule comprises at least one coding sequence comprising at least about 10 AEDs derived from a subject. In some embodiments, the first second and/or third nucleic acid molecule comprises at least one coding sequence comprising at least about 20 AEDs derived from a subject. In some embodiments, the first second and/or third nucleic acid molecule comprises at least one coding sequence comprising at least about 30 AEDs derived from a subject. In some embodiments, the first second and/or third nucleic acid molecule comprises at least one coding sequence comprising at least about 40 AEDs derived from a subject. In some embodiments, the first second and/or third nucleic acid molecule comprises at least one coding sequence comprising at least about 50 AEDs derived from a subject. In some embodiments, the first second and/or third nucleic acid molecule comprises at least one coding sequence comprising at least about 60 AEDs derived from a subject.

In some embodiments, the first second and/or third nucleic acid molecule comprises at least one coding sequence comprising at least about 10 AEDs that are each independently a tumor associated antigen that is not derived from a subject. In some embodiments, the first second and/or third nucleic acid molecule comprises at least one coding sequence comprising at least about 20 AEDs that are each independently a tumor associated antigen that is not derived from a subject. In some embodiments, the first second and/or third nucleic acid molecule comprises at least one coding sequence comprising at least about 30 AEDs that are each independently a tumor associated antigen that is not derived from a subject. In some embodiments, the first second and/or third nucleic acid molecule comprises at least one coding sequence comprising at least about 40 AEDs that are each independently a tumor associated antigen that is not derived from a subject. In some embodiments, the first second and/or third nucleic acid molecule comprises at least one coding sequence comprising at least about 50 AEDs that are each independently a tumor associated antigen that is not derived from a subject. In some embodiments, the first second and/or third nucleic acid molecule comprises at least one coding sequence comprising at least about 60 AEDs that are each independently a tumor associated antigen that is not derived from a subject. Any ratio of nucleic acid sequence encoding a neoantigen:nucleic acid sequence encoding a tumor associated antigen not derived from the subject may be included in the embodiments, such as 1:1, 2:1, 1:2, 1:4, 4:1, 5:1, 1:5, 1:3, 3:1, etc.

In some embodiments, the nucleic acid sequence comprises at least 2, 3, 4, 5, 6, 7, 8, 9, or 10 or more linker domains and the nucleic acid sequence comprises

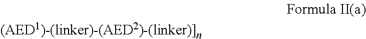

wherein each AED is independently selectable from any one or plurality of tumor associated antigens from a subject or one or a plurality of tumor antigens not derived from the subject and wherein n is any positive integer from about 1 to about 100 and wherein each “linker” is a nucleic acid sequence encoding one or a plurality of amino acid cleavage sites. In some embodiments, the nucleic acid sequence comprises at least one linker domain between each AED and the nucleic acid sequence comprises

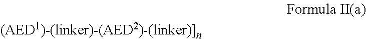

wherein each AED is independently selectable from any one or plurality of tumor associated antigens from a subject or one or a plurality of tumor antigens not derived from the subject and wherein n is any positive integer from about 25 to about 60 and wherein each “linker” is a nucleic acid sequence encoding one or a plurality of amino acid cleavage sites.

In some embodiments, the nucleic acid sequence comprises at least one linker domain between each AED and the nucleic acid sequence comprises

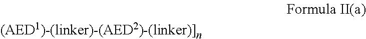

wherein each AED is independently selectable from any one or plurality of tumor associated antigens from a subject or one or a plurality of tumor antigens not derived from the subject and wherein n is any positive integer from about 35 to about 50 and wherein each “linker” is a nucleic acid sequence encoding one or a plurality of amino acid cleavage sites. In some embodiments, the nucleic acid sequence comprises at least one linker domain between each AED and the nucleic acid sequence comprises

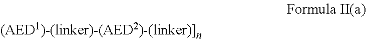

wherein each AED is independently selectable from any one or plurality of tumor associated antigens from a subject or one or a plurality of tumor antigens not derived from the subject and wherein n is any positive integer from about 40 to about 50 and wherein each “linker” is a nucleic acid sequence encoding one or a plurality of amino acid cleavage sites.

In some embodiments, tumor associated antigens not derived from the subject comprise one or a combination of amino acids comprising at least about 70%, 75%, 80%, 85%, 90%, 91%, 92%, 93%, 94%, 95%, 96%, 97%, 98%, 99% or 100% to Survivin, MAGE A10, gp100, EGFRvIII, calreticulin and WT1.

```
Survivin
(SEQ ID NO: 61)
MGAPTLPPAW QPFLKDHRIS TFKNWPFLEG CACTPERMAE AGFIHCPTEN
EPDLAQCFFCFKELEGWEPD DDPIEEHKKH SSGCAFLSVK KQFEELTLGE
FLKLDRERAK NKIAKETNNK KEFEETAEK VRRAIEQLAA MD
MAGE
(SEQ ID: 62)
    1 matsqadiet dpgisepdga taqtsadgsq aqnlesrtii rgkrtrkinn lnveenssgd
  61 qrraplaagt wrsapvpvtt qnppgappnv lwqtplawqn psgwqnqtar qtpparqspp
121 arqtppawqn pvawqnpviw pnpviwqnpv iwpnpivwpg pvvwpnplaw qnppgwqtpp
181 gwqtppgwqg ppdwqgppdw plppdwplpp dwplptdwpl ppdwipadwp ippdwqnlrp
241 spnlrpspns rasqnpgaaq prdvallqer anklvkylml kdytkvpikr semlrdiire
301 ytdvypeiie racfvlekkf giqlkeidke ehlyilistp eslagilgtt kdtpklglll
361 vilgvifmng nraseavlwe alrkmglrpg vrhpllgdlr klltyefvkq kyldyrrvpn
421 snppeyeflw glrsyhetsk mkvlrfiaev qkrdprdwta qfmeaadeal daldaaaaea
481 earaeartrm gigdeavsgp wswddiefel ltwdeegdfg dpwsripftf waryhqnars
541 rfpqtfagpi igpggtasan faanfgaigf fwve
A10
(SEQ ID NO: 63)
    1 mtdktekvav dpetvfkrpr ecdspsyqkr qrmallarkq gagdsliags amskekklmt
  61 ghaippsqld sqiddftgfs kdgmmqkpgs napvggnvts nfsgddlecr giasspksqq
121 einadikcqv vkeirclgrk yekifemleg vqgptavrkr ffesiikeaa rcmrrdfvkh
181 1kkklkrmi
gp100
(SEQ ID NO: 64)
    1 mdlvlkrell hlavigalla vgatkvprnq dwlgvsrqlr tkawnrqlyp ewteaqrldc
  61 wrggqvslkv sndgptliga nasfsialnf pgsqkvlpdg qviwvnntii ngsqvwggqp
121 vypqetddac ifpdggpcps gswsqkrsfv yvwktwgqyw qvlggpvsgl sigtgramlg
181 thtmevtvyh rrgsrsyvpl ahsssaftit dqvpfsvsvs qlraldggnk hflrnqpltf
241 alqlhdpsgy laeadlsytw dfgdssgtli sralvvthty lepgpvtaqv vlqaaiplts
301 cgsspvpgtt dghrptaeap nttagqvptt evvgttpgqa ptaepsgtts vqvpttevis
361 tapvqmptae stgmtpekvp vsevmgttla emstpeatgm tpaevsivvl sgttaaqvtt
421 tewvettare lpipepegpd assimstesi tgslgplldg tatlrlvkrq vpldcvlyry
481 gsfsvtldiv qgiesaeilq avpsgegdaf eltvscqggl pkeacmeiss pgcqppaqrl
541 cqpvlpspac qlvlhqilkg gsgtyclnvs ladtnslavv stqlimpgqe aglgqvpliv
601 gillvlmavv lasliyrrrl mkqdfsvpql phssshwlrl prifcscpig enspllsgqq
661 v
EGFRvIII
(SEQ ID NO: 65)
    1 mrpsgtagaa llallaalcp asraleekkg nyvvtdhgsc vracgadsye meedgvrkck
  61 kcegpcrkvc ngigigefkd slsinatnik hfknctsisg dlhilpvafr gdsfthtppl
121 dpqeldilkt vkeitgflli qawpenrtdl hafenleiir grtkqhgqfs lavvslnits
181 1glrslkeis dgdviisgnk nlcyantinw kklfgtsgqk tkiisnrgen sckatgqvch
241 alcspegcwg peprdcvscr nvsrgrecvd kcnllegepr efvenseciq chpeclpqam
301 nitctgrgpd nciqcahyid gphcvktcpa gvmgenntlv wkyadaghvc hlchpnctyg
361 ctgpglegcp tngpkipsia tgmvgallll lvvalgiglf mrrrhivrkr tlrrllqere
421 lvepltpsge apnqallril ketefkkikv lgsgafgtvy kglwipegek vkipvaikel
481 reatspkank eildeayvma svdnphvcrl lgicltstvq litqlmpfgc lldyvrehkd
541 nigsqyllnw cvqiakgmny ledrrlvhrd laarnvlvkt pqhvkitdfg lakllgaeek
601 eyhaeggkvp ikwmalesil hriythqsdv wsygvtvwel mtfgskpydg ipaseissil
661 ekgerlpqpp ictidvymim vkcwmidads rpkfreliie fskmardpqr ylviqgderm
721 hlpsptdsnf yralmdeedm ddvvdadeyl ipqqgffssp stsrtpllss 1satsnnstv
781 acidrnglqs cpikedsflq ryssdptgal tedsiddtfl pvpeyinqsv pkrpagsvqn
841 pvyhnqplnp apsrdphyqd phstavgnpe ylntvqptcv nstfdspahw aqkgshqisl
901 dnpdyqqdff pkeakpngif kgstaenaey Irvapqssef iga
Calreticulin
(SEQ ID NO: 66)
    1 mllsvplllg llglavaepa vyfkeqfldg dgwtsrwies khksdfgkfv lssgkfygde
  61 ekdkglqtsq darfyalsas fepfsnkgqt lvvqftvkhe qnidcgggyv klfpnsldqt
121 dmhgdseyni mfgpdicgpg tkkvhvifny kgknvlinkd irckddefth lytlivrpdn
181 tyevkidnsq vesgsleddw dflppkkikd pdaskpedwd erakiddptd skpedwdkpe
241 hipdpdakkp edwdeemdge weppviqnpe ykgewkprqi dnpdykgtwi hpeidnpeys
301 pdpsiyaydn fgvlgldlwq vksgtifdnf litndeayae efgnetwgvt kaaekqmkdk
361 qdeeqrlkee eedkkrkeee eaedkedded kdedeedeed keedeeedvp gqakdel
WT1
(SEQ ID NO: 67)
    1 mdflllqdpa stcvpepasq htlrsgpgcl qqpeqqgvrd pggiwaklga acasaerlqg
  61 rrsrgasgse pqqmgsdvrd Inallpavps lgggggcalp vsgaaqwapv ldfappgasa
121 ygslggpapp papppppppp phsfikqeps wggaepheeq clsaftvhfs gqftgtagac
181 rygpfgpppp sqassgqarm fpnapylpsc lesqpairnq gystvtfdgt psyghtpshh
241 aaqfpnhsfk hedpmgqqgs lgeqqysvpp pvygchtptd sctgsqalll rtpyssdnly
301 qmtsqlecmt wnqmnlgatl kghstgyesd nhttpilcga qyrihthgvf rgiqdvrrvp
361 gvaptlvrsa setsekrpfm caypgenkry fklshlqmhs rkhtgekpyq cdfkdcerrf
421 srsdqlkrhq rrhtgvkpfq cktcqrkfsr sdhlkthtrt htgekpfscr wpscqkkfar
481 sdelvrhhnm hqrnmtklql al.
```

The disclosure relates to a nucleic acid sequence comprising one or a plurality of nucleic acid sequence encoding one or a plurality of neoantigens and one or a plurality of nucleic acid sequences encoding one or a plurality of tumor associated antigens. In some embodiments, the tumors associated antigens not derived from a subject are chosen from one or a combination of amino acid sequences comprising at least about 70%, 75%, 80%, 85%, 90%, 91%, 92%, 93%, 94%, 95%, 96%, 97%, 98%, 99% or 100% sequence identity to SEQ ID NO:61, 62, 63, 64, 65, 66 or 67, or functional fragments thereof.

The disclosure relates to a nucleic acid sequence comprising one or a plurality of nucleic acid sequence encoding one or a plurality of neoantigens and one or a plurality of nucleic acid sequences encoding one or a plurality of tumor associated antigens, wherein the one or plurality of neoantigens are chosen from one or a plurality of amino acid sequences comprising at least about 70%, 75%, 80%, 85%, 90%, 91%, 92%, 93%, 94%, 95%, 96%, 97%, 98%, 99% or 100% sequence identity to SEQ ID NO: 1-20, or functional fragments thereof.

Routes of administration include, but are not limited to, intramuscular, intranasally, intradermally, intraperitoneal, intradermal, subcutaneous, intravenous, intraarterially, intraocularly and oral as well as topically, transdermally, by inhalation or suppository or to mucosal tissue such as by lavage to vaginal, rectal, urethral, buccal and sublingual tissue. Preferred routes of administration include intramuscular, intraperitoneal, intradermal and subcutaneous injection. Genetic constructs may be administered by means including, but not limited to, traditional syringes, needleless injection devices, “microprojectile bombardment gone guns”, or other physical methods such as electroporation (“EP”), “hydrodynamic method”, or ultrasound.

Examples of electroporation devices and electroporation methods preferred for facilitating delivery of the DNA vaccines of the present invention, include those described in U.S. Pat. No. 7,245,963 by Draghia-Akli, et al., U.S. Patent Application Publication No. 2005/0052630 submitted by Smith, et al., the contents of which are hereby incorporated by reference in their entirety. Also preferred, are electroporation devices and electroporation methods for facilitating delivery of the DNA vaccines provided in co-pending and co-owned U.S. patent application Ser. No. 11/874,072, filed Oct. 17, 2007, which claims the benefit under 35 USC 1 19(e) to U.S. Provisional Application Ser. No. 60/852,149, filed Oct. 17, 2006, and U.S. Provisional Application Ser. No. 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Application Publication No. 2005/0052630, incorporated by reference in its entirety herein, describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk.

The electrode arrays and methods described in U.S. Pat. No. 7,245,963 and U.S. Patent Application Publication No. 2005/0052630 are adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes. The electrodes described in U.S. Pat. No. 7,245,963 and U.S. Patent Application Publication No. 2005/005263 are preferably 20 mm long and 21 gauge.

In certain exemplary embodiments, electroporation devices can be configured to deliver to a desired tissue of a mammal a pulse of energy producing a constant current similar to a preset current input by a user. The electroporation device comprises an electroporation component and an electrode assembly or handle assembly. The electroporation component can include and incorporate one or more of the various elements of the electroporation devices, including: controller, current waveform generator, impedance tester, waveform logger, input element, status reporting element, communication port, memory component, power source, and power switch. The electroporation component can function as one element of the electroporation devices, and the other elements are separate elements (or components) in communication with the electroporation component. In some embodiments, the electroporation component can function as more than one element of the electroporation devices, which can be in communication with still other elements of the electroporation devices separate from the electroporation component. The present invention is not limited by the elements of the electroporation devices existing as parts of one electromechanical or mechanical device, as the elements can function as one device or as separate elements in communication with one another. The electroporation component is capable of delivering the pulse of energy that produces the constant current in the desired tissue, and includes a feedback mechanism. The electrode assembly includes an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism can receive the measured impedance and can adjust the pulse of energy delivered by the electroporation component to maintain the constant current.

In some embodiments, the plurality of electrodes can deliver the pulse of energy in a decentralized pattern. In some embodiments, the plurality of electrodes can deliver the pulse of energy in the decentralized pattern through the control of the electrodes under a programmed sequence, and the programmed sequence is input by a user to the electroporation component. In some embodiments, the programmed sequence comprises a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

In some embodiments, the feedback mechanism is performed by either hardware or software. Preferably, the feedback mechanism is performed by an analog closed-loop circuit. In certain embodiments, this feedback occurs every 50 μs, 20 μs, 10 μs or 1 μs, but is preferably a realtime feedback or instantaneous (i.e., substantially instantaneous as determined by available techniques for determining response time). In some embodiments, the neutral electrode measures the impedance in the desired tissue and communicates the impedance to the feedback mechanism, and the feedback mechanism responds to the impedance and adjusts the pulse of energy to maintain the constant current at a value similar to the preset current. In some embodiments, the feedback mechanism maintains the constant current continuously and instantaneously during the delivery of the pulse of energy.

For therapeutic or immunization purposes, nucleic acid molecules of the invention can also be administered to the patient. A number of methods are conveniently used to deliver the nucleic acids to the patient. For instance, the nucleic acid can be delivered directly, as “naked DNA”. This approach is described, for instance, in Wolff et al., Science 247:1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles.

The nucleic acids can also be delivered complexed to cationic compounds, such as cationic lipids. Lipid-mediated gene delivery methods are described, for instance, in WO1996/18372; WO 1993/24640; Mannino & Gould-Fogerite, BioTechniques 6(7): 682-691 (1988); U.S. Pat. No. 5,279,833; WO 1991/06309; and Feigner et al., Proc. Natl. Acad. Sci. USA 84:7413-7414 (1987).

RNA encoding the peptide of interest can also be used for delivery (see, e.g., Kiken et al, 2011; Su et al, 2011).

A pharmaceutically acceptable carrier or excipient can include such functional molecules as vehicles, adjuvants, carriers or diluents, which are known and readily available to the public.

In some embodiments, the pharmaceutically acceptable carrier is an adjuvant. In some embodiments, the pharmaceutically acceptable excipient is a transfection facilitating agent. Preferably, the transfection facilitating agent is a polyanion, polycation, or lipid, and more preferably poly-L-glutamate. In some embodiments, the nucleic acid molecule, or DNA plasmid, is delivered to the cells in conjunction with administration of a polynucleotide function enhancer or a genetic vaccine facilitator agent (or transfection facilitating agent). Polynucleotide function enhancers are described in U.S. Pat. Nos. 5,593,972, 5,962,428, and International Patent Application No. PCT/US94/00899 filed Jan. 26, 1994, which are each incorporated herein by reference in their entireties. Genetic vaccine facilitator agents are described in U.S. Patent Application Ser. No. 021,579 filed Apr. 1, 1994, which is incorporated herein by reference in its entirety. The transfection facilitating agent can be administered in conjunction with nucleic acid molecules as a mixture with the nucleic acid molecule or administered separately simultaneously, before or after administration of nucleic acid molecules. Examples of transfection facilitating agents includes surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA plasmid vaccines may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

In some preferred embodiments, the DNA plasmids are delivered with genes for proteins which further enhance the immune response. Examples of such genes are those which encode other cytokines and lymphokines such as alpha-interferon, gamma-interferon, platelet derived growth factor (PDGF), TNF α, TNFβ, GM-CSF, epidermal growth factor (EGF), IL-1, IL-2, IL-4, IL-5, IL-6, IL-10, IL-12, IL-18, MHC, CD80, CD86 and IL-15 including IL-15 having the signal sequence deleted and optionally including the signal peptide from IgE. Other genes which may be useful include those encoding: MCP-1, MIP-1a, MIP-Ip, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

When the agents described herein are administered as pharmaceuticals to humans or animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, agents or pharmaceutical compositions of the invention are administered in an amount sufficient to reduce or eliminate symptoms associated with viral infection and/or autoimmune disease.

The composition comprising one or a plurality of nucleic acid molecules described herein preferably comprise DNA quantities of from about 1 nanogram to 10 milligrams; about 1 microgram to about 10 milligrams; or preferably about 0.1 microgram to about 10 milligrams; or more preferably about 100 microgram to about 1 milligram. In some preferred embodiments, DNA plasmid vaccines according to the present invention comprise about 5 nanograms to about 1000 micrograms of DNA. In some preferred embodiments, the DNA plasmid vaccines contain about 10 nanograms to about 800 micrograms of DNA. In some preferred embodiments, the DNA plasmid vaccines contain about 0.1 to about 500 micrograms of DNA. In some preferred embodiments, the DNA plasmid vaccines contain about 1 to about 350 micrograms of DNA. In some preferred embodiments, the DNA plasmid vaccines contain about 25 to about 250 micrograms of DNA. In some preferred embodiments, the DNA plasmid vaccines contain about 100 microgram to about 1 milligram DNA.

The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

Preferably the DNA formulations for use with a muscle or skin EP device described herein have high DNA concentrations, preferably concentrations that include microgram to tens of milligram quantities, and preferably milligram quantities, of DNA in small volumes that are optimal for delivery to the skin, preferably small injection volume, ideally 25-200 microliters (μL). In some embodiments, the DNA formulations have high DNA concentrations, such as 1 mg/mL or greater (mg DNA/volume of formulation). More preferably, the DNA formulation has a DNA concentration that provides for gram quantities of DNA in 200 μL of formula, and more preferably gram quantities of DNA in 100 μL of formula.

The DNA plasmids for use with the electroporation devices of the present invention can be formulated or manufactured using a combination of known devices and techniques, but preferably they are manufactured using an optimized plasmid manufacturing technique that is described in U.S. Patent Application Publication No. 20090004716, incorporated by reference in its entirety herein. In some examples, the DNA plasmids used in these studies can be formulated at concentrations greater than or equal to 10 mg/mL. The manufacturing techniques also include or incorporate various devices and protocols that are commonly known to those of ordinary skill in the art, in addition to those described in U.S. Patent Application Publication No. 20090004716 and those described in U.S. Pat. No. 7,238,522, incorporated by reference in their entireties herein. The high concentrations of plasmids used with the skin electroporation devices and delivery techniques described herein allow for administration of plasmids into the ID/SC space in a reasonably low volume and aids in enhancing expression and immunization effects.

Preferred unit dosage formulations are those containing a daily dose or unit, daily sub-dose, as hereinabove recited, or an appropriate fraction thereof, of the administered ingredient.

The dosage regimen for treating a disorder or a disease with the tumor specific neo-antigenic peptides of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The amounts and dosage regimens administered to a subject will depend on a number of factors, such as the mode of administration, the nature of the condition being treated, the body weight of the subject being treated and the judgment of the prescribing physician.

The quantity of DNA included within therapeutically active formulations according to the present invention is an effective amount for treating the disease or condition. Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., reduce or eliminate symptoms associated with viral infection or autoimmune disease) is observed in the treated subject, with minimal or acceptable toxic side effects. Applicable methods for determining an appropriate dose and dosing schedule for administration of a pharmaceutical composition of the present invention are described, for example, in Goodman and Gilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

In certain embodiments, the pharmaceutical composition is administered once daily; in other embodiments, the pharmaceutical composition is administered twice daily; in yet other embodiments, the pharmaceutical composition is administered once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

In some embodiments, several divided dosages, as well as staggered dosages, can be administered daily or sequentially, or the dose can be continuously infused, or can be a bolus injection. Further, the dosages of the compound(s) of the disclosure can be proportionally increased or decreased as indicated by the exigencies of the therapeutic or prophylactic situation.

In some embodiments, the present disclosure also relates to methods for administration of the pharmaceutical compositions described herein using a prime-boost regimen. The term of “prime-boost” refers to the successive administrations of two different immunogenic or immunological composition types having at least one immunogen in common. The priming administration (priming) is the administration of a first immunogenic or immunological composition type and may comprise one, two or more administrations. The boost administration is the administration of a second immunogenic or immunological composition type and may comprise one, two or more administrations, and, for instance, may comprise or consist essentially of annual administrations. The “boost” may be administered from about 2 weeks to about 32 weeks after the “priming”, or from about 4 to about 30 weeks after the priming, or from about 8 to about 28 weeks after the priming, advantageously from about 16 to about 24 weeks after the priming, and more advantageously, about 24 weeks after the priming.

The pharmaceutical compositions described herein can be used to treat diseases and disease conditions that are acute, and may also be used for treatment of chronic conditions. In certain embodiments, the pharmaceutical composition of the invention are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the pharmaceutical composition of the invention to be administered for the remainder of the patient's life. In preferred embodiments, the patient is monitored to check the progression of the disease or disorder, and the dose is adjusted accordingly. In preferred embodiments, treatment according to the invention is effective for at least two weeks, three weeks, one month, two months, three months, four months, five months, six months, one year, two years, three years, four years, or five years, ten years, fifteen years, twenty years, or for the remainder of the subject's life.

### Combination Therapy

According to embodiments of the disclosure, the pharmaceutical compositions described herein may be administered with one or more additional therapeutic agents. Various combination therapies contemplated by the present invention are described throughout.

In certain embodiments, any of the additional therapeutic agents is administered chronologically after or simultaneously with the DNA vaccine. In certain embodiments, the additional therapeutic agent is administered 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 month, or any combination thereof, before the DNA vaccine or immunogenic compositions is administered. In certain embodiments, the additional therapeutic agent is administered 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 month, or any combination thereof, after the DNA vaccine or immunogenic compositions is administered.

### Adjuvants

In a further embodiment, the method further comprises administering an adjuvant to the subject. Administration may be either prior to, simultaneously with, or after treatment with the DNA vaccine or immunogenic compositions described herein.

Effective vaccine or immunogenic compositions described herein may include a strong adjuvant to initiate an immune response.

In certain embodiments, the adjuvant is selected from the group consisting of poly-ICLC, 1018 ISS, aluminum salts, Amplivax. AS15, BCG, CP-870,893, CpG7909, CyaA, GM-CSF, IC30, IC31, Imiquimod, ImuFact 1MP321, IS Patch, ISS, ISCOMATRIX, JuvImmune, Lipo Vac, monophosphoryf lipid A, Montanide IMS 1312, Montanide ISA 206, Montanide ISA 50V, Montanide ISA-51, OK-432, OM-174, OM-197-MP-EC, ONTAK, PEPTEL, vector system, PLGA micropartieles, resiquimod, S L172, Virosomes and other Virus-like particles, YF-17D, VEGF trap, R848, beta-gluean, Pam3Cys, acrylic or methacrylic polymers, copolymers of maleic anhydride and Aquila's QS21 stimulon

As described herein, poly-ICLC, an agonist of TLR3 and the RNA helicase-domains of MDA5 and RIG3, has shown several desirable properties for a vaccine or immunogenic composition adjuvant. These properties include the induction of local and systemic activation of immune cells in vivo, production of stimulatory chemokines and cytokines, and stimulation of antigen-presentation by DCs. Furthermore, poly-ICLC can induce durable CD4+ and CD 8+ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly-ICLC in combination with a NY-ESO-1 peptide vaccine (in addition to Montanide) showed induction of CD4+ and CD8+ T cell, as well as antibody responses to the peptide in a recent phase 1 study. At the same time, poly-ICLC has been extensively tested in more than 25 clinical trials to date and exhibited a relatively benign toxicity profile. In addition to a powerful and specific immunogen the neoantigen vaccines of the present disclosure may be combined with an adjuvant (e.g., poly-ICLC). Without being bound by theory, these neoantigens are expected to bypass central thymic tolerance (thus allowing stronger anti-tumor T cell response), while reducing the potential for autoimmunity (e.g., by avoiding targeting of normal self-antigens). An effective immune response advantageously includes a strong adjuvant to activate the immune system (Speiser and Romero, Molecularly defined vaccines for cancer immunotherapy, and protective T cell immunity Seminars in Immunol 22:144 (2010)). For example, Toll-like receptors (TLRs) have emerged as powerful sensors of microbial and viral pathogen “danger signals”, effectively inducing the innate immune system, and in turn, the adaptive immune system (Bhardwaj and Gnjatic, Cancer J. 16:382-391 (2010)). Among the TLR agonists, poly-ICLC (a synthetic double-stranded RNA mimic) is one of the most potent activators of myeloid-derived dendritic cells. In a human volunteer study, poly-ICLC has been shown to be safe and to induce a gene expression profile in peripheral blood cells comparable to that induced by one of the most potent live attenuated viral vaccines, the yellow fever vaccine YF-17D (Caskey et al, Synthetic double-stranded RNA induces innate immune responses similar to a live viral vaccine in humans J Exp Med 208:2357 (201 1)). In other embodiments, other adjuvants described herein are envisioned. For instance oil-in-water, water-in-oil or multiphasic W/O/W; see, e.g., U.S. Pat. No. 7,608,279 and Aucouturier et al, Vaccine 19 (2001), 2666-2672, and documents cited therein.

A combination of any one or more (e.g. 1, 2, 3, 4, 5 or more) adjuvants can be used in combination with the DNA vaccine or immunogenic compositions described herein.

In some embodiments, the pharmaceutical composition comprising a first plasmid encoding one or a plurality of neoantigens and a second and/or third and/or fourth plasmid, each second, third or fourth plasmid comprising a nucleic acid sequence encoding a cytokine or functional fragment thereof.

### Checkpoint Inhibitors

In a further embodiment, the method further comprises administering a checkpoint inhibitor to the subject. Administration may be either prior to, simultaneously with, or after treatment with the DNA vaccine or immunogenic compositions described herein.

Immune checkpoints regulate T cell function in the immune system. T cells play a central role in cell-mediated immunity. Checkpoint proteins interact with specific ligands which send a signal into the T cell and essentially switch off or inhibit T cell function. Cancer cells take advantage of this system by driving high levels of expression of checkpoint proteins on their surface which results in control of the T cells expressing checkpoint proteins on the surface of T cells that enter the tumor microenvironment, thus suppressing the anticancer immune response. As such, inhibition of checkpoint proteins would result in restoration of T cell function and an immune response to the cancer cells.

Checkpoint inhibitors include any agent that blocks or inhibits the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8+ (αβ) T cells), CD160 (also referred to as BY55), CGEN-15049, CHK 1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). Checkpoint protein ligands include, but are not limited to PD-L1, PD-L2, B7-H3, B7-H4, CD28, CD86 and TIM-3.

In some embodiments, the present disclosure covers the use of a specific class of checkpoint inhibitor are drugs that block the interaction between immune checkpoint receptor programmed cell death protein 1 (PD-1) and its ligand PDL-1. See A. Mullard, “New checkpoint inhibitors ride the immunotherapy tsunami,” Nature Reviews: Drug Discovery (2013), 12:489-492. PD-1 is expressed on and regulates the activity of T-cells. Specifically, when PD-1 is unbound to PDL-1, the T-cells can engage and kill target cells. However, when PD-1 is bound to PDL-1 it causes the T-cells to cease engaging and killing target cells. Furthermore, unlike other checkpoints, PD-1 acts proximately such the PDLs are overexpressed directly on cancer cells which leads to increased binding to the PD-1 expressing T-cells.

One aspect of the present disclosure provides checkpoint inhibitors which are antibodies that can act as agonists of PD-1, thereby modulating immune responses regulated by PD-1. In some embodiments, the anti-PD-1 antibodies can be antigen-binding fragments. Anti-PD-1 antibodies disclosed herein are able to bind to human PD-1 and agonize the activity of PD-1, thereby inhibiting the function of immune cells expressing PD-1.

In some embodiments, the present disclosure covers the use of a specific class of checkpoint inhibitor are drugs that inhibit CTLA-4. Suitable anti-CTLA4 antagonist agents for use in the methods of the invention, include, without limitation, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B1. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al, Proc. Natl. Acad. Sci. USA, 95 (17): 10067-10071 (1998); Camacho et al, J. Clin. Oncology, 22 (145): Abstract No. 2505 (2004) (antibody CP-675206); Mokyr et al, Cancer Res., 58:5301-5304 (1998), and U.S. Pat. Nos. 5,977,318, 6,682,736, 7,109,003, and 7,132,281.

Additional anti-CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway; antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway, among other anti-CTLA4 antagonists.

In some embodiments, the present disclosure covers the use of a specific class of checkpoint inhibitor drugs that inhibit TIM-3. Blocking the activation of TIM-3 by a ligand, results in an increase in Th1 cell activation. Furthermore, TIM-3 has been identified as an important inhibitory receptor expressed by exhausted CD8⁺ T cells. TIM-3 has also been reported as a key regulator of nucleic acid mediated antitumor immunity. In one example, TIM-3 has been shown to be upregulated on tumor-associated dendritic cells (TADCs).

The combination of a check point inhibitor and DNA vaccine or immunogenic composition described herein can be more effective in treating cancer in some subjects and/or can initiate, enable, increase, enhance or prolong the activity and/or number of immune cells (including T cells, B cells, NK cells and/or others) or convey a medically beneficial response by a tumor (including regression, necrosis or elimination thereof).

A combination of any one or more (e.g. 1, 2, 3, 4, 5 or more) checkpoint inhibitors can be used in combination with the DNA vaccine or immunogenic compositions described herein.

### Immunostimulatory Agents

In a further embodiment, the method further comprises administering one or more immunostimulatory agents to the subject. Administration may be either prior to, simultaneously with, or after treatment with the DNA vaccine or immunogenic compositions described herein.

In some embodiments, the present invention is directed to the use of immunostimulatory agents, including T cell growth factors and interleukins. Immunostimulatory agents are substances (drugs and nutrients) that stimulate the immune system by inducing activation or increasing activity of any of its components. Immunostimulants include bacterial vaccines, colony stimulating factors, interferons, interleukins, other immunostimulants, therapeutic vaccines, vaccine combinations and viral vaccines.

T cell growth factors are proteins which stimulate the proliferation of T cells. Examples of T cell growth factors include 11-2, IL-7, IL-15, IL-17, IL-21 and IL-33.

Interleukins are a group of cytokines that were first seen to be expressed by white blood cells. The function of the immune system depends in a large part on interleukins, and rare deficiencies of a number of them have been described, all featuring autoimmune diseases or immune deficiency. The majority of interleukins are synthesized by helper CD4 T lymphocytes, as well as through monocytes, macrophages, and endothelial cells. They promote the development and differentiation of T and B lymphocytes, and hematopoietic cells. Examples of interleukins include IL-1, IL-2, IL-3, IL-4, IL-5, IL-6, IL-7, IL-8, IL-9, IL-10, IL-11, IL-12, IL-13, IL-14, IL-15 and IL-17.

In some embodiments, the interleukin is IL-12.

In some embodiments, the DNA plasmids are delivered with immunostimulatory agents that are genes for proteins which further enhance the immune response against such target proteins. Examples of such genes are those which encode other cytokines and lymphokines such as alpha-interferon, gamma-interferon, platelet derived growth factor (PDGF), TNF α, TNFβ, GM-CSF, epidermal growth factor (EGF), IL-1, IL-2, IL-4, IL-5, IL-6, IL-10, IL-12, IL-18, MHC, CD80, CD86 and IL-15 including IL-15 having the signal sequence deleted and optionally including the signal peptide from IgE. Other genes which may be useful include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

A combination of any one or more (e.g. 1, 2, 3, 4, 5 or more) immunostimulatory agents can be used in combination with the DNA vaccine or immunogenic compositions described herein.

### Chemotherapeutic Agents

In a further embodiment, the method further comprises administering a chemotherapeutic agent, targeted therapy or radiation to the subject. Administration may be either prior to, simultaneously with, or after treatment with the DNA vaccine or immunogenic compositions described herein.

Examples of cancer therapeutic agents or chemotherapeutic agents include alkylating agents such as thiotepa and cyclophosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb Oncology, Princeton, N.J.) and doxetaxel (TAXOTEPvE™, Pvhne-Poulenc Rorer, Antony, France); chlorambucil; gemcitabine; 6-thioguanine; mercaptopurine; methotrexate; platinum analogs such as cisplatin and carboplatin; vinblastine; trastuzumab, docetaxel, platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylomithine (DMFO); retinoic acid derivatives such as Targretin™ (bexarotene), Panretin™ (alitretinoin); ONTAKT™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 1 17018, onapristone, and toremifene (Fareston); and anti-androgens such as flutamide, nilutamide, bicalutamide, leuprolide, and goserelin; and pharmaceutically acceptable salts, acids or derivatives of any of the above. Further cancer therapeutic agents include sorafenib and other protein kinase inhibitors such as afatinib, axitinib, bevacizumab, cetuximab, crizotinib, dasatinib, erlotinib, fostamatinib, gefitinib, imatinib, lapatinib, lenvatinib, mubritinib, nilotinib, panitumumab, pazopanib, pegaptanib, ranibizumab, ruxolitinib, trastuzumab, vandetanib, vemurafenib, and sunitinib; sirolimus (rapamycin), everolimus and other mTOR inhibitors.

Examples of additional chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea). Moreover, exemplary chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and other inhibitors of proteins or enzymes known to be upregulated, over-expressed or activated in cancers, the inhibition of which downregulates cell replication.

A combination of any one or more (e.g. 1, 2, 3, 4, 5 or more) chemotherapeutic agents can be used in combination with the DNA vaccine or immunogenic compositions described herein.

In certain embodiments, the subject nucleic acid molecules, and compositions comprising the nucleic acid molecules, of the disclosure can be used alone.

### Vaccines

In an exemplary embodiment, the present invention is directed to an immunogenic composition, e.g., a vaccine, composition comprising the nucleic acid molecules described herein, capable of raising an immune response, and in particular a specific T-cell response.

DNA vaccines are described in U.S. Pat. Nos. 5,593,972, 5,739,118, 5,817,637,

U.S. Pat. Nos. 5,830,876, 5,962,428, 5,981,505, 5,580,859, 5,703,055, 5,676,594, and the priority applications cited therein, which are each incorporated herein by reference. In addition to the delivery protocols described in those applications, alternative methods of delivering DNA are described in U.S. Pat. Nos. 4,945,050 and 5,036,006, which are both incorporated herein by reference.

In certain embodiments, the vaccine composition comprises mutant neo-antigenic nucleic acid molecules as described herein (e.g. comprising a nucleic acid sequence comprising the formula: [(antigen expression domain 1)-(linker)-(antigen expression domain 2)-(linker)] n), corresponding to tumor specific neo-antigens identified by the methods described herein. A suitable vaccine will preferably contain a plurality of tumor specific neo-antigenic nucleic acid molecules. In an embodiment, the vaccine will include between about 1 to about 200 nucleic acid molecules, between about 2 to about 100 nucleic acid molecules, between about 2 to about 58 nucleic acid molecules, between about 2 to about 29 nucleic acid molecules. In certain embodiments, the vaccine will include about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleic acid molecules. In certain embodiments, the vaccine will include about 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleic acid molecules. In certain embodiments, the vaccine will include about 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 nucleic acid molecules. In certain embodiments, the vaccine will include about 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 nucleic acid molecules. In certain embodiments, the vaccine will include about 81, 82, 83, 84, 85, 86, 87, 88, 89, 90, 91, 92, 93, 94, 95, 96, 97, 98, 99 or 100 nucleic acid molecules.

In certain embodiments, the vaccine composition is capable of enhancing a CD8+ T cell immune response in a subject. In some embodiments, enhancing the CD8+ T cell immune response comprises activating CD8+ T cells. In another embodiment, enhancing the CD8+ T cell immune response comprises expanding CD8+ T cells. In other embodiments, the vaccine composition is capable of raising a specific cytotoxic T-cells response and/or a specific helper T-cell response.

The vaccine composition can further comprise an adjuvant and/or a carrier.

Adjuvants are described herein, and are any substance whose admixture into the vaccine composition increases or otherwise modifies the immune response to the mutant peptide. Carriers are scaffold structures, for example a polypeptide or a polysaccharide, to which the neo-antigenic peptides, is capable of being associated. Optionally, adjuvants are conjugated covalently or non-covalently to the peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response. Suitable adjuvants are described herein.

A vaccine composition according to the present invention may comprise more than one different adjuvant. Furthermore, the invention encompasses a therapeutic composition comprising any adjuvant substance including any of the above or combinations thereof. It is also contemplated that the nucleic acid molecule, and the adjuvant can be administered separately in any appropriate sequence.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and APC is present. Therefore, in some embodiments the vaccine composition according to the present invention additionally contains at least one antigen presenting cell.

The antigen-presenting cell (or stimulator cell) typically has an MHC class I or II molecule on its surface, and In some embodiments is substantially incapable of itself loading the MHC class I or II molecule with the selected antigen.

Preferably, the antigen presenting cells are dendritic cells. In some embodiments, the dendritic cells are autologous to a subject. In some embodiments of the present invention the antigen presenting cell comprises an expression construct comprising the nucleic acid molecules of the present invention. The nucleic acid molecules are capable of transducing the dendritic cell, thus resulting in the presentation of a peptide and induction of immunity.

In one aspect, the disclosure features a method of making an individualized cancer vaccine for a subject suspected of having or diagnosed with a cancer, comprising identifying a plurality of mutations in a sample from the subject; analyzing the plurality of mutations to identify one or more neoantigen mutations; and producing, based on the identified subset, a personalized cancer vaccine.

In some embodiments, identifying comprises sequencing the cancer. Methods for carrying out sequencing are described herein.

In some embodiments, identifying comprises sequencing the cancer.

In another embodiment, analyzing further comprises determining one or more binding characteristics associated with the neoantigen mutation, the binding characteristics selected from the group consisting of binding of the subject-specific peptides to T-cell receptor, binding of the subject-specific peptides to a HLA protein of the subject and binding of the subject-specific peptides to transporter associated with antigen processing (TAP); and ranking, based on the determined characteristics, each of the neo-antigenic mutations.

In some embodiments, the method further comprises cloning nucleic acid sequences encoding the one or plurality of neoantigen mutations into a nucleic acid molecule.

In some embodiments, the nucleic acid molecule is a plasmid. In another embodiment, the nucleic acid molecule comprises a nucleic acid sequence of Formula I that is positioned within the multiple cloning site of a plasmid selected from the group consisting of selected from the group consisting of pGX4501, pGX4503, pGX4504, pGX4505, and pGX4506. In some embodiments, the nucleic acid sequence of Formula I is positioned with the multiple cloning site of pGX4501. In some embodiments, the nucleic acid sequence of Formula I is positioned with the multiple cloning site of a plasmid selected from the group consisting of pGX4503. In some embodiments, the nucleic acid sequence of Formula I is positioned with the multiple cloning site of a plasmid selected from the group consisting of ppGX 4504. In some embodiments, the nucleic acid sequence of Formula I is positioned with the multiple cloning site of a plasmid selected from the group consisting of pGX4505. In some embodiments, the nucleic acid sequence of Formula I is positioned with the multiple cloning site of a plasmid selected from the group consisting of pGX4506. In some embodiments, the plasmid is pGX4505. In some embodiments, the plasmid comprises the backbone and linker sequence of pGX4505 with at least two or more AED nucleotide sequence encoding one or more neoantigens from a subject.

### Kits

The present disclosure provides a kit comprising a pharmaceutical composition comprising one or a plurality of nucleic acid molecules as described herein. The components of the kit are preferably formulated in pharmaceutically acceptable carriers.

Also included in the kit are instructions for use in methods of treating cancer in a subject or enhancing a CD8+ T cell immune response in a subject.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Wei, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

Other embodiments are described in the following non-limiting Examples.

### EXAMPLES

### Example 1. Materials and Methods

The experiments carried out in the following Examples were performed using, but not limited to, the following materials and methods.

### Animals and Cell Lines

C57Bl/6 mice were purchased from Jackson labs. TC1 and LLC tumors were generated by injecting 100,000 cells subcutaneously. ID8 tumors were generated by injecting 2 million cells intraperitoneally. Mice were treated by injecting 25 μg of DNA resuspended in 30 μL of water into the tibialis anterior muscle followed by electroporation with the CELLECTRA-3P device (Inovio Pharmaceuticals). For each immunization, mice were delivered two 0.1 Amp electric constant current square-wave pulses.

### DNA and RNA Sequencing

The mouse exome and RNA sequencing were performed on Illumina HiSeq-2500 platform. The SureSelect Mouse All Exon Kit (Agilent Technologies, USA) was used. All samples generated greater 13 Gb of data, with greater than 98% of the exomes covered at ≥150X. Overall, 99% of the reads aligned to the mouse reference genome. Mapping quality for 80% of the aligned reads was ≥Q60. Duplicate % was low 4-6%. Somatic variant calling was performed using Strelka program v1.0.14. The identified somatic variants were further filtered and only passed and on-target variants were considered for further analysis.

The RNA sequencing was performed using TrueSeq RNA library prep kit v2 (Illumina, USA). All samples generated >100 million reads. Reads mapping to ribosomal and mitochondrial genome were removed before performing alignment. The reads were aligned using STAR (2.4.1) aligner. Overall 96-98% of the total pre-processed reads mapped to the reference gene model/genome. The gene expression was estimated using Cufflinks v2.2.1.

Neoepitopes were prioritized from non-synonymous coding missense and frameshift mutants, where the mutant allele expression >1 FPKM. MHC class I binding analysis was performed for all coding missense and frameshift mutations. For this 9-mer peptides were processed using NetMHCons v1.1 on the C57Bl/6 MHC alleles (H-2-Kb,H-2-Db). Peptides were further prioritized based on lower proteasomal processing score using NetChop3.1 (IEDB). Peptides showing a score ≥0.7 were selected. Peptides were scored for TAP binding and peptides having binding affinity <0.5 were prioritized.

### Design of Neoantigen Vaccines

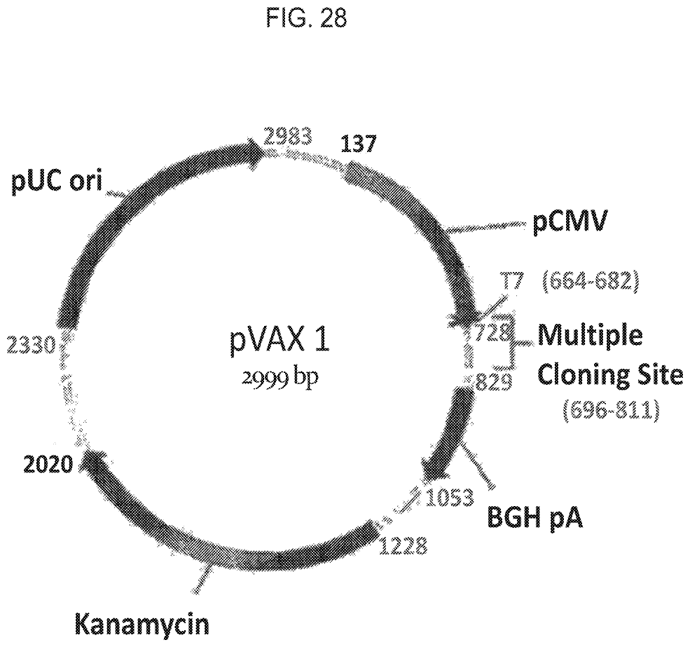

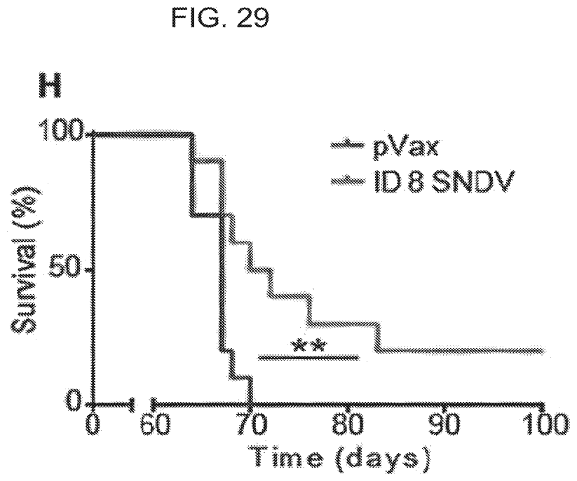

Neoantigen vaccines were designed by selecting the predicted neoantigens from the DNA and RNA sequencing data obtained from the TC1, LLC and ID8 established tumors. Twelve epitopes were included, defined as the predicted sequence that would bind to H2-K(b) or H2-D(b), keeping the mutated amino acid in the central position and keeping 12 non-mutated amino acids flanking on each side. Twelve 33-mers were concatenated with furin cleavage sites and each construct was subcloned into the pVax1 plasmid (GenScript). FIG. 28 is a map of the 2999 basepair backbone vector plasmid pVAX1 (Invitrogen, Carlsbad Calif.). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993.

### Flow Cytometry

BD LSRII flow cytometer (BD Biosciences) was used. Anti-mouse antibodies used were directly fluorochrome conjugated. The following antibodies were used: CD3e (17A2), CD4 (RM4-5), CD8b (YTS156.7.7), Interferon-γ (XMG1.2), TNFα (MP6-XT22), Interleukin-2 (JES6-5H4), and T-bet (4B10), all from Biolegend. Live/dead exclusion was done with Violet viability kit (Invitrogen). For the determination of intracellular cytokine production, 2 million splenocytes were cultured in the presence of peptides (5 μg/mL) derived from the corresponding wild-type or mutated neoantigen, Golgi-stop protein transport inhibitor (BD biosciences) and CD107a antibody (1D4B) for 4-5 hours prior to surface and intracellular staining. The neoantigen peptides consisted of 15mer peptides overlapping by 9 amino acids. These peptides spanned the entire 33mer used for immunization.

### Elispot

Splenocytes were harvested and coincubated with each neoantigen derived peptide pool comprising 15-mers overlapping by 9 amino acids. After a 24 hour incubation, mouse interferon-γ ELISPOT was performed according to the manufacturer's instructions (Mabtech). Spots were read using an ImmunoSpot CTL reader, and spot forming units (SFU) were calculated by subtracting media alone wells from stimulated wells. Concanavalin A was used as a positive control (not shown) to ensure spot development.

### T-Cell Expansion and Activation

Splenocytes were harvested from vaccinated mice and pulsed with 5 μg/ml of neoantigen specific peptides and 30 UI/ml of IL-2. Peptides and IL-2 (Peprotech) were refreshed with irradiated (4000 rad) splenocytes from naïve mice (1:3-10 T-cell: splenocyte ratio) once a week. 4-6 weeks after initiating the T-cell expansion, splenocytes were cocultured with the tumor cells for performing in vitro cytotoxicity experiments.

### In Vitro Cytotoxicity

10,000 luciferase transduced TC1 or ID8 cells were plated per well in a 96-well plate. 18 hours later, the cells were coincubated for 24 hours with 10 or 50,000 in vitro expanded T-cells. Cytotoxicity was measured using CYTOTOX-GLO Cytotoxicity Assay (Promega), according to the manufacturer's instructions. Cytotoxicity was reported as a ratio of luciferase expression in the T cell containing study wells divided by luciferase expression in the wells with tumor cells only (no T cells).

### Statistical Analysis

Differences between the means of experimental groups were calculated using a two-tailed unpaired Student's t test. Comparisons between two groups with repeated measures were done using Two-way ANOVA. Error bars represent standard error of the mean. For mouse survival analysis, significance was determined using a Gehan-Brelow-Wilcoxon test. All statistical analyses were done using Graph Pad Prism 7.0. p<0.05 was considered statistically significant.

### Example 2. DNA Vaccine Neo-Epitope Dodecamers Induce Frequent Immune Responses in Mice

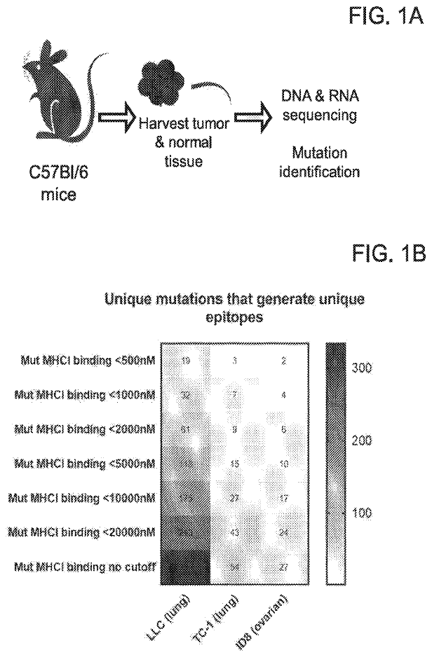

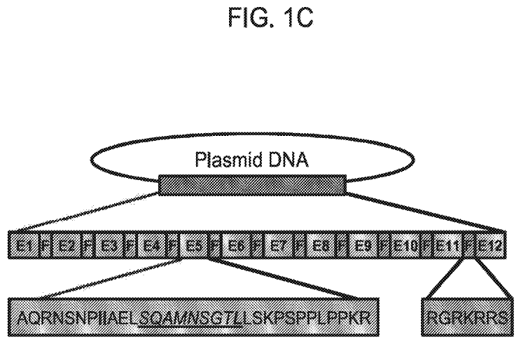

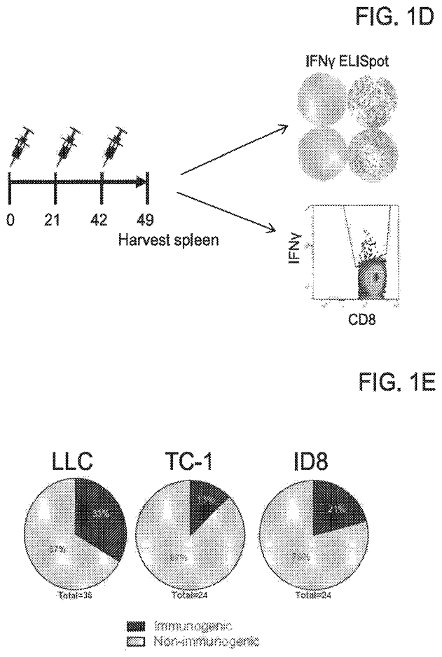

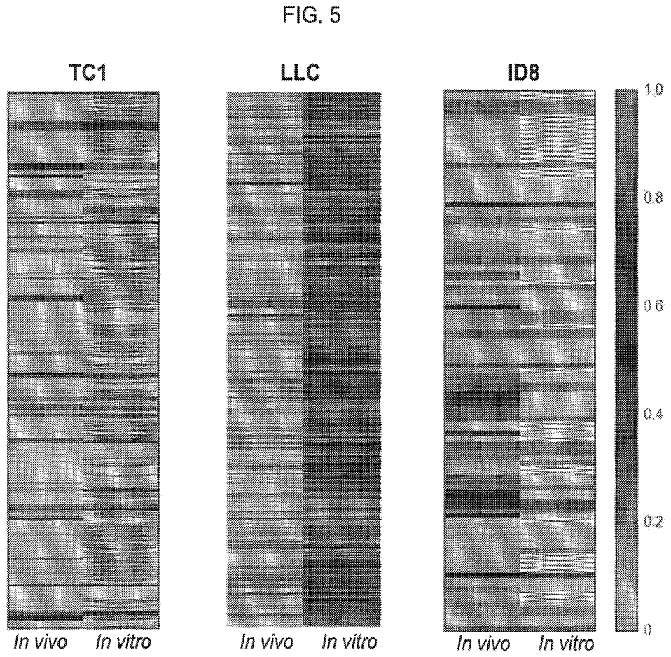

C57Bl/6 mice were implanted with three different tumor types: LLC and TC1, mouse lung tumor models, as well as ID8, a mouse ovarian tumor model. LLC and TC1 tumor cells were injected subcutaneously, and ID8 tumor cells into the peritoneum. After 3 weeks, tumors were harvested and DNA and RNA isolation was performed, as well as exome and RNA sequencing (FIG. 1A). DNA and RNA sequencing of cell lines cultured in vitro to the same cell lines implanted into mice (FIG. 5). Mutations that were expressed with an Alt allele depth in RNA-seq≥1, a proteasomal cleavage score of ≥10, and a TAP processing score≤0.5 (see methods for more detail) were included. As previously reported (13), a substantial proportion of mutations were differently expressed in tumors compared to cell lines, indicating differential expression of these genes in the context of the three-dimensional tumor microenvironment (FIG. 5).

There were a total of 334, 54, and 27 non-synonymous, expressed mutations that generate unique neoepitopes in the LLC, TC1 and ID8 tumor models, respectively (FIG. 1B). Only 19, 3 and 2 epitopes from LLC, TC1 and ID8, respectively, had less than 500 nM binding affinity to H2-K(b) or H2-D(b), predicted using NetMHCons v1.1 (FIG. 1B). 36 epitopes were chosen from LLC, 24 epitopes were chosen from TC1 and 24 epitopes were chosen from ID8 to test for immunogenicity using a DNA vaccine platform. All of the highest affinity epitopes (<500 nM) were included, as well as some low affinity epitopes to assess the value of the MHCI prediction programs. 7 total DNA vaccine neo-epitope dodecamers were designed, which included 12 total 33-mer epitopes per plasmid, linked together with furin cleavage sites (FIG. 1C). The 33-mer epitopes were designed to include the predicted immunogenic 9mer surrounded by 12 amino acids on either side, in order to supply potential CD4⁺ T cell responses for the epitope (FIG. 1C).

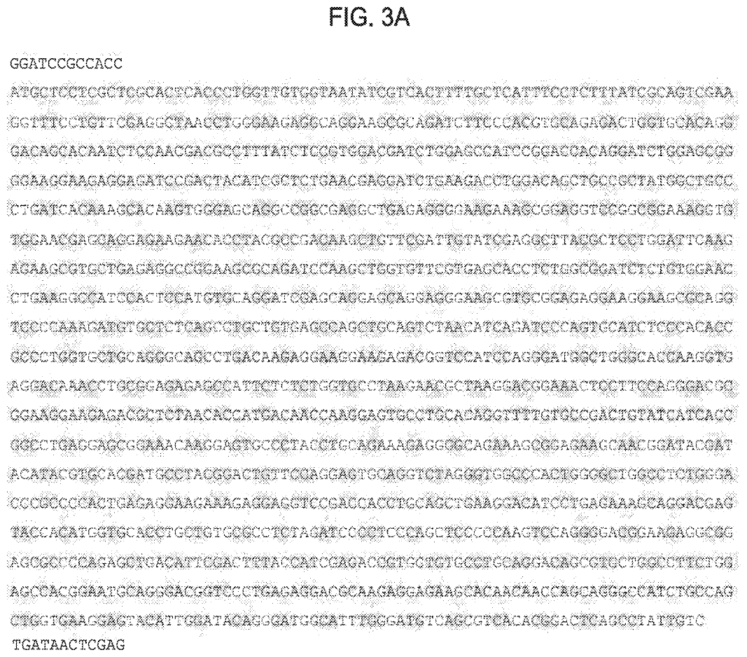

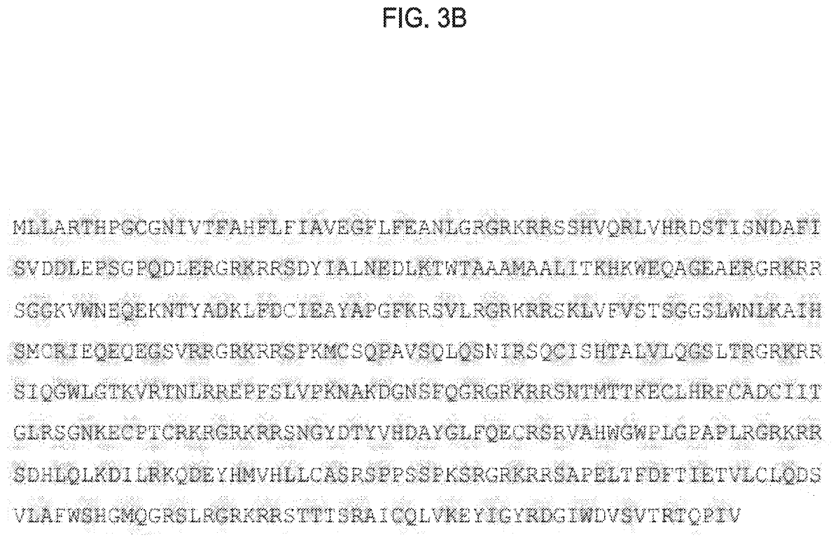

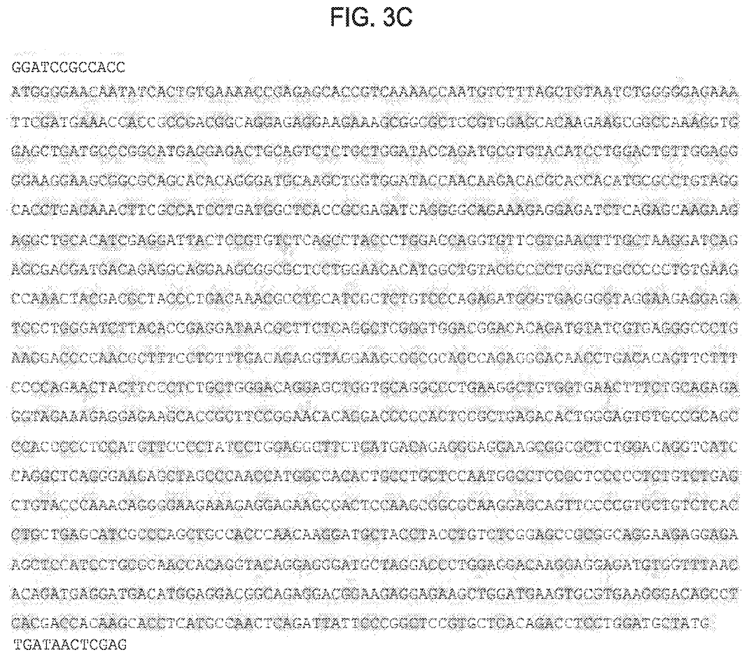

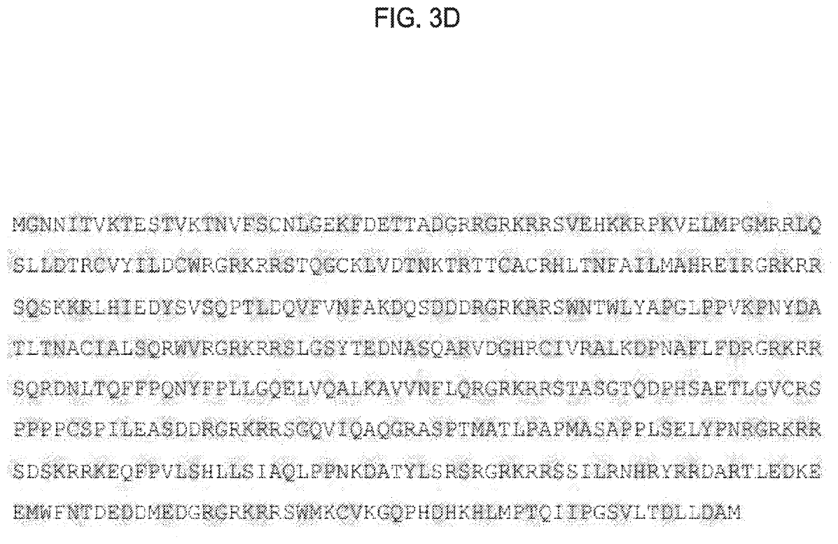

The seven plasmids are as follows:
(1) LLC Plasmid #1 is shown in FIG. 2A (nucleotide sequence) and FIG. 2B (amino acid sequence).
(2) LLC Plasmid #2 is shown in FIG. 2C (nucleotide sequence) and FIG. 2D (amino acid sequence).
(3) LLC Plasmid #3 is shown in FIG. 2E (nucleotide sequence) and FIG. 2F (amino acid sequence). LL Plasmid #3 binds to HLA proteins with an IC50 of less than about 500 nM.
(4) TC1 Plasmid #1 is shown in FIG. 3A (nucleotide sequence) and FIG. 3B (amino acid sequence).
(5) TC1 Plasmid #2 is shown in FIG. 3C (nucleotide sequence) and FIG. 3D (amino acid sequence).
(6) ID8 Plasmid #1 is shown in FIG. 4A (nucleotide sequence) and FIG. 4B (amino acid sequence).
(7) ID8 Plasmid #2 is shown in FIG. 4C (nucleotide sequence) and FIG. 4D (amino acid sequence).

These 7 plasmids, containing 84 neo-epitopes total, were tested by immunizing C57Bl/6 mice with 25 μg of DNA followed by electroporation with the CELLECTRA-3P device, at 3-week intervals for a total of three immunizations (FIG. 1D). Mice were sacrificed one week following the final immunization, and spleens were harvested for both IFNγ ELISpot analysis, as well as intracellular cytokine staining to identify CD4⁺ versus CD8⁺ reactive epitopes (FIG. 1D). The threshold for immunoreactivity was set to be ≥30 spot forming units per million splenocytes by IFNγ ELISpot against the peptides corresponding to the mutated peptide. In addition, epitopes classified as immunogenic required responses to the mutant peptide that were statistically significantly higher than those induced by mice immunized with control p Vax plasmid. Using these criteria, 12/36 epitopes were identified from the LLC model, 3/24 epitopes were identified from the TC1 tumor model, and 5/24 epitopes were identified from the ID8 model that were immunogenic when delivered using DNA vaccines (FIG. 1E). Overall, 24% (20/84) of epitopes generated immune responses, which is similar to what has been reported for RNA and SLP vaccines in pre-clinical studies without selection for high affinity MHCI binding.

### Example 3. DNA Vaccines Generate Predominantly CD8⁺ T Cell Responses to Neoantigens

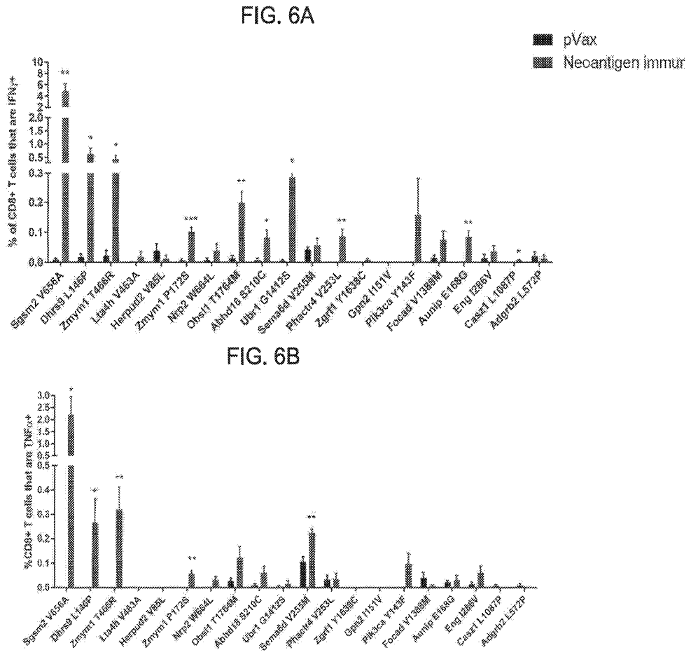

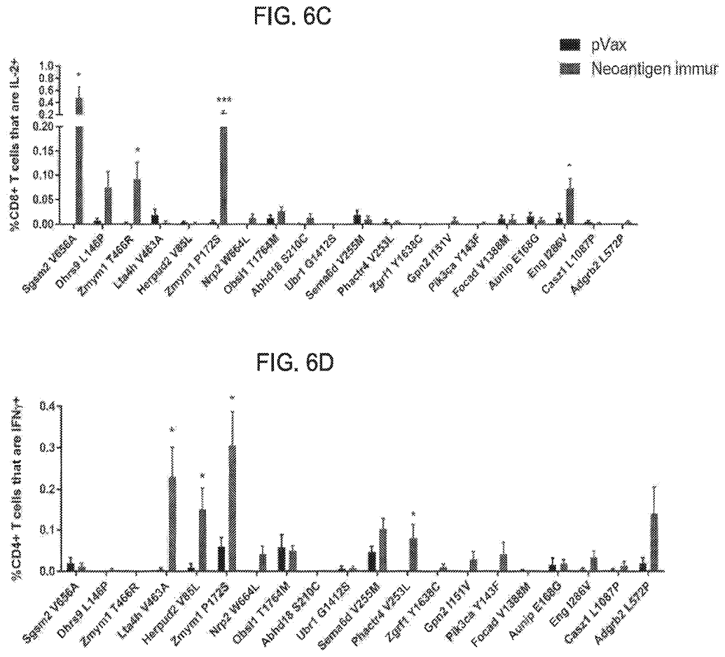

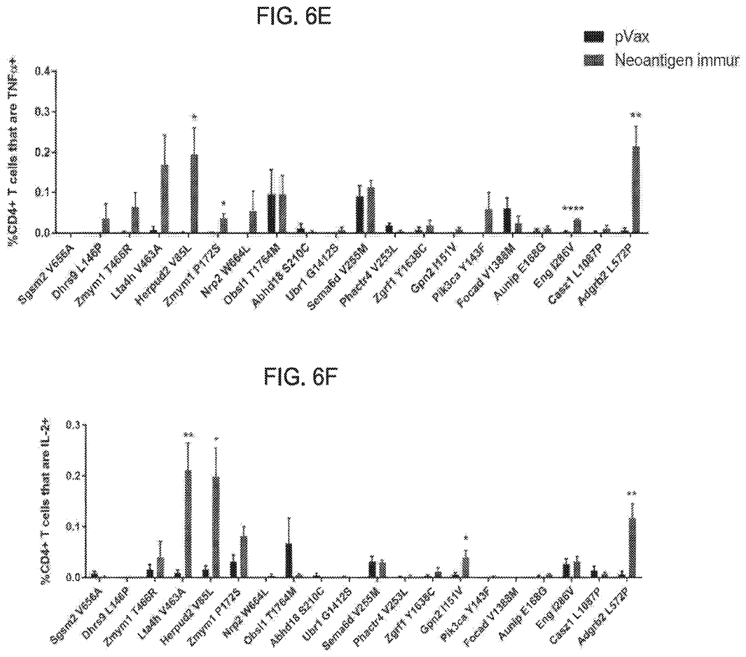

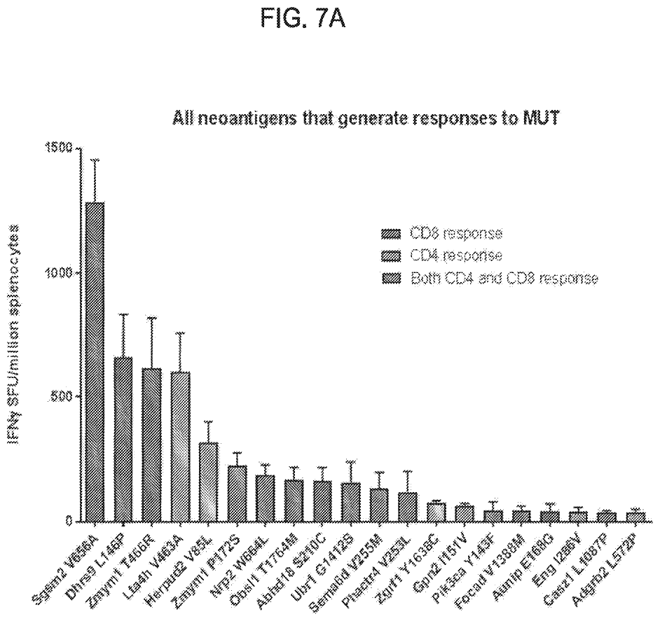

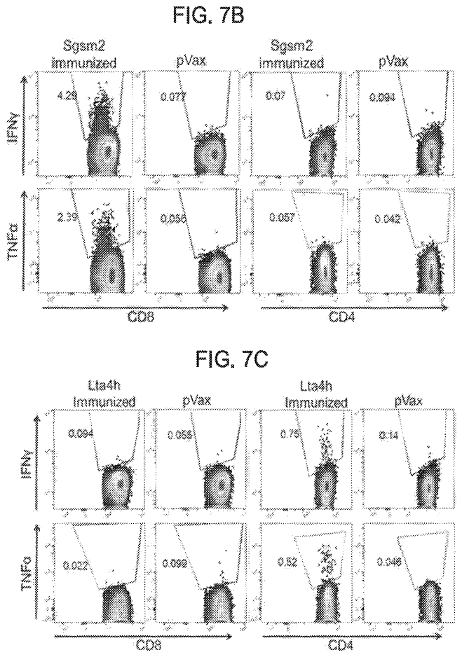

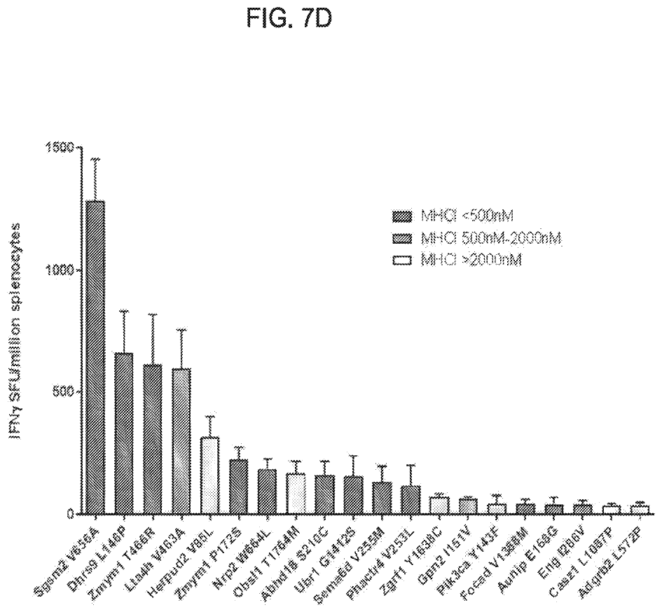

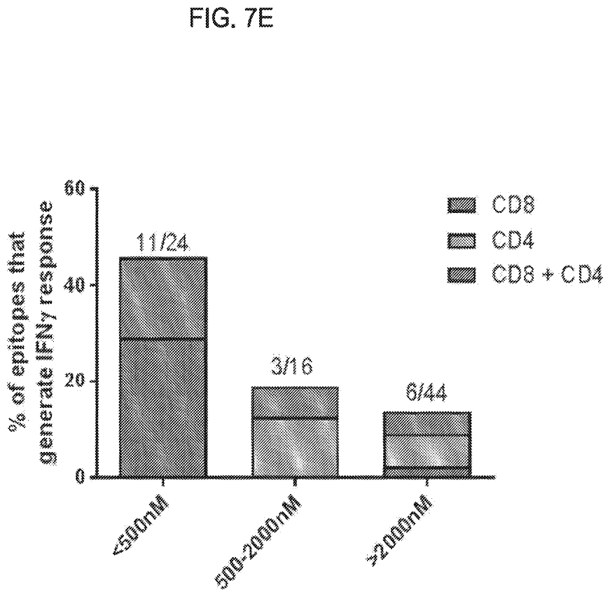

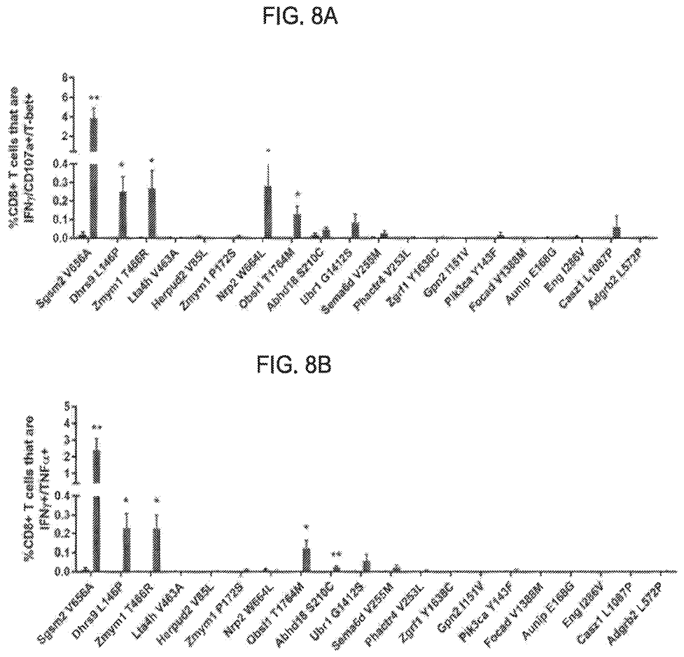

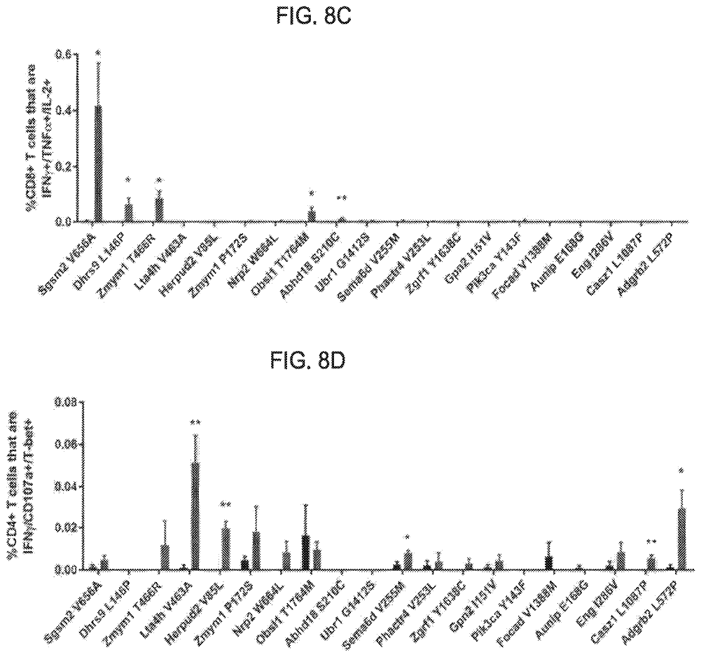

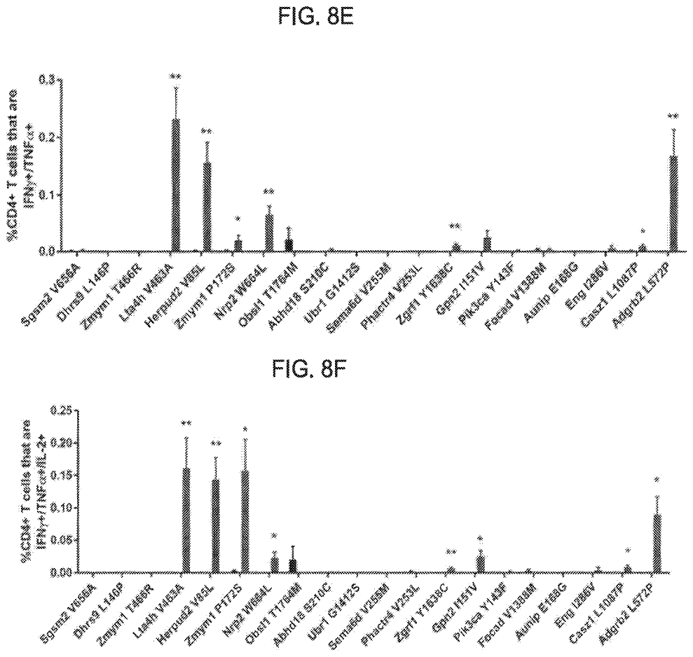

Intracellular staining and flow cytometry were performed to determine which of these epitopes generated CD8⁺ T cell responses, CD4⁺ T cell responses, or both (FIG. 6A-F). Strikingly, 40% of the responses generated were mediated by CD8⁺ T cells, 35% of the responses were mediated by both CD8⁺ and CD4⁺ T cells, and only 25% of responses were mediated by CD4⁺ T cells alone (FIG. 7A, FIG. 6A-F). There was a wide range in individual epitope immune responses, ranging from a mean of 36-1286.5 SFU (FIG. 2A). The strongest CD8⁺ (Sgsm2) and CD4+ (Lta4h) T cell epitopes generated both IFNγ and TNFα cytokine production exclusively in CD8⁺ and CD4⁺ T cells, respectively (FIG. 7B and FIG. 7C). Many other epitopes generated polyfunctional responses as well, with expression of multiple cytokines (IFNγ, TNFα and IL-2) simultaneously, in addition to expression of T-bet and CD107a, indicating cytolytic potential (FIG. 8A-F).

Next, the ability of the MHC class I binding affinity to predict immunogenic epitopes was assessed. NetMHCcons v1.1 were found to have some prediction power, and epitopes that were immunogenic were selected for (FIG. 7D and FIG. 7E). In fact, 46% of the epitopes that had <500 nM binding affinity generated immune responses, which is superior to the 24% of immunogenic epitopes without MHC class I affinity selection (FIG. 1E, FIG. 7E). Strikingly, 100% of the high affinity epitopes generated either CD8⁺ or CD8⁺/CD4⁺ T cell responses (FIG. 7E). These data suggest that the type of response elicited by the neoantigen may depend on the immunization platform, and that DNA vaccines are capable of inducing robust CD8⁺ T cell responses to neoantigens as opposed to other modes of vaccination that elicit minimal CD8+ T cell responses.

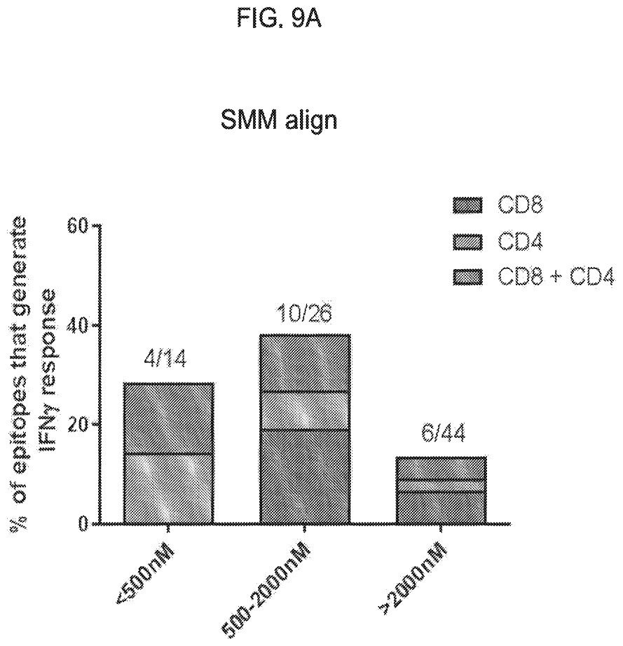

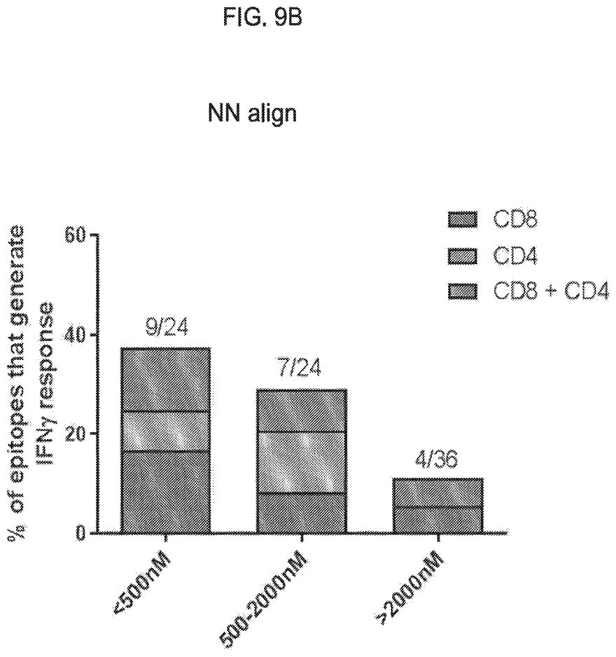

Next, the ability of MHC class II binding affinity to predict immunogenic epitopes was tested (FIG. 9A and FIG. 9B). Both netMHCII-1.1 (SMM align) and netMHCII-2.2 (NN align) prediction programs were tested, and it was found that neither program had demonstrable power to specifically predict CD4⁺ T cell epitopes. The SMM align prediction program did not predict immunogenic epitopes (28.6% of the epitopes with <500 nM binding affinity generated immune responses, FIG. 9A). While the NN align program did select for immunogenic epitopes (37.5% of epitopes with <500 nM binding affinity generated immune responses), this program did not specifically select for CD4⁺ T cell responses (FIG. 9B). Therefore, as reported by others (Lin et al. BMC Bioinformatics. 2008; 9 Suppl 12: S22), these MHC class II prediction programs have limited power to predict CD4⁺ T cell epitopes.

### Example 4. DNA Vaccine Primed T Cells Selectively Kill Mutated Cells

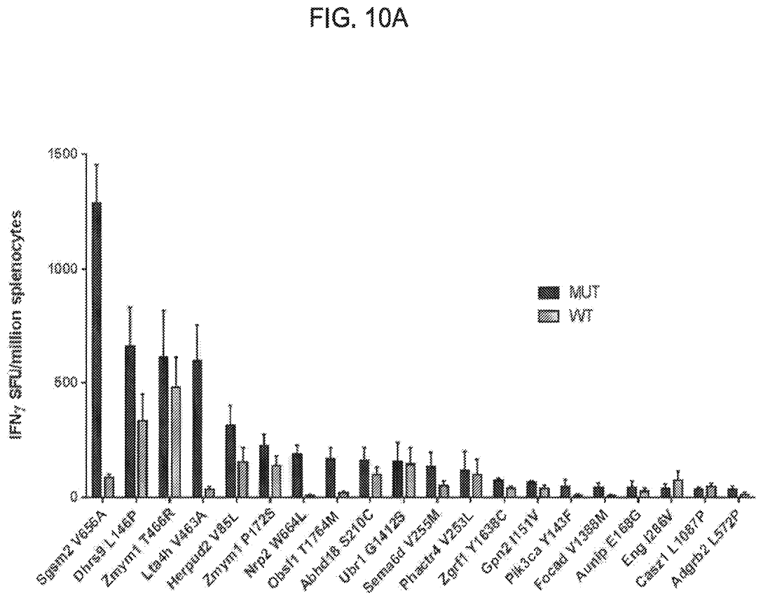

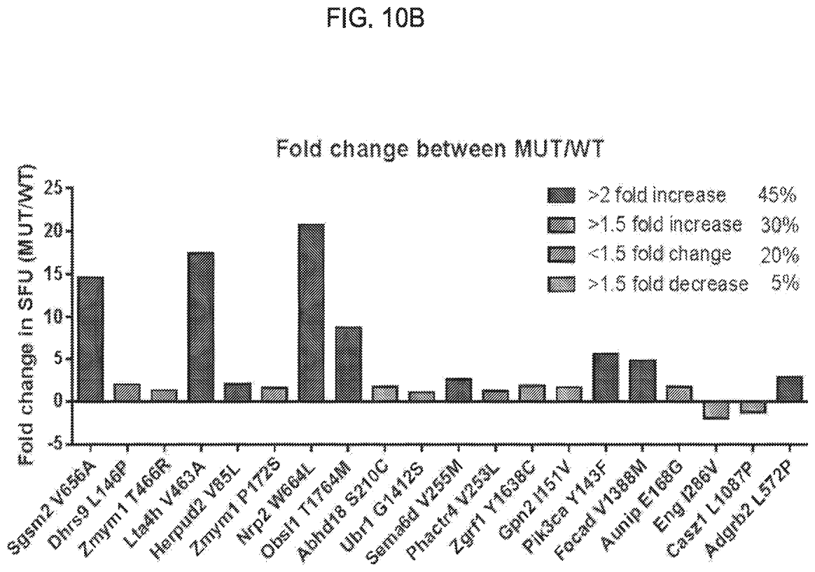

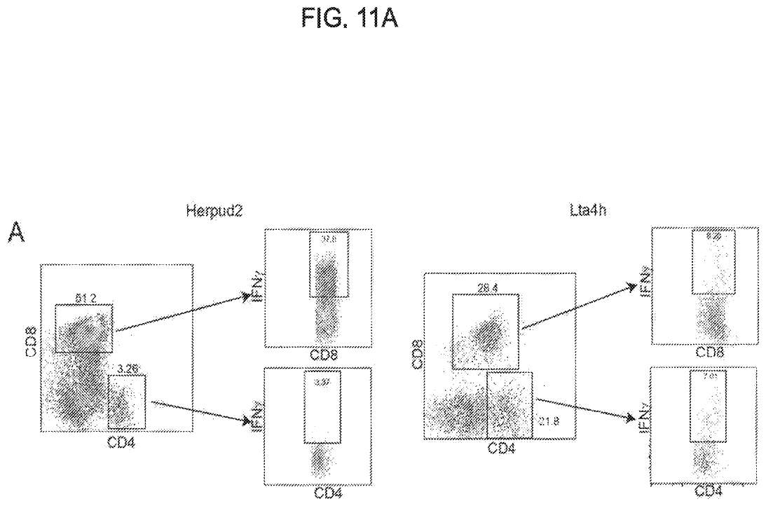

Immune responses generated from the immunized mice against the corresponding wild-type (non-mutated) neoantigen epitope were compared. It was found that the majority of immune responses are specific to the mutated epitope rather than the wild-type epitope (FIG. 10A and FIG. 10B). 75% of immune responses generated were at least 1.5 fold higher for the mutated epitope compared to the wild-type epitope. The remaining 25% of immune responses were similar when comparing mutant versus wild-type epitopes (FIG. 10B). These results are similar to what has been previously reported for neoantigen SLP vaccines, in which 68.8% of responses were specific to the mutated epitope (Castle et al. American Association for Cancer Research; 2012; 72:1081-91).

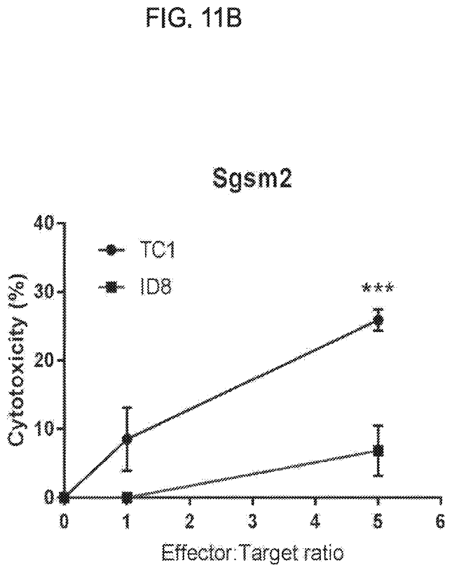

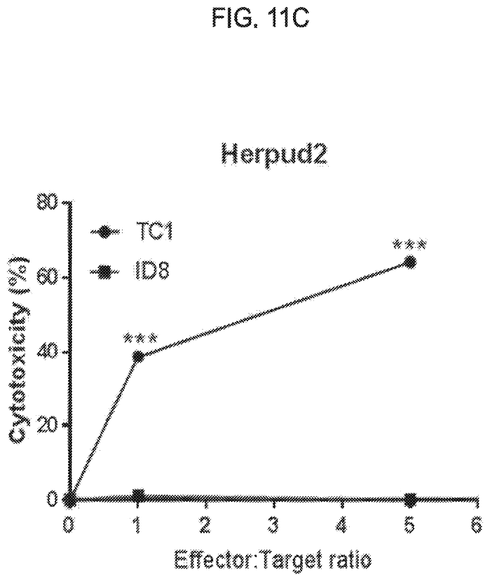

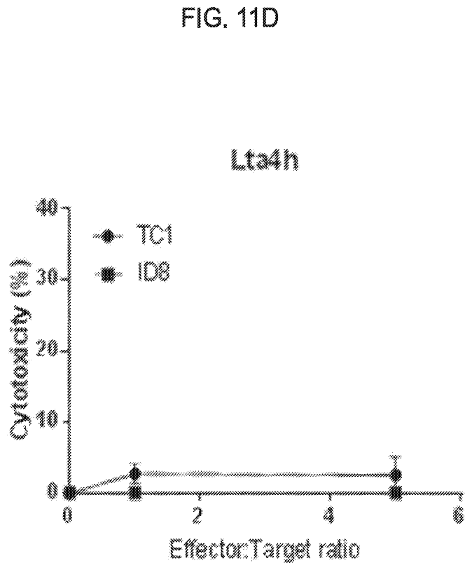

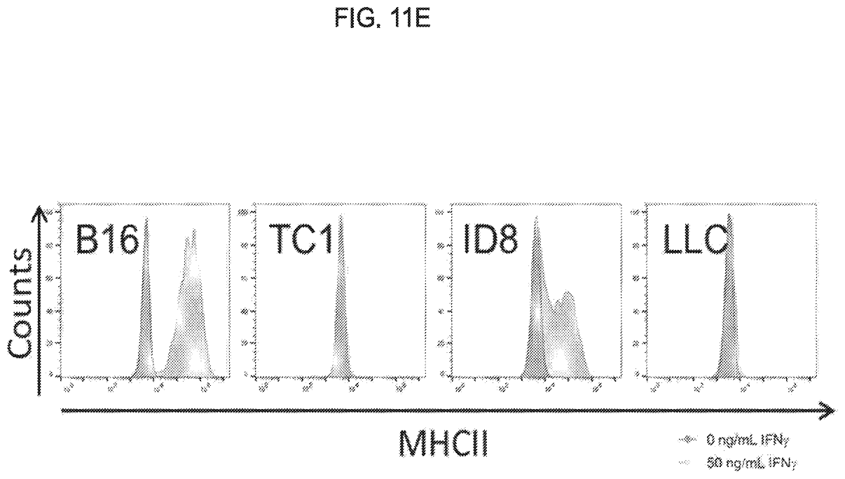

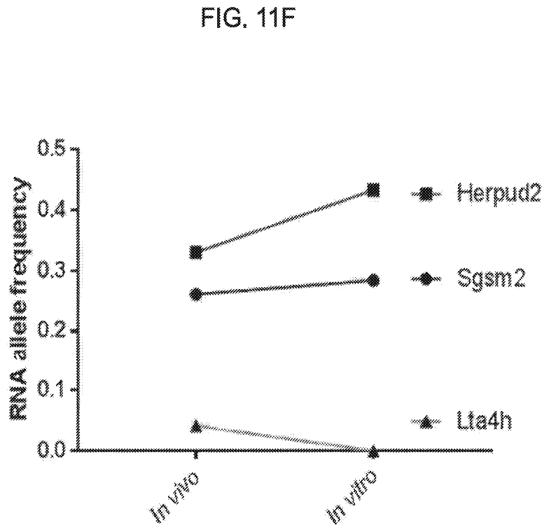

To determine the cytotoxic functionality and specificity of the T cells primed by our neoantigen DNA vaccines, the best responding T cells to the different TC1 neoantigens (Sgsm2, Herpud2 and Lta4h) were expanded ex vivo. Interestingly, after ex vivo expansion the majority of T cells expanded with the Herpud2 and Lta4 h peptides (originally CD4⁺) were in fact CD8⁺ T cells (FIG. 11A), indicating that these epitopes can generate a CD8⁺ T cell response in mice in addition to the CD4⁺ T cell response.

These expanded T cells were cocultured with TC1 cells or ID8 cells, which did not carry the neoantigen mutations in the selected genes. Co-incubation of the T cells with the tumor cells showed specific cytotoxicity of the TC1 neoantigen T cells against TC1 in the cases of Herpud2 and Sgsm2 (FIG. 11B and FIG. 11C). However, no cytotoxic activity of Lta4 h restricted T cells against TC1 was found (FIG. 11C). One hypothesis to explain this result is that TC1 cells may not express Lta4 h in vitro. The RNA sequencing data generated from the TC1 cultured cells and tumor were examined (FIG. 5), and it was found that Lta4 h is only expressed at the RNA level in vivo in tumor tissue, and is not expressed in cultured TC1 cells (FIG. 11D).

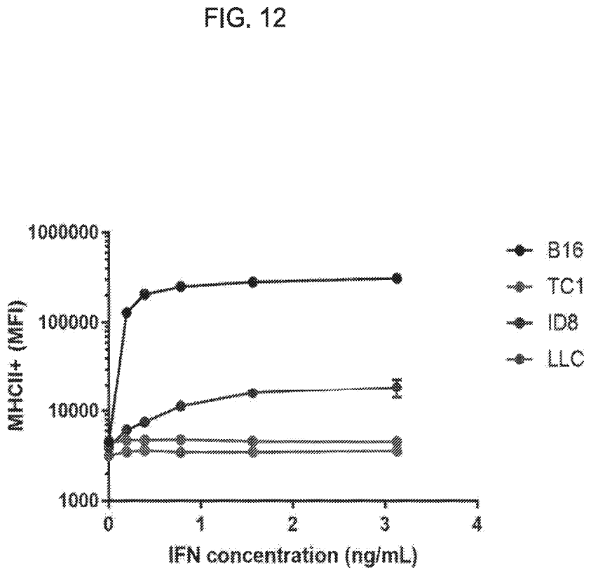

Although physiological expression of MHC class II is restricted to antigen presenting cells, some tumors in both humans and mice express this presenting protein complex, allowing for CD4⁺ direct recognition. To determine if there was potential direct recognition and potential cytotoxicity by CD4⁺ T cells, MHC class II was measured in the tumor cells. It was found that, unlike B16 melanoma or ID8, TC1 and LLC cells did not have MHC class II expression upon incubation with various doses of IFNγ (FIG. 11E and FIG. 12), and therefore tumor killing must occur through an MHC class I restricted mechanism.

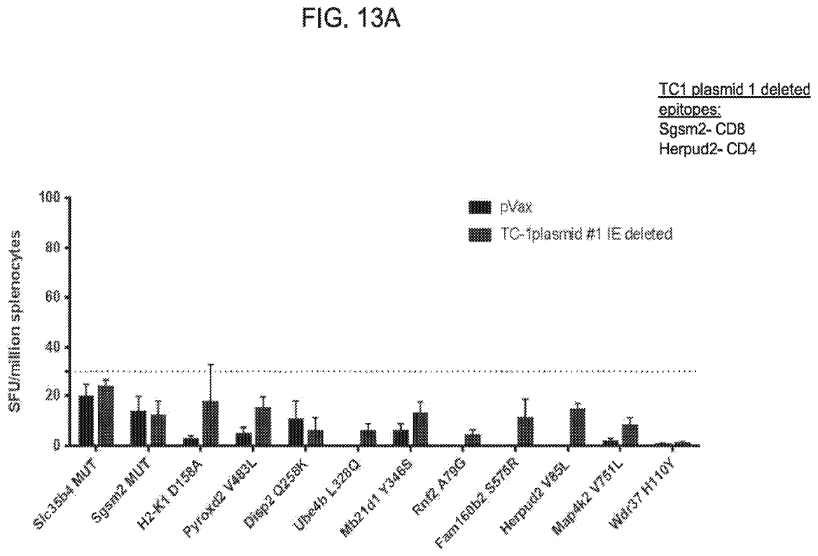

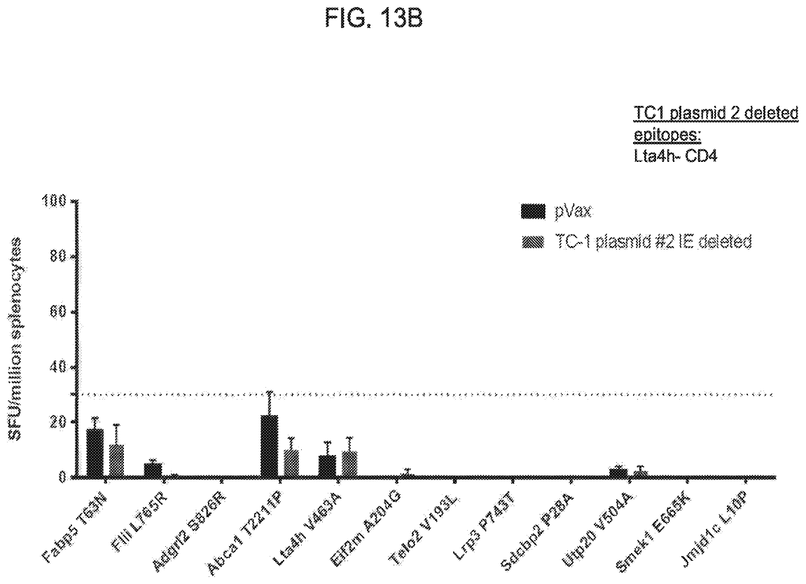

Next, the hierarchy of immunodominance for the epitopes within the TC1 neoantigen plasmids was examined. It was tested if the strong responses observed for the Sgsm2, Herpud2 and Lta4 h epitopes could be masking potential sub-dominant immune responses from other epitopes within the same plasmid by deleting the immunodominant epitopes from each plasmid (FIG. 13A and FIG. 13B). It was found that, for each plasmid, deleting the immunodominant epitopes did not result in generation of immune responses from sub-dominant epitopes (FIG. 13A and FIG. 13B).

### Example 5. DNA Neoantigen Vaccines Delay Tumor Progression

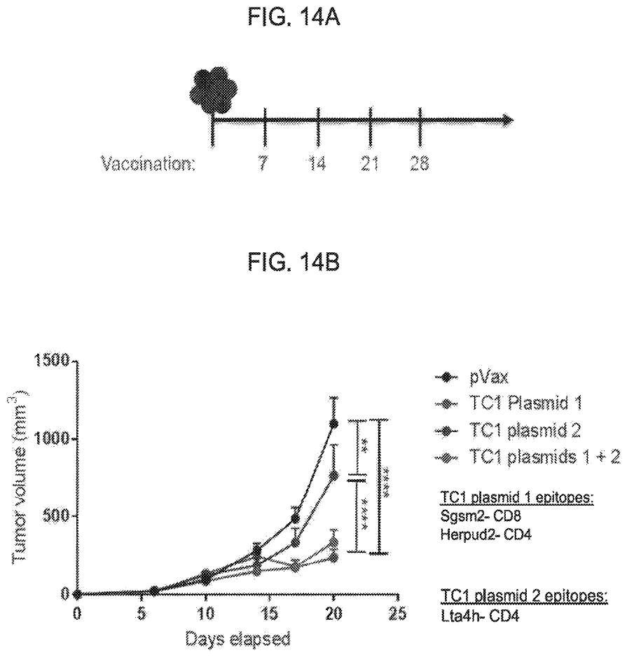

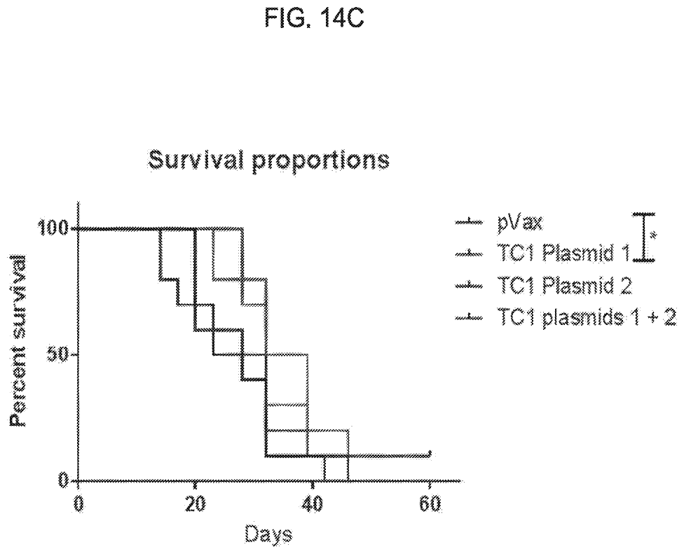

The in vivo anti-tumor effect of the TC1 vaccine was examined. To do this, mice were implanted with the TC1 cell line, and 7 days later the mice were vaccinated with weekly doses of the dodecamer vaccine Plasmid 1 (containing the immunogenic neoantigens Sgsm2 and Herpud2), Plasmid 2 (containing the immunogenic neoantigen Lta4h), both or the empty pVax vector (FIG. 14A). A profound delay in tumor progression associated with a longer survival was found when the mice were treated with the Plasmid 1 alone or the combination (FIG. 14B). A significant, although less intense, tumor delay was found with Plasmid 2 (FIG. 14B). FIG. 14C Survival curve of mice bearing TC1 treated TC1 plasmid 1, 2, both or pVax (n=10 mice per group). While the Lta4 h epitope was highly immunogenic, it generated primarily CD4⁺ T cell responses (FIG. 7A). These neoantigen-specific CD4⁺ T cells were likely not as effective at delaying tumor progression in this TC1 tumor model, because the TC1 cells do not express MHC class II (FIG. 11E). In addition, the level of expression of the Lta4 h neoantigen was relatively low (FIG. 11D) compared to the neoantigens present in Plasmid 1.

Examples 1-5 describe for the first time the possibility of generating effective anti-tumor immune responses against cancer neoantigens using DNA based vaccines. The DNA vaccine platform has several a priori advantages over other assayed platforms for generating effective anti-tumor neoantigen vaccines such as speed of vaccine production, vaccine stability and manufacturing cost. Time is a critical factor in cancer treatment. Even patients with surgically resectable tumors are at risk for development of micrometastases with potentially immune suppressive microenvironments. Therefore, the speed in which a personalized vaccine can be manufactured is of paramount importance. DNA vaccine production is more rapid than other platforms because it does not require complex peptide synthesis or RNA in vitro transcription. Furthermore, DNA is stable at room temperature, and, unlike RNA and peptide vaccines, does not require a cold chain. This ease of manufacturing and transport contributes to the much lower cost of DNA vaccines, making it an ideal technology for personalized patient specific therapeutic applications.

CD8⁺ T cells are thought to be the major mediators of anti-tumor T cell responses. As we have previously shown in preclinical and clinical studies (Trimble et al. Lancet (London, England). 2015; Duperret et al. Cell. 2017). DNA vaccines are able to generate robust CD8⁺ T cell responses. Examples 1-5 show that, as expected, DNA encoded neoantigens predicted for high MHCI binding generated CD8⁺ or CD8⁺/CD4⁺ neoantigen specific immune responses. RNA and peptide-based vaccines generated based on predicted MHCI restricted responses have surprisingly resulted in MHCII-restricted responses (Ott et al. 2017; Sahin et al. 2017). MHCI presentation of epitopes requires intracellular protein synthesis and proteasomal degradation (Rock et al. Trends Immunol. 2017). Synthetic long peptides are injected directly into tissues, and therefore are primarily engulfed and presented by antigen presenting cells, which will skew them towards a class II presentation. In the case of RNA and DNA, peptide synthesis occurs in the cell and can be cleaved as necessary by the proteasome and enter the MHC class I pathway. The strong CD4⁺ bias from RNA vaccines could be due to the pro-inflammatory properties of RNA. RNAis sensed by multiple innate immune receptors such as TLR3,7,8 and RIG-I like receptors which promote pro-inflammatory cytokine production (for instance, IL-6 or TNFα) (Pardi et al. Nat Rev Drug Discov. 2018). This inflammatory response can be detrimental to CD8⁺ activation and cytokine production (Wu et al. Sci Rep. 2015), and can additionally inhibit mRNA replication, stall translation and result in RNA degradation (Pardi et al. 2018; J Virol. 2012; 86:2900-10).

It has been shown that CD4⁺ T cells can generate anti-tumor cytotoxicity. CD4⁺ T cells can exert direct cytotoxicity based on granule exocytosis or Fas-Fas ligand pathway upon recognition of MHC class II-peptide complex (Fang et al. Proc Natl Acad Sci USA. 201; 109:9983-8; Janssens et al. J Immunol. 2003; 171:4604-12). However, it is not common for solid tumors to express MHCII (41% in melanoma, 0% in small cell lung cancer) (He et al. Lung Cancer. 2017; 112:75-80; Johnson et al. Nat Commun. 2016; 7:10582). CD4⁺ T cells have also been suggested to induce killing of tumor cells that do not express MHC class II by activating macrophage killing of tumor cells (Laurtizen et al. Cell Immunol. 1993; 148:177-88).

The number of neoantigens in cancer varies widely according to the tumor type but has been defined to be approximately between 33 and 163 expressed, non-synonymous mutations (Vogelstein 2013). Using the described DNA platform to generate neoantigen based vaccines allows the encoding of a high number of neoantigens in each plasmid. Considering the insert size (33aa) and the linker (7aa), a vaccine with 50 neoantigens would only require an insert of 6150 bp. This would permit possible immunization of all identified neoantigens in most cancer patients using only one plasmid. For those with very high mutation loads, all neoantigens could be included using 2 or 3 plasmids. This strategy would increase exponentially the ability to generate a wide range of anti-tumor T-cell responses. Because driver mutations are rarely immunogenic, and immunogenic neoantigens typically occur as passenger mutations, immunizing against a larger number of neoantigens per tumor would prevent or delay tumor immune escape. This approach of immunizing against all potential neoantigens also eliminates the need to validate experimentally the real (vs predicted) value of each individual epitope, shortening valuable time before the vaccine can be produced and administered to the patient. In addition, because humans have 6 different MHC class I molecules (more than the 2 different molecules present in inbred mice), it is likely that a higher proportion of epitopes will be able to bind to human HLA and generate responses to the vaccine.

In conclusion, Examples 1-5 show for the first time that DNA vaccines against tumor neoantigens are able to generate CD8⁺ T-cell anti-tumor specific responses and delay tumor progression. This promising technology possesses advantages such as rapid manufacturing time, lower manufacturing costs, higher stability and the ability to target most, if not all, of the neoantigens present in each patient.

### Example 6. Design of Poly-Neoepitope DNA Vaccine Against B16 Model

### Design of Neoantigen Vaccines

We designed the pGX4501, pGX4503, pGX4504, pGX4505, and pGX4506 vaccines by encoding the previously identified 27mer B16 and CT26 neoantigens (M27, M30, and M33 for B16, and M19, M20 and M26 for CT26) (Kreiter et al. 2015). We separated each epitope with no linker, a furin cleavage site or a furin cleavage site separating 2 epitopes and a P2A cleavage site separating 2 epitopes. These sequences were subcloned into the p Vax1 plasmid (GenScript).

The methods of designing the vAED portion of the vector were identical to the methods performed in above Example 1 except that the peptides were 15mers overlapping by 11 (instead of 15mers overlapping by 9 as indicated above).

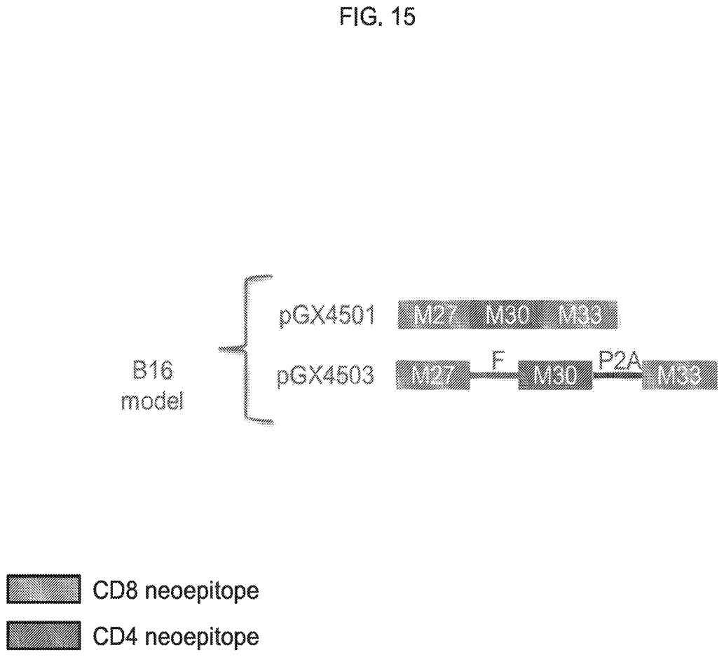

FIG. 15 shows a schematic of the design of poly-neoepitope DNA vaccine against B16 model. pGX4501 and pGX4503 plasmids were used. F indicates a furin cleavage site, or linker. P2A indicates a porcine teschovirus-1 cleavage site, or linker.

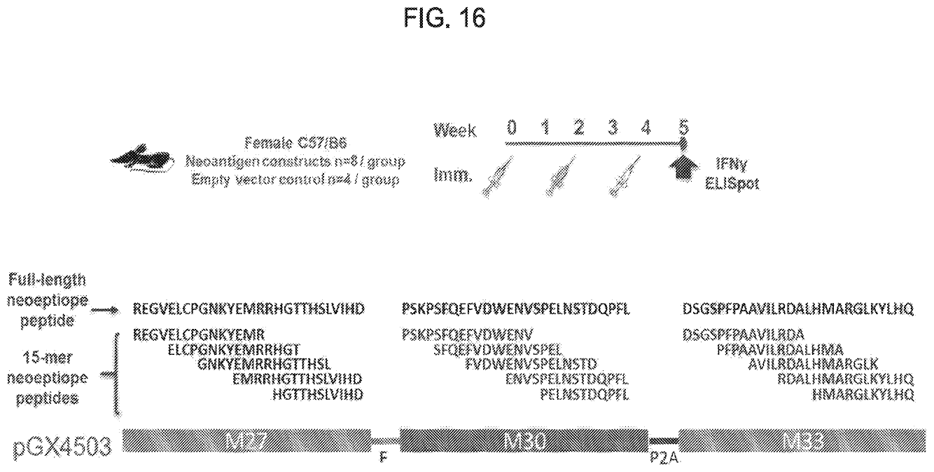

FIG. 16 shows the strategy for assessment of immune responses induced by a DNA vaccine against B16. Female C57/B6 mice were immunized with neoantigen constructs (n=8/group) or empty vector control (n=4). Immunization was carried out at weeks 0, 2, and 4. Splenic lymphocytes were stimulated for 18 hours with peptides for the full neoepitope sequence (MHC II) or 15-mer peptides overlapping by 11 amino acids (MHC I). Peptides were design to not include cleavage sites. IFNγ ELISpot assay was carried out at week 5.

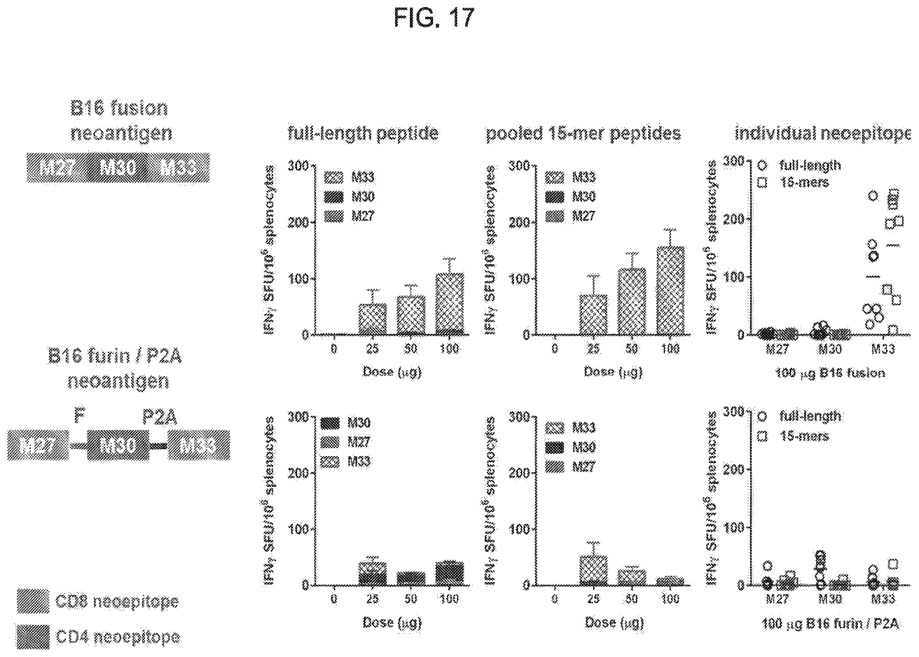

FIG. 17 shows a panel of graphs that show the results from the ELISpot assay, measuring the amount of IFNγ SFU/10e6 splenocytes, which corresponds to T cell activation, in response to the full length peptide, pooled 15-mer peptides and individual neoepitopes. As shown in FIG. 17, the individual neoepitopes presented in the B16 fusion neoantigen generated the strongest response. The B16 fusion neoantigen construct (top) and the B16 furin/P2A neoantigen construct (bottom) were tested.

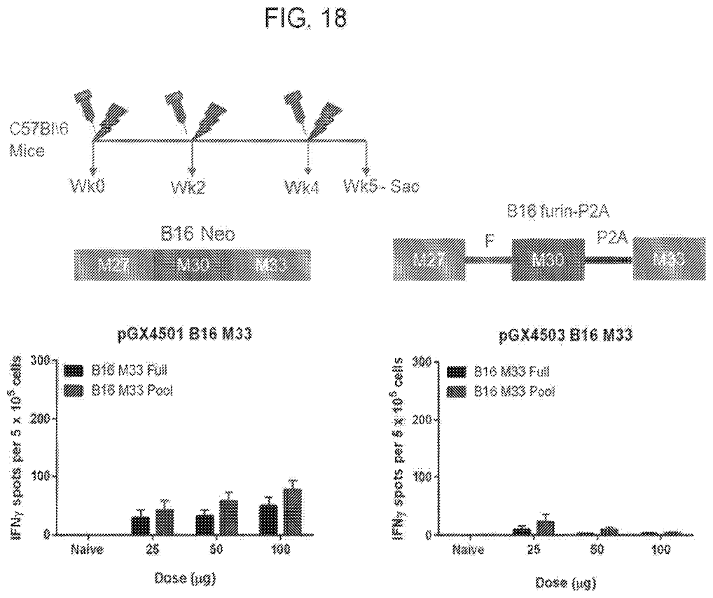

FIG. 18 shows proof of concept neoantigen studies: B16 neoantigens. In the absence of an adjuvant, pGX4501 can induce an immune response in mice, particularly in the M33 region, which is a CD8 epitope.

### Example 7. Design of Poly-Neoepitope DNA Vaccine Against CT26 Model

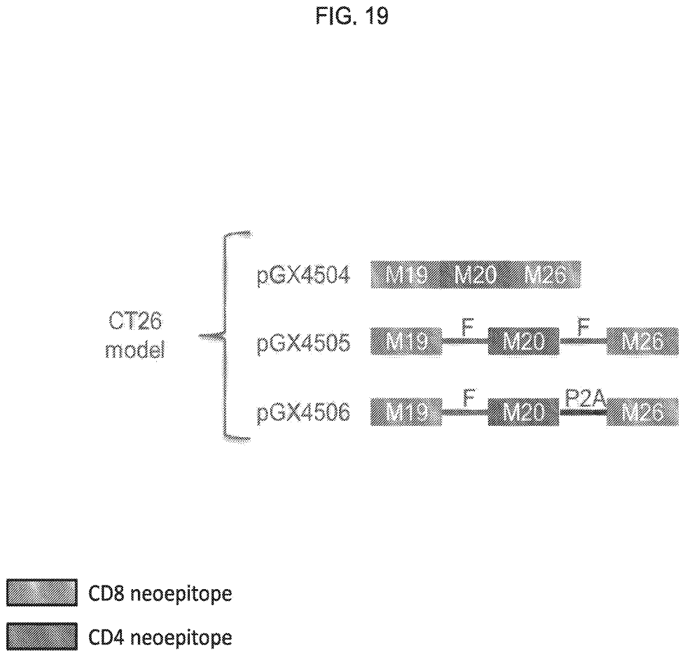

FIG. 19 shows a schematic of the design of poly-neoepitope DNA vaccine against B16 model. pGX4504, pGX5405 and pGC4506 plasmids were used. F indicates a furin linker. P2A indicates a porcine teschovirus-1 linker.

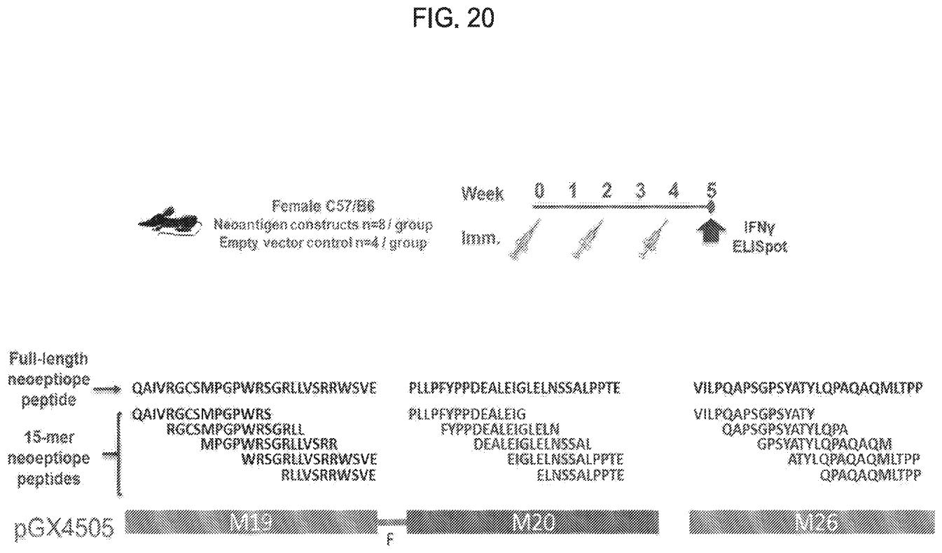

FIG. 20 shows the strategy for assessment of immune responses induced by a DNA vaccine against B16. Female C57/B6 mice were immunized with neoantigen constructs (n=8/group) or empty vector control (n=4). Immunization was carried out at weeks 0, 2, and 4. Splenic lymphocytes were stimulated for 18 hours with peptides for the full neoepitope sequence (MHC II) or 15-mer peptides overlapping by 11 amino acids (MHC I). Peptides were design to not include cleavage sites. IFNγ ELISpot assay was carried out at week 5.

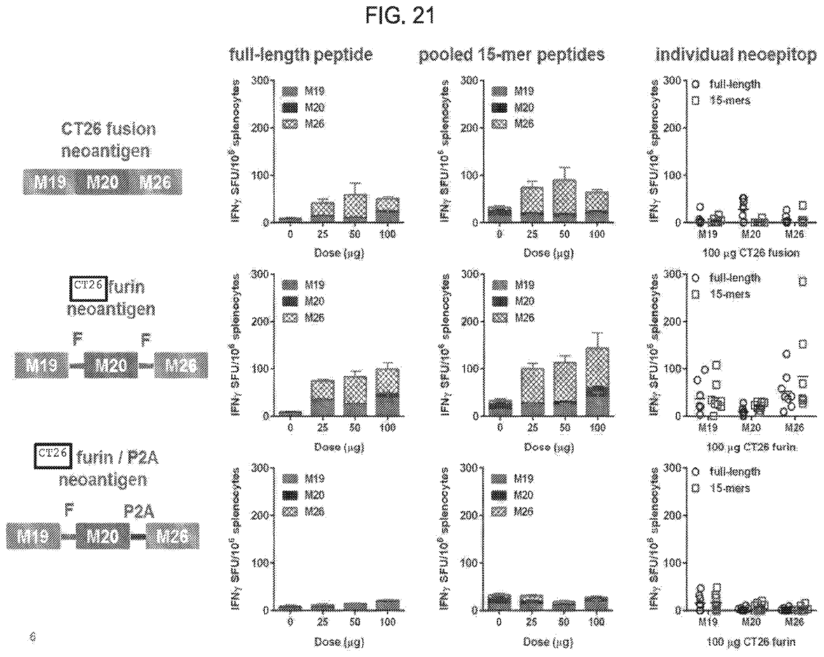

FIG. 21 shows a panel of graphs that show the results from the ELISpot assay, measuring the amount of IFNγ SFU/10e6 splenocytes, corresponding to T cell activation, for the tested full length peptide, pooled 15-mer peptides and individual neoepitopes. As shown in FIG. 21, the individual neoepitopes presented in the CT26 furin neoantigen generated the strongest response. The CT26 fusion neoantigen construct (top), the CT26 furin neoantigen (middle) and the CT26 furin/P2A neoantigen construct (bottom) were tested.

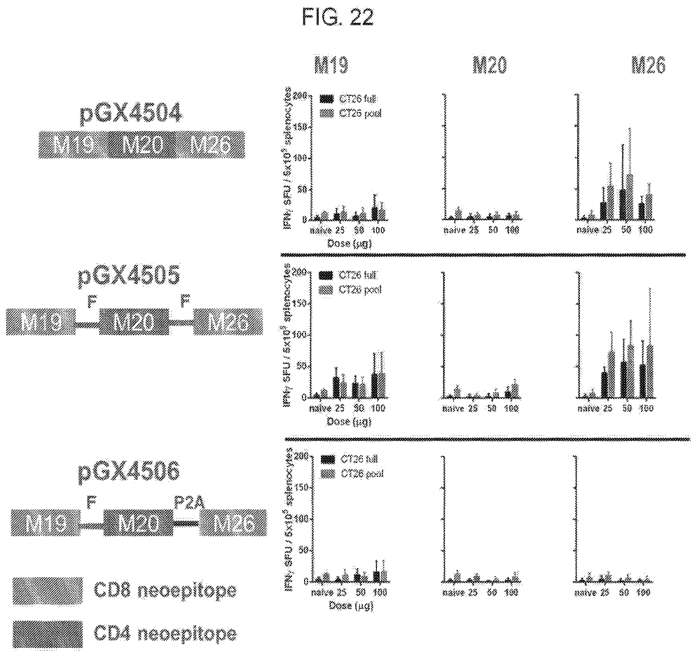

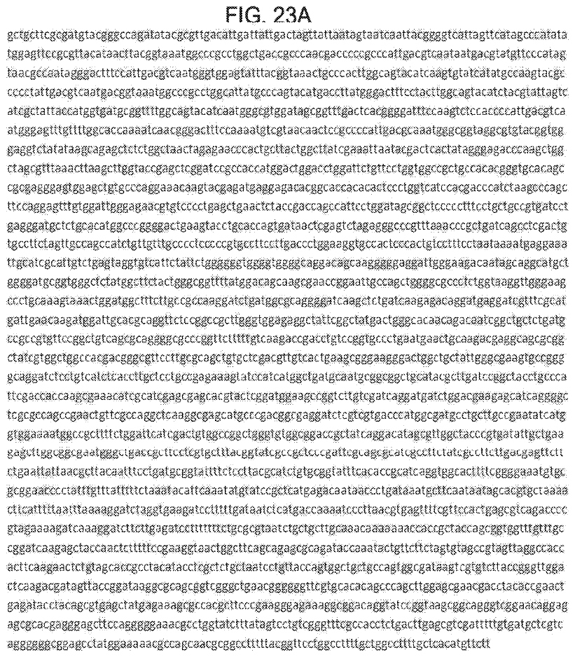

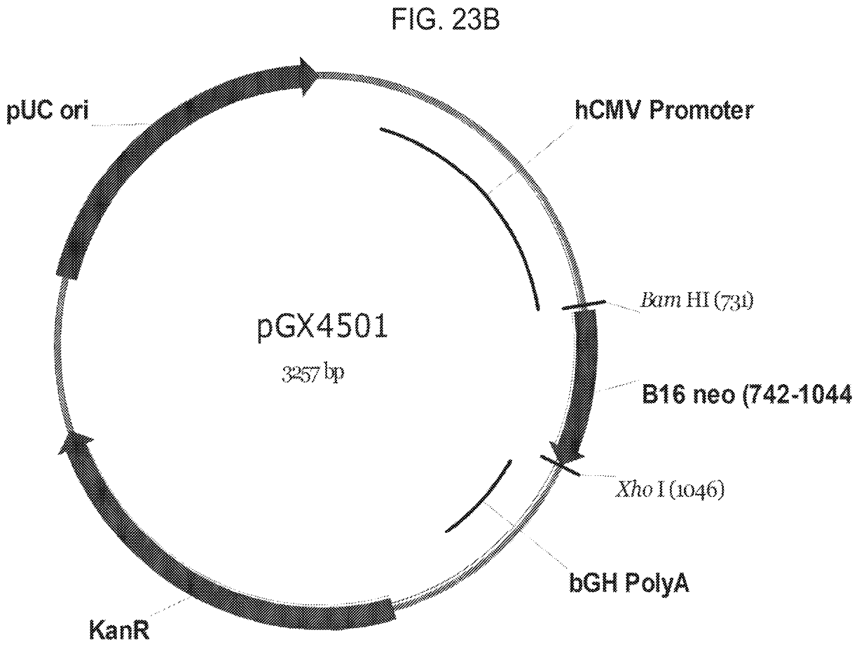

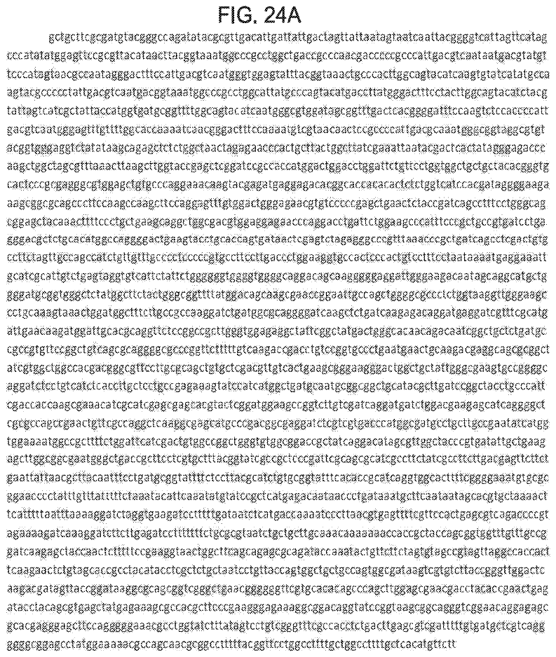

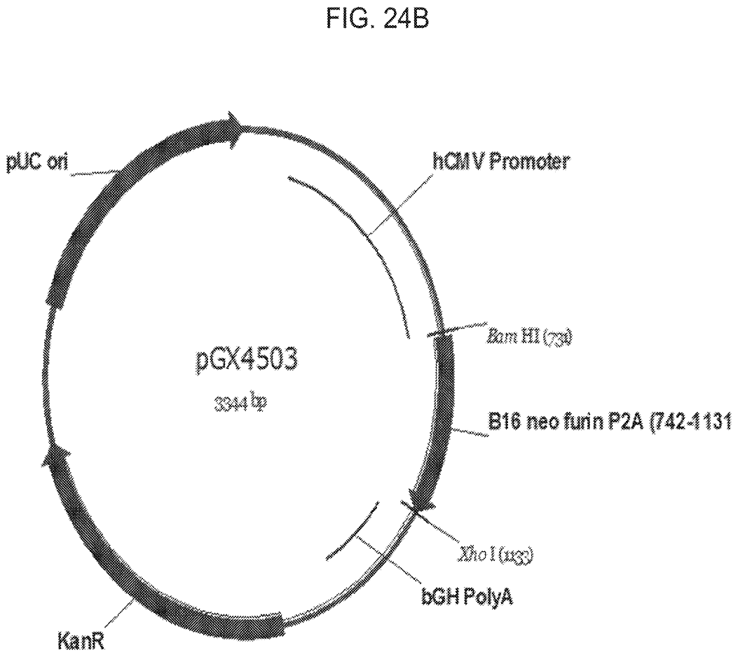

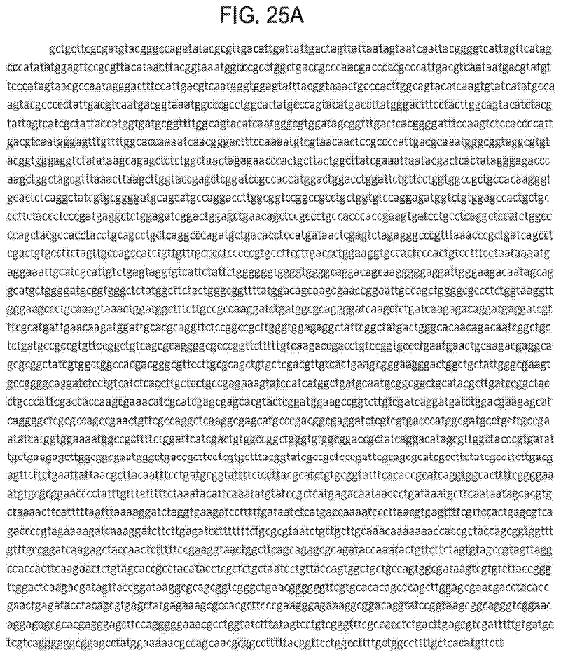

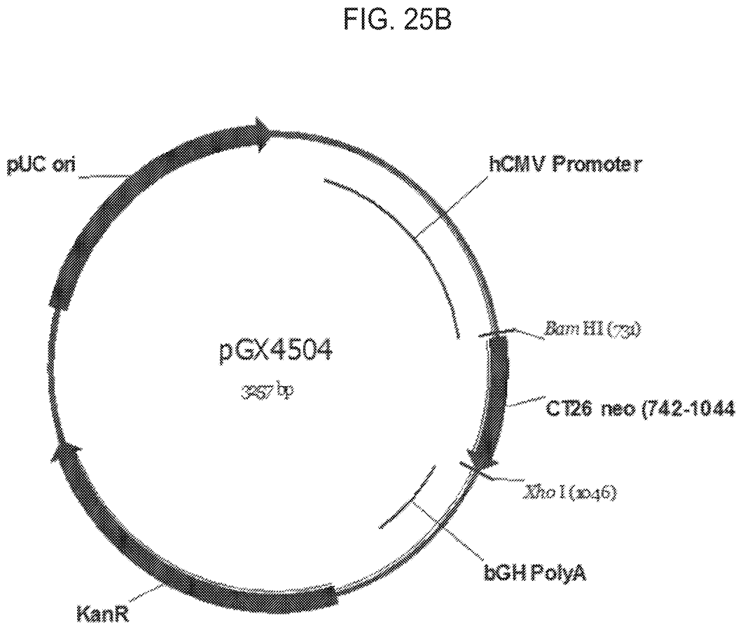

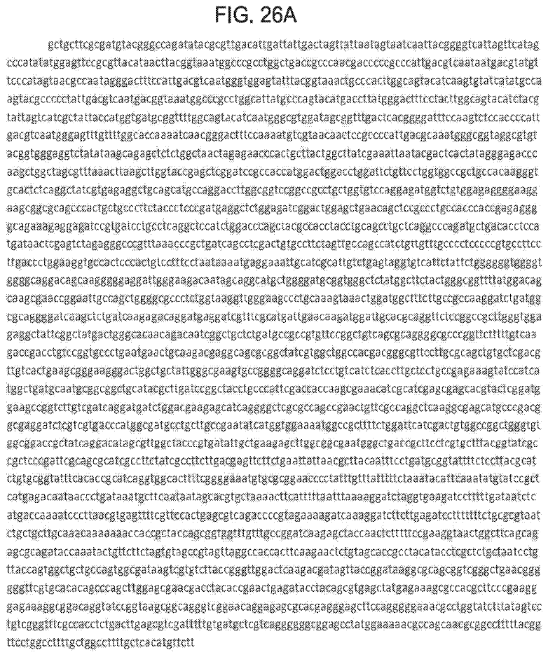

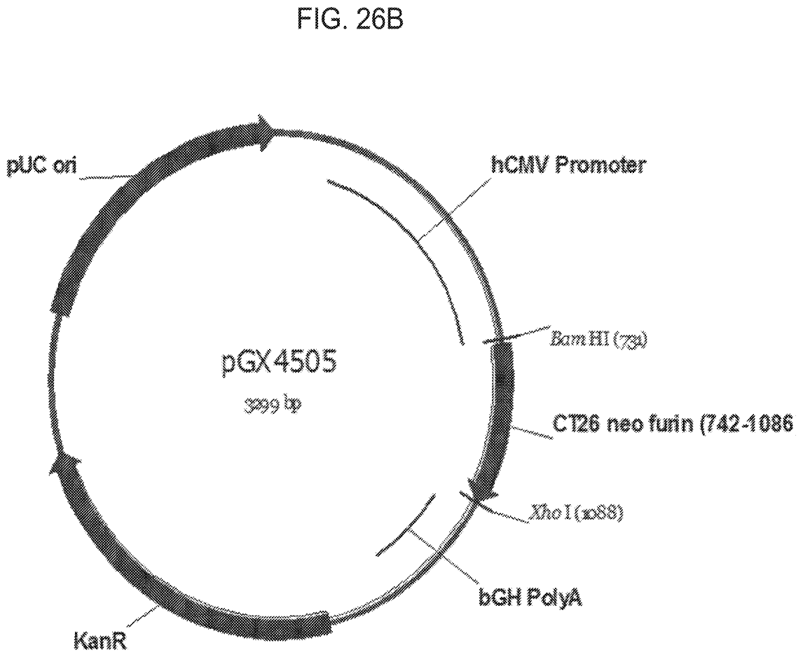

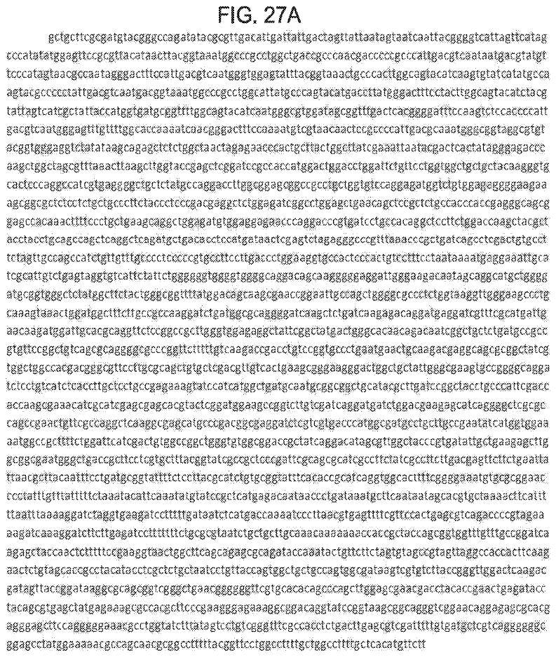

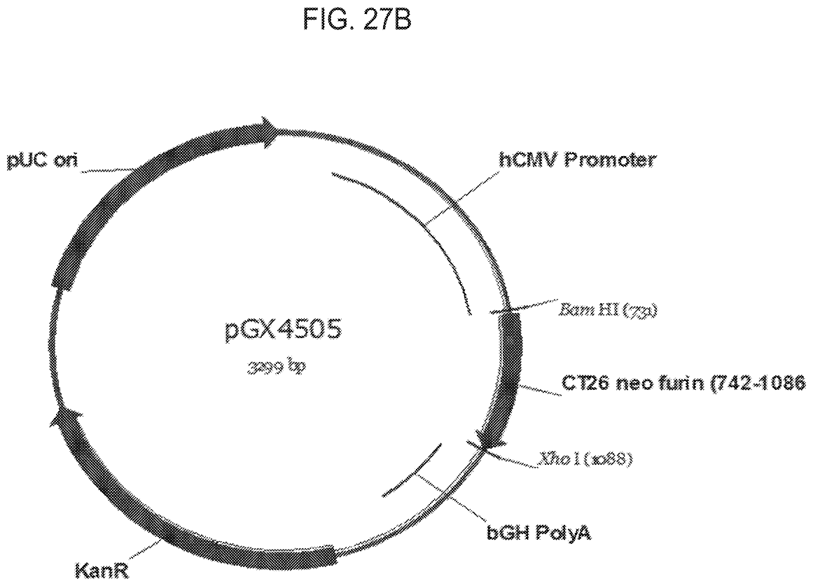

FIG. 22 shows proof of concept neoantigen studies: CT26 neoantigens.

### Example 8. Expression of DNA Vaccines for Ex Vivo Priming Using Dendritic Cells

Neoantigen DNA vaccines can also be used to prime T cells against neoantigens ex vivo. Dendritic cells are differentiated from the host, and are transfected with the DNA plasmids. T cells are added to the culture to obtain a selective expansion of T cells that can detect the neoantigen MHC-peptide complex.

Briefly, the procedure is as follows:

Thaw or Isolate bone marrow BM (get one female B6, flush bone marrow cells, lyse red blood cells, plate 2.5 million per well in 10 cm dish and add 10 ml RPMI, add 40 ng/ml of GMCSF. At day 3 refresh GMCSF by adding 10 ml of RPMI with 40 ng/ml of GMCSF.

At day 6, refresh GMCSF by spinning down 10 ml of RPMI and resuspend in 10 ml of fresh RPMI with 40 ng/ml of GMCSF.

On day 7: collect expanded dendritic cells and plate 0.5 million in a 6 well plate. Following, transfect neoantigen vaccine plasmids using lipofectamine reagent.

On day 8: plate T-cells at 10 (Tcells):1 (BMDC) ratio (so 5 million per well) with 20IU IL-2/ml and 1 ng/ml IL7.

Refresh transfected dendritic cell culture weekly for 4-6 weeks and use T cells in desired experiments.

### Example 9: Delivery of a Plurality of Epitopes in One Formulation and Effect on Tumor Challenge Efficacy

The purpose of this study is to demonstrate the ability to deliver 60 epitopes in one formulation (5 plasmids), and to demonstrate that delivery of more epitopes does not impact tumor challenge efficacy.

For this study, 5 groups of mice are implanted with tumors (TC1 tumors specifically) and are delivered the following vaccine combinations:
1. pVax (25 μg)
2. TC1 Plasmid #1 alone (25 μg)
3. 3 LLC Plasmids (25 μg per plasmid)+pVax (50 μg)
4. 2 TC1 plasmids (25 μg per plasmid)+pVax (75 μg)
5. 3 LLC Plasmids (25 μg per plasmid)+2 TC1 Plasmids (25 μg per plasmid)

In this study, it is expected that group #2, group #4 and group #5 will have similar anti-tumor activity, and that group #1 and group #3 will have no anti-tumor activity.

This study will demonstrate that adding irrelevant immunogenic epitopes from the LLC tumors does not impact the ability of the TC1 plasmids to generate anti-tumor immunity to TC1 tumors.

Full-Length pVAX Sequence is as Follows:

(SEQ ID NO: 68)
gctgcttcgcgatgtacgggccagatatacgcgttgacattgattattgactagttattaatagtaatcaattacggggtcattagttcatagcccatatatgga
gttccgcgttacataacttacggtaaatggcccgcctggctgaccgcccaacgacccccgcccattgacgtcaataatgacgtatgttoccatagtaacgc
caatagggactttccattgacgtcaatgggtggagtatttacggtaaactgcccacttggcagtacatcaagtgtatcatatgccaagtacgccccctattga
cgtcaatgacggtaaatggcccgcctggcattatgcccagtacatgaccttatgggactttcctacttggcagtacatctacgtattagtcatcgctattacca
tggtgatgcggttttggcagtacatcaatgggcgtggatagcggtttgactcacggggatttccaagtctccaccccattgacgtcaatgggagtttgttttg
gcaccaaaatcaacgggactttccaaaatgtcgtaacaactccgccccattgacgcaaatgggcggtaggcgtgtacggtgggaggtctatataagcag
agctctctggctaactagagaacccactgcttactggcttatcgaaattaatacgactcactatagggagacccaagctggctagcgtttaaacttaagcttg
gtaccgagctcggatccactagtccagtgtggtggaattctgcagatatccagcacagtggcggccgctcgagtctagagggcccgtttaaacccgctg
atcagcctcgactgtgccttctagttgccagccatctgttgtttgcccctcccccgtgccttccttgaccctggaaggtgccactcccactgtcctttcctaat
aaaatgaggaaattgcatcgcattgtctgagtaggtgtcattctattctggggggtggggggggcaggacagcaagggggaggattgggaagacaatag
caggcatgctggggatgcggtgggctctatggcttctactgggcggttttatggacagcaagcgaaccggaattgccagctggggcgccctctggtaag
gttgggaagccctgcaaagtaaactggatggctttcttgccgccaaggatctgatggcgcaggggatcaagctctgatcaagagacaggatgaggatcg
tttcgcatgattgaacaagatggattgcacgcaggttctccggccgcttgggtggagaggctattcggctatgactgggcacaacagacaatcggctgct
ctgatgccgccgtgttccggctgtcagcgcaggggcgcccggttctttttgtcaagaccgacctgtccggtgccctgaatgaactgcaagacgaggcag
cgcggctatcgtggctggccacgacgggcgttccttgcgcagctgtgctcgacgttgtcactgaagcgggaagggactggctgctattgggcgaagtg
ccggggcaggatctcctgtcatctcaccttgctcctgccgagaaagtatccatcatggctgatgcaatgcggcggctgcatacgcttgatccggctacctg
cccattcgaccaccaagcgaaacatcgcatcgagcgagcacgtactcggatggaagccggtcttgtcgatcaggatgatctggacgaagagcatcagg
ggctcgcgccagccgaactgttcgccaggctcaaggcgagcatgcccgacggcgaggatctcgtcgtgacccatggcgatgcctgcttgccgaatatcatggtg
gaaaatggccgcttttctggattcatcgactgtggccggctgggtgtggcggaccgctatcaggacatagcgttggctacccgtgatattgctgaagagcttg
gcggcgaatgggctgaccgcttcctcgtgctttacggtatcgccgctcccgattcgcagcgcatcgccttctatcgccttcttgacgagttcttctgaattat
taacgcttacaatttcctgatgcggtattttctccttacgcatctgtgcggtatttcacaccgcatcaggtggcacttttcggggaaatgtgcgcggaacccc
tatttgtttatttttctaaatacattcaaatatgtatccgctcatgagacaataaccctgataaatgcttcaataatagcacgtgctaaaacttcatttttaa
tttaaaaggatctaggtgaagatcctttttgataatctcatgaccaaaatcccttaacgtgagttttcgttccactgagcgtcagaccccgtagaaaagatcaa
aggatcttcttgagatcctttttttctgcgcgtaatctgctgcttgcaaacaaaaaaaccaccgctaccagcggtggtttgtttgccggatcaagagctaccaa
ctctttttccgaaggtaactggcttcagcagagcgcagataccaaatactgttcttctagtgtagccgtagttaggccaccacttcaagaactctgtagcacc
gcctacatacctcgctctgctaatcctgttaccagtggctgctgccagtggcgataagtcgtgtcttaccgggttggactcaagacgatagttaccggataa
ggcgcagcggtcgggctgaacggggggttcgtgcacacagcccagcttggagcgaacgacctacaccgaactgagatacctacagcgtgagctatg
agaaagcgccacgcttcccgaagggagaaaggcggacaggtatccggtaagcggcagggtcggaacaggagagcgcacgagggagcttccaggggga
aacgcctggtatctttatagtcctgtcgggtttcgccacctctgacttgagcgtcgatttttgtgatgctcgtcaggggggcggagcctatggaaaaac
gccagcaacgcggcctttttacggttcctggccttttgctggccttttgctcacatgttctt

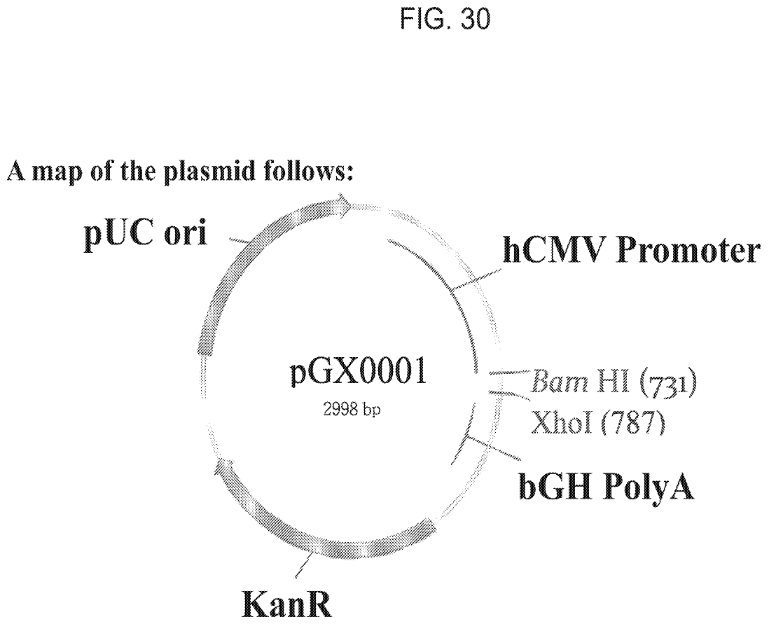

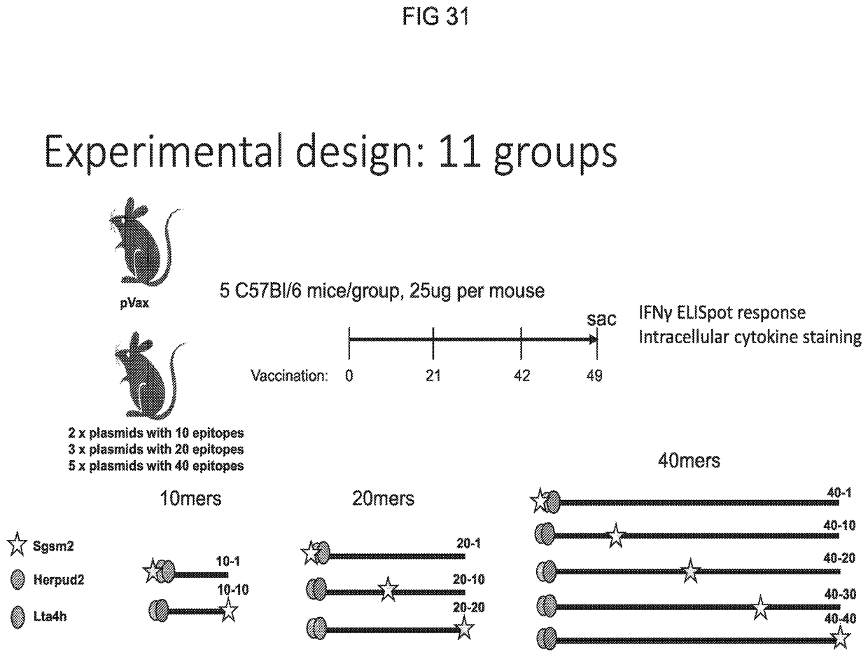

A map of the plasmid is depicted in FIG. 30.

### Example 10

Here are the studies that we have currently ongoing:
1. We are testing whether or not the IL-12 immune plasmid adjuvant can boost immune responses generated by LLC Plasmid #3.
2. We are testing the relative contributions of CD4 and CD8 T cells for the anti-tumor impact of the TC1 Plasmid #1 vaccine through immune depletion studies.
3. We are testing the anti-tumor activity of the 3 LLC plasmids in the LLC tumor model.
4. We are testing the anti-tumor activity of the 2 ID8 Plasmids either alone or in combination with immune checkpoint inhibitors targeting CTLA4 and PD1 in the ID8 ovarian tumor model.
5. We are looking for neo-antigen competition in the TC1 tumor model by delivering non-TC1 targeted neo-epitope plasmids (from LLC) in combination with the TC1 Plasmids and comparing to TC1 Plasmid #1 alone.

### Example 11—40 Antigen Plasmid Construction and Vaccination

Purpose #1: Compare the efficacy of a 12-epitope plasmid versus 24-epitope plasmid versus 40-epitope plasmid.

Purpose #2: Alter the position of Sgsm2 within each plasmid to determine if that has an impact.

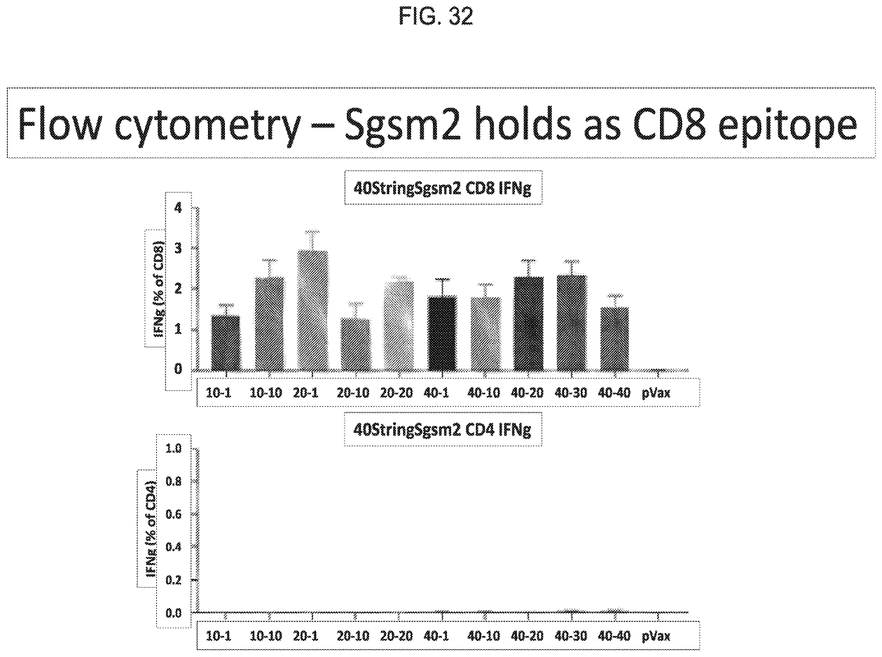

Plasmid sequences of pVAX1 were created comprising nucleic acid constructs that encode the epitopes below. As indicated nucleic acid sequences encoding the amino acid sequence provided were cloned into the pVAX1 vector multiple cloning site with 10 antigens, 20 antigens or 40 antigens. Animals were vaccinated using the protocol depicted in FIG. 31. The labels of each plasmid depict the number and order in which antigens are presented within the plasmid. After vaccination, ELISPOT assays with the protocol identified in Example 1 above were performed and CD8+ cells as well as CD4+ cells were isolated using the above flow cytometry protocol of Example 1. Results of the how many IFN-gamma producing cells of those particular subsets were calculated and depicted in FIG. 32.

All immunogenic epitopes:
1. Sgsm2 V656A:
(SEQ ID NO: 1)
SHVQRLVHRDSTISNDAFISVDDLEPSGPQDLE
2. Herpud2 V85L:
(SEQ ID NO: 2)
DHLQLKDILRKQDEYHMVHLLCASRSPPSSPKS
3. Lta4h V463A:
(SEQ ID NO: 3)
WNTWLYAPGLPPVKPNYDATLTNACIALSQRWV
4. Phactr4 V253L:
(SEQ ID NO: 4)
AQRNSNPIIAELSQAMNSGTLLSKPSPPLPPKR
5. Aunip E168G:
(SEQ ID NO: 5)
GESKGPLDSSFSQYLGRSCLLDQREAKRKGEGL
6. Pik3ca Y143F:
(SEQ ID NO: 6)
QGKYILKVCGCDEYFLEKFPLSQYKYIRSCIML
7. Gpn2 1151V:
(SEQ ID NO: 7)
KFISVLCTSLATMLHVELPHVNLLSKMDLIEHY
8. Eng 1286V:
(SEQ ID NO: 8)
LRPSTLSQEVYKTVSMRLNVVSPDLSGKGLVLP
9. Zmym1 P172S:
(SEQ ID NO: 9)
ACSSSYNSAVMESSSVNVSMVHSSSKENLCPKK
10. Sema6d V255M:
(SEQ ID NO: 10)
REIAVEHNNLGKAVYSRMARICKNDMGGSQRVL
11. Ubr1 G1412S:
(SEQ ID NO: 11)
PGLLSVDLFHVLVSAVLAFPSLYWDDTVDLQPS
12. Zgrf1 Y1638C:
(SEQ ID NO: 12)
LCLMGHKPVLLRTQCRCHPAISAIANDLFYEGS
13. Casz1 L1087P:
(SEQ ID NO: 13)
TSAAETKPPLAPSSPPAPPGTMVAGSSLEGPAP
14. Adgrb2 L572P:
(SEQ ID NO: 14)
RSLQELLARRTYYSGDPLFSVDILRNVTDTFKR
15. Obsl1 T1764M:
(SEQ ID NO: 15)
GGHVCWMREGVELCPGNKYEMRRHGTTHSLVIH
16. Dhrs9 L146P:
(SEQ ID NO: 16)
EPIEVNLFGLINVTPNMLPLVKKARGRVINVSS
17. Zmym1 T466R:
(SEQ ID NO: 17)
VDFNKICGQAYDSATNFRVKLNEVVAEFKKEEP
18. Nrp2 W664L:
(SEQ ID NO: 18)
NCNFDFPEETCGWVYDHAKLLRSTWISSANPND
19. Abhd18 S210C:
(SEQ ID NO: 19)
ISMGGHMASLAVCNWPKPMPLIPCLSWSTASGV
20. Focad V1388M:
(SEQ ID NO: 20)
GLSLNIKKYLLVSMPLWAKHMSDEQIQGFVENL
RGRKRRS (SEQ ID NO: 41)- furin cleavage site
10-epitope plasmid:
1. Sgsm2 V656A:
(SEQ ID NO: 1)
SHVQRLVHRDSTISNDAFISVDDLEPSGPQDLE
2. Herpud2 V85L:
(SEQ ID NO: 2)
DHLQLKDILRKQDEYHMVHLLCASRSPPSSPKS
3. Lta4h V463A:
(SEQ ID NO: 3)
WNTWLYAPGLPPVKPNYDATLTNACIALSQRWV
9. Zmym1 P172S:
(SEQ ID NO: 9)
ACSSSYNSAVMESSSVNVSMVHSSSKENLCPKK
11. Ubr1 G1412S:
(SEQ ID NO: 11)
PGLLSVDLFHVLVSAVLAFPSLYWDDTVDLQPS
15. Obsl1 T1764M:
(SEQ ID NO: 15)
GGHVCWMREGVELCPGNKYEMRRHGTTHSLVIH
16. Dhrs9 L146P:
(SEQ ID NO: 16)
EPIEVNLFGLINVTPNMLPLVKKARGRVINVSS
17. Zmym1 T466R:
(SEQ ID NO: 17)
VDFNKICGQAYDSATNFRVKLNEVVAEFKKEEP
18. Nrp2 W664L:
(SEQ ID NO: 18)
NCNFDFPEETCGWVYDHAKLLRSTWISSANPND
19. Abhd18 S210C:
(SEQ ID NO: 19)
ISMGGHMASLAVCNWPKPMPLIPCLSWSTASGV
20-epitope plasmid:
1. Sgsm2 V656A:
(SEQ ID NO: 1)
SHVQRLVHRDSTISNDAFISVDDLEPSGPQDLE
2. Herpud2 V85L:
(SEQ ID NO: 2)
DHLQLKDILRKQDEYHMVHLLCASRSPPSSPKS
3. Lta4h V463A:
(SEQ ID NO: 3)
WNTWLYAPGLPPVKPNYDATLTNACIALSQRWV
4. Phactr4 V253L:
(SEQ ID NO: 4)
AQRNSNPIIAELSQAMNSGTLLSKPSPPLPPKR
5. Aunip E168G:
(SEQ ID NO: 5)
GESKGPLDSSFSQYLGRSCLLDQREAKRKGEGL
6. Pik3ca Y143F:
(SEQ ID NO: 6)
QGKYILKVCGCDEYFLEKFPLSQYKYIRSCIML
7. Gpn2 1151V:
(SEQ ID NO: 7)
KFISVLCTSLATMLHVELPHVNLLSKMDLIEHY
8. Eng 1286V:
(SEQ ID NO: 8)
LRPSTLSQEVYKTVSMRLNVVSPDLSGKGLVLP
9. Zmym1 P172S:
(SEQ ID NO: 9)
ACSSSYNSAVMESSSVNVSMVHSSSKENLCPKK
10. Sema6d V255M:
(SEQ ID NO: 10)
REIAVEHNNLGKAVYSRMARICKNDMGGSQRVL
11. Ubr1 G1412S:
(SEQ ID NO: 11)
PGLLSVDLFHVLVSAVLAFPSLYWDDTVDLQPS
12. Zgrf1 Y1638C:
(SEQ ID NO: 12)
LCLMGHKPVLLRTQCRCHPAISAIANDLFYEGS
13. Casz1 L1087P:
(SEQ ID NO: 13)
TSAAETKPPLAPSSPPAPPGTMVAGSSLEGPAP
14. Adgrb2 L572P:
(SEQ ID NO: 14)
RSLQELLARRTYYSGDPLFSVDILRNVTDTFKR
15. Obsl1 T1764M:
(SEQ ID NO: 15)
GGHVCWMREGVELCPGNKYEMRRHGTTHSLVIH
16. Dhrs9 L146P:
(SEQ ID NO: 16)
EPIEVNLFGLINVTPNMLPLVKKARGRVINVSS
17. Zmym1 T466R:
(SEQ ID NO: 17)
VDFNKICGQAYDSATNFRVKLNEVVAEFKKEEP
18. Nrp2 W664L:
(SEQ ID NO: 18)
NCNFDFPEETCGWVYDHAKLLRSTWISSANPND
19. Abhd18 S210C:
(SEQ ID NO: 19)
ISMGGHMASLAVCNWPKPMPLIPCLSWSTASGV
20. Focad V1388M:
(SEQ ID NO: 20)
GLSLNIKKYLLVSMPLWAKHMSDEQIQGFVENL
40-epitope plasmid:
20 immunogenic epitopes + 20 non-immunogenic epitopes
1. Sgsm2 V656A:
SEQ ID NO: 1)
SHVQRLVHRDSTISNDAFISVDDLEPSGPQDLE
2. Herpud2 V85L:
(SEQ ID NO: 2)
DHLQLKDILRKQDEYHMVHLLCASRSPPSSPKS
3. Lta4h V463A:
(SEQ ID NO: 3)
WNTWLYAPGLPPVKPNYDATLTNACIALSQRWV
4. Phactr4 V253L:
(SEQ ID NO: 4)
AQRNSNPIIAELSQAMNSGTLLSKPSPPLPPKR
5. Aunip E168G:
(SEQ ID NO: 5)
GESKGPLDSSFSQYLGRSCLLDQREAKRKGEGL
6. Pik3ca Y143F:
(SEQ ID NO: 6)
QGKYILKVCGCDEYFLEKFPLSQYKYIRSCIML
7. Gpn2 1151V:
(SEQ ID NO: 7)
KFISVLCTSLATMLHVELPHVNLLSKMDLIEHY
8. Eng 1286V:
(SEQ ID NO: 8)
LRPSTLSQEVYKTVSMRLNVVSPDLSGKGLVLP
9. Zmym1 P172S:
(SEQ ID NO: 9)
ACSSSYNSAVMESSSVNVSMVHSSSKENLCPKK
10. Sema6d V255M:
(SEQ ID NO: 10)
REIAVEHNNLGKAVYSRMARICKNDMGGSQRVL
11. Ubr1 G1412S:
(SEQ ID NO: 11)
PGLLSVDLFHVLVSAVLAFPSLYWDDTVDLQPS
12. Zgrf1 Y1638C:
(SEQ ID NO: 12)
LCLMGHKPVLLRTQCRCHPAISAIANDLFYEGS
13. Casz1 L1087P:
(SEQ ID NO: 13)
TSAAETKPPLAPSSPPAPPGTMVAGSSLEGPAP
14. Adgrb2 L572P:
(SEQ ID NO: 14)
RSLQELLARRTYYSGDPLFSVDILRNVTDTFKR
15. Obsl1 T1764M:
(SEQ ID NO: 15)
GGHVCWMREGVELCPGNKYEMRRHGTTHSLVIH
16. Dhrs9 L146P:
(SEQ ID NO: 16)
EPIEVNLFGLINVTPNMLPLVKKARGRVINVSS
17. Zmym1 T466R:
(SEQ ID NO: 17)
VDFNKICGQAYDSATNFRVKLNEVVAEFKKEEP
18. Nrp2 W664L:
(SEQ ID NO: 18)
NCNFDFPEETCGWVYDHAKLLRSTWISSANPND
19. Abhd18 S210C:
(SEQ ID NO: 19)
ISMGGHMASLAVCNWPKPMPLIPCLSWSTASGV
20. Focad V1388M:
(SEQ ID NO: 20)
GLSLNIKKYLLVSMPLWAKHMSDEQIQGFVENL
Non-immunogenic epitopes:
1. Hdac4 W1020C:
(SEQ ID NO: 21)
ANAVHSMEKVMDIHSKYCRCLQRLSSTVGHSLI
2. Gmppa H24Y:
(SEQ ID NO: 22)
EVPKPLFPVAGVPMIQYHIEACAQVPGMQEILL
3. Fras1 L3135P:
(SEQ ID NO: 23)
SNEDREWHESFSLVLGPDDPVEAVLGDVTTATV
4. Padi3 L193P:
(SEQ ID NO: 24)
MSVMVLRTQGPEAPFEDHRLILHTSSCDAERAR
5. Pdia3 Y264C:
(SEQ ID NO: 25)
CPHMTEDNKDLIQGKDLLTACYDVDYEKNAKGS
6. Pnisr R476G:
(SEQ ID NO: 26)
TRGLAYLHTELPQGDHYKPAISHRDLNSRNVLV
7. Capg F167L:
(SEQ ID NO: 27)
IHREQNSLSLLEASEADGDAVNDKKRTPNEAPS
8. Spen N2066S:
(SEQ ID NO: 28)
EPKRDRRDPSTDKSGPDTFPVEVLERKPPEKTY
9. Usp21 V229M:
(SEQ ID NO: 29)
SGHVGLRNLGNTLPQCFLNAMLQCLSSTRPLRD
10. Clspn A101V:
(SEQ ID NO: 30)
AEDTQENLHSGKSQSRSFPKVLADSDESDMEET
11. Nckap1 K344T:
(SEQ ID NO: 31)
ECKEAAVSHAGSMHRERRTFLRSALKELATVLS
12. Map7d1 R577W:
(SEQ ID NO: 32)
RMREEQLAREAEAWAEREAEARRREEQEAREKA
13. C77080 S770A:
(SEQ ID NO: 33)
LSQTPPPAPPPSAGSEPLARLPQKDSVGKHSGA
14. Stard9 R1686M:
(SEQ ID NO: 34)
NTQIQKLTGSPFRSREYVQTMESESEHSYPPPG
15. Wdr37 H110Y:
(SEQ ID NO: 35)
TTTSRAICQLVKEYIGYRDGIWDVSVTRTQPIV
16. Fam160b2 S575R:
(SEQ ID NO: 36)
NGYDTYVHDAYGLFQECRSRVAHWGWPLGPAPL
17. Mb21d1 Y346S:
(SEQ ID NO: 37)
IQGWLGTKVRTNLRREPFSLVPKNAKDGNSFQG
18. Pyroxd2 V483L:
(SEQ ID NO: 38)
GGKVWNEQEKNTYADKLFDCIEAYAPGFKRSVL
19. Jmjd1c L1715P:
(SEQ ID NO: 39)
WMKCVKGQPHDHKHLMPTQIIPGSVLTDLLDAM
20. Smek1 E665K:
(SEQ ID NO: 40)
SILRNHRYRRDARTLEDKEEMWENTDEDDMEDG

The entire length of encoded amino acid sequence in the plasmids is presented below.

** indicates stop codon and should not be included in each sequence identifier even if SEQ ID number appears after the “**”

10-epitope Sgsm2 pos 1:
(SEQ ID NO: 42)
MSHVQRLVHRDSTISNDAFISVDDLEPSGPQDLERGRKRRSDHLQLKDILRKQDEYHMVHLLC
ASRSPPSSPKSRGRKRRSWNTWLYAPGLPPVKPNYDATLTNACIALSQRWVRGRKRRSACSS
SYNSAVMESSSVNVSMVHSSSKENLCPKKRGRKRRSPGLLSVDLFHVLVSAVLAFPSLYWDD
TVDLQPSRGRKRRSGGHVCWMREGVELCPGNKYEMRRHGTTHSLVIHRGRKRRSEPIEVNLF
GLINVTPNMLPLVKKARGRVINVSSRGRKRRSVDFNKICGQAYDSATNFRVKLNEVVAEFKKEE
PRGRKRRSNCNFDFPEETCGWVYDHAKLLRSTWISSANPNDRGRKRRSISMGGHMASLAVC
NWPKPMPLIPCLSWSTASGV**
10-epitope Sgsm2 pos 10:
(SEQ ID NO: 43)
MDHLQLKDILRKQDEYHMVHLLCASRSPPSSPKSRGRKRRSWNTWLYAPGLPPVKPNYDATL
TNACIALSQRWVRGRKRRSACSSSYNSAVMESSSVNVSMVHSSSKENLCPKKRGRKRRSPG
LLSVDLFHVLVSAVLAFPSLYWDDTVDLQPSRGRKRRSGGHVCWMREGVELCPGNKYEMRR
HGTTHSLVIHRGRKRRSEPIEVNLFGLINVTPNMLPLVKKARGRVINVSSRGRKRRSVDFNKICG
QAYDSATNFRVKLNEVVAEFKKEEPRGRKRRSNCNFDFPEETCGWVYDHAKLLRSTWISSAN
PNDRGRKRRSISMGGHMASLAVCNWPKPMPLIPCLSWSTASGVRGRKRRSSHVQRLVHRDS
TISNDAFISVDDLEPSGPQDLE**
20-epitope Sgsm2 pos 1:
(SEQ ID NO: 44)
MSHVQRLVHRDSTISNDAFISVDDLEPSGPQDLERGRKRRSDHLQLKDILRKQDEYHMVHLLC
ASRSPPSSPKSRGRKRRSWNTWLYAPGLPPVKPNYDATLTNACIALSQRWVRGRKRRSAQR
NSNPIIAELSQAMNSGTLLSKPSPPLPPKRRGRKRRSGESKGPLDSSFSQYLGRSCLLDQREA
KRKGEGLRGRKRRSQGKYILKVCGCDEYFLEKFPLSQYKYIRSCIMLRGRKRRSKFISVLCTSL
ATMLHVELPHVNLLSKMDLIEHYRGRKRRSLRPSTLSQEVYKTVSMRLNVVSPDLSGKGLVLP
RGRKRRSACSSSYNSAVMESSSVNVSMVHSSSKENLCPKKRGRKRRSREIAVEHNNLGKAVY
SRMARICKNDMGGSQRVLRGRKRRSPGLLSVDLFHVLVSAVLAFPSLYWDDTVDLQPSRGRK
RRSLCLMGHKPVLLRTQCRCHPAISAIANDLFYEGSRGRKRRSTSAAETKPPLAPSSPPAPPGT
MVAGSSLEGPAPRGRKRRSRSLQELLARRTYYSGDPLFSVDILRNVTDTFKRRGRKRRSGGH
VCWMREGVELCPGNKYEMRRHGTTHSLVIHRGRKRRSEPIEVNLFGLINVTPNMLPLVKKARG
RVINVSSRGRKRRSVDFNKICGQAYDSATNFRVKLNEVVAEFKKEEPRGRKRRSNCNFDFPEE
TCGWVYDHAKLLRSTWISSANPNDRGRKRRSISMGGHMASLAVCNWPKPMPLIPCLSWSTAS
GVRGRKRRSGLSLNIKKYLLVSMPLWAKHMSDEQIQGFVENL**
20-epitope Sgsm2 pos 10:
(SEQ ID NO: 45)
MDHLQLKDILRKQDEYHMVHLLCASRSPPSSPKSRGRKRRSWNTWLYAPGLPPVKPNYDATL
TNACIALSQRWVRGRKRRSAQRNSNPIIAELSQAMNSGTLLSKPSPPLPPKRRGRKRRSGESK
GPLDSSFSQYLGRSCLLDQREAKRKGEGLRGRKRRSQGKYILKVCGCDEYFLEKFPLSQYKYI
RSCIMLRGRKRRSKFISVLCTSLATMLHVELPHVNLLSKMDLIEHYRGRKRRSLRPSTLSQEVY
KTVSMRLNVVSPDLSGKGLVLPRGRKRRSACSSSYNSAVMESSSVNVSMVHSSSKENLCPKK
RGRKRRSREIAVEHNNLGKAVYSRMARICKNDMGGSQRVLRGRKRRSSHVQRLVHRDSTISN
DAFISVDDLEPSGPQDLERGRKRRSPGLLSVDLFHVLVSAVLAFPSLYWDDTVDLQPSRGRKR
RSLCLMGHKPVLLRTQCRCHPAISAIANDLFYEGSRGRKRRSTSAAETKPPLAPSSPPAPPGT
MVAGSSLEGPAPRGRKRRSRSLQELLARRTYYSGDPLFSVDILRNVTDTFKRRGRKRRSGGH
VCWMREGVELCPGNKYEMRRHGTTHSLVIHRGRKRRSEPIEVNLFGLINVTPNMLPLVKKARG
RVINVSSRGRKRRSVDFNKICGQAYDSATNFRVKLNEVVAEFKKEEPRGRKRRSNCNFDFPEE
TCGWVYDHAKLLRSTWISSANPNDRGRKRRSISMGGHMASLAVCNWPKPMPLIPCLSWSTAS
GVRGRKRRSGLSLNIKKYLLVSMPLWAKHMSDEQIQGFVENL**
20-epitope Sgsm2 pos 20:
(SEQ ID NO: 46)
MDHLQLKDILRKQDEYHMVHLLCASRSPPSSPKSRGRKRRSWNTWLYAPGLPPVKPNYDATL
TNACIALSQRWVRGRKRRSAQRNSNPIIAELSQAMNSGTLLSKPSPPLPPKRRGRKRRSGESK
GPLDSSFSQYLGRSCLLDQREAKRKGEGLRGRKRRSQGKYILKVCGCDEYFLEKFPLSQYKYI
RSCIMLRGRKRRSKFISVLCTSLATMLHVELPHVNLLSKMDLIEHYRGRKRRSLRPSTLSQEVY
KTVSMRLNVVSPDLSGKGLVLPRGRKRRSACSSSYNSAVMESSSVNVSMVHSSSKENLCPKK
RGRKRRSREIAVEHNNLGKAVYSRMARICKNDMGGSQRVLRGRKRRSPGLLSVDLFHVLVSA
VLAFPSLYWDDTVDLQPSRGRKRRSLCLMGHKPVLLRTQCRCHPAISAIANDLFYEGSRGRKR
RSTSAAETKPPLAPSSPPAPPGTMVAGSSLEGPAPRGRKRRSRSLQELLARRTYYSGDPLFSV
DILRNVTDTFKRRGRKRRSGGHVCWMREGVELCPGNKYEMRRHGTTHSLVIHRGRKRRSEPI
EVNLFGLINVTPNMLPLVKKARGRVINVSSRGRKRRSVDFNKICGQAYDSATNFRVKLNEVVAE
FKKEEPRGRKRRSNCNFDFPEETCGWVYDHAKLLRSTWISSANPNDRGRKRRSISMGGHMA
SLAVCNWPKPMPLIPCLSWSTASGVRGRKRRSGLSLNIKKYLLVSMPLWAKHMSDEQIQGFVE
NLRGRKRRSSHVQRLVHRDSTISNDAFISVDDLEPSGPQDLE**
40-epitope Sgsm2 pos 1:
(SEQ ID NO: 47)
MSHVQRLVHRDSTISNDAFISVDDLEPSGPQDLERGRKRRSANAVHSMEKVMDIHSKYCRCLQ
RLSSTVGHSLIRGRKRRSDHLQLKDILRKQDEYHMVHLLCASRSPPSSPKSRGRKRRSEVPKP
LFPVAGVPMIQYHIEACAQVPGMQEILLRGRKRRSWNTWLYAPGLPPVKPNYDATLTNACIALS
QRWVRGRKRRSSNEDREWHESFSLVLGPDDPVEAVLGDVTTATVRGRKRRSAQRNSNPIIAE
LSQAMNSGTLLSKPSPPLPPKRRGRKRRSMSVMVLRTQGPEAPFEDHRLILHTSSCDAERAR
RGRKRRSGESKGPLDSSFSQYLGRSCLLDQREAKRKGEGLRGRKRRSCPHMTEDNKDLIQG
KDLLTACYDVDYEKNAKGSRGRKRRSQGKYILKVCGCDEYFLEKFPLSQYKYIRSCIMLRGRK
RRSTRGLAYLHTELPQGDHYKPAISHRDLNSRNVLVRGRKRRSKFISVLCTSLATMLHVELPHV
NLLSKMDLIEHYRGRKRRSIHREQNSLSLLEASEADGDAVNDKKRTPNEAPSRGRKRRSLRPS
TLSQEVYKTVSMRLNVVSPDLSGKGLVLPRGRKRRSEPKRDRRDPSTDKSGPDTFPVEVLER
KPPEKTYRGRKRRSACSSSYNSAVMESSSVNVSMVHSSSKENLCPKKRGRKRRSSGHVGLR
NLGNTLPQCFLNAMLQCLSSTRPLRDRGRKRRSREIAVEHNNLGKAVYSRMARICKNDMGGS
QRVLRGRKRRSAEDTQENLHSGKSQSRSFPKVLADSDESDMEETRGRKRRSPGLLSVDLFHV
LVSAVLAFPSLYWDDTVDLQPSRGRKRRSECKEAAVSHAGSMHRERRTFLRSALKELATVLSR
GRKRRSLCLMGHKPVLLRTQCRCHPAISAIANDLFYEGSRGRKRRSRMREEQLAREAEAWAE
REAEARRREEQEAREKARGRKRRSTSAAETKPPLAPSSPPAPPGTMVAGSSLEGPAPRGRKR
RSLSQTPPPAPPPSAGSEPLARLPQKDSVGKHSGARGRKRRSRSLQELLARRTYYSGDPLFS
VDILRNVTDTFKRRGRKRRSNTQIQKLTGSPFRSREYVQTMESESEHSYPPPGRGRKRRSGG
HVCWMREGVELCPGNKYEMRRHGTTHSLVIHRGRKRRSTTTSRAICQLVKEYIGYRDGIWDV
SVTRTQPIVRGRKRRSEPIEVNLFGLINVTPNMLPLVKKARGRVINVSSRGRKRRSNGYDTYVH
DAYGLFQECRSRVAHWGWPLGPAPLRGRKRRSVDFNKICGQAYDSATNFRVKLNEVVAEFKK
EEPRGRKRRSIQGWLGTKVRTNLRREPFSLVPKNAKDGNSFQGRGRKRRSNCNFDFPEETC
GWVYDHAKLLRSTWISSANPNDRGRKRRSGGKVWNEQEKNTYADKLFDCIEAYAPGFKRSVL
RGRKRRSISMGGHMASLAVCNWPKPMPLIPCLSWSTASGVRGRKRRSWMKCVKGQPHDHK
HLMPTQIIPGSVLTDLLDAMRGRKRRSGLSLNIKKYLLVSMPLWAKHMSDEQIQGFVENLRGRK
RRSSILRNHRYRRDARTLEDKEEMWENTDEDDMEDG**
40-epitope Sgsm2 pos 10:
(SEQ ID NO: 48)
MANAVHSMEKVMDIHSKYCRCLQRLSSTVGHSLIRGRKRRSDHLQLKDILRKQDEYHMVHLLC
ASRSPPSSPKSRGRKRRSEVPKPLFPVAGVPMIQYHIEACAQVPGMQEILLRGRKRRSWNTW
LYAPGLPPVKPNYDATLTNACIALSQRWVRGRKRRSSNEDREWHESFSLVLGPDDPVEAVLG
DVTTATVRGRKRRSAQRNSNPIIAELSQAMNSGTLLSKPSPPLPPKRRGRKRRSMSVMVLRTQ
GPEAPFEDHRLILHTSSCDAERARRGRKRRSGESKGPLDSSFSQYLGRSCLLDQREAKRKGE
GLRGRKRRSCPHMTEDNKDLIQGKDLLTACYDVDYEKNAKGSRGRKRRSSHVQRLVHRDSTI
SNDAFISVDDLEPSGPQDLERGRKRRSQGKYILKVCGCDEYFLEKFPLSQYKYIRSCIMLRGRK
RRSTRGLAYLHTELPQGDHYKPAISHRDLNSRNVLVRGRKRRSKFISVLCTSLATMLHVELPHV
NLLSKMDLIEHYRGRKRRSIHREQNSLSLLEASEADGDAVNDKKRTPNEAPSRGRKRRSLRPS
TLSQEVYKTVSMRLNVVSPDLSGKGLVLPRGRKRRSEPKRDRRDPSTDKSGPDTFPVEVLER
KPPEKTYRGRKRRSACSSSYNSAVMESSSVNVSMVHSSSKENLCPKKRGRKRRSSGHVGLR
NLGNTLPQCFLNAMLQCLSSTRPLRDRGRKRRSREIAVEHNNLGKAVYSRMARICKNDMGGS
QRVLRGRKRRSAEDTQENLHSGKSQSRSFPKVLADSDESDMEETRGRKRRSPGLLSVDLFHV
LVSAVLAFPSLYWDDTVDLQPSRGRKRRSECKEAAVSHAGSMHRERRTFLRSALKELATVLSR
GRKRRSLCLMGHKPVLLRTQCRCHPAISAIANDLFYEGSRGRKRRSRMREEQLAREAEAWAE
REAEARRREEQEAREKARGRKRRSTSAAETKPPLAPSSPPAPPGTMVAGSSLEGPAPRGRKR
RSLSQTPPPAPPPSAGSEPLARLPQKDSVGKHSGARGRKRRSRSLQELLARRTYYSGDPLFS
VDILRNVTDTFKRRGRKRRSNTQIQKLTGSPFRSREYVQTMESESEHSYPPPGRGRKRRSGG
HVCWMREGVELCPGNKYEMRRHGTTHSLVIHRGRKRRSTTTSRAICQLVKEYIGYRDGIWDV
SVTRTQPIVRGRKRRSEPIEVNLFGLINVTPNMLPLVKKARGRVINVSSRGRKRRSNGYDTYVH
DAYGLFQECRSRVAHWGWPLGPAPLRGRKRRSVDFNKICGQAYDSATNFRVKLNEVVAEFKK
EEPRGRKRRSIQGWLGTKVRTNLRREPFSLVPKNAKDGNSFQGRGRKRRSNCNFDFPEETC
GWVYDHAKLLRSTWISSANPNDRGRKRRSGGKVWNEQEKNTYADKLFDCIEAYAPGFKRSVL
RGRKRRSISMGGHMASLAVCNWPKPMPLIPCLSWSTASGVRGRKRRSWMKCVKGQPHDHK
HLMPTQIIPGSVLTDLLDAMRGRKRRSGLSLNIKKYLLVSMPLWAKHMSDEQIQGFVENLRGRK
RRSSILRNHRYRRDARTLEDKEEMWFNTDEDDMEDG**
40-epitope Sgsm2 pos 20:
(SEQ ID NO: 49)
MANAVHSMEKVMDIHSKYCRCLQRLSSTVGHSLIRGRKRRSDHLQLKDILRKQDEYHMVHLLC
ASRSPPSSPKSRGRKRRSEVPKPLFPVAGVPMIQYHIEACAQVPGMQEILLRGRKRRSWNTW
LYAPGLPPVKPNYDATLTNACIALSQRWVRGRKRRSSNEDREWHESFSLVLGPDDPVEAVLG
DVTTATVRGRKRRSAQRNSNPIIAELSQAMNSGTLLSKPSPPLPPKRRGRKRRSMSVMVLRTQ
GPEAPFEDHRLILHTSSCDAERARRGRKRRSGESKGPLDSSFSQYLGRSCLLDQREAKRKGE
GLRGRKRRSCPHMTEDNKDLIQGKDLLTACYDVDYEKNAKGSRGRKRRSQGKYILKVCGCDE
YFLEKFPLSQYKYIRSCIMLRGRKRRSTRGLAYLHTELPQGDHYKPAISHRDLNSRNVLVRGRK
RRSKFISVLCTSLATMLHVELPHVNLLSKMDLIEHYRGRKRRSIHREQNSLSLLEASEADGDAV
NDKKRTPNEAPSRGRKRRSLRPSTLSQEVYKTVSMRLNVVSPDLSGKGLVLPRGRKRRSEPK
RDRRDPSTDKSGPDTFPVEVLERKPPEKTYRGRKRRSACSSSYNSAVMESSSVNVSMVHSSS
KENLCPKKRGRKRRSSGHVGLRNLGNTLPQCFLNAMLQCLSSTRPLRDRGRKRRSREIAVEH
NNLGKAVYSRMARICKNDMGGSQRVLRGRKRRSAEDTQENLHSGKSQSRSFPKVLADSDES
DMEETRGRKRRSSHVQRLVHRDSTISNDAFISVDDLEPSGPQDLERGRKRRSPGLLSVDLFHV
LVSAVLAFPSLYWDDTVDLQPSRGRKRRSECKEAAVSHAGSMHRERRTFLRSALKELATVLSR
GRKRRSLCLMGHKPVLLRTQCRCHPAISAIANDLFYEGSRGRKRRSRMREEQLAREAEAWAE
REAEARRREEQEAREKARGRKRRSTSAAETKPPLAPSSPPAPPGTMVAGSSLEGPAPRGRKR
RSLSQTPPPAPPPSAGSEPLARLPQKDSVGKHSGARGRKRRSRSLQELLARRTYYSGDPLFS
VDILRNVTDTFKRRGRKRRSNTQIQKLTGSPFRSREYVQTMESESEHSYPPPGRGRKRRSGG
HVCWMREGVELCPGNKYEMRRHGTTHSLVIHRGRKRRSTTTSRAICQLVKEYIGYRDGIWDV
SVTRTQPIVRGRKRRSEPIEVNLFGLINVTPNMLPLVKKARGRVINVSSRGRKRRSNGYDTYVH
DAYGLFQECRSRVAHWGWPLGPAPLRGRKRRSVDFNKICGQAYDSATNFRVKLNEVVAEFKK
EEPRGRKRRSIQGWLGTKVRTNLRREPFSLVPKNAKDGNSFQGRGRKRRSNCNFDFPEETC
GWVYDHAKLLRSTWISSANPNDRGRKRRSGGKVWNEQEKNTYADKLFDCIEAYAPGFKRSVL
RGRKRRSISMGGHMASLAVCNWPKPMPLIPCLSWSTASGVRGRKRRSWMKCVKGQPHDHK
HLMPTQIIPGSVLTDLLDAMRGRKRRSGLSLNIKKYLLVSMPLWAKHMSDEQIQGFVENLRGRK
RRSSILRNHRYRRDARTLEDKEEMWFNTDEDDMEDG**
40-epitope Sgsm2 pos 30:
(SEQ ID NO: 50)
MANAVHSMEKVMDIHSKYCRCLQRLSSTVGHSLIRGRKRRSDHLQLKDILRKQDEYHMVHLLC
ASRSPPSSPKSRGRKRRSEVPKPLFPVAGVPMIQYHIEACAQVPGMQEILLRGRKRRSWNTW
LYAPGLPPVKPNYDATLTNACIALSQRWVRGRKRRSSNEDREWHESFSLVLGPDDPVEAVLG
DVTTATVRGRKRRSAQRNSNPIIAELSQAMNSGTLLSKPSPPLPPKRRGRKRRSMSVMVLRTQ
GPEAPFEDHRLILHTSSCDAERARRGRKRRSGESKGPLDSSFSQYLGRSCLLDQREAKRKGE
GLRGRKRRSCPHMTEDNKDLIQGKDLLTACYDVDYEKNAKGSRGRKRRSQGKYILKVCGCDE
YFLEKFPLSQYKYIRSCIMLRGRKRRSTRGLAYLHTELPQGDHYKPAISHRDLNSRNVLVRGRK
RRSKFISVLCTSLATMLHVELPHVNLLSKMDLIEHYRGRKRRSIHREQNSLSLLEASEADGDAV
NDKKRTPNEAPSRGRKRRSLRPSTLSQEVYKTVSMRLNVVSPDLSGKGLVLPRGRKRRSEPK
RDRRDPSTDKSGPDTFPVEVLERKPPEKTYRGRKRRSACSSSYNSAVMESSSVNVSMVHSSS
KENLCPKKRGRKRRSSGHVGLRNLGNTLPQCFLNAMLQCLSSTRPLRDRGRKRRSREIAVEH
NNLGKAVYSRMARICKNDMGGSQRVLRGRKRRSAEDTQENLHSGKSQSRSFPKVLADSDES
DMEETRGRKRRSPGLLSVDLFHVLVSAVLAFPSLYWDDTVDLQPSRGRKRRSECKEAAVSHA
GSMHRERRTFLRSALKELATVLSRGRKRRSLCLMGHKPVLLRTQCRCHPAISAIANDLFYEGS
RGRKRRSRMREEQLAREAEAWAEREAEARRREEQEAREKARGRKRRSTSAAETKPPLAPSS
PPAPPGTMVAGSSLEGPAPRGRKRRSLSQTPPPAPPPSAGSEPLARLPQKDSVGKHSGARG
RKRRSRSLQELLARRTYYSGDPLFSVDILRNVTDTFKRRGRKRRSNTQIQKLTGSPFRSREYV
QTMESESEHSYPPPGRGRKRRSGGHVCWMREGVELCPGNKYEMRRHGTTHSLVIHRGRKR
RSTTTSRAICQLVKEYIGYRDGIWDVSVTRTQPIVRGRKRRSSHVQRLVHRDSTISNDAFISVDD
LEPSGPQDLERGRKRRSEPIEVNLFGLINVTPNMLPLVKKARGRVINVSSRGRKRRSNGYDTY
VHDAYGLFQECRSRVAHWGWPLGPAPLRGRKRRSVDFNKICGQAYDSATNFRVKLNEVVAEF
KKEEPRGRKRRSIQGWLGTKVRTNLRREPFSLVPKNAKDGNSFQGRGRKRRSNCNFDFPEET
CGWVYDHAKLLRSTWISSANPNDRGRKRRSGGKVWNEQEKNTYADKLFDCIEAYAPGFKRSV
LRGRKRRSISMGGHMASLAVCNWPKPMPLIPCLSWSTASGVRGRKRRSWMKCVKGQPHDH
KHLMPTQIIPGSVLTDLLDAMRGRKRRSGLSLNIKKYLLVSMPLWAKHMSDEQIQGFVENLRGR
KRRSSILRNHRYRRDARTLEDKEEMWFNTDEDDMEDG**
40-epitope Sgsm2 pos 40:
(SEQ ID NO: 51)
MANAVHSMEKVMDIHSKYCRCLQRLSSTVGHSLIRGRKRRSDHLQLKDILRKQDEYHMVHLLC
ASRSPPSSPKSRGRKRRSEVPKPLFPVAGVPMIQYHIEACAQVPGMQEILLRGRKRRSWNTW
LYAPGLPPVKPNYDATLTNACIALSQRWVRGRKRRSSNEDREWHESFSLVLGPDDPVEAVLG
DVTTATVRGRKRRSAQRNSNPIIAELSQAMNSGTLLSKPSPPLPPKRRGRKRRSMSVMVLRTQ
GPEAPFEDHRLILHTSSCDAERARRGRKRRSGESKGPLDSSFSQYLGRSCLLDQREAKRKGE
GLRGRKRRSCPHMTEDNKDLIQGKDLLTACYDVDYEKNAKGSRGRKRRSQGKYILKVCGCDE
YFLEKFPLSQYKYIRSCIMLRGRKRRSTRGLAYLHTELPQGDHYKPAISHRDLNSRNVLVRGRK
RRSKFISVLCTSLATMLHVELPHVNLLSKMDLIEHYRGRKRRSIHREQNSLSLLEASEADGDAV
NDKKRTPNEAPSRGRKRRSLRPSTLSQEVYKTVSMRLNVVSPDLSGKGLVLPRGRKRRSEPK
RDRRDPSTDKSGPDTFPVEVLERKPPEKTYRGRKRRSACSSSYNSAVMESSSVNVSMVHSSS
KENLCPKKRGRKRRSSGHVGLRNLGNTLPQCFLNAMLQCLSSTRPLRDRGRKRRSREIAVEH
NNLGKAVYSRMARICKNDMGGSQRVLRGRKRRSAEDTQENLHSGKSQSRSFPKVLADSDES
DMEETRGRKRRSPGLLSVDLFHVLVSAVLAFPSLYWDDTVDLQPSRGRKRRSECKEAAVSHA
GSMHRERRTFLRSALKELATVLSRGRKRRSLCLMGHKPVLLRTQCRCHPAISAIANDLFYEGS
RGRKRRSRMREEQLAREAEAWAEREAEARRREEQEAREKARGRKRRSTSAAETKPPLAPSS
PPAPPGTMVAGSSLEGPAPRGRKRRSLSQTPPPAPPPSAGSEPLARLPQKDSVGKHSGARG
RKRRSRSLQELLARRTYYSGDPLFSVDILRNVTDTFKRRGRKRRSNTQIQKLTGSPFRSREYV
QTMESESEHSYPPPGRGRKRRSGGHVCWMREGVELCPGNKYEMRRHGTTHSLVIHRGRKR
RSTTTSRAICQLVKEYIGYRDGIWDVSVTRTQPIVRGRKRRSEPIEVNLFGLINVTPNMLPLVKK
ARGRVINVSSRGRKRRSNGYDTYVHDAYGLFQECRSRVAHWGWPLGPAPLRGRKRRSVDFN
KICGQAYDSATNFRVKLNEVVAEFKKEEPRGRKRRSIQGWLGTKVRTNLRREPFSLVPKNAKD
GNSFQGRGRKRRSNCNFDFPEETCGWVYDHAKLLRSTWISSANPNDRGRKRRSGGKVWNE
QEKNTYADKLFDCIEAYAPGFKRSVLRGRKRRSISMGGHMASLAVCNWPKPMPLIPCLSWSTA
SGVRGRKRRSWMKCVKGQPHDHKHLMPTQIIPGSVLTDLLDAMRGRKRRSGLSLNIKKYLLVS
MPLWAKHMSDEQIQGFVENLRGRKRRSSILRNHRYRRDARTLEDKEEMWFNTDEDDMEDGR
GRKRRSSHVQRLVHRDSTISNDAFISVDDLEPSGPQDLE**

### Follow-Up Study #2:

Determine if the epitopes can be repeated (without cloning issues), and if this can allow for higher immunogenicity for patients with only a few epitopes.

*1^{st} 5 from the previous study

1. Sgsm2 V656A:
SEQ ID NO: 1)
SHVQRLVHRDSTISNDAFISVDDLEPSGPQDLE
2. Herpud2 V85L:
(SEQ ID NO: 2)
DHLQLKDILRKQDEYHMVHLLCASRSPPSSPKS
3. Lta4h V463A:
(SEQ ID NO: 3)
WNTWLYAPGLPPVKPNYDATLTNACIALSQRWV
4. Phactr4 V253L:
(SEQ ID NO: 4)
AQRNSNPIIAELSQAMNSGTLLSKPSPPLPPKR
5. Aunip E168G:
(SEQ ID NO: 5)
GESKGPLDSSFSQYLGRSCLLDQREAKRKGEGL
5-epitope Sgsm2 pos 1:
(SEQ ID NO: 52)
MSHVQRLVHRDSTISNDAFISVDDLEPSGPQDLERGRKRRSDHLQLKDILRKQDEYHMVHLLC
ASRSPPSSPKSRGRKRRSWNTWLYAPGLPPVKPNYDATLTNACIALSQRWVRGRKRRSAQR
NSNPIIAELSQAMNSGTLLSKPSPPLPPKRRGRKRRSGESKGPLDSSFSQYLGRSCLLDQREA
KRKGEGL**
40-epitope repeat:
(SEQ ID NO: 53)
MSHVQRLVHRDSTISNDAFISVDDLEPSGPQDLERGRKRRSDHLQLKDILRKQDEYHMVHLLC
ASRSPPSSPKSRGRKRRSWNTWLYAPGLPPVKPNYDATLTNACIALSQRWVRGRKRRSAQR
NSNPIIAELSQAMNSGTLLSKPSPPLPPKRRGRKRRSGESKGPLDSSFSQYLGRSCLLDQREA
KRKGEGLRGRKRRSSHVQRLVHRDSTISNDAFISVDDLEPSGPQDLERGRKRRSDHLQLKDIL
RKQDEYHMVHLLCASRSPPSSPKSRGRKRRSWNTWLYAPGLPPVKPNYDATLTNACIALSQR
WVRGRKRRSAQRNSNPIIAELSQAMNSGTLLSKPSPPLPPKRRGRKRRSGESKGPLDSSFSQ
YLGRSCLLDQREAKRKGEGLRGRKRRSSHVQRLVHRDSTISNDAFISVDDLEPSGPQDLERGR
KRRSDHLQLKDILRKQDEYHMVHLLCASRSPPSSPKSRGRKRRSWNTWLYAPGLPPVKPNYD
ATLTNACIALSQRWVRGRKRRSAQRNSNPIIAELSQAMNSGTLLSKPSPPLPPKRRGRKRRSG
ESKGPLDSSFSQYLGRSCLLDQREAKRKGEGLRGRKRRSSHVQRLVHRDSTISNDAFISVDDL
EPSGPQDLERGRKRRSDHLQLKDILRKQDEYHMVHLLCASRSPPSSPKSRGRKRRSWNTWL
YAPGLPPVKPNYDATLTNACIALSQRWVRGRKRRSAQRNSNPIIAELSQAMNSGTLLSKPSPPL
PPKRRGRKRRSGESKGPLDSSFSQYLGRSCLLDQREAKRKGEGLRGRKRRSSHVQRLVHRD
STISNDAFISVDDLEPSGPQDLERGRKRRSDHLQLKDILRKQDEYHMVHLLCASRSPPSSPKSR
GRKRRSWNTWLYAPGLPPVKPNYDATLTNACIALSQRWVRGRKRRSAQRNSNPIIAELSQAM
NSGTLLSKPSPPLPPKRRGRKRRSGESKGPLDSSFSQYLGRSCLLDQREAKRKGEGL**

1. A composition, comprising a nucleic acid molecule, wherein the nucleic acid molecule comprises:
(i) a deoxyribonucleic acid (DNA) backbone region, and
(ii) an expressible nucleic acid sequence operably linked to a regulatory sequence;
wherein the expressible nucleic acid sequence is a DNA comprising Formula I that encodes a chimeric polypeptide comprising a concatemer of a linked series of neoantigens, Formula I comprising independently selected, non-repetitive antigen expression domains (AED):
(AED¹)-(linker)₁-(AED₂)-(linker)₂-(AED₃)-(linker)₃ . . . (AEDₙ)-(linker)ₙ-(AEDₙ₊₁),
wherein each AED encodes one of the neoantigens,
each linker has a length independently selectable from about 15 nucleotides to about 300 nucleotides and encodes a furin cleavage site,
the length of each AED is independently selectable from about 24 to about 250 nucleotides, and
n is any positive integer from about 19 to about 58.

2. The composition of claim 1, wherein the length of each AED is independently selectable from about 75 to about 250 nucleotides.

3. The composition of claim 1, wherein the length of each AED is independently selectable from about 90 to about 250 nucleotides.

4. The composition of claim 1, wherein the nucleic acid further comprises a leader sequence.

5. The composition of claim 4, wherein the leader sequence is an IgE leader sequence.

6. The composition of claim 5, wherein the IgE leader or functional fragment thereof comprises a sequence at least about 95% sequence identity to SEQ ID NO: 69.

7. The composition of claim 1, further comprising an adjuvant.

8. The composition of claim 1, further comprising a second nucleic acid molecule encoding IL-12.

9. The composition of claim 8, wherein the IL-12 comprises a human IL-12 alpha subunit and a human IL-12 beta subunit.

10. The composition of claim 1, wherein the DNA backbone region comprises SEQ ID NO: 68 or a functional fragment thereof comprising about 90% sequence identity to SEQ ID NO: 68.

11. The composition of claim 10, wherein the DNA backbone region comprises a human cytomegalovirus (hCMV) promoter and a bovine growth hormone poly-A (polyA BGH).

12. The composition of claim 10, wherein the length of each AED is independently selectable from about 75 to about 250 nucleotides.

13. The composition of claim 10, wherein the length of each AED is independently selectable from about 90 to about 250 nucleotides.

14. The composition of claim 10, wherein the nucleic acid further comprises a leader sequence.

15. The composition of claim 14, wherein the leader sequence is an IgE leader sequence.

16. The composition of claim 15, wherein the IgE leader sequence or functional fragment thereof comprises a sequence with at least about 95% sequence identity to SEQ ID NO: 69.

17. The composition of claim 10, further comprising an adjuvant.

18. The composition of claim 10, further comprising a second nucleic acid molecule encoding IL-12.

19. The composition of claim 18, wherein the IL-12 comprises a human IL-12 alpha subunit and a human IL-12 beta subunit.

20. The composition of claim 15, further comprising an adjuvant.

21. The composition of claim 15, further comprising a second nucleic acid molecule encoding IL-12.

22. The composition of claim 21, wherein the IL-12 comprises a human IL-12 alpha subunit and a human IL-12 beta subunit.
